SEQUENCE LISTING (61 sequences)

SEQ ID NO: 1 - DNA, 3215 bp, Hepatitis B virus
  source 1..3215: mol_type = other DNA
ctccacaacattccaccaagctctgctagatcccagagtgaggggcctatactttcctgctggtggctccagttccggaacagtaaaccctgttccgactactgcctcacccatatcgtcaatcttctcgaggactggggaccctgcaccgaacatggagaacacaacatcaggattcctaggacccctgctcgtgttacaggcggggtttttcttgttgacaagaatcctcacaataccacagagtctagactcgtggtggacttctctcaattttctagggggagcacccacgtgtcctggccaaaattcgcagtccccaacctccaatcactcaccaacctcttgtcctccaatttgtcctggctatcgctggatgtgtctgcggcgttttatcatattcctcttcatcctgctgctatgcctcatcttcttgttggttcttctggactaccaaggtatgttgcccgtttgtcctctacttccaggaacatcaactaccagcacgggaccatgcaagacctgcacgattcctgctcaaggaacctctatgtttccctcttgttgctgtacaaaaccttcggacggaaactgcacttgtattcccatcccatcatcctgggctttcgcaagattcctatgggagtgggcctcagtccgtttctcctggctcagtttactagtgccatttgttcagtggttcgtagggctttcccccactgtttggctttcagttatatggatgatgtggtattgggggccaagtctgtacaacatcttgagtccctttttacctctattaccaattttcttttgtctttgggtatacatttgaaccctaataaaaccaaacgttggggctactcccttaacttcatgggatatgtaattggaagttggggtactttaccacaggaacatattgtactaaaaatcaagcaatgttttcgaaaactgcctgtaaatagacctattgattggaaagtatgtcaaagaattgtgggtcttttgggctttgctgccccttttacacaatgtggctatcctgccttaatgcctttatatgcatgtatacaatctaagcaggctttcactttctcgccaacttacaaggcctttctgtgtaaacaatatctgaacctttaccccgttgcccggcaacggtcaggtctctgccaagtgtttgctgacgcaacccccactggatggggcttggccataggccatcggcgcatgcgtggaacctttgtggctcctctgccgatccatactgcggaactcctagcagcttgttttgctcgcagccggtctggagcgaaacttatcggaaccgacaactctgttgtcctctctcggaaatacacctcctttccatggctgctagggtgtgctgccaactggatcctgcgcgggacgtcctttgtctacgtcccgtcggcgctgaatcccgcggacgacccgtctcggggccgtttgggactctaccgtccccttcttcatctgccgttccggccgaccacggggcgcacctctctttacgcggtctccccgtctgtgccttctcatctgccggaccgtgtgcacttcgcttcacctctgcacgtcgcatggagaccaccgtgaacgcccaccaggtcttgcccaaggtcttacataagaggactcttggactctcagcaatgtcaacgaccgaccttgaggcatacttcaaagactgtttgtttaaagactgggaggagttgggggaggagattaggttaatgatctttgtactaggaggctgtaggcataaattggtctgttcaccagcaccatgcaactttttcacctctgcctaatcatctcatgttcatgtcctactgttcaagcctccaagctgtgccttgggtggctttggggcatggacattgacccgtataaagaatttggagcttctgtggagttactctcttttttgccttctgacttctttccttctattcgagatctcctcgacaccgcctctgctctgtatcgggaggccttagagtctccggaacattgttcacctcaccatacagcactcaggcaagctattctgtgttggggtgagttgatgaatctggccacctgggtgggaagtaatttggaagacccagcatccagggaattagtagtcagctatgtcaatgttaatatgggcctaaaaatcagacaactattgtggtttcacatttcctgtcttacttttggaagagaaactgttcttgagtatttggtgtcttttggagtgtggattcgcactcctcccgcttacagaccaccaaatgcccctatcttatcaacacttccggaaactactgttgttagacgacgaggcaggtcccctagaagaagaactccctcgcctcgcagacgaaggtctcaatcgccgcgtcgcagaagatctcaatctcgggaatctcaatgttagtatcccttggactcataaggtgggaaactttactgggctttattcttctactgtacctgtctttaatcctgagtggcaaactccctcctttcctcacattcatttacaggaggacattattaatagatgtcaacaatatgtgggccctcttacagttaatgaaaaaaggagattaaaattaattatgcctgctaggttctatcctaaccttaccaaatatttgcccttggacaaaggcattaaaccttattatcctgaacatgcagttaatcattacttcaaaactaggcattatttacatactctgtggaaggctggcattctatataagagagaaactacacgcagcgcctcattttgtgggtcaccatattcttgggaacaagagctacagcatgggaggttggtcttccaaacctcgacaaggcatggggacgaatctttctgttcccaatcctctgggattctttcccgatcaccagttggaccctgcgttcggagccaactcaaacaatccagattgggacttcaaccccaacaaggatcactggccagaggcaaatcaggtaggagcgggagcattcgggccagggttcaccccaccacacggcggtcttttggggtggagccctcaggctcagggcatattgacaacagtgccagcagcacctcctcctgcctccaccaatcggcagtcaggaagacagcctactcccatctctccacctctaagagacagtcatcctcaggccatgcagtggaa

SEQ ID NO: 2 - DNA, 3215 bp, synthetic construct
  misc_feature 1..3215: note = HBV consensus sequence
  source 1..3215: mol_type = other DNA
ctccacaacattccaccaagctctgctagatcccagagtgaggggcctatattttcctgctggtggctccagttccggaacagtaaaccctgttccgactactgcctcacccatatcgtcaatcttctcgaggactggggaccctgcaccgaacatggagaacacaacatcaggattcctaggacccctgctcgtgttacaggcggggtttttcttgttgacaagaatcctcacaataccacagagtctagactcgtggtggacttctctcaattttctagggggagcacccacgtgtcctggccaaaattcgcagtccccaacctccaatcactcaccaacctcttgtcctccaatttgtcctggctatcgctggatgtgtctgcggcgttttatcatattcctcttcatcctgctgctatgcctcatcttcttgttggttcttctggactaccaaggtatgttgcccgtttgtcctctacttccaggaacatcaactaccagcacgggaccatgcaagacctgcacgattcctgctcaaggaacctctatgtttccctcttgttgctgtacaaaaccttcggacggaaactgcacttgtattcccatcccatcatcctgggctttcgcaagattcctatgggagtgggcctcagtccgtttctcctggctcagtttactagtgccatttgttcagtggttcgtagggctttcccccactgtttggctttcagttatatggatgatgtggtattgggggccaagtctgtacaacatcttgagtccctttttacctctattaccaattttcttttgtctttgggtatacatttgaaccctaataaaaccaaacgttggggctactcccttaacttcatgggatatgtaattggaagttggggtactttaccacaggaacatattgtactaaaaatcaagcaatgttttcgaaaactgcctgtaaatagacctattgattggaaagtatgtcaaagaattgtgggtcttttgggctttgctgccccttttacacaatgtggctatcctgccttgatgcctttatatgcatgtatacaatctaagcaggctttcactttctcgccaacttacaaggcctttctgtgtaaacaatatctgaacctttaccccgttgcccggcaacggtcaggtctctgccaagtgtttgctgacgcaacccccactggatggggcttggccataggccatcggcgcatgcgtggaacctttgtggctcctctgccgatccatactgcggaactcctagcagcttgttttgctcgcagccggtctggagcgaaacttatcggaaccgacaactctgttgtcctctctcggaaatacacctcctttccatggctgctagggtgtgctgccaactggatcctgcgcgggacgtcctttgtctacgtcccgtcggcgctgaatcccgcggacgacccgtctcggggccgtttgggactctaccgtccccttcttcatctgccgttccggccgaccacggggcgcacctctctttacgcggtctccccgtctgtgccttctcatctgccggaccgtgtgcacttcgcttcacctctgcacgtcgcatggagaccaccgtgaacgcccaccaggtcttgcccaaggtcttacataagaggactcttggactctcagcaatgtcaacgaccgaccttgaggcatacttcaaagactgtttgtttaaagactgggaggagttgggggaggagattaggttaatgatctttgtactaggaggctgtaggcataaattggtctgttcaccagcaccatgcaactttttcacctctgcctaatcatctcatgttcatgtcctactgttcaagcctccaagctgtgccttgggtggctttggggcatggacattgacccgtataaagaatttggagcttctgtggagttactctcttttttgccttctgacttctttccttctattcgagatctcctcgacaccgcctctgctctgtatcgggaggccttagagtctccggaacattgttcacctcaccatacagcactcaggcaagctattctgtgttggggtgagttgatgaatctggccacctgggtgggaagtaatttggaagacccagcatccagggaattagtagtcagctatgtcaatgttaatatgggcctaaaaatcagacaactattgtggtttcacatttcctgtcttacttttggaagagaaactgttcttgagtatttggtgtcttttggagtgtggattcgcactcctcccgcttacagaccaccaaatgcccctatcttatcaacacttccggaaactactgttgttagacgacgaggcaggtcccctagaagaagaactccctcgcctcgcagacgaaggtctcaatcgccgcgtcgcagaagatctcaatctcgggaatctcaatgttagtatcccttggactcataaggtgggaaactttactgggctttattcttctactgtacctgtctttaatcctgagtggcaaactccctcctttcctcacattcatttacaggaggacattattaatagatgtcaacaatatgtgggccctcttacagttaatgaaaaaaggagattaaaattaattatgcctgctaggttctatcctaaccttaccaaatatttgcccttggacaaaggcattaaaccgtattatcctgaacatgcagttaatcattacttcaaaactaggcattatttacatactctgtggaaggctggcattctatataagagagaaactacacgcagcgcctcattttgtgggtcaccatattcttgggaacaagagctacagcatgggaggttggtcttccaaacctcgacaaggcatggggacgaatctttctgttcccaatcctctgggattctttcccgatcaccagttggaccctgcgttcggagccaactcaaacaatccagattgggacttcaaccccaacaaggatcactggccagaggcaaatcaggtaggagcgggagcattcgggccagggttcaccccaccacacggcggtcttttggggtggagccctcaggctcagggcatattgacaacagtgccagcagcacctcctcctgcctccaccaatcggcagtcaggaagacagcctactcccatctctccacctctaagagacagtcatcctcaggccatgcagtggaa

SEQ ID NO: 3 - protein, 1525 aa, synthetic construct
  REGION 1..1525: note = Vaccine construct
MSLLWGGVTVLAAMLIAGQVASVVFLVKGGGPGGGMQLFHLCLIISCSCPTVQASKLCLGWLWGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRESQCGGGSGGGMPLSYQHFRKLLLLDDEAGPLEEELPRLADEGLNRRVAEDLNLGNLNVSIPWTHKVGNFTGLASSTVPVFNPEWQTPSFPHIHLQEDIINRCQQYVGPLTVNEKRRLKLIMPARFYPNLTKYLPLDKGIKPYYPEHAVNHYFKTRHYLHTLWKAGILYKRETTRSASFCGSPYSWEQELQHGRLVFQTSTRHGDESFCSQSSGILSRSPVGPCVRSQLKQSRLGLQPQQGSLARGKSGRSGSIRARVHPTTRRSFGVEPSGSGHIDNSASSTSSCLHQSAVRKTAYSHLSTSKRQSSSGHAVELHNIPPSSARSQSEGPIFSAWWLQFRNSKPASDYALTHIVNLLEDWGPATEHGEHNIRIPRTPARVTGGVFLVDKNPHNTTESRLVVDFSQFSRGSTHVSWPKFAVPNLQSLTNLLSSNLSWLSLDVSAAFYHIPLHPAAMPHLLVGSSGLPRYVARLSSTSRNINYQHGTMQDLHDSCSRNLYVSLLLLYKTFGRKLHLYSHPIILGFRKIPMGVGLSPFLLAQFTSAICSVVRRAFPHCLAFSYMDDVVLGAKSVQHLESLFTSITNFLLSLGIHLNPNKTKRWGYSLNFMGYVIGSWGTLPQEHIVLKIKQCFRKLPVNRPIDWKVCQRIVGLLGFAAPFTQCGYPALMPLYACIQSKQAFTFSPTYKAFLCKQYLNLYPVARQRSGLCQVFADATPTGWGLAIGHRAMRGTFVAPLPIHTAELLAACFARSRSGAKLIGTDNSVVLSRKYTSFPWLLGCAANWILRGTSFVYVPSALNPAADPSAGRLGLYRPLLHLPFRPTTGRTSLYAVSPSVPSHLPDRVHFASPLHVAWRPPRKRRAPVKQTLNFDLLKLAGDVESNPGPMGGWSSKPRQGMGTNLSVPNPLGFFPDHQLDPAFGANSNNPDWDFNPNKDHWPEANQVGAGAFGPGFTPPHGGLLGWSPQAQGILTTVPAAPPPASTNRQSGRQPTPISPPLRDSHPQAMQWNSTTFHQALLDPRVRGLYFPAGGSSSGTVNPVPTTASPISSIFSRTGDPAPNMENTTSGFLGPLLVLQAGFFLLTRILTIPQSLDSWWTSLNFLGGAPTCPGQNSQSPTSNHSPTSCPPICPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILSPFLPLLPIFFCLWVYI

SEQ ID NO: 4 - protein, 26 aa, unidentified
  REGION 1..26: note = Shark Invariant Chain
SLLWGGVTVLAAMLIAGQVASVVFLV

SEQ ID NO: 5 - protein, 8 aa, synthetic construct
  REGION 1..8: note = Linker peptide
KGGGPGGG

SEQ ID NO: 6 - protein, 183 aa, Hepatitis B virus
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 7 - protein, 7 aa, synthetic construct
  REGION 1..7: note = Linker peptide
GGGSGGG

SEQ ID NO: 8 - protein, 772 aa, Hepatitis B virus
MPLSYQHFRKLLLLDDEAGPLEEELPRLADEGLNRRVAEDLNLGNLNVSIPWTHKVGNFTGLASSTVPVFNPEWQTPSFPHIHLQEDIINRCQQYVGPLTVNEKRRLKLIMPARFYPNLTKYLPLDKGIKPYYPEHAVNHYFKTRHYLHTLWKAGILYKRETTRSASFCGSPYSWEQELQHGRLVFQTSTRHGDESFCSQSSGILSRSPVGPCVRSQLKQSRLGLQPQQGSLARGKSGRSGSIRARVHPTTRRSFGVEPSGSGHIDNSASSTSSCLHQSAVRKTAYSHLSTSKRQSSSGHAVELHNIPPSSARSQSEGPIFSAWWLQFRNSKPASDYALTHIVNLLEDWGPATEHGEHNIRIPRTPARVTGGVFLVDKNPHNTTESRLVVDFSQFSRGSTHVSWPKFAVPNLQSLTNLLSSNLSTFGRKLHLYSHPIILGFRKIPMGVGLSPFLLAQFTSAICSVVRRAFPHCLAFSYMDDVVLGAKSVQHLESLFTSITNFLLSLGIHLNPNKTKRWGYSLNFMGYVIGSWGTLPQEHIVLKIKQCFRKLPVNRPIDWKVCQRIVGLLGFAAPFTQCGYPALMPLYACIQSKQAFTFSPTYKAFLCKQYLNLYPVARQRSGLCQVFADATPTGWGLAIGHRAMRGTFVAPLPIHTAELLAACFARSRSGAKLIGTDNSVVLSRKYTSFPWLLGCAANWILRGTSFVYVPSALNPAADPSAGRLGLYRPLLHLPFRPTTGRTSLYAVSPSVPSHLPDRVHFASPLHVAWRPP

SEQ ID NO: 9 - protein, 28 aa, unidentified
  REGION 1..28: note = Furin 2A
RKRRAPVKQTLNFDLLKLAGDVESNPGP

SEQ ID NO: 10 - protein, 226 aa, Hepatitis B virus
MENTTSGFLGPLLVLQAGFFLLTRILTIPQSLDSWWTSLNFLGGAPTCPGQNSQSPTSNHSPTSCPPICPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILSPFLPLLPIFFCLWVYI

SEQ ID NO: 11 - protein, 1521 aa, synthetic construct
  REGION 1..1521: note = Vaccine construct
MSLLWGGVTVLAAMLIAGQVASVVFLVKGGSMGGWSSKPRQGMGTNLSVPNPLGFFPDHQLDPAFGANSNNPDWDFNPNKDHWPEANQVGAGAFGPGFTPPHGGLLGWSPQAQGILTTVPAAPPPASTNRQSGRQPTPISPPLRDSHPQAMQWNSTTFHQALLDPRVRGLYFPAGGSSSGTVNPVPTTASPISSIFSRTGDPAPNMENTTSGFLGPLLVLQAGFFLLTRILTIPQSLDSWWTSLNFLGGAPTCPGQNSQSPTSNHSPTSCPPICPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILSPFLPLLPIFFCLWVYIRKRRAPVKQTLNFDLLKLAGDVESNPGPMQLFHLCLIISCSCPTVQASKLCLGWLWGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRESQCGGGSGGGMPLSYQHFRKLLLLDDEAGPLEEELPRLADEGLNRRVAEDLNLGNLNVSIPWTHKVGNFTGLASSTVPVFNPEWQTPSFPHIHLQEDIINRCQQYVGPLTVNEKRRLKLIMPARFYPNLTKYLPLDKGIKPYYPEHAVNHYFKTRHYLHTLWKAGILYKRETTRSASFCGSPYSWEQELQHGRLVFQTSTRHGDESFCSQSSGILSRSPVGPCVRSQLKQSRLGLQPQQGSLARGKSGRSGSIRARVHPTTRRSFGVEPSGSGHIDNSASSTSSCLHQSAVRKTAYSHLSTSKRQSSSGHAVELHNIPPSSARSQSEGPIFSAWWLQFRNSKPASDYALTHIVNLLEDWGPATEHGEHNIRIPRTPARVTGGVFLVDKNPHNTTESRLVVDFSQFSRGSTHVSWPKFAVPNLQSLTNLLSSNLSWLSLDVSAAFYHIPLHPAAMPHLLVGSSGLPRYVARLSSTSRNINYQHGTMQDLHDSCSRNLYVSLLLLYKTFGRKLHLYSHPIILGFRKIPMGVGLSPFLLAQFTSAICSVVRRAFPHCLAFSYMDDVVLGAKSVQHLESLFTSITNFLLSLGIHLNPNKTKRWGYSLNFMGYVIGSWGTLPQEHIVLKIKQCFRKLPVNRPIDWKVCQRIVGLLGFAAPFTQCGYPALMPLYACIQSKQAFTFSPTYKAFLCKQYLNLYPVARQRSGLCQVFADATPTGWGLAIGHRAMRGTFVAPLPIHTAELLAACFARSRSGAKLIGTDNSVVLSRKYTSFPWLLGCAANWILRGTSFVYVPSALNPAADPSAGRLGLYRPLLHLPFRPTTGRTSLYAVSPSVPSHLPDRVHFASPLHVAWRPP

SEQ ID NO: 12 - protein, 1490 aa, synthetic construct
  REGION 1..1490: note = Vaccine construct
MQLFHLCLIISCSCPTVQASKLCLGWLWGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRESQCGGGSGGGMPLSYQHFRKLLLLDDEAGPLEEELPRLADEGLNRRVAEDLNLGNLNVSIPWTHKVGNFTGLASSTVPVFNPEWQTPSFPHIHLQEDIINRCQQYVGPLTVNEKRRLKLIMPARFYPNLTKYLPLDKGIKPYYPEHAVNHYFKTRHYLHTLWKAGILYKRETTRSASFCGSPYSWEQELQHGRLVFQTSTRHGDESFCSQSSGILSRSPVGPCVRSQLKQSRLGLQPQQGSLARGKSGRSGSIRARVHPTTRRSFGVEPSGSGHIDNSASSTSSCLHQSAVRKTAYSHLSTSKRQSSSGHAVELHNIPPSSARSQSEGPIFSAWWLQFRNSKPASDYALTHIVNLLEDWGPATEHGEHNIRIPRTPARVTGGVFLVDKNPHNTTESRLVVDFSQFSRGSTHVSWPKFAVPNLQSLTNLLSSNLSWLSLDVSAAFYHIPLHPAAMPHLLVGSSGLPRYVARLSSTSRNINYQHGTMQDLHDSCSRNLYVSLLLLYKTFGRKLHLYSHPIILGFRKIPMGVGLSPFLLAQFTSAICSVVRRAFPHCLAFSYMDDVVLGAKSVQHLESLFTSITNFLLSLGIHLNPNKTKRWGYSLNFMGYVIGSWGTLPQEHIVLKIKQCFRKLPVNRPIDWKVCQRIVGLLGFAAPFTQCGYPALMPLYACIQSKQAFTFSPTYKAFLCKQYLNLYPVARQRSGLCQVFADATPTGWGLAIGHRAMRGTFVAPLPIHTAELLAACFARSRSGAKLIGTDNSVVLSRKYTSFPWLLGCAANWILRGTSFVYVPSALNPAADPSAGRLGLYRPLLHLPFRPTTGRTSLYAVSPSVPSHLPDRVHFASPLHVAWRPPRKRRAPVKQTLNFDLLKLAGDVESNPGPMGGWSSKPRQGMGTNLSVPNPLGFFPDHQLDPAFGANSNNPDWDFNPNKDHWPEANQVGAGAFGPGFTPPHGGLLGWSPQAQGILTTVPAAPPPASTNRQSGRQPTPISPPLRDSHPQAMQWNSTTFHQALLDPRVRGLYFPAGGSSSGTVNPVPTTASPISSIFSRTGDPAPNMENTTSGFLGPLLVLQAGFFLLTRILTIPQSLDSWWTSLNFLGGAPTCPGQNSQSPTSNHSPTSCPPICPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILSPFLPLLPIFFCLWVYI

SEQ ID NO: 13 - protein, 1492 aa, synthetic construct
  REGION 1..1492: note = Vaccine construct
MSLLWGGVTVLAAMLIAGQVASVVFLVKGGGPGGGMQLFHLCLIISCSCPTVQASKLCLGWLWGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRESQCGGGSGGGMPLSYQHFRKLLLLDDEAGPLEEELPRLADEGLNRRVAEDLNLGNLNVSIPWTHKVGNFTGLASSTVPVFNPEWQTPSFPHIHLQEDIINRCQQYVGPLTVNEKRRLKLIMPARFYPNLTKYLPLDKGIKPYYPEHAVNHYFKTRHYLHTLWKAGILYKRETTRSASFCGSPYSWEQELQHGRLVFQTSTRHGDESFCSQSSGILSRSPVGPCVRSQLKQSRLGLQPQQGSLARGKSGRSGSIRARVHPTTRRSFGVEPSGSGHIDNSASSTSSCLHQSAVRKTAYSHLSTSKRQSSSGHAVELHNIPPSSARSQSEGPIFSAWWLQFRNSKPASDYALTHIVNLLEDWGPATEHGEHNIRIPRTPARVTGGVFLVDKNPHNTTESRLVVDFSQFSRGSTHVSWPKFAVPNLQSLTNLLSSNLSTFGRKLHLYSHPIILGFRKIPMGVGLSPFLLAQFTSAICSVVRRAFPHCLAFSYMDDVVLGAKSVQHLESLFTSITNFLLSLGIHLNPNKTKRWGYSLNFMGYVIGSWGTLPQEHIVLKIKQCFRKLPVNRPIDWKVCQRIVGLLGFAAPFTQCGYPALMPLYACIQSKQAFTFSPTYKAFLCKQYLNLYPVARQRSGLCQVFADATPTGWGLAIGHRAMRGTFVAPLPIHTAELLAACFARSRSGAKLIGTDNSVVLSRKYTSFPWLLGCAANWILRGTSFVYVPSALNPAADPSAGRLGLYRPLLHLPFRPTTGRTSLYAVSPSVPSHLPDRVHFASPLHVAWRPPKSPNSNNPDWDFNPNKDHWPEANQVGAGAFGPGFTPPHGGLLGWSPQAQGILTTVPAAPPPASTNRQSGRQPTPISPPLRDSHPQAMQWNSTTFHQALLDPRVRGLYFPAGGSSSGTVNPVPTTASPISSIFSRTGDPAPNGSKGKRKRRAPVKQTLNFDLLKLAGDVESNPGPMGGWSSKPRQGMGTNLSVPNPLGFFPDHQLDPAFGANSNNPDWDFNPNKDHWPEANQVGLEGGSGGMENTTSGFLGPLLVLQAGFFLLTRILTIPQSLDSWWTSLNFLGGAPTCPGQNSQSPTSNHSPTSCPPICPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILSPFLPLLPIFFCLWVYI

SEQ ID NO: 14 - protein, 4 aa, synthetic construct
  REGION 1..4: note = Linker peptide
KGGS

SEQ ID NO: 15 - protein, 138 aa, Hepatitis B virus
NSNNPDWDFNPNKDHWPEANQVGAGAFGPGFTPPHGGLLGWSPQAQGILTTVPAAPPPASTNRQSGRQPTPISPPLRDSHPQAMQWNSTTFHQALLDPRVRGLYFPAGGSSSGTVNPVPTTASPISSIFSRTGDPAPN

SEQ ID NO: 16 - protein, 29 aa, Hepatitis B virus
MQLFHLCLIISCSCPTVQASKLCLGWLWG

SEQ ID NO: 17 - protein, 159 aa, Hepatitis B virus
SKLCLGWLWGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVV

SEQ ID NO: 18 - protein, 226 aa, Hepatitis B virus; identical to SEQ ID NO: 10

SEQ ID NO: 19 - protein, 843 aa, Hepatitis B virus
MPLSYQHFRKLLLLDDEAGPLEEELPRLADEGLNRRVAEDLNLGNLNVSIPWTHKVGNFTGLYSSTVPVFNPEWQTPSFPHIHLQEDIINRCQQYVGPLTVNEKRRLKLIMPARFYPNLTKYLPLDKGIKPYYPEHAVNHYFKTRHYLHTLWKAGILYKRETTRSASFCGSPYSWEQELQHGRLVFQTSTRHGDESFCSQSSGILSRSPVGPCVRSQLKQSRLGLQPQQGSLARGKSGRSGSIRARVHPTTRRSFGVEPSGSGHIDNSASSTSSCLHQSAVRKTAYSHLSTSKRQSSSGHAVELHNIPPSSARSQSEGPIFSCWWLQFRNSKPCSDYCLTHIVNLLEDWGPCTEHGEHNIRIPRTPARVTGGVFLVDKNPHNTTESRLVVDFSQFSRGSTHVSWPKFAVPNLQSLTNLLSSNLSWLSLDVSAAFYHIPLHPAAMPHLLVGSSGLPRYVARLSSTSRNINYQHGTMQDLHDSCSRNLYVSLLLLYKTFGRKLHLYSHPIILGFRKIPMGVGLSPFLLAQFTSAICSVVRRAFPHCLAFSYMDDVVLGAKSVQHLESLFTSITNFLLSLGIHLNPNKTKRWGYSLNFMGYVIGSWGTLPQEHIVLKIKQCFRKLPVNRPIDWKVCQRIVGLLGFAAPFTQCGYPALMPLYACIQSKQAFTFSPTYKAFLCKQYLNLYPVARQRSGLCQVFADATPTGWGLAIGHRRMRGTFVAPLPIHTAELLAACFARSRSGAKLIGTDNSVVLSRKYTSFPWLLGCAANWILRGTSFVYVPSALNPADDPSRGRLGLYRPLLHLPFRPTTGRTSLYAVSPSVPSHLPDRVHFASPLHVAWRPP

SEQ ID NO: 20 - protein, 5 aa, synthetic construct
  REGION 1..5: note = Linker peptide
GSKGK

SEQ ID NO: 21 - protein, 59 aa, Hepatitis B virus
MGGWSSKPRQGMGTNLSVPNPLGFFPDHQLDPAFGANSNNPDWDFNPNKDHWPEANQVG

SEQ ID NO: 22 - protein, 7 aa, synthetic construct
  REGION 1..7: note = Linker peptide
LEGGSGG

SEQ ID NO: 23 - protein, 1539 aa, Hepatitis B virus
MSLLWGGVTVLAAMLIAGQVASVVFLVSKSGPPSGKSNSNNPDWDFNPNKDHWPEANQVGAGAFGPGFTPPHGGLLGWSPQAQGILTTVPAAPPPASTNRQSGRQPTPISPPLRDSHPQAMQWNSTTFHQALLDPRVRGLYFPAGGSSSGTVNPVPTTASPISSIFSRTGDPAPNGSKSGSKMPLSYQHFRKLLLLDDEAGPLEEELPRLADEGLNRRVAEDLNLGNLNVSIPWTHKVGNFTGLASSTVPVFNPEWQTPSFPHIHLQEDIINRCQQYVGPLTVNEKRRLKLIMPARFYPNLTKYLPLDKGIKPYYPEHAVNHYFKTRHYLHTLWKAGILYKRETTRSASFCGSPYSWEQELQHGRLVFQTSTRHGDESFCSQSSGILSRSPVGPCVRSQLKQSRLGLQPQQGSLARGKSGRSGSIRARVHPTTRRSFGVEPSGSGHIDNSASSTSSCLHQSAVRKTAYSHLSTSKRQSSSGHAVELHNIPPSSARSQSEGPIFSAWWLQFRNSKPASDYALTHIVNLLEDWGPATEHGEHNIRIPRTPARVTGGVFLVDKNPHNTTESRLVVDFSQFSRGSTHVSWPKFAVPNLQSLTNLLSSNLSWLSLDVSAAFYHIPLHPAAMPHLLVGSSGLPRYVARLSSTSRNINYQHGTMQDLHDSCSRNLYVSLLLLYKTFGRKLHLYSHPIILGFRKIPMGVGLSPFLLAQFTSAICSVVRRAFPHCLAFSYMDDVVLGAKSVQHLESLFTSITNFLLSLGIHLNPNKTKRWGYSLNFMGYVIGSWGTLPQEHIVLKIKQCFRKLPVNRPIDWKVCQRIVGLLGFAAPFTQCGYPALMPLYACIQSKQAFTFSPTYKAFLCKQYLNLYPVARQRSGLCQVFADATPTGWGLAIGHRAMRGTFVAPLPIHTAELLAACFARSRSGAKLIGTDNSVVLSRKYTSFPWLLGCAANWILRGTSFVYVPSALNPAADPSAGRLGLYRPLLHLPFRPTTGRTSLYAVSPSVPSHLPDRVHFASPLHVAWRPPSKSPGSGPPMQLFHLCLIISCSCPTVQASKLCLGWLWGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRESQCMGGWSSKPRQGMGTNLSVPNPLGFFPDHQLDPAFGANSNNPDWDFNPNKDHWPEANQVGASKGGKSGMENTTSGFLGPLLVLQAGFFLLTRILTIPQSLDSWWTSLNFLGGAPTCPGQNSQSPTSNHSPTSCPPICPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILSPFLPLLPIFFCLWVYI

SEQ ID NO: 24 - DNA, 7143 bp, synthetic construct
  misc_feature 1..7143: note = Vaccine construct
  source 1..7143: mol_type = other DNA
gtaatctattcgatataccgttgctaacagtatactggcccaataactgtggatggaaaatctataataatacattaatatcatccgatggtgctagggttatttggatggatgcgtataaattttcttgcggtttatctttacaagactattgttatcattggggtagcaaaccagagagccgaccattcgatttaataaaaaaatcagatgctaaacgcaattctaaatcgttggtcaaagaatctatggcatccttgaaatccttgtacgaggcattcgagacacaatcaggagcgttagaagttttaatgagtccatgtaggatgttttcgttttctagaatagaagacatgttcttaactagtgtcattaatagagtatccgagaatactggaatggggatgtattatcctaccaacgatataccttctctatttatcgaatcatctatctgtctagattatattatagtaaataatcaggaatccaacaaatatcgtatcaaatctgttctcgatatcatttcttcaaaacaataccctgcaggacgtcccaactacgttaaaaatggtacaaaaggaaagttatatatcgcgttgtgtaaagttaccgtacctactaacgaccatattccagtagtttatcacgatgatgacaatactaccacctttattacagtattgacgtccgtcgatattgaaactgctatcagagcaggatattcgatagtcgaattaggggctttacaatgggataataatattccagaacttaaaaacggtttactggatagtatcaagatgatttatgacttgaacgcagttacaacaaataatttattggaacagctcatagaaaatattaactttaacaactctagtataatttcgttgttttatacatttgccattagttattgccgagcattcatttactcaattatggaaaccatagatccggtgtatatatctcagttcagttataaagaattatacgttagtagctcttataaagatattaatgaatccatgagtcagatggtaaaattataaaaagtgaaaaacaatattatttttatcgttggttgttacactatgtctctgctgtggggcggagtgacagtgctggccgctatgctgattgctggacaggtggccagcgtggtgttcctggtgtctaagtctggacctcctagcggcaagagcaacagcaacaaccccgactgggacttcaaccccaacaaggaccattggccagaggccaatcaagtcggagccggcgcttttggccctggctttacacctcctcatggcggactgcttggatggtcacctcaggctcagggcatcctgacaacagtgcctgctgctccacctcctgccagcaccaatagacagtctggcagacagcccacacctatcagccctccactgagagacagccatcctcaggccatgcagtggaacagcaccacctttcatcaggccctgctggaccctagagtgcggggcctttattttcctgccggcggatctagcagcggcaccgtgaatcctgtgcctacaaccgcctctcctatcagcagcatcttcagcagaaccggcgatcccgctcctaacggcagcaagtctggctccaagatgcccctgagctaccagcacttcagaaaactgctgctgctggacgacgaggccggacctctggaagaagaactgcctagactggccgacgagggcctgaatagaagagtggccgaggacctgaacctgggcaacctgaatgtgtctatcccctggacacacaaagtgggcaacttcacaggcctggccagctctaccgtgcctgtgttcaatcccgagtggcagacccctagcttccctcacatccatctgcaagaggacatcatcaaccggtgccagcagtacgtgggccctctgaccgtgaacgagaagcggagactgaagctgatcatgcccgccagattctaccccaacctgaccaagtacctgcctctggacaagggcatcaagccttactaccctgagcacgccgtgaaccactacttcaagacccggcactacctgcacaccctgtggaaggccggcatcctgtataagagagagacaaccagaagcgccagcttctgcggcagcccttactcttgggagcaagagctgcagcacggcagactggtgttccagacctctacaagacacggcgacgagtctttctgcagccagagcagcggaatcctgagcagatctcctgtgggaccttgtgtgcggagccagctgaaacagagtagactgggcctgcagcctcagcagggatctctggctagaggaaagagcggcagaagcggcagcatcagagccagagtgcaccctaccaccagaagaagctttggcgtcgagcctagcggctccggccacatcgataatagcgccagcagcaccagcagctgcctgcatcagtccgccgttagaaagaccgcctacagccacctgagcacctccaagagacagagcagctctggacacgccgtggaactgcacaacatccctccaagcagcgccagatctcagagcgagggccctatcttttctgcctggtggctgcagttcagaaacagcaagcccgccagcgactacgccctgacacacatcgtgaacctgctggaagattggggccctgccacagaacacggcgagcacaatatcagaatccccagaacacccgccagagtgacaggcggagtgtttctggtggacaagaaccctcacaacaccaccgagagcaggctggtggtggacttcagccagtttagcagaggcagcacccacgtgtcctggcctaaattcgccgtgcctaacctgcagagcctgaccaatctgctgagcagcaacctgagctggctgagcctggatgtgtccgccgccttctatcacatcccactgcatcctgccgccatgcctcatctgcttgtgggaagttctggcctgcctagatacgtggccaggctgtcctctaccagccggaacatcaattaccagcacgggaccatgcaggacctgcacgacagctgtagccggaacctgtacgttagcctgctcctgctgtacaagaccttcggcagaaagctgcacctgtactctcaccccatcatcctgggcttcagaaagatccccatgggcgtgggcctgtctccatttctgctggcccagtttaccagcgccatctgctctgttgtgcggagagcctttcctcactgcctggccttcagctacatggacgatgtggtgctgggcgccaagtctgtgcagcacctggaaagcctgtttacctccatcaccaactttctgctgtccctgggaatccatctgaaccctaacaagaccaagagatggggctacagcctgaacttcatgggctacgtgatcggcagctggggcacactgcctcaagagcacatcgtgctgaaaatcaagcagtgctttcggaagctgcccgtgaacagacccatcgactggaaagtgtgccagcggatcgttggcctgctgggatttgccgctcctttcacacagtgcggctaccccgctctcatgcctctgtatgcctgtatccagagcaagcaggccttcacattcagccctacctacaaggccttcctgtgcaagcagtacctgaatctgtaccccgtggccagacagagaagcggcctgtgtcaagtgttcgccgatgccacacctacaggatggggactcgccattggacacagggccatgagaggaacctttgtggcccctctgcctatccacacagccgaattgctggctgcctgcttcgccagaagtagatccggcgccaagctgatcggcaccgacaattctgtggtcctgagccggaagtacaccagctttccatggctgctgggctgtgccgccaactggattctgagaggcaccagcttcgtgtacgtgcccagcgctcttaaccctgccgctgatccttctgctgggagactcggcctgtacagacctctgctgcatctgcccttcagacccaccacaggcagaaccagcctgtatgccgtgtctcctagcgtgccaagccatctgcctgatcgggtgcactttgcctctccacttcatgtggcttggcggccacctagcaagagccctggatctggaccacctatgcagctgtttcacctgtgcctgatcatctcttgcagctgccctacagtgcaggccagcaagctgtgtctcggatggctctggggcatggacatcgacccctacaaagaattcggcgccagcgtcgagctgctgagcttcctgcctagcgacttcttcccctccatccgggatctgctggatactgctagcgccctgtatcgcgaggccctggaatctcctgagcactgttctccacaccacactgccctgagacaggctatcctgtgttggggcgagctgatgaacctggccacatgggtcggaagcaacctggaagatcccgccagcagagaactggtggtgtcctacgtgaacgtgaacatgggcctgaagatcagacagctgctgtggttccacatctcctgcctgacctttggccgggaaaccgtgctggaatacctggtgtcctttggcgtgtggatcagaacccctccagcctaccgacctcctaacgctcctatcctgtctaccctgcctgagacaacagtcgtgcggcggagaggaagaagccctagaagaaggaccccatctcctcggcggagaagatcacagtcccctcggagaagaagatcccagagccgggaatcccagtgctgatttttgttcgacattaaaaattgaaaataaatacaaaggttcttgagggttgtgttaaattgaaagcgagaaataatcataaatatgattcaggtgacggatccatgggaggctggtccagcaaacccagacaaggcatgggcaccaatctgagcgtgcccaatcctctgggattcttcccagaccaccagctggatcctgccttcggcgccaacagcaacaaccccgactgggacttcaaccccaacaaggaccattggccagaggccaatcaagtgggcgctagcaaaggcggcaagagcggcatggagaataccacctctggctttctgggccctctgctggttctgcaggccggcttctttctgctgacccggatcctgacaatccctcagagcctggatagctggtggacaagcctgaacttccttggcggcgctcctacatgccctggccagaattctcagagccccaccagcaatcacagccccacaagctgccctccaatctgccctggctacagatggatgtgcctgcggcggttcatcatctttctgttcatcctgctgctgtgcctgatcttcctgctggtgctgctggactatcagggcatgctgcctgtgtgtcctctgctgcctggcaccagcacaacatctacaggcccttgcaagacctgcacaatcccagctcagggcacctccatgtttcccagctgctgttgcaccaagcctagcgacggcaactgcacatgcatccccattccttccagctgggccttcgccagatttctgtgggaatgggccagcgtgcggttcagctggctgtctctgcttgtgcctttcgtgcagtggttcgtgggcctgtctcctacagtgtggctgagcgtgatctggatgatgtggtactggggccctagcctgtacaatattctgagccccttcctgcctctgctcccaatcttcttctgcctgtgggtgtacatctgattaaccgagtttctgcattattgtaattcgtatgctggcaccatcaaagaatcacttctaaaagatatcaatatcacacatacaaatattactaccctattgaatgagacagccaaggttatcaagttagtaaaatctctggtagataaagaagatactgatattgtgaataatttcattaccaaagaaattaaaaacagagacaaaatagttaatagtttgtctctatcaaacctggactttcgtttgtaaattggggctttttgtacaataaatgggtgttgccaatgattcatcccctgaatatcaatggatgtctccccatagattatcagatactgttatattaggagactgtttgtattttaacaatataatgtcccaattagatttacaccaaaattgggctccatcagttagattgttaaattattttaagaattttaataaggaaacactactaaagatagaagagaatgattacattaattcatcctttttccaacaaaaggataaacgattttatcctataaacgacgatttttatcacatatctacaggaggatatggtatagtctttaagatagataactatgtagtaaaatttgtattcgaggccacaaaattatatagtcccatggaaactacggcggagttcacagtacccaaatttctatacaacaatctaaagggagatgaaaaaaaattaatcgtgtgtgcgtgggccatgggattaaactataaattaacatttttacatactctgtataaacgtgttcttcatatgttgctattattgatacaaactatggatggtcaggaactatcattgagatattcttctaaagtttttttaaaggcgtttaacgagagaaaggacagtatcaaattcgtgaaattactatcccacttttatccggcagttattaacagtaatattaatgttataaactattttaaccgcatgtttcactttttcgaacatgaaaagagaactaactacgaatacgaaagaggaaatattataatttttcccctagcactgtattcggcagataaagtagataccgagctagctatcaaattaggatttaaatctttggtacaatacataaagtttatctttttacagatggctctgttatacattaaaatttacgaactaccatgctgcgacaactttttacacgcagatcttaaacccgataatatcttactttttgattccaatgaaccaataataattcatctaaaggataaaaagtttgtttttaatgaacgtattaaatcggcattaaacgactttgacttttcccaag

SEQ ID NO: 25 - protein, 1524 aa, synthetic construct
  REGION 1..1524: note = Vaccine construct
MSLLWGGVTVLAAMLIAGQVASVVFLVKGGGPGGGMQLFHLCLIISCSCPTVQASKLCLGWLWGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRESQCGGGSGGGMPLSYQHFRKLLLLDDEAGPLEEELPRLADEGLNRRVAEDLNLGNLNVSIPWTHKVGNFTGLASSTVPVFNPEWQTPSFPHIHLQEDIINRCQQYVGPLTVNEKRRLKLIMPARFYPNLTKYLPLDKGIKPYYPEHAVNHYFKTRHYLHTLWKAGILYKRETTRSASFCGSPYSWEQELQHGRLVFQTSTRHGDESFCSQSSGILSRSPVGPCVRSQLKQSRLGLQPQQGSLARGKSGRSGSIRARVHPTTRRSFGVEPSGSGHIDNSASSTSSCLHQSAVRKTAYSHLSTSKRQSSSGHAVELHNIPPSSARSQSEGPIFSAWWLQFRNSKPASDYALTHIVNLLEDWGPATEHGEHNIRIPRTPARVTGGVFLVDKNPHNTTESRLVVDFSQFSRGSTHVSWPKFAVPNLQSLTNLLSSNLSTFGRKLHLYSHPIILGFRKIPMGVGLSPFLLAQFTSAICSVVRRAFPHCLAFSYMDDVVLGAKSVQHLESLFTSITNFLLSLGIHLNPNKTKRWGYSLNFMGYVIGSWGTLPQEHIVLKIKQCFRKLPVNRPIDWKVCQRIVGLLGFAAPFTQCGYPALMPLYACIQSKQAFTFSPTYKAFLCKQYLNLYPVARQRSGLCQVFADATPTGWGLAIGHRAMRGTFVAPLPIHTAELLAACFARSRSGAKLIGTDNSVVLSRKYTSFPWLLGCAANWILRGTSFVYVPSALNPAADPSAGRLGLYRPLLHLPFRPTTGRTSLYAVSPSVPSHLPDRVHFASPLHVAWRPPKSPNSNNPDWDFNPNKDHWPEANQVGAGAFGPGFTPPHGGLLGWSPQAQGILTTVPAAPPPASTNRQSGRQPTPISPPLRDSHPQAMQWNSTTFHQALLDPRVRGLYFPAGGSSSGTVNPVPTTASPISSIFSRTGDPAPNGSKGKRKRRAPVKQTLNFDLLKLAGDVESNPGPMDAMKRGLCCVLLLCGAVFVSPSQEIHARFRRMGGWSSKPRQGMGTNLSVPNPLGFFPDHQLDPAFGANSNNPDWDFNPNKDHWPEANQVGLEGGSGGMENTTSGFLGPLLVLQAGFFLLTRILTIPQSLDSWWTSLNFLGGAPTCPGQNSQSPTSNHSPTSCPPICPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILSPFLPLLPIFFCLWVYI

SEQ ID NO: 26 - protein, 1571 aa, synthetic construct
  REGION 1..1571: note = Vaccine construct
MSLLWGGVTVLAAMLIAGQVASVVFLVSKSGPPSGKSNSNNPDWDFNPNKDHWPEANQVGAGAFGPGFTPPHGGLLGWSPQAQGILTTVPAAPPPASTNRQSGRQPTPISPPLRDSHPQAMQWNSTTFHQALLDPRVRGLYFPAGGSSSGTVNPVPTTASPISSIFSRTGDPAPNGSKSGSKMPLSYQHFRKLLLLDDEAGPLEEELPRLADEGLNRRVAEDLNLGNLNVSIPWTHKVGNFTGLASSTVPVFNPEWQTPSFPHIHLQEDIINRCQQYVGPLTVNEKRRLKLIMPARFYPNLTKYLPLDKGIKPYYPEHAVNHYFKTRHYLHTLWKAGILYKRETTRSASFCGSPYSWEQELQHGRLVFQTSTRHGDESFCSQSSGILSRSPVGPCVRSQLKQSRLGLQPQQGSLARGKSGRSGSIRARVHPTTRRSFGVEPSGSGHIDNSASSTSSCLHQSAVRKTAYSHLSTSKRQSSSGHAVELHNIPPSSARSQSEGPIFSAWWLQFRNSKPASDYALTHIVNLLEDWGPATEHGEHNIRIPRTPARVTGGVFLVDKNPHNTTESRLVVDFSQFSRGSTHVSWPKFAVPNLQSLTNLLSSNLSWLSLDVSAAFYHIPLHPAAMPHLLVGSSGLPRYVARLSSTSRNINYQHGTMQDLHDSCSRNLYVSLLLLYKTFGRKLHLYSHPIILGFRKIPMGVGLSPFLLAQFTSAICSVVRRAFPHCLAFSYMDDVVLGAKSVQHLESLFTSITNFLLSLGIHLNPNKTKRWGYSLNFMGYVIGSWGTLPQEHIVLKIKQCFRKLPVNRPIDWKVCQRIVGLLGFAAPFTQCGYPALMPLYACIQSKQAFTFSPTYKAFLCKQYLNLYPVARQRSGLCQVFADATPTGWGLAIGHRAMRGTFVAPLPIHTAELLAACFARSRSGAKLIGTDNSVVLSRKYTSFPWLLGCAANWILRGTSFVYVPSALNPAADPSAGRLGLYRPLLHLPFRPTTGRTSLYAVSPSVPSHLPDRVHFASPLHVAWRPPSKSPGSGPPMQLFHLCLIISCSCPTVQASKLCLGWLWGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRESQCMDAMKRGLCCVLLLCGAVFVSPSQEIHARFRRMGGWSSKPRQGMGTNLSVPNPLGFFPDHQLDPAFGANSNNPDWDFNPNKDHWPEANQVGASKGGKSGMENTTSGFLGPLLVLQAGFFLLTRILTIPQSLDSWWTSLNFLGGAPTCPGQNSQSPTSNHSPTSCPPICPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILSPFLPLLPIFFCLWVYI

SEQ ID NO: 27 - DNA, 7239 bp, synthetic construct
  misc_feature 1..7239: note = Vaccine construct
  source 1..7239: mol_type = other DNA
gtaatctattcgatataccgttgctaacagtatactggcccaataactgtggatggaaaatctataataatacattaatatcatccgatggtgctagggttatttggatggatgcgtataaattttcttgcggtttatctttacaagactattgttatcattggggtagcaaaccagagagccgaccattcgatttaataaaaaaatcagatgctaaacgcaattctaaatcgttggtcaaagaatctatggcatccttgaaatccttgtacgaggcattcgagacacaatcaggagcgttagaagttttaatgagtccatgtaggatgttttcgttttctagaatagaagacatgttcttaactagtgtcattaatagagtatccgagaatactggaatggggatgtattatcctaccaacgatataccttctctatttatcgaatcatctatctgtctagattatattatagtaaataatcaggaatccaacaaatatcgtatcaaatctgttctcgatatcatttcttcaaaacaataccctgcaggacgtcccaactacgttaaaaatggtacaaaaggaaagttatatatcgcgttgtgtaaagttaccgtacctactaacgaccatattccagtagtttatcacgatgatgacaatactaccacctttattacagtattgacgtccgtcgatattgaaactgctatcagagcaggatattcgatagtcgaattaggggctttacaatgggataataatattccagaacttaaaaacggtttactggatagtatcaagatgatttatgacttgaacgcagttacaacaaataatttattggaacagctcatagaaaatattaactttaacaactctagtataatttcgttgttttatacatttgccattagttattgccgagcattcatttactcaattatggaaaccatagatccggtgtatatatctcagttcagttataaagaattatacgttagtagctcttataaagatattaatgaatccatgagtcagatggtaaaattataaaaagtgaaaaacaatattatttttatcgttggttgttacactatgtctctgctgtggggcggagtgacagtgctggccgctatgctgattgctggacaggtggccagcgtggtgttcctggtgtctaagtctggacctcctagcggcaagagcaacagcaacaaccccgactgggacttcaaccccaacaaggaccattggccagaggccaatcaagtcggagccggcgcttttggccctggctttacacctcctcatggcggactgcttggatggtcacctcaggctcagggcatcctgacaacagtgcctgctgctccacctcctgccagcaccaatagacagtctggcagacagcccacacctatcagccctccactgagagacagccatcctcaggccatgcagtggaacagcaccacctttcatcaggccctgctggaccctagagtgcggggcctttattttcctgccggcggatctagcagcggcaccgtgaatcctgtgcctacaaccgcctctcctatcagcagcatcttcagcagaaccggcgatcccgctcctaacggcagcaagtctggctccaagatgcccctgagctaccagcacttcagaaaactgctgctgctggacgacgaggccggacctctggaagaagaactgcctagactggccgacgagggcctgaatagaagagtggccgaggacctgaacctgggcaacctgaatgtgtctatcccctggacacacaaagtgggcaacttcacaggcctggccagctctaccgtgcctgtgttcaatcccgagtggcagacccctagcttccctcacatccatctgcaagaggacatcatcaaccggtgccagcagtacgtgggccctctgaccgtgaacgagaagcggagactgaagctgatcatgcccgccagattctaccccaacctgaccaagtacctgcctctggacaagggcatcaagccttactaccctgagcacgccgtgaaccactacttcaagacccggcactacctgcacaccctgtggaaggccggcatcctgtataagagagagacaaccagaagcgccagcttctgcggcagcccttactcttgggagcaagagctgcagcacggcagactggtgttccagacctctacaagacacggcgacgagtctttctgcagccagagcagcggaatcctgagcagatctcctgtgggaccttgtgtgcggagccagctgaaacagagtagactgggcctgcagcctcagcagggatctctggctagaggaaagagcggcagaagcggcagcatcagagccagagtgcaccctaccaccagaagaagctttggcgtcgagcctagcggctccggccacatcgataatagcgccagcagcaccagcagctgcctgcatcagtccgccgttagaaagaccgcctacagccacctgagcacctccaagagacagagcagctctggacacgccgtggaactgcacaacatccctccaagcagcgccagatctcagagcgagggccctatcttttctgcctggtggctgcagttcagaaacagcaagcccgccagcgactacgccctgacacacatcgtgaacctgctggaagattggggccctgccacagaacacggcgagcacaatatcagaatccccagaacacccgccagagtgacaggcggagtgtttctggtggacaagaaccctcacaacaccaccgagagcaggctggtggtggacttcagccagtttagcagaggcagcacccacgtgtcctggcctaaattcgccgtgcctaacctgcagagcctgaccaatctgctgagcagcaacctgagctggctgagcctggatgtgtccgccgccttctatcacatcccactgcatcctgccgccatgcctcatctgcttgtgggaagttctggcctgcctagatacgtggccaggctgtcctctaccagccggaacatcaattaccagcacgggaccatgcaggacctgcacgacagctgtagccggaacctgtacgttagcctgctcctgctgtacaagaccttcggcagaaagctgcacctgtactctcaccccatcatcctgggcttcagaaagatccccatgggcgtgggcctgtctccatttctgctggcccagtttaccagcgccatctgctctgttgtgcggagagcctttcctcactgcctggccttcagctacatggacgatgtggtgctgggcgccaagtctgtgcagcacctggaaagcctgtttacctccatcaccaactttctgctgtccctgggaatccatctgaaccctaacaagaccaagagatggggctacagcctgaacttcatgggctacgtgatcggcagctggggcacactgcctcaagagcacatcgtgctgaaaatcaagcagtgctttcggaagctgcccgtgaacagacccatcgactggaaagtgtgccagcggatcgttggcctgctgggatttgccgctcctttcacacagtgcggctaccccgctctcatgcctctgtatgcctgtatccagagcaagcaggccttcacattcagccctacctacaaggccttcctgtgcaagcagtacctgaatctgtaccccgtggccagacagagaagcggcctgtgtcaagtgttcgccgatgccacacctacaggatggggactcgccattggacacagggccatgagaggaacctttgtggcccctctgcctatccacacagccgaattgctggctgcctgcttcgccagaagtagatccggcgccaagctgatcggcaccgacaattctgtggtcctgagccggaagtacaccagctttccatggctgctgggctgtgccgccaactggattctgagaggcaccagcttcgtgtacgtgcccagcgctcttaaccctgccgctgatccttctgctgggagactcggcctgtacagacctctgctgcatctgcccttcagacccaccacaggcagaaccagcctgtatgccgtgtctcctagcgtgccaagccatctgcctgatcgggtgcactttgcctctccacttcatgtggcttggcggccacctagcaagagccctggatctggaccacctatgcagctgtttcacctgtgcctgatcatctcttgcagctgccctacagtgcaggccagcaagctgtgtctcggatggctctggggcatggacatcgacccctacaaagaattcggcgccagcgtcgagctgctgagcttcctgcctagcgacttcttcccctccatccgggatctgctggatactgctagcgccctgtatcgcgaggccctggaatctcctgagcactgttctccacaccacactgccctgagacaggctatcctgtgttggggcgagctgatgaacctggccacatgggtcggaagcaacctggaagatcccgccagcagagaactggtggtgtcctacgtgaacgtgaacatgggcctgaagatcagacagctgctgtggttccacatctcctgcctgacctttggccgggaaaccgtgctggaatacctggtgtcctttggcgtgtggatcagaacccctccagcctaccgacctcctaacgctcctatcctgtctaccctgcctgagacaacagtcgtgcggcggagaggaagaagccctagaagaaggaccccatctcctcggcggagaagatcacagtcccctcggagaagaagatcccagagccgggaatcccagtgctgatttttgttcgacattaaaaattgaaaataaatacaaaggttcttgagggttgtgttaaattgaaagcgagaaataatcataaatatgattcaggtgacggatccatggacgctatgaagaggggcctgtgctgtgtgctgctgctgtgcggagctgtgtttgtgtcccctagccaagagatccacgccagattcagaagaatgggcggctggtccagcaagcccagacaaggcatgggcaccaatctgagcgtgcccaatcctctgggattcttcccagaccaccagctggatcctgccttcggcgccaacagcaacaaccccgactgggacttcaaccccaacaaggaccattggccagaggccaatcaagtgggcgctagcaaaggcggcaagagcggcatggagaataccacctctggctttctgggccctctgctggttctgcaggccggcttctttctgctgacccggatcctgacaatccctcagagcctggatagctggtggacaagcctgaacttccttggcggcgctcctacatgccctggccagaattctcagagccccaccagcaatcacagccccacaagctgccctccaatctgccctggctacagatggatgtgcctgcggcggttcatcatctttctgttcatcctgctgctctgcctgatcttcctgctggtgctcctggactatcagggcatgctgcctgtgtgtcctctgctgcctggcaccagcacaacatctacaggcccttgcaagacctgcacaatcccagctcagggcacctccatgtttcccagctgctgttgcaccaagcctagcgacggcaactgcacatgcatccccattccttccagctgggccttcgccagatttctgtgggaatgggccagcgtgcggttcagctggctgtctctgcttgtgcctttcgtgcagtggttcgtgggcctgtctcctacagtgtggctgagcgtgatctggatgatgtggtactggggccctagcctgtacaatattctgagccccttcctgcctctgctcccaatcttcttctgcctgtgggtgtacatctgattaaccgagtttctgcattattgtaattcgtatgctggcaccatcaaagaatcacttctaaaagatatcaatatcacacatacaaatattactaccctattgaatgagacagccaaggttatcaagttagtaaaatctctggtagataaagaagatactgatattgtgaataatttcattaccaaagaaattaaaaacagagacaaaatagttaatagtttgtctctatcaaacctggactttcgtttgtaaattggggctttttgtacaataaatgggtgttgccaatgattcatcccctgaatatcaatggatgtctccccatagattatcagatactgttatattaggagactgtttgtattttaacaatataatgtcccaattagatttacaccaaaattgggctccatcagttagattgttaaattattttaagaattttaataaggaaacactactaaagatagaagagaatgattacattaattcatcctttttccaacaaaaggataaacgattttatcctataaacgacgatttttatcacatatctacaggaggatatggtatagtctttaagatagataactatgtagtaaaatttgtattcgaggccacaaaattatatagtcccatggaaactacggcggagttcacagtacccaaatttctatacaacaatctaaagggagatgaaaaaaaattaatcgtgtgtgcgtgggccatgggattaaactataaattaacatttttacatactctgtataaacgtgttcttcatatgttgctattattgatacaaactatggatggtcaggaactatcattgagatattcttctaaagtttttttaaaggcgtttaacgagagaaaggacagtatcaaattcgtgaaattactatcccacttttatccggcagttattaacagtaatattaatgttataaactattttaaccgcatgtttcactttttcgaacatgaaaagagaactaactacgaatacgaaagaggaaatattataatttttcccctagcactgtattcggcagataaagtagataccgagctagctatcaaattaggatttaaatctttggtacaatacataaagtttatctttttacagatggctctgttatacattaaaatttacgaactaccatgctgcgacaactttttacacgcagatcttaaacccgataatatcttactttttgattccaatgaaccaataataattcatctaaaggataaaaagtttgtttttaatgaacgtattaaatcggcattaaacgactttgacttttcccaag

SEQ ID NO: 28 - DNA, 97 bp, synthetic construct
  misc_feature 1..97: note = Promoter
  source 1..97: mol_type = other DNA
tcgacattaaaaattgaaaataaatacaaaggttcttgagggttgtgttaaattgaaagcgagaaataatcataaatatgattcaggtgacggatcc

SEQ ID NO: 29 - DNA, 96 bp, synthetic construct
  misc_feature 1..96: note = TPA sequence
  source 1..96: mol_type = other DNA
atggacgctatgaagaggggcctgtgctgtgtgctgctgctgtgcggagctgtgtttgtgtcccctagccaagagatccacgccagattcagaaga

SEQ ID NO: 30 - protein, 32 aa, synthetic construct
  REGION 1..32: note = TPA peptide
MDAMKRGLCCVLLLCGAVFVSPSQEIHARFRR

SEQ ID NO: 31 - protein, 10 aa, synthetic construct
  REGION 1..10: note = Linker peptide
SKSGPPSGKS

SEQ ID NO: 32 - protein, 7 aa, synthetic construct
  REGION 1..7: note = Linker peptide
GSKSGSK

SEQ ID NO: 33 - protein, 9 aa, synthetic construct
  REGION 1..9: note = Linker peptide
SKSPGSGPP

SEQ ID NO: 34 - protein, 8 aa, synthetic construct
  REGION 1..8: note = Linker peptide
ASKGGKSG

SEQ ID NO: 35 - DNA, 1097 bp, synthetic construct
  misc_feature 1..1097: note = F11 left flank
  source 1..1097: mol_type = other DNA
gtaatctattcgatataccgttgctaacagtatactggcccaataactgtggatggaaaatctataataatacattaatatcatccgatggtgctagggttatttggatggatgcgtataaattttcttgcggtttatctttacaagactattgttatcattggggtagcaaaccagagagccgaccattcgatttaataaaaaaatcagatgctaaacgcaattctaaatcgttggtcaaagaatctatggcatccttgaaatccttgtacgaggcattcgagacacaatcaggagcgttagaagttttaatgagtccatgtaggatgttttcgttttctagaatagaagacatgttcttaactagtgtcattaatagagtatccgagaatactggaatggggatgtattatcctaccaacgatataccttctctatttatcgaatcatctatctgtctagattatattatagtaaataatcaggaatccaacaaatatcgtatcaaatctgttctcgatatcatttcttcaaaacaataccctgcaggacgtcccaactacgttaaaaatggtacaaaaggaaagttatatatcgcgttgtgtaaagttaccgtacctactaacgaccatattccagtagtttatcacgatgatgacaatactaccacctttattacagtattgacgtccgtcgatattgaaactgctatcagagcaggatattcgatagtcgaattaggggctttacaatgggataataatattccagaacttaaaaacggtttactggatagtatcaagatgatttatgacttgaacgcagttacaacaaataatttattggaacagctcatagaaaatattaactttaacaactctagtataatttcgttgttttatacatttgccattagttattgccgagcattcatttactcaattatggaaaccatagatccggtgtatatatctcagttcagttataaagaattatacgttagtagctcttataaagatattaatgaatccatgagtcagatggtaaaattataaaaagtgaaaaacaatattatttttatcgttggttgttacact

SEQ ID NO: 36 - DNA, 1319 bp, synthetic construct
  misc_feature 1..1319: note = F11 right flank
  source 1..1319: mol_type = other DNA
ttaaccgagtttctgcattattgtaattcgtatgctggcaccatcaaagaatcacttctaaaagatatcaatatcacacatacaaatattactaccctattgaatgagacagccaaggttatcaagttagtaaaatctctggtagataaagaagatactgatattgtgaataatttcattaccaaagaaattaaaaacagagacaaaatagttaatagtttgtctctatcaaacctggactttcgtttgtaaattggggctttttgtacaataaatgggtgttgccaatgattcatcccctgaatatcaatggatgtctccccatagattatcagatactgttatattaggagactgtttgtattttaacaatataatgtcccaattagatttacaccaaaattgggctccatcagttagattgttaaattattttaagaattttaataaggaaacactactaaagatagaagagaatgattacattaattcatcctttttccaacaaaaggataaacgattttatcctataaacgacgatttttatcacatatctacaggaggatatggtatagtctttaagatagataactatgtagtaaaatttgtattcgaggccacaaaattatatagtcccatggaaactacggcggagttcacagtacccaaatttctatacaacaatctaaagggagatgaaaaaaaattaatcgtgtgtgcgtgggccatgggattaaactataaattaacatttttacatactctgtataaacgtgttcttcatatgttgctattattgatacaaactatggatggtcaggaactatcattgagatattcttctaaagtttttttaaaggcgtttaacgagagaaaggacagtatcaaattcgtgaaattactatcccacttttatccggcagttattaacagtaatattaatgttataaactattttaaccgcatgtttcactttttcgaacatgaaaagagaactaactacgaatacgaaagaggaaatattataatttttcccctagcactgtattcggcagataaagtagataccgagctagctatcaaattaggatttaaatctttggtacaatacataaagtttatctttttacagatggctctgttatacattaaaatttacgaactaccatgctgcgacaactttttacacgcagatcttaaacccgataatatcttactttttgattccaatgaaccaataataattcatctaaaggataaaaagtttgtttttaatgaacgtattaaatcggcattaaacgactttgacttttcccaag

SEQ ID NO: 37 - DNA, 3741 bp, synthetic construct
  misc_feature 1..3741: note = Vaccine construct
  source 1..3741: mol_type = other DNA
atgtctctgctgtggggcggagtgacagtgctggccgctatgctgattgctggacaggtggccagcgtggtgttcctggtgtctaagtctggacctcctagcggcaagagcaacagcaacaaccccgactgggacttcaaccccaacaaggaccattggccagaggccaatcaagtcggagccggcgcttttggccctggctttacacctcctcatggcggactgcttggatggtcacctcaggctcagggcatcctgacaacagtgcctgctgctccacctcctgccagcaccaatagacagtctggcagacagcccacacctatcagccctccactgagagacagccatcctcaggccatgcagtggaacagcaccacctttcatcaggccctgctggaccctagagtgcggggcctttattttcctgccggcggatctagcagcggcaccgtgaatcctgtgcctacaaccgcctctcctatcagcagcatcttcagcagaaccggcgatcccgctcctaacggcagcaagtctggctccaagatgcccctgagctaccagcacttcagaaaactgctgctgctggacgacgaggccggacctctggaagaagaactgcctagactggccgacgagggcctgaatagaagagtggccgaggacctgaacctgggcaacctgaatgtgtctatcccctggacacacaaagtgggcaacttcacaggcctggccagctctaccgtgcctgtgttcaatcccgagtggcagacccctagcttccctcacatccatctgcaagaggacatcatcaaccggtgccagcagtacgtgggccctctgaccgtgaacgagaagcggagactgaagctgatcatgcccgccagattctaccccaacctgaccaagtacctgcctctggacaagggcatcaagccttactaccctgagcacgccgtgaaccactacttcaagacccggcactacctgcacaccctgtggaaggccggcatcctgtataagagagagacaaccagaagcgccagcttctgcggcagcccttactcttgggagcaagagctgcagcacggcagactggtgttccagacctctacaagacacggcgacgagtctttctgcagccagagcagcggaatcctgagcagatctcctgtgggaccttgtgtgcggagccagctgaaacagagtagactgggcctgcagcctcagcagggatctctggctagaggaaagagcggcagaagcggcagcatcagagccagagtgcaccctaccaccagaagaagctttggcgtcgagcctagcggctccggccacatcgataatagcgccagcagcaccagcagctgcctgcatcagtccgccgttagaaagaccgcctacagccacctgagcacctccaagagacagagcagctctggacacgccgtggaactgcacaacatccctccaagcagcgccagatctcagagcgagggccctatcttttctgcctggtggctgcagttcagaaacagcaagcccgccagcgactacgccctgacacacatcgtgaacctgctggaagattggggccctgccacagaacacggcgagcacaatatcagaatccccagaacacccgccagagtgacaggcggagtgtttctggtggacaagaaccctcacaacaccaccgagagcaggctggtggtggacttcagccagtttagcagaggcagcacccacgtgtcctggcctaaattcgccgtgcctaacctgcagagcctgaccaatctgctgagcagcaacctgagctggctgagcctggatgtgtccgccgccttctatcacatcccactgcatcctgccgccatgcctcatctgcttgtgggaagttctggcctgcctagatacgtggccaggctgtcctctaccagccggaacatcaattaccagcacgggaccatgcaggacctgcacgacagctgtagccggaacctgtacgttagcctgctcctgctgtacaagaccttcggcagaaagctgcacctgtactctcaccccatcatcctgggcttcagaaagatccccatgggcgtgggcctgtctccatttctgctggcccagtttaccagcgccatctgctctgttgtgcggagagcctttcctcactgcctggccttcagctacatggacgatgtggtgctgggcgccaagtctgtgcagcacctggaaagcctgtttacctccatcaccaactttctgctgtccctgggaatccatctgaaccctaacaagaccaagagatggggctacagcctgaacttcatgggctacgtgatcggcagctggggcacactgcctcaagagcacatcgtgctgaaaatcaagcagtgctttcggaagctgcccgtgaacagacccatcgactggaaagtgtgccagcggatcgttggcctgctgggatttgccgctcctttcacacagtgcggctaccccgctctcatgcctctgtatgcctgtatccagagcaagcaggccttcacattcagccctacctacaaggccttcctgtgcaagcagtacctgaatctgtaccccgtggccagacagagaagcggcctgtgtcaagtgttcgccgatgccacacctacaggatggggactcgccattggacacagggccatgagaggaacctttgtggcccctctgcctatccacacagccgaattgctggctgcctgcttcgccagaagtagatccggcgccaagctgatcggcaccgacaattctgtggtcctgagccggaagtacaccagctttccatggctgctgggctgtgccgccaactggattctgagaggcaccagcttcgtgtacgtgcccagcgctcttaaccctgccgctgatccttctgctgggagactcggcctgtacagacctctgctgcatctgcccttcagacccaccacaggcagaaccagcctgtatgccgtgtctcctagcgtgccaagccatctgcctgatcgggtgcactttgcctctccacttcatgtggcttggcggccacctagcaagagccctggatctggaccacctatgcagctgtttcacctgtgcctgatcatctcttgcagctgccctacagtgcaggccagcaagctgtgtctcggatggctctggggcatggacatcgacccctacaaagaattcggcgccagcgtcgagctgctgagcttcctgcctagcgacttcttcccctccatccgggatctgctggatactgctagcgccctgtatcgcgaggccctggaatctcctgagcactgttctccacaccacactgccctgagacaggctatcctgtgttggggcgagctgatgaacctggccacatgggtcggaagcaacctggaagatcccgccagcagagaactggtggtgtcctacgtgaacgtgaacatgggcctgaagatcagacagctgctgtggttccacatctcctgcctgacctttggccgggaaaccgtgctggaatacctggtgtcctttggcgtgtggatcagaacccctccagcctaccgacctcctaacgctcctatcctgtctaccctgcctgagacaacagtcgtgcggcggagaggaagaagccctagaagaaggaccccatctcctcggcggagaagatcacagtcccctcggagaagaagatcccagagccgggaatcccagtgctga

SEQ ID NO: 38 - DNA, 882 bp, Hepatitis B virus
  source 1..882: mol_type = other DNA
atgggaggctggtccagcaaacccagacaaggcatgggcaccaatctgagcgtgcccaatcctctgggattcttcccagaccaccagctggatcctgccttcggcgccaacagcaacaaccccgactgggacttcaaccccaacaaggaccattggccagaggccaatcaagtgggcgctagcaaaggcggcaagagcggcatggagaataccacctctggctttctgggccctctgctggttctgcaggccggcttctttctgctgacccggatcctgacaatccctcagagcctggatagctggtggacaagcctgaacttccttggcggcgctcctacatgccctggccagaattctcagagccccaccagcaatcacagccccacaagctgccctccaatctgccctggctacagatggatgtgcctgcggcggttcatcatctttctgttcatcctgctgctgtgcctgatcttcctgctggtgctgctggactatcagggcatgctgcctgtgtgtcctctgctgcctggcaccagcacaacatctacaggcccttgcaagacctgcacaatcccagctcagggcacctccatgtttcccagctgctgttgcaccaagcctagcgacggcaactgcacatgcatccccattccttccagctgggccttcgccagatttctgtgggaatgggccagcgtgcggttcagctggctgtctctgcttgtgcctttcgtgcagtggttcgtgggcctgtctcctacagtgtggctgagcgtgatctggatgatgtggtactggggccctagcctgtacaatattctgagccccttcctgcctctgctcccaatcttcttctgcctgtgggtgtacatctga

SEQ ID NO: 39 - DNA, 12012 bp, synthetic construct
  misc_feature 1..12012: note = Vector
  source 1..12012: mol_type = other DNA
ccaccgcctcggacctggccgacctcatcttccccgagcgcctcaggctgacgctgcgcaacggcctgcccgactttatgagccaaagcatgttgcaaaaactttcggtaaaaaatgtaattactccctcctgcacaggcagcaaagcccccgctccctccagaaacacatacaaagcctcagcgtccatagcttaccgagcacggcaggcgcaagagtcagagaaaaggctgagctctaacctgactgcccgctcctgtgctcaatatataagccctaacctacactgacgtaaaagggcccaaaagtctaaaaaaataacccgcccaaaatgacacacacgcccagcacacgcccagaaaccggtgacacactcaaaaaaatacgtgcgcttcctcaaacgcccaaaccggcgtcatttccgggttcccacgctacgtcaccgctcagcgactttcaaattccgtcgaccgttaaaaacgtcactcgccccgcccctaacggtcgcccttctctcggccaatcaccttcctcccttcccaaattcaaacgcctcatttgcatattaacgcgcacaaaaagtttgaggtatatatttgaatgatggtttaaacgcggccgccaggcctacccactagtcaattcgggaggatcgaaacggcagatcgcaaaaaacagtacatacagaaggagacatgaacatgaacatcaaaaaaattgtaaaacaagccacagttctgacttttacgactgcacttctggcaggaggagcgactcaagccttcgcgaaagaaaataaccaaaaagcatacaaagaaacgtacggcgtctctcatattacacgccatgatatgctgcagatccctaaacagcagcaaaacgaaaaataccaagtgcctcaattcgatcaatcaacgattaaaaatattgagtctgcaaaaggacttgatgtgtgggacagctggccgctgcaaaacgctgacggaacagtagctgaatacaacggctatcacgttgtgtttgctcttgcgggaagcccgaaagacgctgatgacacatcaatctacatgttttatcaaaaggtcggcgacaactcaatcgacagctggaaaaacgcgggccgtgtctttaaagacagcgataagttcgacgccaacgatccgatcctgaaagatcagacgcaagaatggtccggttctgcaacctttacatctgacggaaaaatccgtttattctacactgactattccggtaaacattacggcaaacaaagcctgacaacagcgcaggtaaatgtgtcaaaatctgatgacacactcaaaatcaacggagtggaagatcacaaaacgatttttgacggagacggaaaaacatatcagaacgttcagcagtttatcgatgaaggcaattatacatccggcgacaaccatacgctgagagaccctcactacgttgaagacaaaggccataaataccttgtattcgaagccaacacgggaacagaaaacggataccaaggcgaagaatctttatttaacaaagcgtactacggcggcggcacgaacttcttccgtaaagaaagccagaagcttcagcagagcgctaaaaaacgcgatgctgagttagcgaacggcgccctcggtatcatagagttaaataatgattacacattgaaaaaagtaatgaagccgctgatcacttcaaacacggtaactgatgaaatcgagcgcgcgaatgttttcaaaatgaacggcaaatggtacttgttcactgattcacgcggttcaaaaatgacgatcgatggtattaactcaaacgatatttacatgcttggttatgtatcaaactctttaaccggcccttacaagccgctgaacaaaacagggcttgtgctgcaaatgggtcttgatccaaacgatgtgacattcacttactctcacttcgcagtgccgcaagccaaaggcaacaatgtggttatcacaagctacatgacaaacagaggcttcttcgaggataaaaaggcaacatttgcgccaagcttcttaatgaacatcaaaggcaataaaacatccgttgtcaaaaacagcatcctggagcaaggacagctgacagtcaactaataacagcaaaaagaaaatgccgatacttcattggcattttcttttatttctcaacaagatggtgaattgactagtgggtagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattgatccggaacccttaatataacttcgtataatgtatgctatacgaagttattaggtccctcgactatagggtcaccgtcgacagcgacacacttgcatcggatgcagcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacrayacgagtgggaycgtggtcccagactaataatcagaccgacgatacgagtgggaccgtggtcccagactaataatcagaccgacgatacgagtgggaccgtggtyccagwctratwatcagaccgacgatacragtggracmgtggkcccagasakaatawtcagrccgagwtaygcwktckggcctgtaacaaaggacattaagtaaagacagatamrmgtgrgactaaaacgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagggcttcgccctgtcgctcaactgcggcgagcactactggctgtaaaaggacagaccacatcatggttctgtgttcattaggttgttctgtccattgctgacataatccgctccacttcaacgtaacaccgcacgaagatttctattgttcctgaaggcatattcaaatcgttttcgttaccgcttgcaggcatcatgacagaacactacttcctataaacgctacacaggctcctgagattaataatgcggatctctacgataatgggagattttcccgactgtttcgttcgcttctcagtggataacagccagcttctctgtttaacagacaaaaacagcatatccactcagttccacatttccatataaaggccaaggcatttattctcaggataattgtttcagcatcgcaaccgcatcagactccggcatcgcaaactgcacccggtgccgggcagccacatccagcgcaaaaaccttcgtgtagacttccgttgaactgatggacttatgtcccatcaggctttgcagaactttcagcggtataccggcatacagcatgtgcatcgcataggaatggcggaacgtatgtggtgtgaccggaacagagaacgtcacaccgtcagcagcagcggcggcaaccgcctccccaatccaggtcctgaccgttctgtccgtcacttcccagatccgcgctttctctgtccttcctgtgcgacggttacgccgctccatgagcttatcgcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacatgagaattggtcgacggcgcgccaaagcttgcatgcctgcagccgcgtaacctggcaaaatcggttacggttgagtaataaatggatgccctgcgtaagcggggcacatttcattacctctttctccgcacccgacatagataataacttcgtatagtatacattatacgaagttatctagtagacttaatcgcgtttaaacccatcatcaataatatacctcaaactttttgtgcgcgttaatatgcaaatgaggcgtttgaatttgggaagggaggaaggtgattggccgagagaagggcgaccgttaggggcggggcgagtgacgttttgatgacgtgaccgcgaggaggagccagtttgcaagttctcgtgggaaaagtgacgtcaaacgaggtgtggtttgaacacggaaatactcaattttcccgcgctctctgacaggaaatgaggtgtttctaggcggatgcaagtgaaaacgggccattttcgcgcgaaaactgaatgaggaagtgaaaatctgagtaatttcgcgtttatgacagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgcgcgagtcagatctacactttgaaaggcgatcgctagcgacatcgatcacaagtttgtacaaaaaagcaggctccaccatgggaacccgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttggtacc

SEQ ID NO: 40 - DNA, 29187 bp, synthetic construct
  misc_feature 1..29187: note = Vector
  source 1..29187: mol_type = other DNA
gcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatcgatcccgtcgagatatctagacccagctttcttgtacaaagtggtgatcgattcgacagatcgcgatcgcagtgagtagtgttctggggcgggggaggacctgcatgagggccagaatgactgaaatctgtgcttttctgtgtgttgcagcatcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcggtggacgcagctgccgccgcagctgctgcatccgccgccagcgccgtgcgcggaatggccatgggcgccggctactacggcactctggtggccaactcgagttccaccaataatcccgccagcctgaacgaggagaagctgctgctgctgatggcccagcttgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcgtggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcccttgtgtccgcccaggttttccatgcactcatccatgatgatggcaatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcagagggggggatcatgtccacctgcggggcgataaagaacacggtttccggggcgggggagatgagctgggccgaaagcaagttccggagcagctgggacttgccgcagccggtggggccgtaaatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagagataggagctcctggagcgaggcgaagtttttcagcggcttgagtccgtcggccatgggcattttggagagggtctgttgcaagagttccaagcggtcccagagctcggtgatgtgctctacggcatctcgatccagcagacctcctcgtttcgcgggttgggacgactgcgggagtagggcaccagacgatgggcgtccagcgcagccagggtccggtccttccagggccgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggaatcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccactttttccagggtatgcaaacacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccagcggagatgcctttcaagagcccctcgtccatctggtcagaaaagacgatttttttgttgtcgagcttggtggcgaaggagccgtagagggcgttggaaaggagcttggcgatggagcgcatggtctggtttttttccttgtcggcgcgctccttggccgcgatgttgagctgcacgtactcgcgcgccacgcacttccattcggggaagacggtggtcatctcgtcgggcacgattctgacctgccaacctcgattatgcagggtgatgaggtccacactggtggccacctcgccgcgcaggggctcgttggtccagcagaggcggccgcccttgcgcgagcagaaggggggcagagggtccagcatgacctcgtcgggggggtcggcatcgatggtgaagatgccgggcaggagatcggggtcgaagtagctgatggaagtggccagatcgtccagggaagcttgccattcgcgcacggccagcgcgcgctcgtagggactgaggggcgtgccccagggcatggggtgggtgagcgcggaggcgtacatgccgcagatgtcgtagacgtagaggggctcctcgaggatgccgatgtaggtggggtagcagcgccccccgcggatgctggcgcgcacgtagtcatacagctcgtgcgagggcgcgaggagccccgggcccaggttggtgcgactgggcttttcggcgcggtagacgatctggcgaaagatggcatgcgagttggaggagatggtgggcctttggaagatgttgaagtgggcgtgggggaggccgaccgagtcgcggatgaagtgggcgtaggagtcttgcagtttggcgacgagctcggcggtgacgaggacgtccagagcgcagtagtcgagggtctcctggatgatgtcatacttgagctggcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaggtgtccctgaccatgaccttgaggaactggtgcttgaaatcgatatcgtcgcagcccccctgctcccagagctggaagtccgtgcgcttcttgtaggcggggttgggcaaagcgaaagtaacatcgttgaaaaggatcttgcccgcgcggggcataaagttgcgagtgatgcggaaaggctggggcacctcggcccggttgttgatgacctgggcggcgagcacgatctcgtcgaaaccgttgatgttgtggcccacgatgtagagttccacgaatcgcgggcggcccttgacgtggggcagcttcttgagctcctcgtaggtgagctcgtcggggtcgctgagaccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcggtcccatttgagctggagggcgagatcgagggcgagctcgacgaggcggtcgtcccctgagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccgttggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcagaggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcctgaaggtctccgcgaccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaagcgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcctcttcttcctcctccactaacatctcttctacttcctcctcaggcggtggtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgttgaacgaaggcttcgagccagtcgcagtcgcaaggtaggctgagcacggtttcttctgccgggtcatgttggggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcctgctggatctgggtgagggtggtctggaagtcgtcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaagcctgcaccaaccctccaggatacggaggcgggtcgttttgcaactttttttggaggccggaaatgaaactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctcctccacagccggcgcttctgcccccgccccagcagcagcagcaacttccagccacgaccgccgcggccgccgtgagcggggctggacagacttctcagtatgatcacctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaggcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtctgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgcgcggtgagcgccagcaggcggcgcgagctgagcgaccaggagctgatgcacagcctgcagcgggccctgaccggggccgggaccgagggggagagctactttgacatgggcgcggacctgcactggcagcccagccgccgggccttggaggcggcaggcggtcccccctacatagaagaggtggacgatgaggtggacgaggagggcgagtacctggaagactgatggcgcgaccgtatttttgctagatgcaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggcgttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaatctggaagccaccctgaactttttgctgaccaaccggtcgcagaagatcccgccccagtacacgctcagcgccgaggaggagcgcatcctgcgatacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtattcgcagcagcgagctgggcaggatcacgcgcccgcgcttgctgggcgaggaggagtacttgaatgactcgctgttgagacccgagcgggagaagaacttccccaataacgggatagagagcctggtggacaagatgagccgctggaagacgtatgcgcaggagcacagggacgatccgtcgcagggggccacgagccggggcagcgccgcccgtaaacgccggtggcacgacaggcagcggggactgatgtgggacgatgaggattccgccgacgacagcagcgtgttggacttgggtgggagtggtaacccgttcgctcacctgcgcccccgcatcgggcgcatgatgtaagagaaaccgaaaataaatgatactcaccaaggccatggcgaccagcgtgcgttcgtttcttctctgttgttgtatctagtatgatgaggcgtgcgtacccggagggtcctcctccctcgtacgagagcgtgatgcagcaggcgatggcggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggtcagctgaaaaccatcatgcacaccaacatgcccaacgtgaacgagttcatgtacagcaacaagttcaaggcgcgggtgatggtctcccgcaagacccccaacggggtgacagtgacagatggtagtcaggatatcttggagtatgaatgggtggagtttgagctgcccgaaggcaacttctcggtgaccatgaccatcgacctgatgaacaacgccatcatcgacaattacttggcggtggggcggcagaacggggtcctggagagcgatatcggcgtgaagttcgacactaggaacttcaggctgggctgggaccccgtgaccgagctggtcatgcccggggtgtacaccaacgaggccttccaccccgatattgtcttgctgcccggctgcggggtggacttcaccgagagccgcctcagcaacctgctgggcattcgcaagaggcagcccttccaggagggcttccagatcatgtacgaggatctggaggggggcaacatccccgcgctcctggatgtcgacgcctatgagaaaagcaaggaggagagcgccgccgcggcgactgcagctgtagccaccgcctctaccgaggtcaggggcgataattttgccagccctgcagcagtggcagcggccgaggcggctgaaaccgaaagtaagatagtcattcagccggtggagaaggatagcaaggacaggagctacaacgtgctgccggacaagataaacaccgcctaccgcagctggtacctggcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgctcacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccccgcggtggtgaaggaggaaagaaagccccgcaaaatcaagcgggtcaaaaaggacaaaaaggaagaagatgacgatctggtggagtttgtgcgcgagttcgccccccggcggcgcgtgcagtggcgcgggcggaaagtgcacccggtgctgagacccggcaccaccgtggtcttcacgcccggcgagcgctccggcagcgcttccaagcgctcctacgacgaggtgtacggggacgaggacatcctcgagcaggcggccgagcgcctgggcgagtttgcttacggcaagcgcagccgccccgccctgaaggaagaggcggtgtccatcccgctggaccacggcaaccccacgccgagcctcaagcccgtgaccctgcagcaggtgctgccgagcgcagcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagatccccacggagcccatggaaacgcagaccgagcccatgatcaagcccagcaccagcaccatggaggtgcagacggatccctggatgccatcggctcctagccgaagaccccggcgcaagtacggcgcggccagcctgctgatgcccaactacgcgctgcatccttccatcatccccacgccgggctaccgcggcacgcgcttctaccgcggtcatacaaccagccgccgccgcaagaccaccacccgccgccgccgtcgccgcacagccgctgcatctacccctgccgccctggtgcggagagtgtaccgccgcggccgcgcgcctctgaccctaccgcgcgcgcgctaccacccgagcatcgccatttaaactttcgcctgctttgcagatggccctcacatgccgcctccgcgttcccattacgggctaccgaggaagaaaaccgcgccgtagaaggctggcggggaacgggatgcgtcgccaccaccatcggcggcggcgcgccatcagcaagcggttggggggaggcttcctgcccgcgctgatccccatcatcgccgcggcgatcggggcgatccccggcattgcttccgtggcggtgcaggcctctcagcgccactgagacacttggaaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgacgcggaggagacgctgctgacgcacacggacgagccgcccccgtacgaggaggcggtgaaactgggtctgcccaccacgcggcccatcgcgcccctggccaccggggtgctgaaacccgaaagtaataagcccgcgaccctggacttgcctcctcccgcttcccgcccctctacagtggctaagcccctgccgccggtggccgtggcccgcgcgcgacccgggggctccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgctgtccgccagaaggaggagtgaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagcctggctcccaagggagcgcccaattccagccagtgggagcaaaaaaaggcaggcaatggtgacactatggaaacacacacatttggtgtggccccaatgggcggtgagaatattacaatcgacggattacaaattggaactgacgctacagctgatcaggataaaccaatttatgctgacaaaacattccagcctgaacctcaagtaggagaagaaaattggcaagaaactgaaagcttttatggcggtagggctcttaaaaaagacacaagcatgaaaccttgctatggctcctatgctagacccaccaatgtaaagggaggtcaagctaaacttaaagttggagctgatggagttcctaccaaagaatttgacatagacctggctttctttgatactcccggtggcacagtgaatggacaagatgagtataaagcagacattgtcatgtataccgaaaacacgtatctggaaactccagacacgcatgtggtatacaaaccaggcaaggatgatgcaagttctgaaattaacctggttcagcagtccatgcccaatagacccaactatattgggttcagagacaactttattgggctcatgtattacaacagtactggcaatatgggggtgctggctggtcaggcctcacagctgaatgctgtggtcgacttgcaagacagaaacaccgagctgtcataccagctcttgcttgactctttgggtgacagaacccggtatttcagtatgtggaatcaggcggtggacagttatgatcctgatgtgcgcattattgaaaaccatggtgtggaagacgaacttcccaactattgcttccccctggatgggtctggcactaatgccgcttaccaaggtgtgaaagtaaaaaatggtaacgatggtgatgttgagagcgaatgggaaaatgatgatactgtcgcagctcgaaatcaattatgcaagggcaacatttttgccatggaaattaacctccaagccaacctgtggagaagtttcctctactcgaacgtggccctgtacctgcccgactcttacaagtacacgccagccaacatcaccctgcccaccaacaccaacacttatgattacatgaacgggagagtggtgcctccctcgctggtggacgcctacatcaacatcggggcgcgctggtcgctggaccccatggacaacgtcaatcccttcaaccaccaccgcaacgcgggcctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgcctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggatacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccgtacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaaattgctacttgcatgatggctgagcccacaggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgtagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaacaatcaagacatgtaaaccgtgtgtgtatgtttaaaatatcttttaataaacagcactttaatgttacacatgcatctgagatgattttattttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagctgcagggcgcccagcaggtcgggcgcggagatcttgaaatcgcagttgggacccgcgttctgcgcgcgagagttgcggtacacggggttgcagcactggaacaccatcagggccgggtgcttcacgctcgccagcaccgccgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcacagccggcatcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagtttgccctcggcctcggtgcacccgtgcagccacagcgcgcacccggtgcactcccagttcttgtgggcgatctgggaatgcgcgtgcacgaacccttgcaggaagcggcccatcatggtcgtcagggtcttgttgctagtgaaggtcaacgggatgccgcggtgctcctcgttgatgtacaggtggcagatgcggcggtacacctcgccctgctcgggcatcagttggaagttggctttcaggtcggtctccacgcggtagcggtccatcagcatagtcatgatttccatgcccttctcccaggccgagacgatgggcaggctcatagggttcttcaccatcatcttagcactagcagccgcggccagggggtcgctctcatccagggtctcaaagctccgcttgccgtccttctcggtgatccgcaccggggggtagctgaagcccacggccgccagctcctcctcggcctgtctttcgtcctcgctgtcctggctgacgtcctgcatgaccacatgcttggtcttgcggggtttcttcttgggcggcagtggcggcggagatgcttgtggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccggcgacgagaagcagcagcagcagaatgaaagcttaaccgccccgccgcccagccccgcctccgacgcagccgcggtcccagacatgcaagagatggaggaatccatcgagattgacctgggctatgtgacgcccgcggagcatgaggaggagctggcagtgcgctttcaatcgtcaagccaggaagataaagaacagccagagcaggaagcagagaacgagcagagtcaggctgggctcgagcatggcgactacctccacctgagcggggaggaggacgcgctcatcaagcatctggcccggcaggccaccatcgtcaaggacgcgctgctcgaccgcaccgaggtgcccctcagcgtggaggagctcagccgcgcctacgagctcaacctcttctcgccgcgcgtgccccccaagcgccagcccaacggcacctgcgagcccaacccccgcctcaacttctacccggtcttcgcggtgcccgaggccctggccacctaccacatctttttcaagaaccaaaagatccccgtctcctgccgcgccaaccgcacccgcgccgacgccctcttcaacctgggtcccggcgcccgcctacctgatatcgcctccttggaagaggttcccaagatcttcgagggtctgggcagcgacgagactcgggccgcgaacgctctgcaaggagaaggaggaggagagcatgagcaccacagcgccctggtcgagttggaaggcgacaacgcgcggctggcggtgctcaaacgcacggtcgagctgacccatttcgcctacccggctctgaacctgcccccgaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctacccctcaaagtttggaagagcggcgcaagctcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccgccgcgactacatccgcgactgcgtctacctctacctctgccacacctggcagacgggcatgggcgtgtggcagcagtgtctggaggagcagaacctgaaagagctctgcaagctcctgcaaaagaacctcaagggtctgtggaccgggttcgacgagcggaccaccgcctcggacctggccgacctcatcttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgagggcgagggagccaaggggggtctgaaactcaccccggggctgtggacctcggcctacttgcgcaagttcgtgcccgaggattaccatcccttcgagatcaggttctacgaggaccaatcccagccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcggccccacagtagatgggacgagaccgggcgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaacttcccccgcaacatcttgcattactaccgtcacctccacagcccctactacttccaagaagaggcagcagcagcagaaaaagaccagaaaaccagctagaaaatccacagcggcggcagcggcaggtggactgaggatcgcggcgaacgagccggcgcagacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccagcaccgccatgagcaaagagattcccacgccttacatgtggagctaccagccccagatgggcctggccgccggcgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggcggtcctgactttggagagttcgtcctcgcagccccgctcgggcggcatcggcactctccagttcgtggaggagttcactccctcggtctacttcaaccccttctccggctcccccggccactacccggacgagttcatcccgaactttgacgccatcagcgagtcggtggacggctacgattgattaattaatcaactaaccccttacccctttaccctccagtaaaaataaagattaaaaatgattgaattgatcaataaagaatcacttacttgaaatctgaaaccaggtctctgtccatgttttctgtcagcagcacttcactcccctcttcccaactctggtactgcaggccccggcgggctgcaaacttcctccacactctgaaggggatgtcaaattcctcctgtccctcaatcttcatttttatcttctatcagatgtccaaaaagcgcgcgcgggtggatgatggcttcgaccccgtgtacccctacgatgcagacaacgcaccgactgtgcccttcatcaaccctcccttcgtctcttcagatggattccaagaaaagcccctgggggtgttgtccctgcgactggccgaccccgtcaccaccaagaatggggctgtcaccctcaagctgggggagggggtggacctcgacgactcgggaaaactcatctccaaaaatgccaccaaggccactgcccctctcagtatttccaacggcaccatttcccttaacatggctgcccctttttacaacaacaatggaacgttaagtctcaatgtttctacaccattagcagtatttcccacttttaacactttaggtatcagtcttggaaacggtcttcaaacttctaataagttgctgactgtacagttaactcatcctcttacattcagctcaaatagcatcacagtaaaaacagacaaaggactctatattaattctagtggaaacagagggcttgaggctaacataagcctaaaaagaggactgatttttgatggtaatgctattgcaacataccttggaagtggtttagactatggatcctatgatagcgatgggaaaacaagacccatcatcaccaaaattggagcaggtttgaattttgatgctaataatgccatggctgtgaagctaggcacaggtttaagttttgactctgccggtgccttaacagctggaaacaaagaggatgacaagctaacactttggactacacctgacccaagccctaattgtcaattactttcagacagagatgccaaatttaccctatgtcttacaaaatgcggtagtcaaatactaggcactgttgcagtagctgctgttactgtaggttcagcactaaatccaattaatgacacagtaaaaagcgccatagtattccttagatttgactctgacggtgtgctcatgtcaaactcatcaatggtaggtgattactggaactttagggaaggacagaccacccaaagtgtggcctatacaaatgctgtgggattcatgcccaatctaggtgcatatcctaaaacccaaagcaaaacaccaaaaaatagtatagtaagtcaggtatatttaaatggagaaactactatgccaatgacactgacaataactttcaatggcactgatgaaaaagacacaacacctgtgagcacttactccatgacttttacatggcagtggactggagactataaggacaagaatattacctttgctaccaactcctttactttctcctacatggcccaagaataaaccctgcatgccaaccccattgttcccaccactatggaaaactctgaagcagaaaaaaataaagttcaagtgttttattgattcaacagttttctcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgattaactttattccaaacggtctcggagcacttcaaaatgcaggtcccggaggtggcacctctcgcccccactgtgttggtggaaaataacagccaggtcaaaggtgacacggttctcgagatgttccacggtggcttccagcaaagcctccacgcgcacatccagaaacaagaggacagcgaaagcgggagcgttttctaattcctcaatcatcatattacactcctgcaccatccccagataattttcatttttccagccttgaatgattcgtattagttcctgaggtaaatccaagccagccatgataaaaagctcgcgcagagcgccctccaccggcattcttaagcacaccctcataattccaagagattctgctcctggttcacctgcagcagattaacaatgggaatatcaaaatctctgccgcgatccctaagctcctccctcaacaataactgtatgtaatctttcatatcatctccgaaatttttagccatagggccgccaggaataagagcagggcaagccacattacagataaagcgaagtcctccccagtgwgcattgccaaatgtaagattgaaataagcatgctggctagaccctgtgatatcttccagataactggacagaaaatcaggcaagcaatttttaagaaaatcaacaaaagaaaagtcgtccaggtgcaggtttagagcctcaggaacaacgatggaataagtgcaaggagtgcgttccagcatggttagtgtttttttggtgatctgtagaacaaaaaataaacatgcaatattaaaccatgctagcctggcgaacaggtgggtaaatcactctttccagcaccaggcaggctacggggtctccggcgcgaccctcgtagaagctgtcgccatgattgaaaagcatcaccgagagaccttcccggtggccggcatggatgattcgagaagaagcatacactccgggaacattggcatccgtgagtgaaaaaaagcgacctataaagcctcggggcactacaatgctcaatctcaattccagcaaagccaccccatgcggatggagcacaaaattggcaggtgcgtaaaaaatgtaattactcccctcctgcacaggcagcaaagcccccgctccctccagaaacacatacaaagcctcagcgtccatagcttaccgagcacggcaggcgcaagagtcagagaaaaggctgagctctaacctgactgcccgctcctgtgctcaatatatagccctaacctacactgacgtaaaggccaaagtctaaaaatacccgccaaaatgacacacacgcccagcacacgcccagaaaccggtgacacactcaaaaaaatacgtgcgcttcctcaaacgcccaaaccggcgtcatttccgggttcccacgctacgtcaccgctcagcgactttcaaattccgtcgaccgttaaaaacgtcactcgccccgcccctaacggtcgcccttctctcggccaatcaccttcctcccttcccaaattcaaacgcctcatttgcatattaacgcgcacaaaaagtttgaggtatatatttgaatgatggtttaaacgcggccgccaggcctacccactagtcaattcgggaggatcgaaacggcagatcgcaaaaaacagtacatacagaaggagacatgaacatgaacatcaaaaaaattgtaaaacaagccacagttctgacttttacgactgcacttctggcaggaggagcgactcaagccttcgcgaaagaaaataaccaaaaagcatacaaagaaacgtacggcgtctctcatattacacgccatgatatgctgcagatccctaaacagcagcaaaacgaaaaataccaagtgcctcaattcgatcaatcaacgattaaaaataattgagtctgacaaaaggacttgatgtg

SEQ ID NO: 41 - DNA, 2661 bp, synthetic construct
  misc_feature 1..2661: note = Vector
  source 1..2661: mol_type = other DNA
ttaattaaccatcttcaataatatacctcaaactttttgtgcgcgttaatatgcaaatgaggcgtttgaatttggggaggaagggcggtgattggtcgagggatgagcgaccgttaggggcggggcgagtgacgttttgatgacgtggttgcgaggaggagccagtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtgtttttcacctaaatttccgcgtacggtgtcaaagtccggtgtttttacgcgatcgctagcgacatcgatcacaagtttgtacaaaaaagcaggctccaccatgggaacccgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttggtacc

SEQ ID NO: 42 - DNA, 37490 bp, synthetic construct
  misc_feature 1..37490: note = Vector
  source 1..37490: mol_type = other DNA
gcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatcgatcccgtcgagatatctagacccagctttcttgtacaaagtggtgatcgattcgacagatcgcgatcgcaagtgagtagtgttctggggcgggggaggacctgcatgagggccagaataactgaaatctgtgcttttctgtgtgttgcagcagcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcgttggacgcagctgccgccgcagctgctgcatctgccgccagcgccgtgcgcggaatggccatgggcgccggctactacggcactctggtggccaactcgagttccaccaataatcccgccagcctgaacgaggagaagctgttgctgctgatggcccagctcgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcctttgtgcccgcccaggttttccatgcactcatccatgatgatggcgatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcggagggggggatcatgtccacctgcggggcgataaagaacacggtttccggggcgggggagatgagctgggccgaaagcaagttccggagcagctgggacttgccgcagccggtggggccgtagatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagggataggagctcctggagcgaggcgaagtttttcagcggcttgagtccgtcggccatgggcattttggagagggtttgttgcaagagttccaggcggtcccagagctcggtgatgtgctctacggcatctcgatccagcagacctcctcgtttcgcgggttgggacggctgcgggagtagggcaccagacgatgggcgtccagcgcagccagggtccggtccttccagggtcgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggactcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccaccttttccagggtatgcaaacacatgtccccctcgtccacatccaggaaggtgattggcttgtaagtgtaggccacgtgaccgggggtcccggccgggggggtataaaagggtgcgggtccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccggcggagatgcctttcaagagcccctcgtccatctggtcagaaaagacgatctttttgttgtcgagcttggtggcgaaggagccgtagagggcgttggagaggagcttggcgatggagcgcatggtctggtttttttccttgtcggcgcgctccttggcggcgatgttgagctgcacgtactcgcgcgccacgcacttccattcggggaagacggtggtcagctcgtcgggcacgattctgacctgccagccccgattatgcagggtgatgaggtccacactggtggccacctcgccgcgcaggggctcattagtccagcagaggcgtccgcccttgcgcgagcagaaggggggcagggggtccagcatgacctcgtcgggggggtcggcatcgatggtgaagatgccgggcaggaggtcggggtcaaagtagctgatggaagtggccagatcgtccagggcagcttgccattcgcgcacggccagcgcgcgctcgtagggactgaggggcgtgccccagggcatgggatgggtaagcgcggaggcgtacatgccgcagatgtcgtagacgtagaggggctcctcgaggatgccgatgtaggtggggtagcagcgccccccgcggatgctggcgcgcacgtagtcatacagctcgtgcgagggggcgaggagccccgggcccaggttggtgcgactgggcttttcggcgcggtagacgatctggcggaaaatggcatgcgagttggaggagatggtgggcctttggaagatgttgaagtgggcgtggggcagtccgaccgagtcgcggatgaagtgggcgtaggagtcttgcagcttggcgacgagctcggcggtgactaggacgtccagagcgcagtagtcgagggtctcctggatgatgtcatacttgagctgtcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaagtgtccctgaccatgaccttgaggaactggtgcttgaagtcgatatcgtcgcagcccccctgctcccagagctggaagtccgtgcgcttcttgtaggcggggttgggcaaagcgaaagtaacatcgttgaagaggatcttgcccgcgcggggcataaagttgcgagtgatgcggaaaggttggggcacctcggcccggttgttgatgacctgggcggcgagcacgatctcgtcgaagccgttgatgttgtggcccacgatgtagagttccacgaatcgcggacggcccttgacgtggggcagtttcttgagctcctcgtaggtgagctcgtcggggtcgctgagcccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcgatcccatttgagctggagggcgagatcgagggcgagctcgacgagccggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccattggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcaggggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcttgaaggtctccgcggccggcgcgctccacggtggccgcgaggtcgttggagatgcggcccatgagctgcgagaaggcgttcatgcccgcctcgttccagacgcggctgtagaccacgacgccctcgggatcgccggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaaacgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcttcttcctcctccactaacatctcttctacttcctcctcaggcggcagtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgctgaacgaaggcttcgagccagtcgcagtcgcaaggtaggctgagcacggtttcttctggcgggtcatgttggttgggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcttgctggatctgggtgagggtggtctggaagtcatcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtattggcactcccgtctcgacccaagcctgcaccaaccctccaggatacggaggcgggtcgttttgcaacttttttttggaggccggatgagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctccacagccggcgcttctgcccccgccccagcagcaacttccagccacgaccgccgcggccgccgtgagcggggctggacagagttatgatcaccagctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaagcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtttgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgtgcggtgagcgccagcaggcggcgcgagctgagcgaccaggagctgatgcatagtctgcagcgggccctgaccggggccgggaccgagggggagagctactttgacatgggcgcggacctgcactggcagcccagccgccgggccttggaggcggcggcaggaccctacgtagaagaggtggacgatgaggtggacgaggagggcgagtacctggaagactgatggcgcgaccgtatttttgctagatgcaacaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggctttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaacctggaagccaccctgaactttttgctgaccaaccggtcgcagaagatcccgccccagtacgcgctcagcaccgaggaggagcgcatcctgcgttacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtatccgcagcagcgagctgggcaggatcacgcgcccgcgcttgctgggcgaagaggagtacttgaatgactcgctgttgagacccgagcgggagaagaacttccccaataacgggatagaaagcctggtggacaagatgagccgctggaagacgtatgcgcaggagcacagggacgatccccgggcgtcgcagggggccacgagccggggcagcgccgcccgtaaacgccggtggcacgacaggcagcggggacagatgtgggacgatgaggactccgccgacgacagcagcgtgttggacttgggtgggagtggtaacccgttcgctcacctgcgcccccgtatcgggcgcatgatgtaagagaaaccgaaaataaatgatactcaccaaggccatggcgaccagcgtgcgttcgtttcttctctgttgttgttgtatctagtatgatgaggcgtgcgtacccggagggtcctcctccctcgtacgagagcgtgatgcagcaggcgatggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcccgcaagacccccaatggggtgacagtgacagaggattatgatggtagtcaggatgagctgaagtatgaatgggtggaatttgagctgcccgaaggcaacttctcggtgaccatgaccatcgacctgatgaacaacgccatcatcgacaattacttggcggtggggcggcagaacggggtgctggagagcgacatcggcgtgaagttcgacactaggaacttcaggctgggctgggaccccgtgaccgagctggtcatgcccggggtgtacaccaacgaggctttccatcccgatattgtcttgctgcccggctgcggggtggacttcaccgagagccgcctcagcaacctgctgggcattcgcaagaggcagcccttccaggaaggcttccagatcatgtacgaggatctggaggggggcaacatccccgcgctcctggatgtcgacgcctatgagaaaagcaaggaggatgcagcagctgaagcaactgcagccgtagctaccgcctctaccgaggtcaggggcgataattttgcaagcgccgcagcagtggcagcggccgaggcggctgaaaccgaaagtaagatagtcattcagccggtggagaaggatagcaagaacaggagctacaacgtactaccggacaagataaacaccgcctaccgcagctggtacctagcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgcttacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggcggacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccctgcggtggtgaaggaggaaagaaagccccgcaaaatcaagcgggtcaaaaaggacaaaaaggaagaagaaagtgatgtggacggattggtggagtttgtgcgcgagttcgccccccggcggcgcgtgcagtggcgcgggcggaaggtgcaaccggtgctgagacccggcaccaccgtggtcttcacgcccggcgagcgctccggcaccgcttccaagcgctcctacgacgaggtgtacggggatgatgatattctggagcaggcggccgagcgcctgggcgagtttgcttacggcaagcgcagccgttccgcaccgaaggaagaggcggtgtccatcccgctggaccacggcaaccccacgccgagcctcaagcccgtgaccttgcagcaggtgctgccgaccgcggcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagattcccacggagcccatggaaacgcagaccgagcccatgatcaagcccagcaccagcaccatggaggtgcagacggatccctggatgccatcggctcctagtcgaagaccccggcgcaagtacggcgcggccagcctgctgatgcccaactacgcgctgcatccttccatcatccccacgccgggctaccgcggcacgcgcttctaccgcggtcataccagcagccgccgccgcaagaccaccactcgccgccgccgtcgccgcaccgccgctgcaaccacccctgccgccctggtgcggagagtgtaccgccgcggccgcgcacctctgaccctgccgcgcgcgcgctaccacccgagcatcgccatttaaactttcgccagctttgcagatcaatggccctcacatgccgccttcgcgttcccattacgggctaccgaggaagaaaaccgcgccgtagaaggctggcggggaacgggatgcgtcgccaccaccaccggcggcggcgcgccatcagcaagcggttggggggaggcttcctgcccgcgctgatccccatcatcgccgcggcgatcggggcgatccccggcattgcttccgtggcggtgcaggcctctcagcgccactgagacacacttggaaacatcttgtaataaacccatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgatgcggaggagacgctgctgacgcacacggacgagccgcccccgtacgaggaggcggtgaaactgggtctgcccaccacgcggcccatcgcgcccctggccaccggggtgctgaaacccgaaaagcccgcgaccctggacttgcctcctccccagccttcccgcccctctacagtggctaagcccctgccgccggtggccgtggcccgcgcgcgacccgggggcaccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgccgccgctgtccaccagaaggaggagtgaagaggcgcgtcgccgagttgcaagatggccaccccatcgatgctgccccagtgggcgtacatgcacatcgccggacaggacgcttcggagtacctgagtccgggtctggtgcagtttgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagtctggcccccaagggagcacccaacacttgtcagtggacatataaagccgatggtgaaactgccacagaaaaaacctatacatatggaaatgcacccgtgcagggcattaacatcacaaaagatggtattcaacttggaactgacaccgatgatcagccaatctacgcagataaaacctatcagcctgaacctcaagtgggtgatgctgaatggcatgacatcactggtactgatgaaaagtatggaggcagagctcttaagcctgataccaaaatgaagccttgttatggttcttttgccaagcctactaataaagaaggaggtcaggcaaatgtgaaaacaggaacaggcactactaaagaatatgacatagacatggctttctttgacaacagaagtgcggctgctgctggcctagctccagaaattgttttgtatactgaaaatgtggatttggaaactccagatacccatattgtatacaaagcaggcacagatgacagcagctcttctattaatttgggtcagcaagccatgcccaacagacctaactacattggtttcagagacaactttatcgggctcatgtactacaacagcactggcaatatgggggtgctggccggtcaggcttctcagctgaatgctgtggttgacttgcaagacagaaacaccgagctgtcctaccagctcttgcttgactctctgggtgacagaacccggtatttcagtatgtggaatcaggcggtggacagctatgatcctgatgtgcgcattattgaaaatcatggtgtggaggatgaacttcccaactattgtttccctctggatgctgttggcagaacagatacttatcagggaattaaggctaatggaactgatcaaaccacatggaccaaagatgacagtgtcaatgatgctaatgagataggcaagggtaatccattcgccatggaaatcaacatccaagccaacctgtggaggaacttcctctacgccaacgtggccctgtacctgcccgactcttacaagtacacgccggccaatgttaccctgcccaccaacaccaacacctacgattacatgaacggccgggtggtggcgccctcgctggtggactcctacatcaacatcggggcgcgctggtcgctggatcccatggacaacgtgaaccccttcaaccaccaccgcaatgcggggctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgtctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggctacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaagctcttgcttcttgcaagccatggccgcgggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaatcaagacatgtaaaccgtgtgtgtatgttaaatgtctttaataaacagcactttcatgttacacatgcatctgagatgatttatttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagttgcagggcgcccagcaggtcgggcgcggagatcttgaaatcgcagttgggacccgcgttctgcgcgcgggagttgcggtacacggggttgcagcactggaacaccatcagggccgggtgcttcacgctcgccagcaccgtcgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcgcacccggcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagcttgccctcggcctcggtgcacccgtgcagccacagcgcgcacccggtgcactcccagttcttgtgggcgatctgggaatgcgcgtgcacgaagccctgcaggaagcggcccatcatggtggtcagggtcttgttgctagtgaaggtcagcggaatgccgcggtgctcctcgttgatgtacaggtggcagatgcggcggtacacctcgccctgctcgggcatcagctggaagttggctttcaggtcggtctccacgcggtagcggtccatcagcatagtcatgatttccatacccttctcccaggccgagacgatgggcaggctcatagggttcttcaccatcatcttagcgctagcagccgcggccagggggtcgctctcgtccagggtctcaaagctccgcttgccgtccttctcggtgatccgcaccggggggtagctgaagcccacggccgccagctcctcctcggcctgtctttcgtcctcgctgtcctggctgacgtcctgcaggaccacatgcttggtcttgcggggtttcttcttgggcggcagcggcggcggagatgttggagatggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccgacgagaagcagcagcagcagaatgaaagcttaaccgccccgccgcccagccccgccacctccgacgcggccgtcccagacatgcaagagatggaggaatccatcgagattgacctgggctatgtgacgcccgcggagcacgaggaggagctggcagtgcgcttttcacaagaagagatacaccaagaacagccagagcaggaagcagagaatgagcagagtcaggctgggctcgagcatgacggcgactacctccacctgagcgggggggaggacgcgctcatcaagcatctggcccggcaggccaccatcgtcaaggatgcgctgctcgaccgcaccgaggtgcccctcagcgtggaggagctcagccgcgcctacgagttgaacctcttctcgccgcgcgtgccccccaagcgccagcccaatggcacctgcgagcccaacccgcgcctcaacttctacccggtcttcgcggtgcccgaggccctggccacctaccacatctttttcaagaaccaaaagatccccgtctcctgccgcgccaaccgcacccgcgccgacgcccttttcaacctgggtcccggcgcccgcctacctgatatcgcctccttggaagaggttcccaagatcttcgagggtctgggcagcgacgagactcgggccgcgaacgctctgcaaggagaaggaggagagcatgagcaccacagcgccctggtcgagttggaaggcgacaacgcgcggctggcggtgctcaaacgcacggtcgagctgacccatttcgcctacccggctctgaacctgccccccaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctagtccccagagtttggaagagcggcgcaaactcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccggcgcgactacatccgcgactgcgtctacctctacctctgccacacctggcagacgggcatgggcgtgtggcagcagtgtctggaggagcagaacctgaaagagctctgcaagctcctgcagaagaacctcaagggtctgtggaccgggttcgacgagcgcaccaccgcctcggacctggccgacctcattttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgaaggcgagggttcagccgccaaggggggtctgaaactcaccccggggctgtggacctcggcctacttgcgcaagttcgtgcccgaggactaccatcccttcgagatcaggttctacgaggaccaatcccatccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggaggaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcgaccacacagtagatgggacgagaccggacgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaactttccccgcaacatcttgcattactaccgtcacctccacagcccctactacttccaagaagaggcagcagcagcagaaaaagaccagcagaaaaccagcagctagaaaatccacagcggcggcagcaggtggactgaggatcgcggcgaacgagccggcgcaaacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccatcatcatgagcaaagagattcccacgccttacatgtggagctaccagccccagatgggcctggccgccggtgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgagtttaaactcacccccttatccagtgaaataaagatcatattgatgatgattttacagaaataaaaaataatcatttgatttgaaataaagatacaatcatattgatgatttgagtttaacaaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgattcctcgggaaaactcatctccaacacggccaccaaggccgccgcccctctcagtttttccaacaacaccatttcccttaacatggatcaccccttttacactaaagatggaaaattatccttacaagtttctccaccattaaatatactgagaacaagcattctaaacacactagctttaggttttggatcaggtttaggactccgtggctctgccttggcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaatggagatgtttcaaaacctatgcttctcactataaccctcaatggtactgatgacagcaacagtacatattcaatgtcattttcatacacctggactaatggaagctatgttggagcaacatttggggctaactcttataccttctcatacatcgcccaagaatgaacactgtatcccaccctgcatgccaacccttcccaccccactctgtggaacaaactctgaaacacaaaataaaataaagttcaagtgttttattgattcaacagtttcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgattaactttattccaaacggtctcggagcacttcaaaatgcaggtcccggaggtggcacctctcgcccccactgtgttggtggaaaataacagccaggtcaaaggtgacacggttctcgagatgttccacggtggcttccagcaaagcctccacgcgcacatccagaaacaagaggacagcgaaagcgggagcgttttctaattcctcaatcatcatattacactcctgcaccatccccagataattttcatttttccagccttgaatgattcgtattagttcctgaggtaaatccaagccagccatgataaaaagctcgcgcagagcgccctccaccggcattcttaagcacaccctcataattccaagagattctgctcctggttcacctgcagcagattaacaatgggaatatcaaaatctctgccgcgatccctaagctcctccctcaacaataactgtatgtaatctttcatatcatctccgaaatttttagccatagggccgccaggaataagagcagggcaagccacattacagataaagcgaagtcctccccagtgwgcattgccaaatgtaagattgaaataagcatgctggctagaccctgtgatatcttccagataactggacagaaaatcaggcaagcaatttttaagaaaatcaacaaaagaaaagtcgtccaggtgcaggtttagagcctcaggaacaacgatggaataagtgcaaggagtgcgttccagcatggttagtgtttttttggtgatctgtagaacaaaaaataaacatgcaatattaaaccatgctagcctggcgaacaggtgggtaaatcactctttccagcaccaggcaggctacggggtctccggcgcgaccctcgtagaagctgtcgccatgattgaaaagcatcaccgagagaccttcccggtggccggcatggatgattcgagaagaagcatacactccgggaacattggcatccgtgagtgaaaaaaagcgacctataaagcctcggggcactacaatgctcaatctcaattccagcaaagccaccccatgcggatggagcacaaaattggcaggtgcgtaaaaaatgtaattactcccctcctgcacaggcagcaaagcccccgctccctccagaaacacatacaaagcctcagcgtccatagcttaccgagcacggcaggcgcaagagtcagagaaaaggctgagctctaacctgactgcccgctcctgtgctcaatatatagccctaacctacactgacgtaaaggccaaagtctaaaaatacccgccaaataatcacacacgcccagcacacgcccagaaaccggtgacacactcaaaaaaatacgcgcacttcctcaaacgcccaaaactgccgtcatttccgggttcccacgctacgtcatcaaaacacgactttcaaattccgtcgaccgttaaaaacgtcacccgccccgcccctaacggtcgcccgtctctcagccaatcagcgccccgcatccccaaattcaaacacctcatttgcatattaacgcgcacaaaaagtttgaggtatattattgatgatggttaattaaggatccttctatagtgtcacctaaatgtcgacggccaggcggccgccaggcctacccactagtcaattcgggaggatcgaaacggcagatcgcaaaaaacagtacatacagaaggagacatgaacatgaacatcaaaaaaattgtaaaacaagccacagttctgacttttacgactgcacttctggcaggaggagcgactcaagccttcgcgaaagaaaataaccaaaaagcatacaaagaaacgtacggcgtctctcatattacacgccatgatatgctgcagatccctaaacagcagcaaaacgaaaaataccaagtgcctcaattcgatcaatcaacgattaaaaatattgagtctgcaaaaggacttgatgtgtgggacagctggccgctgcaaaacgctgacggaacagtagctgaatacaacggctatcacgttgtgtttgctcttgcgggaagcccgaaagacgctgatgacacatcaatctacatgttttatcaaaaggtcggcgacaactcaatcgacagctggaaaaacgcgggccgtgtctttaaagacagcgataagttcgacgccaacgatccgatcctgaaagatcagacgcaagaatggtccggttctgcaacctttacatctgacggaaaaatccgtttattctacactgactattccggtaaacattacggcaaacaaagcctgacaacagcgcaggtaaatgtgtcaaaatctgatgacacactcaaaatcaacggagtggaagatcacaaaacgatttttgacggagacggaaaaacatatcagaacgttcagcagtttatcgatgaaggcaattatacatccggcgacaaccatacgctgagagaccctcactacgttgaagacaaaggccataaataccttgtattcgaagccaacacgggaacagaaaacggataccaaggcgaagaatctttatttaacaaagcgtactacggcggcggcacgaacttcttccgtaaagaaagccagaagcttcagcagagcgctaaaaaacgcgatgctgagttagcgaacggcgccctcggtatcatagagttaaataatgattacacattgaaaaaagtaatgaagccgctgatcacttcaaacacggtaactgatgaaatcgagcgcgcgaatgttttcaaaatgaacggcaaatggtacttgttcactgattcacgcggttcaaaaatgacgatcgatggtattaactcaaacgatatttacatgcttggttatgtatcaaactctttaaccggcccttacaagccgctgaacaaaacagggcttgtgctgcaaatgggtcttgatccaaacgatgtgacattcacttactctcacttcgcagtgccgcaagccaaaggcaacaatgtggttatcacaagctacatgacaaacagaggcttcttcgaggataaaaaggcaacatttgcgccaagcttcttaatgaacatcaaaggcaataaaacatccgttgtcaaaaacagcatcctggagcaaggacagctgacagtcaactaataacagcaaaaagaaaatgccgatacttcattggcattttcttttatttctcaacaagatggtgaattgactagtgggtagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattgatccggaacccttaatataacttcgtataatgtatgctatacgaagttattaggtccctcgactatagggtcaccgtcgacagcgacacacttgcatcggatgcagcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagggcttcgccctgtcgctcaactgcggcgagcactactggctgtaaaaggacagaccacatcatggttctgtgttcattaggttgttctgtccattgctgacataatccgctccacttcaacgtaacaccgcacgaagatttctattgttcctgaaggcatattcaaatcgttttcgttaccgcttgcaggcatcatgacagaacactacttcctataaacgctacacaggctcctgagattaataatgcggatctctacgataatgggagattttcccgactgtttcgttcgcttctcagtggataacagccagcttctctgtttaacagacaaaaacagcatatccactcagttccacatttccatataaaggccaaggcatttattctcaggataattgtttcagcatcgcaaccgcatcagactccggcatcgcaaactgcacccggtgccgggcagccacatccagcgcaaaaaccttcgtgtagacttccgttgaactgatggacttatgtcccatcaggctttgcagaactttcagcggtataccggcatacagcatgtgcatcgcataggaatggcggaacgtatgtggtgtgaccggaacagagaacgtcacaccgtcagcagcagcggcggcaaccgcctccccaatccaggtcctgaccgttctgtccgtcacttcccagatccgcgctttctctgtccttcctgtgcgacggttacgccgctccatgagcttatcgcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacatgagaattggtcgacggcgcgccaaagcttgcatgcctgcagccgcgtaacctggcaaaatcggttacggttgagtaataaatggatgccctgcgtaagcggggcacatttcattacctctttctccgcacccgacatagataataacttcgtatagtatacattatacgaagttatctagtagac

SEQ ID NO: 43 - protein, 24 aa, Foot-and-mouth disease virus
APVKQTLNFDLLKLAGDVESNPGP

SEQ ID NO: 44 - DNA, 143055 bp, synthetic construct
  misc_feature 1..143055: note = Vector
  source 1..143055: mol_type = other DNA
tatttatttagtgtctagaaaaaaatgtgtgaccaacgaccgtaggaaactctagagggtaagaaaaatcaatcgctttatagagaccatcagaaagaggtttaatatttttgtgagaccatcgaaggagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaaggagaaagagataaaacttttttacgactccatcagaaagaggtttaatatttttgtgagaccatcgaaggagaaagagataaaacttttttacgactccatcagaaagaggtttaatatttttgtgagaccatcgaaggagaaagagataaaacttttttacgactccatcagaaagaggtttaatatttttgtgagaccatcgaagagagaaagagataaaacttttttacgactccatcagaaagaggtttaatatttttgtgagaccatcgaaggagaaagagataaaacttttttacgactccatcagaaagaggtttaatatttttgtgagaccatcgaagagagaaagagaataaaaatattttagtgacaccatcagaaagaggtttaatatttttgtgagaccatcgaagagagaaagagataaaacttttttacgactccatcagaaagaggtttaatatttttgtgagaccatcgaaggagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaccatcgaagagagaaagagaaagagatagttagtctagatatttttcttagtacaaaagtcaatgttttaaaatatatggacaagaatttgtctgtataaaaacttgtgtgaaattttgtaccaaagaaaaaatgtgagcagtatcccctacatggattttactagatcatttatataccaaaaaatattatacgatctacgttttattatatgattttaacgtgtaaattataaacattattttatgatatacaattgtctggtaacctagatgggcataggggatgagtatatgttgttggacgttattgtttaagaaatagttgatgcatcagaaagaggtttaatatttttgtgagaccatcgaagagagaaagagataaaacttttttacgactccatcagaaagaggtttaatatttttgtgagaccatcgaagagagaaagagataaaacttttttatgactccattgaagagagaatgagaataaaaatattttagtgacaccatcagaaagaggtttaatattttttatgagaccatcaaagagagaaagagaataaaaatattttatgactccattgaagagagaaagagaaaatgagaataaaaatattttagtgacaccatcagaaagaggtttaatattttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatattttatgactccattgaagagagaaagagaataaaaatattttagtgacaccatcagaaagaggtttaatatttttgtgagaccatcgaagagagaaagagaataaaaatattttatgactccattgaagagagaaagagaataaaaatattttagtgacaccatcagaaagaggtttaatattttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatattttatgactccattgaagagagaatgagaataaaaatattttagtgacaccatcagaaagaggtttaatatttttgtgagaccatcgaagagagaaagagaataaaaatattttatgactccattgaagagagaaagagaataaaaatattttagtgacaccatcagaaagaggtttaatattttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatattttatgactccattgaagagagaatgagaataaaaatattttagtgacaccatcagaaagaggtttaatatttttgtgagaccatcgaagagagaaagagaataaaaatattttatgactccattgaagagagaatgagaataaaaatattttagtgacaccatcagaaagaggtttaatattttttatgagaccatcaaagagagaaagagaataaaaatatttttgtaaaactttttttatgagaccatcaaagagagaaagagaataaaaatatttttgtaaaaattataaacattattttatgatatacaattgtctggtaacctagatgggcataggggatgttgataagctcgacgagtatatgttgttggacgttattgtttaagaaatagttgatgcatcagaaagagaataaaaaatattttagtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaaggagaaagagataaaacttttttacgactccatcagaaagaggtttaatatttttgtgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatattttatgactccattgaagagagaatgagaataaaaatattttagtgacaccatcagaaagaggtttaatatttttgtgagaccatcgaagagagaaagagaataaaaatattttatgactccattgaagagagaatgagaataaaaatattttagtgacaccatcagaaagaggtttaatattttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatattttatgactccattgaagagagaatgagaataaaaatattttagtgacaccatcagaaagaggtttaatatttttgtgagaccatcgaagagagaaagagaataaaaatattttatgactccattgaagagagaaagagaataaaaatattttagtgacaccatcagaaagaggtttaatattttttatgagaccatcaaagagagaaagagaataaaaatatttttgtaaaactttttttatgagaccatcaaagagagaaagagaataaaaatattttatgactccattgaagagagaatgagaataaaaatattttagtgacaccatcagaaagaggtttaatatttttgtgagaccatcgaagagagaaagagaataaaaatattttatgactccattgaagagagaatgagaataaaaatattttagtgacaccatcagaaagaggtttaatatttttgtgagaccatcgaagagagaaagagaataaaaatattttatgactccattgaagagagaatgagaataaaaatattttagtgacaccatcagaaagaggtttaatattttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtgagaccatcaaagagagaaagagaataaaaatatttttgtgagaccatcaaagagagaaagagaataaaaatatttttgtgagaccatcaaagagagaaagagaataaaaatattttatgactccattgaagagagaaagagaataaaaatattttagtgacaccatcagaaagaggtttaatatttttgtgagaccatcgaagagagaaagagaataaaaatattttatgactccattgaagagagaaagagaataaaaatattttagtgacaccatcagaaagaggtttaatattttttatgagaccatcaaagagagaaagagaataaaaatattttttatgagaccatcaaagagagaaagagaataaaaatattttatgagaccatcaaagagagaaagagaataaaaatattttttatgagaccatcaaagagagaaagagaataaaaatattttatgagaccatcaaagagagaaagagaataaaaatattttttatgagaccatcaaagagagaaagagaataaaaatatttttgtatgagaccatcagaaagaggtttaatatttttgtgataccctgaaaggaaataggaatagtgtcataatcgtatcacactattgagacagaaaaagaagaagtcgcgagaggtaactttttgttttgcaaaccggaatatagtgtccggtacacttttttaattcgtggtgtgcctgaatcgttcgattaaccctactcatccaatttcagatgaatagagttatcgattcagacacacgctttgagttttgttgaatcgatgagtgaagtatcatcggttgcaccttcagatgccgatccgtcgacatacttgacctcaagttcagatgattccttgcacatgtctccgatacgaacgctaaactctagattcttgacacattttgtatcgacgatcgttgaaccgatgatatcttcgtaactcactttcttatgagagatgttagacccgagtactggatgggtcttgatgtcgctgtctttctcttcttcgctacatctgatgtcgatagacacctcacagtctttccatcagcggattctgagatggatttaatctgaggacatttggtgaatccaaagttcattctcagacctccaccgatgatggagtaataagtggtaggaggatctacatcctcgactgattccacctcgggatctggatctgactcggactctgtaatttccgttacggattggcaaatcttatcatcggtcggtgtttggtcttgctttgtgactttgataataacatcgattcccatatgatgtttgttttcttcttccgtacacgatgaggatgattgctgaagactggcaggcacatgcatgccagtacgatatattgtttcatgattgctattgattgagtactgttctttatgattctacttccttaccgtgcaataaattagaatatattttctacttttacgagaaattaattattgtatttatgggtgaaaaacttactataaaaagcgggtgggtttggaattagtgatcagtttatgtatatcgcaactaccgggcatatggctacattacccacatgataagagattgtatcagtttcgtagtcttgagtattggtattactatatagtatatagatgtcgacgctagagttactgtctccgaatgcggcatgatagtatcattctttgctttcgttaactgtttggaggaagaatctttgttattgcatttaatctcgaaattcagagtgcacacctttctcctgtaaagaaacctgaagtcgctaccttattaagaagacgggatcgcagtctttatgattcatagtaatagttagttccgacgttgagatggattcgctgagaccggtagtggtcgtccgagtacacgatgtgtcgttaactggatacaggttaatttccacatcgatatagttaaaggtatttctgggtacgggttcgcatttatctgcggaagagacggtgtgagaatatgttccgagaccacacggagaacagatgacgtctccggatactccgtatcctattccacattttgtttgggaaacacatgccttgcatccatgatcgggagagcattcacagattctattgtgagtcgtgttacacgatcgcgtcgacattgttgacagaaacgtgaccttcattcttaccgtcgtccataaatacgttaggtatgtaccacatactgtcgcgaacgatgcgtccatctcataatgatttactttttcataattaaagatgtgaaagaaaaccgaacaatatatttttttagtaatgtttatgcgagacatataaaataaactccgtgtttatgatgccggtaaatgtttttatcatcttggacggaatcgattttgtaatatgccatggaaacaggacattatcactccatgataaattatttaatggagtcgatcctctcattgttctttgcgtatctcaatctgtggcgtttgcttcgtttaaataatatatcaaacatggagacgcctgatatgtaggcattcttcattctattaatgtctgctctatagcgctttagttccttatgacgaccggcgatatcatacttactttagaaggaaaatcatcatctaggattaaggcgtatctgatacaggcgaataatggttcaggatatagatagcgtatatctctattaaatgcgtcaatcatagtctctagagtgggatggtaactcagtaataaatcaactagcttctctttggtaactgcttttctggatggccgtattgattatcgagcgtgacactcgctccatattccaataaccgctttgcaaattgtatattattgacatcgaccgcgtaatatagtagagttatcgatcatatctatatcatccatgtacttgcttagtatatcaaatacatcttcataacagtgatacccgcaattattaaatctcgataatatcagaccgtacatacatagacggccattgttagatatgtgatttacagccgcgtgtccatattttccacgataaaccttacgacgtttacatcgacgagattattattaacaaagttgttgtccgtcgtcttatccaacatgcattgaatgataggtatacttaccatatcgccgtaatgtaagtagtttatcagtatggcttgtacgatggattcatcctgttgtctaaatctctttagaatgttatcgatgatgtagtggttatattctctggaatcgtacgaagtaatactacgcattacgtcgacaagagtatgacgtctctcaataagaagattaacgatttccatgtctacattatatggggttactctaaatcgcttgtttagataatacgcctctaatatagggctgacgtcgtatactctacacgtgtccacatcctttattaataatctctatatctatggttgagcaagaccagtagtattggatggaaacattgttatcgatcaaacatttaattacatccttggatagagattctctatgagacgatatatagtaatgaagagagttcttacacatatcactgttgtacatacaggtacgaaatacgtaaccggtgctgtaacattctgatttaagaagccatagcaatacttctggtctcggattaggcgtcgttacgtatatatccaccaatccgagaccattgattgcataattcgtattcttggacggacgtatccgtttatccacaattaggtattttagcagacgtaagtcgaaatcatttatattcgacttgagttcgttagaggaattcgaatagctggatatcagtagatgcacaatctgagattttacgtatctatgcttactgtatgctcctagcggagttaatccttcgttgtttctacaaagtctctcgactccgcgagagagtaacagtcgaacaatcttaatgtctgtatcgcatttattggagacgtaacaatgtagcgcattgtttcctcgtctatctatatgttttgataagttgtgacacgtttcaatttctagttttatttttttgtacgtcacatcttcatccagtagacgacatagaatacatgtgcaatccatagctattctggtgctaattattcctcataagatgataaaaagtgtagtgagagagcatgaaggagatttagtatttagcagtgcggatatgatccaagagggtgagatagtcgttctcgttcagaatctttcgcagcataagtagtatgtcgatatacttatcgttgaagactcttccagagacgatagctgattgagtacaaagtccaatgattgcacgaagttcttcggcggttttcatggagtcatttaatgatctccacggaagtgaatccttcaactcaccaccaaagagctccgttgcatcagttctgaaagagatgagaagcctgtagagagaccctgcgctttctctatgggtccatctatgagaaacccacaggatgtattcagtcagacaatgtctgacgtcggccacggtattcagggagtccttagtagcgtggcaatgacagggtctgaactgggcacaaggaaaggccattgtaaaggtagacctgtagccgtttatgctaatagagggctttaatttccattttttaatgggttgtggatgaggaatgagagtgatatcatattgagatacgtagttatgtagaggtgtatttcctatattatttactttcggtttcatattttaccaactctttaataaatttcttttcacgatgcatcttattaaatgacgttttctcataagtggacatatagatgcagaagtattacctctatcatctacataattagggtctgctcctaacttatacagtacgtagtagtagtttatcggttttaaatcaagtctagaatatatagtggattaatatatttttatattagctaaagcatatcattctcaacttcatcatgagttaaatatttgtgtctactagtttgtttatatcacagcattctacaaacagtctaaacaatagagaagacggacagactttaacgtataaatgacacatgttatcgatattcgttgataaatgattctaacgacatctctcgctagagataaaatctagtatcgtatcatactcgcatagcatagtttttcataattaatacaatatttaaaagacttattcggaaagtattttaatacatgtatcatcgatggagatccatatgaggagtcacttgtagttcttcagtagtgctatcatcgatagtataattatatgttgttgtaattggagtaactgttggtagttcttccgtggaatcaataattatactaacagcaatagtataattatataaatatgttccgttgatatcacatattttaatgaactcatttctaaagccgtacatccacatctattaggatctgatattttacacaactgtttaatagagtctacatttatatgttctctatcggtgagatacaaatacctagatagtcgcgttatagcacaaatacgatataaataataataataattattcagtaatgtatataaaaatgcattgtgtatttactccaatactactgtagttgtaagggttttttcaaaataatagttgtgtccacgacatttatatgtattacctatatattcttcagtaacattttcaaagtacaaggtcgcctcggtgataccgcctctactagttaaaacagagtatacatcaaaatcgaatcctataagccatccggatggattttcccattcaatcagtacatcgtcaatcaataatgacgttgacacagcagtgcatgttatattggcaggttctcctatcgttacgttgatttttggatctagtattagtttaaacctgtggtcttgcgacggtataaccgtaagtattttacatcttgattctaacgtcgtcgtaatgaacgtaacagttgtatcttccactatcttctaactctggattatgaataattaatttctttcccgtttgtgaatacttaatatcgtcgatattaatttccttattatctttataccaagttatattattataatgttttgcgtaaagaattccacagtataagtctatgccatacttatcatgagtacctagttcatatgtttttggaatgcatgaaggaggttttttaatatgagatctaactataccctgaacacagtcaccattctttgtagttacggtacacaaatacctacggttactgaatttagatgtatagttggctatccataaatcaccatgtttaacacgtttattagaaacctgtcgccgtctatttttttctagcctctcccatttaaccacatagtctttatatctgtgcgataaaagactgtcttcaataggaggacactttgcgctgaatggctctcctagagcggctatatcattcattgtgcctccgaacatacatgctggattcaaccatttagagtctttagctggtagagtatctctcattttattgaagaattctgtgatttcattttcgatgtctatggcgtaactgtggaatagcaataacaataatgatacgaaatatatatgtaccatcattttcatcgtcatcttcattatacggcactaaaatatttatataatatcagtttttttacacacatcgcatgagaaaaatacaactatactttggtaggtggatacgatatattataaagatcgttaattgtcagcatgtataatatttcgtatataatttctgaaggtagtaatgttagtagacaatttttatctgtgaaaacaggaaatagtttatcatatacctttgacacgtacatatctttataatatacattaaggtatctctcattcataatattagaatatattttaaaggacttatcgttattatatttttttaactcgttgaggtatcttcttaactttttcgaattatggcatcttaaaactgcatatagtgtagtgtctagaacagtggatagtttgagcaatattgtttctttatgacatttagttattataccatcgtatttatcaaacgacatgtattttgaaaatgcgtatcgatacgttttagaattgcgattcctaacgtgtctattgtaaaatgtgtcatctaaaagtatacagtaggatatgaatttctcaaatgattttatatcgaagataggtagagatggaagttgatccaccaacgatcttatcacattcttatctacaatatatctaagtatatacagtaaacaattaatggtatatggggaattttctttgttatctataatgagttgtgtagcgtgtttgttattgtcggttaacgcatttatatctccgttgatacgtataagataatctattacgtgtgtattattatattcacatgctacgtgtaataatgtctttccttcatagcgggtataaatatctatatttttaaacctattcatcaaataagttatcgaatctgccggacgtgtagaatatgttttgataaactcgaatacgattatacgacgctcatctatcggcatttctccaacatttgtttcatagtgatgtattaacataacaagaatatcgtgttgtatgtagtcggaagttatataacatagtataggcgttaatccatgattattacatattttgaaattcggattaaaagtcaacagcattttagtcatatgaatattattacacatttcaatactaagatacaaatggaatggtgtattaccacaattatctaacgcgtttagattagctccatgttgtataaacaaagaaataattcgcttggccttttttatcgttatcggttttctacactcacctggtttataaacgtgtgcgagacacaaataataataatgtaacgctgtatatccgtcttgtttaaagttaggatcgattcccttatctaataaagtggatactatgttctcatcgatatcctcttccttcaataacatgtaacgagattttatatactctagtagtaggttggaatagtctctatgcgataagtggtttttatctttgtctatcatatctatcactacatcatgtgaaatattacaacatctagtaagcaatatataaacaggcatacgtccgctactggttttcatcgttacatccactccatgatttaataatatcttcagtacatcgtttgtaaagtaattattatacaaatagcagtgtagtgcagtatacccagttacaggttgtcgtctgtttagatcatagtttttaactacaaaatcaagttctgtagaagtatggtctatattgcaatgactaaaatatgtatatatttcattctcttgtattttatgagttgattcgcggttgatatttaaaacataaatcagacgacgactcatttttatgatgctttgtggtaaaagtcctcatataattgtttaatattcattattatagacgattcccattaactaatctaacatctttgatatacccgtaaatatgtaaatatgatcctaaaataacacggattgtaagatgtctagaaagtttatgcaggtgtatgaatatgacagagagcaatatctcgatgagttcattgaagacagatataacgatagttttatcactagtccagaatactatagtgcggaaaaatacatgtgtagatatactacactaaatcacaattgtataaacgtacgacgatgcgcgttagactccaagttattacatgatatcataaccaattgtaaaatatataacaatatagaattagttagggcgacaaaatttgtttattatctggatctgataaaatgtaattgggtatctaaggtaggtgattcagttctatatcccgttatatttataacacatacaagcactagaaatttagataaagtctctgtaaaaacatacaagggcgttaaagtaaaaaaacttaatagatgcgcggatcatgctattgtaattaatccattcgtcaagtttaaactaacgttgccgaacaaaacaagtcatgcaaaggtattggttacattttgtaagttaagaacggatataacgcagatagaggcaccgctttcgggcaatgttttagtttatacatttcctgacattaataaaagaattcctggatatatacatgtcaacatagaaggatgtatcgatggaatgatttatataaattcttcaaagttcgcgtgtgttttaaaactacatagatcaatgtatcgcattccaccctttcctatagatatctgctcttgttgttcacaatatattaacgatgacatagaaattcccattcatgatttaataaaggatgtggcaatttttaaaaataaggagacggtatattatctaaaattaaataataaaactatagctagatttacgtactttaataatatagataccgcaattacacaagaacatgagtatgtcaaaatagcactaggtatagtctgcaagttaatgattaataatatgcatagtatcgtgggagttaatcatagcaatacgttcgtcaattgtttgttggaagataatgtataaaaattcttataaactcaattgacatggaaatgtaacaacatacattcacgttatactaacagtaactgttttgctgatgctaggaatagtaaacgccatacacgtaaacgttgtagcatcttcttccttgacaggattaatgtttaaccgggatgtaataacacgtctcttatcggtcatatagattttatttgctacacttattctaccgtctccgtccccatcatcttcttcgtaatacataccattacttatccaaaagacgtctgcatccgttgtgggaggtctcaacgatactctacacgcaatagtcaaattactacctattgaagttacaacaccttctggtaattgcatagtagaaggtattattttatcccgtacctctaattttacaattctggttacgttatatgttttgccaccgtatatatattctaaaacacatgtataataaccagcatcattttttctaacatcttctatggtaataattccaggtgtccgttgtttaagtctcttatttctaaggcgtcgatgcccgctccatataatatctgcgtttacgttactagcaataaatgcattaatattgggacataccatttcgccagtagatctctcatttactatttgtggatacgagataagatctatatttgattctgagacagacacgattgtcaaatttaacgacatcatgtcacagtaggtttcgttcgtggtaatgcatatataaataccagagtctgattgtgtcgggttcagaattagcatattgctaccattatctatcggtataattctatcattatccgctcctcgtttttcccataaaatatctaatatattatatccggatgatagcgtatttatttgaggacatggtaagattactggctcgttttctaactccatgaatgatgcaaaatattgccctttgtcgatacattcaggcgcgttaaaagtctgaacgaatgaagaataaaaaaatatagaaagaaatataacaggtagtatactcattttattttatagtgggtaaaaaaagtttgtttctgggaaagggagaagagaagaaagaagaaaatgtcaagatgtacaactcatcaattcatacgccggaatatgatgttataatccacgtaatttagcatctcaagcatcacaaacaatgcgttcagactgttactagtgggatggtcttcacctccccagtaagtagcagcatatgcacaaagtccgatgatggttttttgctcatccggtatataacaacggatgatttctacacaatctttgacatcatcccacggcatattttttaccagctcgcgaatgtcgatgaacactaattccgcatcatctctaaaagagaggagtgtggaatacaattgtccagcgcattgcctgttgcggtatgcataggacgaccaataaatatattcagtcaaacattgtttgacgtcatcaatactggtcagtctgtcacatccacagtgctgtggtgaaaagacgtgggtactaaagttggccgtcatcttaccaattgcaattggaagaataacgacctgcactaataccacatgttttttctatttttttaaaaaaaatggttaacaattagttgtcggagagcaatatctaccaacgaaaaaaatttttccatcaacatgcctaatcacatagatgaacggatgatctacacagaactcatttgtaattgttgatgcacagtctgacaccagtgcacaagttgctgcagctgcttctgtatactcttcattgacatctatatacgttttgtggatcatagcgtcgacactcacatctaaattacacatattgctataatctccagttgaaccgaacacctctgtcagtcctgactttactagagtatccaccagattatacgagcctgttaccttaaacttgggaatgtgaacatcgataaacgtagcttccagagagttacaccatttcttaaaatttgtatctgttagattttgttctatggattctaatccatcaatcttgtctggaagaatgaccatcatactagtatctccaacatatggcagttctatgattgaaaagttaccgaatgattcttttacagatgcgtgattaaatagctcgccgtacatagacatcatacttacgtctaccatttccgttggtgatacgtaaaagggataatcactggtaaattccttttcgaatggcgtcaaccattttgctttaaagtatacggcactaattgctaggagacaattgttcatccaatagtggattgattttcccctcagtaaagatatctacacacttgttgattgcatctatagtgcgacaatcagtgaagtcaacagtttgaaacttatcgccaatttttctcaaaaaggaatctttaaacacggcagaatatcgcccatatactttattcatggatttgaatgagatattctgagcgctaaccttatccgtgttctcctccttttctacatattttgatagctgttcagcagtggatccattagctccataatacagtattgtcaatactgacgagattgacgctggagaaatgaatacattctctcctttcatagaagatatccatagtaatcgatattggtcgtgtagcgcgatagagatagtctaatattaatattagatatccgtaacactaccacactctataaaaaaagaatatttcaatcttgtataaacagtctacgtagtctgtcataattaggagtttgagaataatctaacgtgtatactaattctatatatctaactaattccagaggttcatttccttcaggaaaacagtcctcaaagaaagtagctataaattttttatattcttttttttgttttattacttttatactactttcgtttttccatggaagtttgccaccgaaccattctatcatgcaatatcccaacatttctaaatctcctcgttttgaaactgttgctccaagatgattgtctacacacatataattgatatttcctgaagttatcatgtcctcgttgtaatctatatgtgagtttccactcttgtatagtttgttagttctagaatagtcaattagtgaaagacgtttatttctaatcagtatattcctcggttctatttttccatgggtaaatcctcgagagtgtataaactctaacgtgtttatcatagtgacgcatgcttcgaagactgattcagtatcctttggggcgaatactcttcccaaattctttataacgaagaacatataatcatcggtttctccaattccatacagatctggaatagccaaatactttataccacgttcccgtgtccagttgtcgatagtcaaaggatccaatacagatatatagaatcgtatttcagacaacaatggcttgtgtgatttgtgatctatcttcattacgtaattgtagaacgaactagtgaaattttttctgaccttatagagtattgaattaccagaatataaagtatttccgataatccatttcttgccatcgttatcaaaacaatacttgaaggattccatgcctgctatgtaataacgagttgttttttatgttttgtattgattataaaaattagtagatatgatctatattcctacatcgtgactgatacattctataacttctttatcaatggaatcactattactacttgtattaataacgagtgcttctctaatatattcattgggatgattcttgttaatgtttttattattttcaatttctacattttttagtatttcttctttctctctatctatttcattcatgatatcttctacatttgtaacatctgtatctgtatccatgttagtagtaagcaataaaactatagggtaatactgctatacataaaaactatttatattttcattttcaatcaataaggagaagttgataatgcctttatagcggggagaccatcatacttttccacatatccaatgtccttactaaatacatagaagacgttatttagtttacacaatgatgatatggatatcttataaatagtatatgaaatatcaaaccacttattcgttctaggattgtaacaagatatctcgttgtttttctctaaattaccatattgttcggtaacggatagacctccggcaaaatataacattccaaatgtcgagtcgatgccgtgatataccctagccataggcatttctataatcgaccattgtttgtctttataaatgtcaagtttttccatattgttaaccacgatcgtgctattagagttatttatgaataatccgccagttacgaaaatagtattattaacggaaacagcggacacattgctttttttataatttatcggtgtttcgtatatccctgaatccattgccttcacttgcagttgggagatgaacactgcaatcagttctttcgggatagaaatctttggcaattatacacaaccctattttcaatccccatgtttcctcttcagtcttctcacatcgtctaatagacatggagtagatgatagaggggaacagaaggactataatcagggacctcatcttgaaaatggttagagcctataagggcgttaaccagtgtataatatgcagttttatttcgtttttgtatcattaataaaaattagttatgaatatttagtcaagttaagcatgctaagaaaagtggtaacatcatttgatgtgctaccgagaaatttagttaaattttctcttgataactctggccattttgtccttgaaattgggaacatcttctttggatctaattgcgcatgtatatacactctttttaggtgtcagacatactttcgaactccatggagtaattttttcgagaaacccttctgtggctccctgtgctgtcacgcacactttttctgttgtgacaaagtctatgctgcaacctgtagacgtgcaatcgtagtcatcgatatcatatggtactggttctttagaatctccgaatgtgaagttagccgtatacaacaagtagatatcacacatgtcaccgcgtttgtaaataggaatctttttgctacctctagtagcatacggatggtcgtcgtattcagttaatgttacagtcggtggtcccgtacaatactcttccactttataatcgccttgttcaaatttaacccagaaagatacgaaatcattctttggaaatgcggatatgtcgtatgttccttctggtacatttgtatacattgtttgccacgtcttgatgccataatttttaagggatatattgtatagaccatccccagtccattcaattttggaatcaaaattgacggattcaaacttataactagttattttagcgtgtatactattaatgaacaaaactgcgagaattataatatatctcatggtgttgtttgttatttgactactgtcactgaagtgataatatattttaaatttttaaaaatcatattttgaataatatgtattactatgtccatgacgatcaaatatataagtagatccaattttaagagtatgttttctcaatagagtatcatcgttgatcatacttatttcatattttattcctttatagcattttccctctgcagtaatagaatattgtcgctcatcaccatcatatttgtgaatataagggtacttatcataatggcatgataatttggtagatattctacacgtgtgacctctgcgttgaatagtagacatatcaataattttatagtcgtttataacatctatatctatggaatcaaatccgacgtcgcatgactctgtaataatagaaattgtgtactctttaaaatttaacaccaattcattattatcatcagtatatgaattttctatattatctactccatctatatagagttttacactcagtttattaaacggagtgtactcattattcgaataggatgctaatgcacctattacaaataaaaacgttattagttttttatacatttaaaatcttaaaattttaactagtatattgaaataattatttatacaaactaactagatgcatcaacagataataacaaacacctccagcgatcgtgccaatagtagttagagatgcgtatccgtacaacttgttaatcatatttctacggatgtatataccatcgtcgttaaaagcgcttctatatctctcattagctagaattatagatacgctattaattatatcatacattagttgattgttgatactatttttattgtaatcgaaaaacatttccatgaatagtattccggtgccaacgcttacagaaacatcgcgttcattcatcattataccattaagtgtatccatcacccattgtttaacacgtgattcatttggcatagtacgtactatatcatcgaacggtataacggcgcatcttgtaaatagcctgtatatattatgtaatatggcgttacttcttccatacacaataaatctcttgttataatagtgcctgagtaaaaatgctctaacggcatcgtagatatcaacatccactgaagaagacatctcaattgattctagctataagtctttaatcttttgatacttgtttgttattaaattattaattattaattattcattattttaacggatttatattcacggtagcaatttatggaacttatattggtcattatttttgtcacaggaacaaactaatactataacggagattaaaaatatgacgcccataattgttaacgccactatgattatatgataagttgcttctaacgattctatttcttgttcatattgtacaacgtctttcgagagtttgctcaaatctgtctcatcgtcgggaccatcatccactggatcaaatttttcgttagatcgtacacatgttgggagtatgggattccatttaccgtcgatacatgtggatgatggagatcccgttagtataaaaccacttttacaactaagatgtataacgccaccgatagaaaatgtagatccggaaattaatccattagatagagacggtatatcacatttttgttgacatgatggaataacattccaagaattagctgtacaacttatgtacgaagcaccaataacctcatatccaacatcacagttgatagttatatattccccaaatgagtatttttctttaactggttgacacgatccgtgttctaattgaagaggttgacattccgcattaggacacgtaacagtatcattccaagaagtatttccatttttttcttcgcaacgaaaatattttgtttcgccgttgcaacttagtgtcatggtggaattcacttcgtatagcggtttattatatagttcagagatgtaatcagaaactgtgcacatttttttgcatggattttcgtatttccatttatctgtttcgcagacagcatttggatccgaagaatgatatccctgatcacatgtaaacgtaactttctggttattattaaacgatgtttcggtagacgttaatttagcgttattcatagtgggtacagtacatgttgaataaacaacagcaggtagtacgcataacaacgtaacaacggaaatcgttttcatttttatttatgagcgttaaaaatagtatacactgtcgagcactaaaaggaaacaatgatgtagtgtgattttatattttaatagtgtttataagatttttagatatgtggacagttcgttatcgttatattttatttcgttaggaaaacacgaccatttatctccaggatccagcttcttggataatagactaattaatctctgacgatatctaatagatgctatgatattacgtacacgacttccgtatacgtcgagactagtgcacttcacgaaagaaggatgtttaccgtatctcattaaagtattaatgtctttgatacaaaaaactaattggaatacagttttattttttatataacatctcttcatgatatctatttcatcgaaacatcttaaaatataccatttatattgctgtagtgattgtacatctataagagtatcataatcggtcatacacgcagtatattttatacacattttcaataaatctgcattatgctgtttatgtttagtaattgctatcatagactgtatcataattttcaaagatggtcgtttagacaatagtacttccattattattttgttgttgtttgcgactgcttccgaaatacatgtacatccactagtagtaatcgtctcaaaatcaccatttctgtttaatagatagaccaacgtattatacgcattataactgacagcgtcgtaaatagtacgatagttaatatcaataactcctctggataatagataatctaaagattcaacagcatcaaaaattattaaatgacgaactatatgttcattatctatttctaactctgtgtctgattccaaatatagttttataagcgagataccgtgtataattccacaataaaatggagtagttccgaatttattacgcgcattaacatttgctcctttagaaattaataactttacaaaatcatatttattacgacatgagacataatgaagaggtgtatatctattaaagtctacagagtctatattagcattctctaaacatatctcaaacaattccacgttgttgatatcgtgtttacataataggataaaaggtgtgtttccgtacgtatcttttttatctacatctgatccattatttaacaatatcttaattaattcattgtatgattctgattctgattctgatttcttatagtgatatatacatctatgtaataatgtacaaccgtaactgtcttcggcatctatataaaatacatatatagatattaactgtatctgatatataggacaaaaaatgtcttgttctacgtccattttcaagcattagtcttatcctattatctggatgatcattattaactaattgatatacatattcttttatattaatggatgatgataacctgagattgtgtataaatgacccgttcttattaatatttaatactctatctagaaaaaaaattataatattcgtattagcatcatccatagaataaatatgtagaatattttccccatattctaaaatatggaataaatttggcaacctagataaaaaatcaactattgttttatccactttctcgtatgttcgaacgagattataatcctgtattatatgggatgtggaaaaattggaaaacacgcgtgctatataatgaagagataaatatacactccagtcaagtatttcctttttaaaaaaaatccatatataatttatattctgtaacatgttatcccttttcaattaacaatgttggtttataaaaaattaaagaagcgaatcaatgattaatagatgttaagaactataattacgatgtattaataggtatagttagttagttaaaaagagataacagttactaattaattgttagttattgtctatatgatattacaacctattatttgttctctatagttacattaattaaaattttatatgtgacacctattcatctggagaatacttcttgataccatgattctggccaatctgcaaacacagcacaacagcatctttccactttgatagcgcacacgtatgtcgaggtagcctcatccccaggtttatataccttgatgaatcgacacgtgtacttgatgtcctctttcttctcacagaaatacacaacacaaagtcttttgatatgcttttctatatcattctctatcagtctgaggtagtcgtaacccaccgtgaatccaaatactttgtgtgtattattatcaactccaatgaacaaatatccaccctctgtgttggcaaaagatgagagtatacgtgttcttagttgcttagctgaaacagatgtatgtttaacattaatagatttaccagcctgaagttctgatctattgaagaactcctctaccaatctctcaattgattcagtgtcttccactccatctggatattcaaattcctgcatttctggtctgggactccatccacctgattccttcagttcgctaagatactcatttgaatgcatgatcccatagtcttcatgtacaaatttgtttttacagtactcggtataagattttgattagacaaatgcgtatgcacataacagcattctttaccattcacaatatcaaaatgatcatatacaacagaccacatatggcgtatagattccaaacgtttcatgagtacatgattcacactgtcttccagagaggtggttacctcgatggtgccgccgatatagtagaagaaagattttctgtatcattctccccaagtttaacttttaccagatttgggatcggaagcaccgcaccctttttgaatctcattctcataatttcctttccatgtacgtccacagcagtaacttgacagatacaatctcctatccagacgtggtaatcgatgataggtcctgaactgagtgcattcatttctttggccttctctttgatagtagaataatcatgttgaacttttccatatacagcgttttctgtttcaagtacgtgatgatgaatccctactccttccaaacacaagtctagactctcgtacccgattccgcttagagcatagtagtttttgtggcgatatgtgatttccttcttgatcatggatcggatctccaatttgtagatattcattgcactcaaacataggcagcagtgctccaatatatctcttgttcacctgactgtaacaccacatatatttgtcttttacaatgtcatacttgttattataaactgacatcattggcaacagtcgatgatattccaggaaaggcatgaagattctcgtcgtacccaccgagagcgtgtgcgtaaaacatcgccatgatttcggttgtacacacgagatcaataataaattaagttattttttaatttttatcgacaaaaattttacatcaaccaaacaccacacttaataatatacaccctgcattaatatgtgccgaaacttgtcgtaattgggttcctcaaaatatgtcaaagagtttaccatggtaatatattgcagcaattctctaggtgcatattgcaaactggtcattaacaaagtcgcagtattgttaacatatttctgttttgtggcacttactaatgcacaattctttgtttcagatatcttagtccatggcaagatacctcccaaccatctaatcatacaatatccaagtgtttctagatctccacgtctagatacaacgtatcctttatgcgaatctataggtgtaaattctagagtaccgttatccattttatttggatttcttataaatggaacatgttcgccattagacatgaatttagaaaccaatccgtaatccactagatataatttattcttatctatttgatccaagactatattactcgctttaatatctccgtgagaatatccttgctcgtgcataaattgtatggtatttaagatttcgattccgatcaacatcaccgacctttttggtaatctattattattggctctgatcaccgcatctagatctgcacctaatctattaattaccaagaatcgatattccacattaatggatttgtatagaccaaatgccttgcacgtgataagacctacgtgctttatattgtgagattttttccattcttcgataacggatggtttaagtactctagtataaaatgcctgttcggtaaataatgatccgttagctttgggctctatttttactacataattattgtcattagtagtataaatactaccaaatccaccttttcctattaatggtccaacgacccattgatttttgcaattgtcagttaacacaagtccttgaaagttcataatgtgtgatctatctgtcaatgaaatatcattttaaattttaagttttacgtggtaagttttaatatttaactaatacattagacgttgaaatagccacatataaaaacgagttatattattaattatcaagttttaagtcttaagtctctaattagtgttaaaatacattctaatacggtcctgtagtatctgaattaacttactatatgctaaattcacatcatcttcaatgataatagtgtcgaatagaccggcttcacctgcctcatccatgtcagtttttgccaacatcacacgacgatgaatctcatcatccgcttcagtgtttctacaacgaagcttggtctcaaccattttaagagaggtaggtcttatatacaccgagtaaggcattaggtaagtatttttaaaacttctaacaccgtcgatgtttagatccatcacacaaatacgattattaatagccgctgtattcacagcagttttagaagttccgtaaatatttcctaaaaactcagtatgttccggcggctattcccttccagatggcctctctgttaacgtaatggtaatcgacaccttctcgttccataggacgaggaaatctagtggtatgggacaccacaaatccaaatatattcccatagtcttttggctatggctgtctttcctaaaccagatggcccgctcaatatgatagattttactatcccagacatttatgtaagtcaaaaatctagactttgttctctgttttgtatttacgtgaacgtttattatatatataatatgtaatacagaatattgccacggccgacaatataattaatgcggtaataccaaatatttctacaaagtccttggttttataattgctaatagaggttgtactaccgcctacagtagttgatggtactgtatcattatcattgtacgtatcataaagatccgcatcatcggtggttgatttagtagtgacaattccagatgatgtacttactgtagtgtatgagacagtgtctgtaactgtatgatcttcttctttatcagtaattggttccggagtctcgtctgttgtggattctccagatgatgcacttactgtattaatgctatcactagtgtatgtgacggtgtctgtatgatcttctacattatcagtaattggttccggagtcgcgatttcgaataccgacgagcaattagaattatctatataatcaggtttctcagaactagtttccggtgaatgtgtagatccagatagtattatgtctatagtcgattcactatctgtatttacaatcaactctgtggagtattcttcataatctactttatcagtgtcatttgtaggcgatgtcataaagaatgcacatacataagtaccggcatctctagcagtcaatgatttaattgtgatagttgtaactagatcatcgtatggagagtcgtaagatattttatccttggtataattatcaaaatacaagacgtcgcttttagcagctaaaagaataatggaattgggctccttataccaagcactcataacaacgtagtcatttgtattatttcgattacatgataaagttgcatcatcacctatttttttagatgtctgaggaaaaggtgtagcgtatactaatgatattagtaacaaaagtattggtaatcgtgtcatattagtataaaaagtgatttatttttacaaaattatgtattttgttctatcaactacctataaaactttccaaataccagccaccgaaagagcaatcttaatcatgtcaggttcatatttccccaacatactagatccaaattcgcctacatcaggtaaattcatcataatacaatgtctgttcatatcacacgatgatccattgagcatctcatctctatcaagaatcctaatctgtggttcaaaataacagcatctacactcatcgttaattgtagtattgtctaatatatttttgctaatatttgcgtaagttctattatcagctattgcatgcatcacagatccatcaacaaccatatataatatagaacaatagtcggactttatacttatgtaaaacttgaaccaattggaactcggaagctcgtcatgtagacgctggtgtctagatataataacattattatcggttacacttcttaagagaggtgccgcatcgatagagaaatcaaacaggagaataatcaatgatgcatttcctttggtaaaaaaggaaacatccatgggaagaatggctactttatatgaatttaactctatacacgcacacgctttatcagatgagattaatagttcacaaacatctctatcctttcctatggatataataacaggaatggcatctttaggtttaaaataattatatacaccagtaggagtcttgtcatcgtcatctatctttatcaaattagcaaatctggatattcttgatacattctttttatacagtgaattgcatacatcggataccgcattatccatatatggcaaatctgcaatcactgtattgtttttagattgtccgccaatgtgaacgttcttgactttttcacaacatggtttaatcatgaaatcattttttatatgatttatttcctcgccatgttttactaacgcgtttagacagtatacaataacaccatccatggcgaccaccaactaaatagtgttattttaatttgtatgtaactattaagatttagtcaagtttactaaatcgtttagatgagtagattctttccacgttttatcctctcttatcctagtaaatctaggaaatctaatggatattccatttgcggtatgggacttggaagatgtaaactctgctcctgaaatttcccatatctgagattgttttggatcctctactacaaaatcgggaatatagattttattaactactaaccactctggaatttttttgggatccttgttaattttaatcatctttaattggtcttgcaactcccttaacgtattatcatcgtgtcctgaacacttggtaaccgtcttccatttaccggattcatcgtcgtaacaacccattagaaagactgccatgataccaccctttgctcctttaccatagtaagcacctagtactactaaatcggcagaatctgccatggaaccctcgttcaaatagtctcgctttatttttaaccatcttctctttcccggttcgtatactccattaatatcttttaagaccaatccttctaattttctcgttagtgcatcatccaatacgtcagttaactgagactcgttactaatattcgtcaactctgagaatactattctattgggtatttcaaccataacatctttgagaaaagatcttcgttcgtacaatggaatgtccgtcatatcgaatccatcaaagtacaaacagtcaaacacgaacaaacacatgttagagtttttatattctttctttttgtgtatacctaaacttccaaacgggagcggtacattatgttcgtctacaagaacaatttcagaatccaatacgatagacgtagcttttttaaatgctttcggtatgtattctttgagataatccactttatgagagagtactggtttcatgtttctactaaagaaggcaaactcgttattatttttatgaacttgtactctttcaccatcgtatttgacttccgcaaacattcctgatggaaattttttaaacgccttattgacagaatcacacgattccgctaacatgggattaattggagtcataacagaaatagatatagagtctagattttgtttagatgcattttctattatctctttcaagttattagattttctaaacacatcataggcatgaggactaatagcgttaagtacgtaccgagggcccgctttaatttttagatctttatcaataagcatgactacacattttaaatcattacatgtacaaacggatgcgatatcagtcaataattttatttgatgcgattctttagttacggatgataacgtagttaaaaaactatccacttcttctaaagttaaaatgcttttatctcgtggacggatttctgtgttctctttgaagaaagtcctaatagtgtctcctatatatccgtatcctaaatcttgtagcatatcttcctgagattgtttaaatattatactatataattttataatttgtttatcgttcatgttataaattctatcgtctaatccgggaagcaatagcttaatgatcaaatatttatcatccctatctgttataaagtctctaattaatttagatttttctttatatcctgatgcgtgatatatatcacagcataattttctaaattcgcgaagcgacgtcatttaataaaaaaagtatttttttttaatattttcacaaatatcgttcgcggatattattagacaattgtagtatattctcacgtatcacttgtttaatatctatatccgctattctggaaccgagtacttcggcatacgtagttttaaaatctggattattcattatatattttagagtaatgatagcatctactatattttctaggttatccttataaaagctcataaacttgttaactatacaatcgataatttcattgtcgtctaatgaattaatcaattcggtctgatagatctcttgatctataatagtatatacagtatctaggaaagagataaatggattatccaagtttgatctacacatgataaaatttacaatgtccttgtttaatatatcattatctatagtttctatcttggcaataactaattgagatattgatgcgagttcggtatgcatatcggacacgtatccgagtactgattccaagttgccagagtaatatgcttcatccatttataaaaaatgtaatttcactattacatccacagttgccccactggtccagtaaccgtgtgtatagcctctataactatattcttaatcaactccttctttacatcttcctcgaattcagaagaaataatttgccaatgaatatctccttcttcaatcatttttttatattcttgtaataccttttgttggaatgtaacatcttcataaatttcctcgccgacgtttctattaatttctttgctaccagattccaagaatataactaagtcgggtttaggcaatccagattcataactcttactgagagtcattgacgcgcctttagcggcggcatacgctactccagagaatgcgtatctatcaactattaaagtaattccctgttctagttgttcttgtataaaagatgcaaactcccatctatttgcacaaaataatagattaactatatgatcattataggtttttttacgagttagatagtcatctatcatctttccagtgacagtggatctctgaggaaagttaagatattttatcgtgtttgccggtatagattccatgatgttcatacattgtgttgtttttccagatttgtccaatccttcaaaaacgattaatgccccacgagacatttttgtaaacctaacatattttttacaatttatgcgtataataaaactgaaaataaatatatggtcatccgagacgattaggtctacctttatagatcatgggtaacaaaaatattaaaccatctaaggaaaatagactgtccatcttgtccaaggataagatggattcatttaagagaggatcctttagagaaaagtcgcgtgcaaccatccaaagattttcatctcttagacgagaacatattaaagtagaccatcctgacaagttcctggagttaaagagagggatatatgaaataattcagaaatcgtcgtctatagatgtggacaaacggactaagctcatgtccaacataaaaacgatgatgataaatccattcatgatcgagggtttaatgacatctttagaaaacttggatcccgataacaagatgagctactcatcggtgatgatattgggagaattcgacatcatcaatataagcgacaatgaggcggcattcgagttcataaacagtctgttgaaatctcttctcttggaatactccattagtaatgacttgttgtatgcccacataaatgcgttggagtatatcataaaaaatacatttaatgttccagaacggcaactgattctgagaggtcaatacctaactccaattttcagtgatttgttaaagtatgcgggtctaaccataaagtcaaacatacttatgtggaataaacagtttatcaaaccagtatctgacctctatacatctataagactccttcattgtgttacagaatcatataaggtgattggaatgggataaatacaacaattatattttttatcatatccttcacgctataaaaaaaattatacatccgtttccctgtcggttacggcagaacatgtagacgaatcatcgtcttcaaaataatcaaattcatcctctatgctctcagtgtgcccgcgtagatacttacatagattgtatcgtagtctagatagctcagatattggaatactatcagatatatgttcggctaatgccatacacctatattttttatttgtcaattcccaatgtttagatactagactagatagagctactactattctagaatcacaacatatcatatcctttacaaagttgacaaagtcaaatagtcccgaatcggagtctactttaagtttctcattaaataatagatcaatgtctccatactgtttaatagccaaatcatctagtgtcatcaatgccgaataaatattgccggatatagtatttctgtgtcttatatattctgctaaaatcagtttaaaactatacttattatacaagtaagtcatactaaccggcgtattaaccgcgcaaccaatattagtttcctctggaagcacccctcttatctctatggagtcattatgtctattatatgaaataaacgtgtcaactactttgtccctatcaaattcgacagtgtagtgtgtatcattaacttcattacgtatgactaatttattttgctgagtagaaaagtaaactaatgcatttattgttttagacgcattaactgatatatcaaacgccattctcgttaattgtaagaaaatgtattatcttttcatttatgtaagaaataccaataactccacacgctaatctctgatcattttttgaaatagataacgcctttccaataattgtagatatatttacatctgtatctaaataaacatatgctacaccatatctgtttacaaagatgttaccgataaatatttctggactgcctatggaatcacatccttgactaatatctccgtaacgatgaattatcaaactatacgttccggattttaatccaataactttatcttttccatggactggttcaaagtaaataactcctctgatattatcgtggtctattatacaaacagccattatttagtaaaatagaataagtagtctgatattatgagtggcagcaatggccgtgtacgcggttactggtggtgccggatttctaggcaggtatatagtaaaactgttaattagtgcagatgatgttcaagaaatcagagtcatagatattgtagaagatccacaaccaataacctcgaaagttaaggttataaactatatacaatgtgatataaacgactttgataaggtaagagaagcgctagatggggtaaatctgattattcatacagctgctctagtggatgtatttggaaaatacaccgataatgaaatcatgaaagtaaactattatggaacacagactatattggcagcttgtgtggacctaggaatcaagtatttgatctatactagtagcatggaagcaataggacccaataaacacggtgatccattcatcggccatgagcataccctttatgatatatcaccaggacatgtatacgcaaaaagtaaacgtatggccgagcaactggttatgaaagccaataattccgtaatcatgaatggagcaaaattgtatacttgttgcctaagacccactggaatttacggagaaggagacaaattgatgaaagtcttttacgagcaatgtaagcaacacggtaacattatgtatcgtacagtcgatgataacgcggtacatagccgggtatatgtaggaaatgctgcatggatgcacgtgttggctgcaaaatatatccagtatccgggatctgagattaaaggaaatgcttacttttgctacgattactctccatcgtgttcgtacgatatgtttaatcttctattgatgaaaccattgggaatagaacaaggatctaggattccaagatggatgctaaaaatgtacgcgtgcaagaatgatatgaagagaattctatttagaaaaccatcactactcaacaactatacgttgaagatatccaacactacatttgaggtgcgtaccaacaatgcagaactagatttcaactactcccctatctttaacgtcgatgtggcattcgaacgaacacggaaatggctagaagaatcagaataagtatttttttttaaaaaaataatcgagtagggtatcaccactacacagtaagagtacagtaaataaaaagtatttatatacttatttttatagatcgtattatgcgctttctacatccatatcatcgtcatcttcaatatcgataatatttttttcttctaccttgacatcttttgcctcttcgacagcaactgattgcgctaataatacatccttttcttttttctttttaggtgtgacatcaatagttatcttttccaagttatacgatatggtaacaacattaccaatttttgaatcaaccgtaattttgattttatccatttctaatagcatagcataattaagagttcatttttatttttttaaacacgtatatggtcactatcaatgtagacgctattagcatacatattgcagtaaaatcatagatgtcatttatatctttataccatataatagaatcgtatcctattatagtaatacacgtggaccgatgtagagaataagtcttatgtttagatgttaatatagtagatgtactcgttgccaaatttttaatgagacacgatacatttactgagcattgtatatcattatattttaagtttcctgataatgagacaatcaaactaggattaagtgaaccatatttgtgttttaaaaaataaccatgtgtaaaaaagacattatcgtctagttcatctacggttaacgtgcgattaataaaattatatctatatggaatatgcggattatattttatatttactacattatagatcctatcaaaatacaaatgcccataacataattccacatcctctgaatgaaatctaaaaatcgatgaactgtatgccaataccggcattattgacatcgtcaatatggatattatccatttcatcatcatcatttaaaattgacactacatatgaatattactttttattttaattaccagttgcacgtacacgggtagcgtgatctctacacgttctatacacgtcacgtctggatgttgttggcttacccatcaaaaatataagtgcggtatgagttctcgctataacgatcggagatacaggggcgtacccagtaagcaactccatagcatacgtgttctcatccattagagagttagtatatacaatacaatatttctgaccaataagaggttttagaatattacgattagtgatgagaggattaatcttggcaaatgttctgttaggaatagcagctagaacattctttgtagtcttgtaatcaacgatggcggcatcctcgaacttattattttttgagatatcctcgataattttatgccattcagccattgttatttattatatatgttttttaataagacatctattggaataaacttgacattagcattctattcttactacaaaatataaaatataaaataaaatatacaatccaatactcacataatccaactcactcgaacactatttttccaattacgataacaatattgcagaatgtactcgttattatttattattttgatgtgtataccatttagttttcaaacagtgtatgatgataaatcggtatgcgattctgacaataaagaatatatgggaatcgaagtttacgtagaagcaacactagacgaacacctcagacaaacaacgtgtgaatccgaaatccataaatatggcgcatctgtatcaaacggaggattaaatatttctgttgatttattaaactgttttcttaattttcatacagttggtgtatacactaatcgcgataccgtatacgcgaagtttgctagtttggatccatggactacggaacctataaattctatgacccatgacgatctagtaaaattaacagaagaatgtatagtggacatttatttaaaatgtgaagtggataaaacaaaggatttcatgaaaactaatggcaatagattaaaaccaagagactttaaaactgttcctccttctgatgtaggaagcatgatagaactacagtctgactattgcgtaaacgatgtgactgcatacgtcaaaatatacgatgagtgcggaaacattaaacagcattccattccaacactaagagattattttaccaccaagaatggtcaaccacgtaaaatattaaagaaaaaatttgataattgttaattgttatttttataaaaacaagaacggtacggcgatatttatttttttctaaaacatctaaccgaagtagtggtatgataaaaatgtagtttgggagtattcgttgtactacaaatatatttccgttttttagttccattcttcgtggcatttttagctataaaattatatggggttgtctggtttaatatttcggattctcctacccaatatccgcgtctaatgcttcttaaaaaacttaactcgtttggagtctctatcttaattagatccgaatttggatttagagctttgcatgcattacgtccttcctcccaggtttttcgatcagtagataaatggatacatttattattatagcttatccagtcagtaggacaatcttctctaatatatgcatcttttatcgttttatctattgatggtgtatgaactaatttacgttctacaacttttaatagtgtcgctgtaaaaataattccaacaattagaccgcatattacgcagcaagcataaccagcataatctgtcttatgtttgttcattgtcgtgcggtgtactcgattaagattacattttaagtatttttttcatttgtggcattcctactcctatccctatatctgctataaataacggtttcgaagttaccattactgaaaatagtcgcgcatcgacttgcgagctacgagtttgtttagattttgtatcgaacgaatatatagaattttcttttatgaagtatattctatattcccgatgttcattttccttctcctttatatcgaaccatttaacaccagacggtccttcgaagataggttggttagataaaggctttataatatcatctagtacattatatttttctatgacttcaaacgtggtatgtggaacaacttttccagctggtaaacatataccaggagctggagacggcaattgctttgtatatccttccaattttgacgtagaaaaagattgtttaatggtattcatagaataggtacataatgcggacttggaataaggactatcgaagaatacatatagtatcgtatcattatctgtttttatagttctagaatgaataatttgtctataacttcttccgtcgatatcacattctaattcgactttgagaaacgtcgaccatctatgactagacaatgatgatggaccaccttcgtcgtttaggcacatttgtgctatatacggaattttgacaattctctttgagccgatagtatcagtgaaaagaatgtaaactttgtcataagtaccatctttatcgacgaatgctcctcgtaaaccatcttttggaattacgttatccgccgtgtataaatcataaccacatggtccgtcaaatcttctccatcgtttaattccttcttttgatatgtttatgtcagatagtacacatccgttgtgactgattatcgttactttgctattttgataaggagcgtatcctctacctctatgttttgggtcgtctgaaccgtctattttccaacatttgggatttccgttattggttccgcatcctctacttttatagatgttgttatataattattattagttaaaccagttttgtttagtttattatttgaaaatgtgtataccgccccattaacacccgtgtataatacgtcgtctaataagtaagtagaaattatttcttcactcgtttcaaacttatgccattcaataccgttagcaaaatagaataaaataaataacaaaggtatcattttaaataaataaaaaatgtcaagagttagaatatcgttgatatacctctatacgttggttgttataacaactacaaagaccatagagtatacagcatgtaatgataccatcattattccgtgtactatagataatccgacaaagtatattagatggaaattggataaccatgatattttaacttataataaaacttccaagacgacaatattaagtaaatggcatactagtgctagacttcattcgttatcagatagtgatgtctcattgattatggaatataaagatatcttaccaggtacttatacatgcggggataatactggaataaaatctactgtgaaattagttcaacttcatactaattggtttaatgattaccaaacaatgttgatgtttatctttacgggcattactttattcttattatttctcgagatcacttatacatcgatatccgttgtattttctactaatttaggaatcttacaagtatttggttgtgttattgccatgatagagttatgcggagcatttttgttttatccgtcaatgtttactctccggcatattattggattgttgatgatgacgttaccatctatatttcttataattactaaagtattttcattttggttactgtgtaaatcatcatgcgctgtacacctcattatctactatcaattggccggatacattttaacggttttgggtttgggattgagtttaaaggaatgtgttgatggtactctgttattatctgggttgggaactattatggtgtctgaacattttagcctgttatttctagtctgctttccgtcaacgcaaagagactattactaggcatgtgtataataccacgagctagtagacgtggaagaggaaccaggaactattgttattttataataatccttgatcttattcttattctttttatcagattttttgcttccagttttatcagtagatatacaacagagtttcataaaaagagaagagaatgaatccattccgtacagtatttgataatagcttttttttcgttttatgaggtatttagagattagagatgattaatgatccccatactagaaatctattaatccacagaaatactaatgctcatatttatattatcaattctaccgtacaatatttgttccttgataggtacagaattgtacgactgcatttttttgtatactaagtcgaatggatgctcgaggttacaaacatgtattaaagtatcttgctcgatgctagaatttatgtacttggcagaggacatacctatacagtttgatggaataggaaactgcttattgtcgatggttataatggaataccacttattattcgcaaatatctgatcgcatatgtgtctatccaaatagtcggtaactcgtctaataatgaatacagacaacggcgaataattaatcagtgatagattgtttacatacagtatgtcgtttgtaaacgatgttaccaatcgtttgctaggtatatccgctttaacaaccaatatggttgtaggacccttgctaactacgacacatttactcagtggggatatgattacagcatgatcaatgggaaaggttagaatatttttgccattgacgtcattatctatagagataacacatcctccgacagaactaatacagagtataatcgtcgataatctatgaggaataagatggtacatctcattgtcatttacaacttctacgcttaagatgttggtaagaagtaccgtttcatccatctgtctattgatctcatcagatataattttttgatgttctgtatctatataatatctacagtcattattagcaataaaattgctaattttagaaatgccgaatacagggaatatctccatttgtacaacgtgacggcagcaataatactaaatattacttcaattttataaaaaggaaactaatcacaccaatgattctatatcatcgtgttcatgatcttgttcttgcttattaagataatcatcgctatgtgctcgtctaaacggattattctcactaaatttattgttgagttctgtaatgaacggatttgacttgctacaaatactggttttattataatttacgaaaggattggatgaatagttaggattctgtttggtatcttcattaagtactactgtagtgttctgataaatagtctgttcattacgatcattatttattagcagcgtgcttccagcaacactatcgtaaatgtgttctgtgctaggcgccataacattggattcgttatcccatattaaactaccagcgaagctatcatcgtccaatatctcattcctagaaatattatctacgtcattgttttgttccatagcactacagtgatcctcccagtctgattcgctatcagttgatttactagatttgctggaattagaactctttatcttttttaattttgtcatgataattttattgtcattatagacagtacgtatctttttcctacaaatatataatatataacatactaatattgttcccgcaactacggtcaccgtgataagaggtaccagcatcatttctgattgctcaatatacgtactactagttcaatatttattgttttattatttactttacaacttttatgataaaaaattactattgcatttcccatcaattatgatccatccattgccgtctgatatgcagaatgaagaattaaacgacaatttcattttcagattatctgaaggattgatgattagatgataatctccagctgtatacaggttatacgcttctatgattgtagcgttaaaaacatcaatagatgggaatagataaaatggctgatgtttatcttctatatctaacatgatatcatagtcgcatgctatgatgatggtattttttgatctgtatgcggcacaaatgctataaatgttgaatctagccaaatcattagaatcctgaggaactattttacccttatagctaaagtagcatttttgcatgtcggaatattttatatcccgtacatctatttggacagtttttatgttacctataatgtatggtcctataaagtccatgattgagatatcgagatcatcatacaatgtatctgttatagtcaacacacccattggagtaataacaaacgcggcgtccatggcggcgtacgttaacgacttattattaattcattttttgttgtcacttgtagaattttttaacacatagtacagattgagtacttttacatactgttttaaccagttttccagacttgtatatataacacgcttcagcatccgtcgtactgtttagttgtttaaaatttgttaatttactaatatctatatctttatcattattaatatctaaccatttatcattggtcttttttaaacttacccaataatctttataaaaaatactaaacaatactctcagatgtctagtatccggtctaggcaatctggctcgtaatttacgacactgataaaccgcattatctgtagacattttaatgttagtatctaaataacaatgtttatcgtattgtatccatccattggcgcaagcactaggcatcagttcttctttgtaatgcagaaatgttcctatgccactaataatggttgagagtatcatcattatagcgaccggcaccgacaacttcttaaacctacttacagtttgtctattaagcgatttcatttattaatgtacaaaaataaatattagttcattgttttaacacaaaaatactttctaacttcttgtgatacatcagaatcttgataatcggatgtagtttttgtaattggattaccatcagatccccatgtatccttaacataatcaatgagccaggtagtcaagacatcggatttattgggtagtgttgatgattccgcagtgcaatttgctttagcatccgagaataactggtagtctgaatgtaatatataacaagaaccctggtaatataaaccattacagctttctttgtgatcatattgcgtagtgctagacgcaacctttctatgagtagatgatgcagcagcaacggcaacagcggcgtcagtaatagcagcctcgttagcagacatgcattgatttaggcgcactatgagaaacgcggacatggtaatcatagatagtagtgaaataaccatagatattctaatacatagaccaatcacgcgtttgcgtttattctttccctgaattttgtctctgtaaacagtagcggagaacacagatgtctgctcttcgtcgttttctggtgtcatcatgataataaatgttatttatgtcacgatgtgccttctttgttctcctccctactaacgaccttagttttccatattttgatttattatcaaattaatttagtaactgtaaatataattatgaattgtttccaagaaaaacaattttcaagagaaaatttattaaaaatgccgtttagaatggttttaacgggaggatctggatctggaaaaactatctatttactatctctgttttctacactagttaaaaaatataaacatgtattcttgtttacacccgtttataatccagattatgatggatacatttggccaaatcatattaatttcgttagtagtcaggaatctctagaatataatctgatacgaactaaaagtaacatagaaaaatgtattgctgtcgcacaaaatcataaaaaatcagcacactttttacttatttttgatgatgtaggcgataaactatcaaaatgcaatactctaatagaattcttaaactttggaaggcatttaaacacgtctattattctactatgccaaacttatagacacgtaccaatattaggacgggctaacattacgcatttttgtagttttaacatttccatctcagacgcggaaaatatgctacgatcgatgcctgtaaaggggaaacgaaaggatatattaaacatgttgaatatgatacagacagctagatccaataatcgattggctattattatcgaagactccgtattttgtgaaggtgaattacgtatatgtaccgataccgccgataaggacgttatagaacaaaagttaaacatagatattttagtaaatcaatattcgcacatgaaaaagaatctaaacgctatattagaaagtaaaaaaacaaaattgtgcaatagcgatcaatcatcatcgtcaaaaaatgtatcataattataattattaacttttgtaacaatagtcctatttagagaaagtctatcgatagacgatcccaatttgtaaattgttccactacttgtcactccatgatatgataaatccatgtaaaatagcatcatctttagatcattaattgttaccttccccaatacaaccaaatcatcatgatatatacctcctccagacaagtatttaacaacggtagaatgctttggcttataaaatacaaatgacattcccttatgtttaatcttaatcttttctttagttattgaatcgttacaattataaaatgatgttttttccaaaaacctaagtgtatttaaaatagatgccatgttaaaaatgtccgccgtcgactttttggaacgattgataaaagctggtgtttatatttatgttttacggacaaagtgtgtaattgcagctttactagtaaaaaactattccataaaagacgaataagatacaaacacaaatgtttatataatatttaaatggaagaccttaacgaggcaaacttctcacatttattgataaatttatctaataataaagatatcgatgcgcaatacgcgtctacattatccgtggtacatgaattgctatccgctataaattttaaaatatttaatataaacaaaaagtcgaaaaagaattccaaatcaatcgaacaacatcccgtcgttcatcatgcagcatccgcgggaagagaattcaatcgtcgttgaactcgaaccctcattggctacatttatcaaacaaggatttaataatctcgtaaaatggcccttgttaaacattggaatagttttgtctaatacatctaccgctgtcaatgaggaatggctaactgcggtagagcatattcccaccatgaagatattttacaaacatatacataagatacttactagagaaatggggtttttagtctatttgaaaagatcccaatctgaacgcgataattatataactttatacgattttgattattatattatagataaggatacaaattctgtaactatggtagataaaccgaccgagttaaaggaaactttgttacatgtatttcaagaatatcgtttaaagagttctcaaacaatagagcttatagcgtttagttcaggtacggtaataaacgaagacatagtttcaaaattaacatttttagatgtggaggtatttaatagagaatataataatgttaaaactatcatagatccggattttgtatttagatctccatttatagttatttctcctatgggtaaactaactttcttcgtagaagtatattcgtggtttgattttaaatcgtgtttcaaagatattatagatttcttagaaggtgctctaatagccaatattcataatcacatgattaaggtaggtaattgtgacgaaacagtatcgtcttataatccagagtctggaatgttgtttgttaatgacttaatgactatgaacatagtcaactttttcggatgtaattctaggttagaatcataccatcggttcgatatgacaaaagtagatgttgaactatttattaaagcattgtctgatgcgtgtaaaaaaattttgtcagcttctaatagattataaatgaactctctatcaattttttttattgtggtagctacggctgcggtgtgtttactttttatccagggttactcaatatatgaaaattatggcaatattaaggaatttaatgctactcatgcagcattcgaatattcaaaatctataggtggaacaccggcattagataggagagttcaagatgtcaacgacacaatttctgatgtaaagcaaaagtggagatgtgtggtttatccaggaaacggttttgtatccgcttccatatttggatttcaggcagaagttggacccaataatactagatccattagaaaatttaacacgatgcaacaatgtatagactttacattttctgatgttattaacatcaatatttataatccatgtgttgtaccaaatataaataacgcagagtgtcagtttctaaaatctgtactttaaatggacggaactcttttccccggagatgacgatcttgcaattccagcaactgaatttttttctacaacggctgctaaaaagccagaggctaaacgcgaagcaattgttaaagccgatgaagacgacaatgaggaaactctcaaacaacggctaactaatttggaaaaaaagattactaatgtaacaacaaagtttgaacaaatagaaaagtgttgtaaacgcaacgatgaagttctatttaggttggaaaatcacgctgaaactctaagagcggctatgatatctctggctaaaaagattgatgttcagactggacggcgtccatatgagtaacttaactcttttgttaattaaaagtatattcaaaaaatgagttatataaatggcgaacattataaatttatggaacggaattgtaccaacggttcaagatgttaatgttgcgagcattactgcgtttaaatctatgatagatgaaacatgggataaaaaaatcgaagcaaatacatgcatcagtagaaaacatagaaacattattcacgaagttattagggactttatgaaagcctatcctaaaatggatgagaataaaaaatctccattaggagccccaatgcaatggctaacacaatattatattttaaagaatgaatatcataagaccatgctagcgtatgataatggatcattgaatacaaaatttaaaacgttaaacatttacatgattactaacgtgggtcaatatattttatatatagtattttgtataatatctggtaagaatcacgatggtactccttatatatacgattctgaaataacgagcaatgataaaaatcttattaatgatcgtatcaagtatgcatgtaagcaaatattacacggtcaattaactatagctctgagaattagaaataaattcatgtttataggatcacccatgtatttatggtttaacgtaaacggatcacaggtatatcacgacatatatgatcgtaatgccggttttcataataaagagataggtagactactatacgcatttatgtactatctatctatctataagtggtagatttttgaatgatttcgcactattaaagtttacgtatttaggagaatcctggacatttagtttgagtgttcctgaatatatattatatggtttaggatattctgttttcgatactattgaaaaatttagcaatgatgctatactcgtttatattagaacaaacaatagaaatggatatgattatgtagagtttaataaaaaaggaattgctaaggtgacagaagctaaacccgataacgataagcgaattcatgctataagacgcatgaaggctgaacgtgaaatcgctcgtaaaaactgcggaggtaacccatgcgaacgtgaagaggttggaatatcaactagatgcggagaaagaaaaagttaagttctacaaaagagaactagaacgtgatcggtatctttctagtagatatcttacctcttcttcagatccacatgagaaaccattaccaaattatacatttcctcgcattgaagtagctccgttgatgactgaggataaagaaccagaacctgtagaagtggtgcctccatcgtccacagacgttaccgaaccgattagtgatgtgacaccatcggtggatgtcgaaccagaacatcccccagctttctgaatatcagacttcagtatcccaagtagcagttacacctccaccaaaacctaaaactccacagattttcgaatatcagacgtccgattctatagttaacaatccacgcccattttataattcggatctcgaatttgatgatattgatatgtatctactaccaaactagaatattacaccagaaaagacggtttgagatcaactttatctaatggtttataaaacgaaggaggccttcgttcgaaatctaatttaacttttacgcctctggcgttcatttgagtaagaaatactttagatacgtgcgtggtatcaatttttgttaagagaggagagagattaagttttgaacatctaagacatgtattaatacccttgatttgtgctgctatgtctccacaattttcacaaacatacacatcttgataatcttcttccgaatctttcaaaacttctgtaatagtattggctgcaccatgcgctattaaacagtctctctccatttctccgaacttgatacctcctccacgttttcgtccctcattcgcctgtctaatgagcttcgtctttttacctctacatctaacggttgccttgtcctgagttaaatgcctcagacgcaagtaataaattggtccaaaaaatactttggatgcataaggcttatccgtttcaggatcatagagaatcttttcacaaaagattttatccgataattcttcatcagacaatttcggatttgaatgctcataacattgtttagcgaattgcatatatgtatcgatggatgtttcgttactactaggaaaacagacaggtcggttttctcccttattgttgtacggcttagcagaatatgcggctgttaaaataacctctatcaacatagatatagtttttctagagaagatggatgtagaattaataatgacatctggtgtgataccattttcatcgtatggaagttccgtttcatccgcgatatacgcgactgttcccttttgactcgttctagtggtaaatttgtctcctagaatgggtcttctttctttcatggttaatactcgtaccttaactttgtcagtaagttctacttgtactcgttcaacgcgagatttgtacatatcggtatatttttcggaaacatcaaagctaatctgattatctctagcaaaatcatcttcaagagttcttgatgagatatttcgtgctatagcgtctccggattccaagaaagcattcagtctaactaatccattactttctaattttgaataggcgttagacctatctctttctttgttattaaagttttccaacggaatttctacttgatgtttcttggctgtaacaatatcgagaccgcctctctgaataaattgttttttgatgataattccatcctcttgattgatacctttgtacgacattaatgctatagtaacatgttggccgaagcaattagccgcaatctttgaagtttctaaagccttactaatcacgattggcctctctggatacatcaaatgaattccattgtctattttatttcgtatatccgagctcagacaagagatagcttgtttagcttgagcacatccaagaatagctctgggtccagaattgtgattgattcccactagtgaagatgctacatatccatctctaaattcggcaggaaagtcacataaatcgtattgctttctttcatcctttgacatcattctaaatttttgaaccgattcacatacgttactaaaagtaaattgttctatatctaccatttcgatgacatgcggaaactctttctgaatgtcagagaatgtcatgtcgtctaatctgctttctaactccggacacacatccatcatgagctctccgttatccacaaccaagaatggtctgactaatcttcccgctccaatattaatgcgaatttcattcatgtgatccctaactaaagtaatacctacctccaagttaccgaagaatcccatccgttttctacgtctaaagtcagttacaaaatcacatatcatatttggattaagagatgcgactagagcattttctatagtgattggaaatcctgtttcaaagtaacttatatcatctttataatatgatctgatatactcacaaattttcttttccaaatccaaatactcagacgttagtatatttgtaatggaactcaagacagacaattgagatactaaacctacctgaggacctctttccggaacgtccgatgaacaaaagtaaagatattgacttggatggtattttctgacagaaaacatctttgaaatctttacttgatccggataaaatccaacactcctaggaatagaaatattctgcatccatgaatagtgaggatgtgttcgataactaccgtctgactttttgaactttccactcaatagactagaaaatgcatgattaagtccaggagttgttagtacatgaatgtttaccgcgtaagtgcctctgttcttgtgattgttcatgatatcgtttcggatgttgcctatgtaattctctaattcatcatgtgccaacgtctcaaaatatttgccgtacgttaggatgcgatgacaaaccatcgaatctctatctggatatctagaagtgtggtagatacagtagagaaactttcttagtaaagatatcatataaaatccctttagttgatcgggtgtatagttcatatgaggtaaaaagttatgcagcatttcatatttgaactcgttgatcgttaactgagatttttgtttaatgtgttcagcttccaccaggtcattaatgtatgtatcaatattaaagtcagtaatagattcagtaattcgtttagcactatctattatcgcgtttactagatagacaacttctggaggcatatcgtatgataaactacgtctaatgaattctagactcactctagataaatattgagacagcaaaacaatattgaccgttataaatgtctttgtcgatgatatattcaacgcttccaattgtgatatatccagagatatcttgtaatggcgatatcttgtcggtaatacattaggaggggatacggaggaaaacgagaaagtgaatgagtttggcttaacaaccctaaattttggccatgttgtaatcttttctactagattaattcctactttttcgacagattgtttattgataaagactccacctatgacattaggaacaagatacttggctgtatcaagtggatttttattcccgtatcctattagtagcggaatttttatcaaatgagaatcttttccctcataactacttattttagtgatggatataccctctttggtcattacctctttaaagatatttacagtgaacgtggctaatgcgtcataactcttacctttaatactcgcgattataggtgagtaatctggaggagtcactttaacattgctaatttctatcataattcgttctgtattattcttaaacgataagagtggccgcttgacggtcaagatttcatgcaatcgatgcaatataaaattagaatacgatacatattggaaatgtaacggtctataaaaaactccggctttaggatcaggcaccaaaaacttatatccgagtcgttgatccatttctgaattagtgtttttttttcattttagaagcaattcttttagacgatctaccgattccgtttctatatctatgctatttaaaatagtcgatctttccttcatcatttctatcatattctcggatttagtcacgtaatttatacaatcctccagagatcgtaatgctacaatattgagctttttccccttaaaaaactttattttgctatcctcatctaacagagaggggaaaacggtatgatccaacgacttaataaatgtcaggttaaacatccctattttgaaattgctaactccataaacatcgtgttcagccattttcttaattaggaataatttctcatctaatgccgtagccgtgagtacgtggtttacaacattagctacttctttaaagtactcgttttggatgatatcctcgaacaacagaaataaattataatagacatagaataatttgtcaatcatgtgtatacaccccttttcatccttgtccttggatagaataatgtgcatagtagaatccttgaatattttcacacatgttctattagtaaaaataatttctatagtttgtaatatagagctttttccacgcgttactatagagtgattaatgatatctactctcattctcttagaaatggttttgatccaaggttgattaatgttggacctgactgtagtgctcagatactcatcagactttttaccggaaaagtaatttgcattagtgcctccatgtacagttttctttatttccccataattgacctttctcttcttattggtagttttaatatatgatacaagagatgacttttccctctcaaatatctctactgtacgagagctaatattgctattatctgagtttatatcgaaataatgacaacatttcacttgtgaagaatcgttaaataccggatacgtattctgattaataatgtgtttagtaagttggatgtcgcaatacactagtttcttaatggcatgatgtatatctttattatacactatattatcaaacatatcctctgaggaaatgcgttctttcataagaccatatatatctcctatttcatcgcaacttattagatgaaagttaaaaatagttctggcatatctatcttctatttcatgtagaaaggtaaatagattatccataatggcgttactaacgttattacattttttttatgtaatttctagatttacacctattataaggtgtataattagtattccatttatctaatacgtatctcatagccaaattgaaactatccgctacatcgtctaatttgcgtctatccggaacggagtctcgcaatccgaatgtgtccatccaatcaagaaatgcttcgaccgatctcttttttcgatctctatatgaattaccagacatgacaggcgagacgcaaataactttggcagccgatgtatgatataaaaagcctttaataaaatagataaatttgacgtacggcgaccttctaggctgacgttctagaagaactgtagtgtattcatattgtgacaaatctttagctatgtgcctttcccaatcagaactccagtctaattttgatatatccaataccctaacggagttatccttgacttctaaaacagttctggcaggattttttgcacctatatcaaaggcgcatattatttcactcgaataatctttttttgacatcggtgacgatattaaagattgagacgaactggttaaagtttccatatatagtctcaatactcgggttatttaaacaattagtattttcaatattgaaatttacaaccttacaattttctattttgaaccgcatgttattaactaacgttataaaatccgcttctggatatcccaaacattctctgcttaaatcaaatgctgattggttttttatttttcgttttacagttccggatttattaatctcatcaactaactcatcgacagttgcgcactcattaatatctaatttattagtcaattgctttaagcctgcactaccaaccttagagaaatactttccgttaacataatagtctcgtcctatagtgtctataattcgttttattacttctcctctagtttctgttccgtttccatcatagtcgtataaaatggaaaatgtgtttttcttttttacttttacaaatgtatgttctctaaccttggatcgtattaaatggtctacatcttttactaatatcttttttcctgattttgttataacgctaggaataaaaatattatctcctatacgatctaccttgatggactcaatatacatgaatgagaatttgaaaaagtctacaacttgccgcctaagttctcccaacttaatatacgatatggatatttttggaaatttatcatcgaaagagcgttctataatagagtataactcgtcgtcaaataatgtgtcttcctctatttcaatttctaaatatttgggtatgtttaatccgtactcactagctagtttaaatatattaactggagactgatattttgatatagcatcttccattatccatctattgtttaacaaaacaaatctcagaaagtctattccgttatcgctgtctaatactttgttacacgcacttcgcatatcatctggtatttgaaaagagtttccagatccaagttctaccatatttttacccttcgaataaataaagacgctcccaaaatataccggtaagaaaatgtaatagcccggatcaatattaaacggtttattttttacctcatcataataatcagatattgacgtctcgacattagctaccttttgcacgcctattttccagtaagtagatgttccccattctaccaaagaattatacttttctatagccgcagaatctgaaaatttcaaacttttgtacagactaagtaattcttttaagttagttaaatcagcgctagaagtcatgataactttatttttaatcctatgttatttcattcttatttttaatattatagtacctgcaatatctgaaaaaatgagacgcgaacgagccgcatacgtaaactacaaacgtctaaacaagaatttcatttgtgtcgatgatagactgtttagttataattttacaacatctggaattaaggcaaaggtggccgttgataacaaaaatgttcccattccatgttccaagataaacgaggtcaataataataaagatgtcgatacactatattgtgataaagatagagacgatataccaggttttgcacgatcgtgctatagggcatattctgacttattttttactacctaaatggatagcaccaatgtgcgttccggaatgaagagccgcaaaaagaagcccaagactacagttatcgatgacgatgatgattgcatgacgtgttctgcctgtcagtctaaattggttaagatttccgacatcacaaaagtatcattggattatattaatactatgaggggtaatacactggcctgcgcagcatgcggatcgtcgcttaaacttcttaacgattttgcgagttaaatattctatttaatacacgttcttctctggaggatgttgtacatacagaagtgggatcggattcttgatgtttccgataacttttttgtttaaatcctagtttatctacagacagactaataattcgttgaacgaaatttcctatcatgtactttatttctttgatggatgtgctaggaaatacatataccgtcctatctaatagttctgtttctcgacataccctccctagtaattgctctatttgcatattgttgattactgccgagcaaatgaacaaagaatccaaactaggaatatctaaaccagtaccggaataaaataaggtggatacgaatataaatctatttagttccttgattgatttgaccatatctggagtacgtctattttgggcgtctcctataaatacaacctctgatccgaaaaaatctaataatcgtttgtagaagaataccatatgttcacgtagtttagtaataactaaaatgcgattaatagttcctgacttgaattcttctaccagggtattaagaataagttgatttctaggctcgtctacagataataacttctcggtatatatatgatatttattagatggtccatctaatcgttttatcatatgtctaatattatctgtggaatatggctcaaaaaaactatctactgcatagatagttttttttagatcggataacttggcaatattaataatactgttacaataaattctgttagatggtctaggtgtagcagttaaaaaataacacatcatcggaggataatacgctaaaaatcttgtaactgctgtattgttcatcagattatacgtatgtgattcatccaagatgaacaaatcataatgcttattgatatatttacaaaaggcatcgtttgtcagatgtctactgactactattaatacatccggactttgagtctttagttcctttaatagactacttactccatctatggatatcttatgttccaatccgactgcctctacctgtgtcttccattgatgtattaacattttattgggtacgcaaatgacggtttttctaccgtgtgtagccataagataacacgtggtaatagtcttaccaaatccacacgccaagtgaagagtgatgtatagaggacgttttgattcgatcattttcctcatattagaaactacttcagatacgactttgcgttgaagaggatataactttggcgcatttgttggattgatactagtaattttattatcggtcgttgttaatgtttcaaatactagactcgtctttacggaaggagaggcgtaaaatcccttaggtatcagaaacttaaaggatgatcccggttcaacttctacgaaatccccgtcttcgttaaaaagacttaggggttgaccacaagtcattttttttagttcggcataaagattatactccatctttaatagtgacattttttaatatataaatgagttatttaagatattacaatatgcttgacgacttctctgcgggtgctggagtgcttgataaagatttatttacagaggaacagcagcaatcgtttatgcctaaagatggaggtatgatgcaaaacgattatggaggaatgaatgattatttgggaatcttcaaaaataatgatgttagaacgttactcggtttgattttgttcgtcttggctctatatagccctcctctaatctctatattgatgatatttatctcatcttttctattgcctcttactagcttagttattacctattgcttagtaactcaaatgtatcgtggaggtaatggcaacactgtgggaatgtctattgtatgtattgtagctgctgtaattattatggcaatcaatgtatttacgaattcacagatatttaatattatttcttacattattttgtttattctgttctttgcatatgtgatgaacatcgaaagacaggactatagaaaaagtataaatgtaaccattcctgaacagtatacctgcaacaaaccttatactgcgggaaataaggtaaatgttgatataccaacatttaacagtttaaatactgacgattattaaaaatgggggcagccgttactcttaatagaatcaaaatagcaccaggaatagcagatatacgagacaaatatatggaattaggttttaattatcctgaatataatagagctgttaagtttgcagaagaaagttatacgtactattatgaaacatctccgggagaaattaaacccaagttttgtttgatagatggtatgtcgatagatcattgtagtagttttatagttcctgaatttgctaaacaatatgtattaattcatggagaaccatgtagttctttcaaatttcgtcctggatcattaatctattatcagaacgaggtaactcctgaatatattaaggatttaaaacacgctactgattatatagcatccgggcaacgatgtcattttataaaaaaggattatctcctgggcgatagtgatagcgtggcaaaatgttgttctaagacaaataccaaacactgtccaaaaatatttaataataattacaagacagaacattgtgatgatttcatgactggattttgtagaaacgatcctggaaaccccaattgtttagaatggttacgtgcaaaacggaaaccggctatgtctacttattccgatatttgctccaaacatatggatgcgagatattgttctgagtttattagaattattcgtcctgattattttacttttggggatacggcattatacgtcttttgtaacgatcataaaggaaatagaaattgttggtgcgcgaattatccaaaatctaattccggagataaatatttaggacctagggtatgttggttacatgagtgcaccgacgaatctagagatagaaaatggttatattataatcaagatgttcaaagaactagatgtaaatacgttgggtgcacgattaacgttaactctttagcgttaaaaaattcccaagcggaacttacgtctaattgtactagaactacgtccgccgttggtgacgtacatccaggagaacctgtagtaaaagataaaataaaactgcctacctggttgggcgcggccataacattggttgtaatatctgttattttctattttatatctatttattcgcgtactaaaattaaaacaaatgatataaatgttcgtagacgataattcgttgataatttattctacatggcccagtacattgtccgattcatcgggtagagtcatcgttatgccagataatagatcattcacgtttaaggaagggtttaaattagatgaatcgataaaatctatattgttggtaaacccgtcgtctatagatctattaaagattagagtatataaacatcgcataaaatggatgggtgatatattcgtattatttgagcaagaaaatatcccaccaccttttcgtctagtaaatgataagtaattacgagccgttgctgctgttagttataacatgctgtgtactactatttaattttaccatatcttcgaaaacaaaaatagatattatttttgcagtacaaactattgtttttatatggtttatattccactttgttcattcggcgatttaaaatttttattagttaaatggatatgatgcttatgattggaaattatttttccggcgtgctaatcgctggaatcattcttttgattctttcgtgtatcttcgcctttattgactttagtaagtctaccagtcccactcgtacatggaaagtattgagtattatggcgtttatacttggtattattatcacagtcggaatgctaatttattctatgtggggaaagcactgcgcgccccacagagttagcggagtcattcataccaatcatagcgatatttccatgaactaaattattatcgtccatatatctcgacattgttgaatcattattactacttatttagtgaaaagatgatatattgcatacttgatcaatagtgaagttattgtcaataaatgattggtattcttttgttgatcggtatttgcgtagcagttaccgtcgccatcctatatgcgctgtataataagatcaagaacccacaaaatccaaatccaagtccgaatttaaattcgcctcctccagaaccaaaaaataccaagtttgtaaataatctggaaaaggatcatattagttcattgtataatctagttaaatcttctgcataaataaaaatatttttagcttctaaatggcggataaaaaaaatttagccgttagaagcagttacgatgattatatcgaaacagttaataagattacaccacagcttaaaaatctactagcgcaaatcggtggagatgcagccgtcaaaggaggcaacaataatcttaattctcaaacagatgtgactgccggcgcatgtgatacaaaatgtattacatgtaaaccaaaatcaaaatcctcgtcttcttctacatcaacatccaagggctccaaaaatacttctggtgctcctagacgtagaacaacagttactactacatcgtacaatgcgatggatggtcagattgtccaagctgttactaatgctggtaaaatagtttatggtaccgtcagagacggccaattagaagttcgtggaatggtcggagagatcaatcacgatcttctaggtatcgactcagttaatgctgggaaaaagaaaccatctaaaaagatgcctactaataaaaagattaatatgtcgtccggtatgagacgacaggaacagattaatccagacgattgttgtctggatatgggaatgtattaaattaaataattttaattcgtttaacgaatatcttgagtataaacaatacaatattaagaaccggactgttaccgattcctagttttgatgctacagcatcgcagtcatctccgatctccacatcgataatttctgaagtgatatcggtactcagtcctttgatttcttcaaatccgagttttagcaatgcttgctcgataatatttatgacaataataatcacgttagtgacgataatttttcgttttctactttcgtgatattgtttaatcgttttatagattctatctatttcatcctctgaacaaacatccaagtcctcgactgataagggagacccgttcaaatttgcataattaacgatggctatacgcttattaacttctgttgatttatacggagtcttaccaaatagtattctggtaaaattatctaaattatcagatgattttgtattagatcgttgatttctaagattaaccacttcatcctccaacatctgaattcttttatctttatcataaactactcccaaggatggtgtttgtgttggcatagaagatatcatagatctagattccattattgcttcagcatttttcgatactgtaatattaaatcttttttctcgttgttgacgttgttccgatatcaataactctctacgaaacgtaggaggtggagtatcgtcaataatagtagtatgaggttgtactcgttcgataatagtggccggagaatcatcatctggaaatagatcttcattaagttgtgaggatatttcctctggagatgacactacggcggcattatgttctctatctaccacgttgttaacgtgagttagaatagacatttgacgaagagttatttcataatttttgttagatggataattatcttctgaaaactctgtaattaaatcgttttgtatatccgtcactggtacggtcgtcatttaatactaaataaatgatgcctattaagtcaatagttactcttgatcaattagaggactctgaatatttatttcgtatagtttctaccgttcttccgcatctatgtctagattacaaagtatgtgaccaacttaaaacaaccttcgttcatccgttcgatatattgcttaataactcattaggatccgtaactaaacaagatgagcttcaggctgctatatccaaattgggcattaattatttaattgataccacgtcacgtgaattaaaactgtttaatgttacacttaacgctggaaatatagatattattaataccccaattaacattagttcggaaactaatcctatcattaatactcacagcttttacgatcttccacctttcactcaacaccttcttaatattagattgacggatacagaatacagagctagatttatcggtggttatattaaaccagatggctccgactcaatggatgttctagcagaaaagaaatatccagatcttaactttgataacacttatttgtttaacatcctctataaggatgttattaatgcaccaataaaagaattcaaggcaaaaattgttaacggtgtattaagcagacaagattttgataatcttataggtgttagacaatatataacagcacaagatcgaccccgctttgacaacgcttataacatcgcagatgctgctagacattatggagttaatcttaatacattgccattaccaaacgtagatctcactactatgccaacatataaacatctcatcatgtttgaacagtacttcatttatacatatgacagagtggatatttattacaatggtaacaaaatgctcttagatgatgagattatgaacttttgtatttctatgcgatatcaatctcttattcctagactggtagaattctttccagatataccagtaaacaataacatcgtacttcatactcgcgatcctcaaaatgctgcagtgaatgtaaccgtggcgcttccaaacgtgcaatttgtagacataggtagaaaccacaaattctttattaatttctttaacctgttggcgaaggaacaaagatctacggctatcaaagttaccaaatccatgttttgggacggtatggattacgaggaatacaagtctaaaaaccttcaggacatgatgtttataaattctacctgttatgtattcggtctttataatcacaataatactacttattgctctatcctttctgatattatctccgcagagaaaacacctattagagtttgtttgttacctagagtagtcggaggtaagactgttactaatcttatttcagaaactttgaagagtatttcatctatgactatacgagagtttcccaggaaagataaatctatcatgcatataggactttctgagacaggattcatgagattcttccaactactcaggctcatggctgataaacctcatgaaacggctattaaagaggttgttatggcttatgtgggtataaagttgggtgacaaaggtagtccgtactatattagaaaggagtcataccaagactttatctatctgctatttgcatcaatgggctttaaggtgactactagaagatccattatgggaagtaataatatctctatcatcagtattagaccaagagtaactaaacaatacatcgtcactacattgatgaaaactagttgtagtaaaaacgaggcagaaaaattaattacttcagcgtttgatcttctcaatttcatggtatcagttagtgactttagagattatcagagttacagacagtatagaaactattgtcctagatatttctatgcaggatctcccgaaggagaggaaaccattatctgtgactcggaaccgataagtatcttggatagaattgatactcgtggtatcttttctgcgtatactattaatgaaatgatggacactgatatcttttctccagagaataaggcatttaagaataatctgagtagatttatcgagagtggaaatattacaggagaagatattttctgcgcaatgccatacaacatcttagataggattattacaaatgctggtacgtgtaccgtatccataggtgatatgttggataacattacaacccagtcagactgtaatatgactaacgaaatcacagatatgataaacgcctcattgaagaatacaatttctaaagataataatatgctagtcagccaagcattggactctgtagctaatcgttctaaacaaacgattggagacttgaggcaatcatcgtgtaaaatggcattgttgtttaaaaatcttgctacatccatctacacaatagaacgtattttcaatgctaaagtaggcgatgatgttaaggcatcgatgttggagaagtataaagtattcacagatatttccatgtcattgtataaagacttgatagctatggagaatctcaaagcgatgctatacattattcgacgaagcggatgcagaatagacgatgcacaaattactactgacgatctagtcaagtcttactcattgatccgtcctaaaattctaagtatgataaactattataatgaaatgagtagaggatactttgaacacatgaaaaaaaatctaaatatgacagatggtgactctgtctcttttgatgatgaataaatgtcatgttatacagctatattaaaatctgtaggaggactggcgctatttcaagtagccaatggcgccatagatttatgtagacatttctttatgtatttttgtgaacaaaagctacgaccaaattcattttggttcgtcgttgttagagccattgcaagcatgataatgtatttagtattaggcatagcattgctgtatatttctgaacaagataacaagaagaatactaataatgataaacgaaatgagtcgtctataaattctaactccagtcctaagtaaaatattttagtagcgtatccttatcgcgtttgcttatcttgaatgaactggtcttttttaactcgttgatgaattttgtaaataattcatttctatctttaagtaattttattggaagttttgattccacagccaatgaacatgtaaattgtgaatcattttctatgaccagttgtaacttttcatacactgtaatcaatacagtatttaccatttgattaattacagcgcccgtactagcactccattgaacaagatcagattttagattaattagataaaatctatatgttatttcagataatgaacttaccagataactctcttgacggatattaaaaatgccagatgaatgttcatctcgaatagccagagctactgtagagtaatacggttcaaaatcataaacgtgatagtttccaaactggtatccgagtttttcttggatgatggatacttgagagttgaatttaaatttcttactatgttcattatatagttctggaaagaatgcttcaatcattgtttctgtaagtttgcatccttttgatgcgatagttaaaagtgctatataaggcgccacgataatgttattttttttggtaatagtgacgtcttttacagcgtcaacgcatctgaacaaataatgaatctctctatgatctaaaaaataaagtctttgtaaaaagaatcgtagtgctagttttctctcatcatccttagatctatgggcgaatagacgtctactacgggatataaaaccgctattttcctttatcataggtgttgtttgatctatatttacctcccctagttctacggaggcctccaaattaagatctggtactggttcgaacattgtaagacttacatcatcggtagtagattttcactttaccccacgatataaatatgcgatatatagtaagtccgcaattggtattacaggtgggtaaggggcaggaggtagaacgagccctatatctcactccatatgattacatagatgagaagtcgcccatatattattttttacgaagtcatttaaatatacaacagccggaaatagttaagagacatattctattgacgcttcgaatgactcaattaaagggatatttaggaaatttgttagatattaaggacgatattattatctattctcataagaataatttggaatatagttacgttgataatactatttttaatcccttcgtatatactcagaaaaaaacactactaaaaaacgatagctttttatacaatgtatatcctggagcgtgtgactttttggttatctgggtggccagagcgtgcgatacatctattccggaatttggatcgtatgaagatgtagataataatattatcaagtttgaaacaatgttgatggaagtatttccacaactagatttggacattactgtagaatcaaagtttaacaatatatttcgtaccaatctaaaactaactgggttaaaaaagatcattcagcgagttcaagacttggacattaattataagtcgttgttatctagatacgatgaacactttattaatatgaccggtaatcattttattctaaacgatgaacagttaaatctctccatttgggacttggatggtacattagcgttatctagcgacggcgataccgtgatgattaataacgtaaaactatttacagatcttgtgtccgatatagatacacaaatggaacgcatcaagggagatataacgtataaggtacatttggcgactcctatcaattctagaataaaattggatatcgagactagcttcatttttatagagacggcgactaataatattttactatcctcggataaaaaaatatctatcattttggccaaaaaccatatatctattaaagtgaaaaaccatattcctaacatagaaaaatattttacatttttagttattgccattaatgccatgtttaatagcgttcaaaagtctgctgattttaccaaagtggaaactgtttactggtctaggatatgccaaaatacaaagaataagaatagaaaacccatcattattaattatctagatcctggaatgaaaaaaatcagtaacaacttttacagatccgatgagaaagaagtctttattaatgataacggcataatgtttacatgcatggatcctttggggaaatataataaggtgggatttcttaatatatttcatgatatgtggaaatattgtatcccttgttgttttctacatgatcaatctcatcgaagtacattttcatcgtgtgttcatcaaatcgacgttgagaaaaagatagtaagtccgtatatccttaattttggtaaagttgtaacagaatccaaaatgtcatttctccctattatctttgacgccttcttaaatgatggaatgactgctaatatggaacaagataataaacgactaaaggaaactagtggatatcatatagttagatgttgtgctggtgatgatatagttcgtttacgaactacatctgatattattcagtttgtaaacgaggataaaaatattcttatagttaacgacatggtatattttccaatgaacgcgtccgatataggaaagaaaatacatatactcattcaagaaatagttcatgaggtaatgatagtaaaaaagaaagagtctagtgataaaatcgattttttcccacccaactataagttattgaaggatctatttccaaaacaaactattcaaactcctattcaatctgacgcgggaatggtgttaacaaccgatggattctacatagatggaaaactttttaacgaagatctgtcgtctaaatacgttacatttacaaaaaatgttattgcgtctgatgccgtagctaaatatttttctcctttgtttaaatacgttatttcagaagctaaagatagatttatcaaaacgtggatgattaatattatgatacatatgaacgtagatcctaataatataataccgacgttagaaaaatactatcccaactctggaagagcacaaataaattaaacaactaaatctgtaaataaataatggacaaacttagagttctatacgatgagttcgtcaccattagtaaagataatcttgaacgcgagactggtcttagcgcatcagatgttgatatggattttgatttaaacatttttatgacgttggttccagtcttggagaaaaaggtatgcgctattacaccaactatagaagatgataaaatcgtaactatgatgaaatattgtagttatcagagtttttcattctggttccttaaatctggtgccgttgtgaaatcggtatataataaactagatgatgtggaaaaggaaaagtttgcagccacatttagagacatgttgcttaatgtacaaactctaatttctcttaactctatgtatactagattgcgtcaagataccgaagatattgtatccgattccaaaaaaataatggagattgtttcccatttgagagcgtcgactacagagaacgcggcgtatcaagttctccaacaaaacaatagttttatcatatctacactaaataaaatcttatctgatgaaaactatctcttgaaaattattgcagtattcgactctaaactaatttctgaaaaagagacattgaatgaatacaaacaattgtacaccatttcttctgaaagtttggtatatggaatcagatgcgttagtaatctggatatatcatctgttcaactgagtaacaataaatacgttctctttgttaagaaaatgctacctaaaatcatactgtttcagaataacgacatcaatgcacaacaattcgctaatgttatttctaaaatttatacgttgatttatagacaattgacgtcgaatgtcgatgttggatgtctattgacagatacgatagaatctgccaaaactaaaatatctgtagaaaaaattaaacagacgggtatcaataatgttcaaagtcttatcaaattcatatctgataacaagaaagaatataagacaataatctctgaagaatatctatcgaaggaagatagaatcattactattttgcaagatatcgttaatgaacacgatataaagtacgacaataaattgctgaacatgcgagacttgattgtgacatttagagaacgatattcgtataaattctaatatcgtttttgaagtttccaaattttgataatatagtctagatggaattttagaccatctttgtcaaaatcgtttaccgacaacaattctccgttcttttcgatgactataggacaagaaccctcctctatctcttgtataattacatgtagcatattttgcttcatcgtttcgtcagaaaattctgaaactagaggtaatcttcctcgttgaagcaagttatagctttctgctattatacccgctatttcaaatagacttatacgtctagtatagcgtcgtttaagagcagatatatgattccctatatgtttaagattggaatgcgcatcttctatatgagtggatgctgattctacaatcttataagaagatttgggatctatatcactagtttctagtgactctccatcttcttcctcttcttcatcatcctcgtattcggtgagatcatcggattcatagtcgataatatcgtctgtgtctgccatttaatcttataatcgcaattcaattttaaagccttaaatggacttctttaacaagttctcacaggggctggcagaatcctctacaccaaagtcgtcaatctattattctgaagaaaaggatccggatacgaaaaaggatgaagcgattgaaataggactaaagtctcaagagtcgtattatcaaagacagttgcgagaacaactagctagagataatatgatggccgccagcagacagcctatccaaccgctacaaccaactattcatataactccacagccggttccaacagctacaccggctcctacaccaaaaccacgacaacaaactaatacatcatctgatatgtctaatctttttgattggctgtctgcagatactgatgcgccggcgagttcactccttccagcgttgacgccgagcaatgctgttcaggatattatctctaaatttaataaagatcaaaagacgacgacgccgccatctacccaaccttctcagacgttaccaacaactacatgtacacaacaatcggatggaagtatttcttgtactactccaacggttacacctcctcaacctcctattgtggccactgtatgtactcctacacctactggtggtacagtatgtacaacagcacaacaaaatccaaatccaggagcagcatctcaacaaaatctagacgatatggcccttaaggatctcatgtcgaatgttgaaagagatatgcaccaacttcaggccgaaacaaacgatctggtgacgaacgtatatgatgcaagggagtatacgcgtagggcaatagatcaaattctacaattagtcaaaggttttgaacgattccaaaagtaataagattgaatattaaaatcacgctttcgagtaaaaactacgaatataaataatggaagccgtggtcaatagcgatgtttttttaacatctaacgcaggactaaaatctagttatactaatcaaactctttctttggtagatgaagatcatattcacacttctgataaatctttgtcttgtagtgtatgcaattcattatccaaaattgtagacgatgactttatatccgcaggggctagaaatcaacgtaccaaacctaaacgtgcaggaaataatcaatctcaacagcctatcaaaaaggattgtatggtttccatcgacgaagtagcatccacgcatgattggagtacgagattgagaaatgatgggaatgcaattgctaaatatctaactactaacaagtatgacacatctaactttactattcaggatatgcttaacattatgaataaactaaatattgtcagaacaaatagaaacgagctatttcaactccttacccatgtaaagagcacattgaacaatgctagtgtttctgtgaaatgtactcatcctttagtacttattcattctcgagctagtcctagaatcggtgaccaactcaaagagttagataaaatatactctccatctaatcatcatattcttctgtcgactacacgattccaatccatgcattttaccgatatgtctagttcacaagatttgtcttttatttatagaaaaccagaaactaattactatattcatcctattctgatggcactattcggtattaaacttcctgcgctcgagaacgcgtatgtacatggagacacctatagcctaatccagcaactttatgaatttagaaaagtaaagtcttataattatatgttgttggttaatcgtcttacggaggataatccgatagtgattacaggtgtatcagatctaatttccacagagattcagagagcaaacatgcataccatgattagaaaagcaattatgaacattagaatgggaattttttattgtaacgatgatgatgcggtagatccccatctaatgaagattattcatactggatgctctcaagttatgacagatgaagaacagatattggcttctattttgtctatagttggatttagacctacgttggtttctgtggctagacctataaacggcatcagttacgatatgaaacttcaggcggcaccatacatagttgttaatcctatgaagatgatcacaacatccgacagtccgatttctatcaattccaaggatatttattctatggcattcgatggcaatagtggaagagtggtgttcgctcctcctaacataggatatggaagatgttctggagttacacacattgatccattgggaactaatgtgatgggtagtgctgttcattcccctgttatcgttaatggagcaatgatgttttatgtagaacgacgtcagaataagaatatgtttggtggagaatgttacaccggctttagatctctaatagatgatactccgattgacgtatcaccagaaatcatgctaaacggtatcatgtataggttaaagtccgcagtttgttacaaactcggagaccaattctttgattgtggatcgtctgatatcttcttgaagggacattatacgattctatttacagaaaatggaccctggatgtacgatcctctttctgttttcaatccgggagctagaaatgctagattgatgcgagctctcaaaaaccagtacaagaaattatcaatggattcagacgatggtttttatgaatggttgaatggcgacggttcagtatttgctgcctcaaaacagcaaatgttgatgaatcacgttgctaactttgacgacgatcttctaactatggaagaagccatgtcgatgatttcgagacattgttgtatcttaatttatgcacaggattatgatcaatatattagcgctagacatattacagaactattttagattatgatatttaaatgagttggtacgaaaaatataacattgtactgaatccgcctaagcggtgttcttctgcatgtgcggataatttaactactatattggcggaggacggtaaccatattagggcgatactttattcacagcccaaaaaactaaaaatattacaggattttctggcaacgtctagaaataaaatgtttttatataaaatattggacgacgagatacgtagagtgttaacatgaatctacgattatgtagcggttgtagacacaacggtatagtatctgaacaaggatatgaatattgtattttttgcgagtctgtatttcaaaaatgtacaaaggtacaaaaaaagtcaaacttccatgtgtctaataaacttattcatttgagaaatgtattgcggagattattgtctcatcaatgttctggagaaattatctcggaactcttggatatcatggaaaaaaatcaaatatccacggatgatgtagatgcaaattttgtatctagttttcttaaggctaacgagagaataaataaaaaggattataagttagtctttgaaataatcaatcaagtaaaagatgagaaactgaatctgagtacagaaaagattaatgaagtagtagaaatatttaagcacttggtattcttttgccaagaaaacactccttctaagacaattaattattcattctttttggataaaatattcgatatcacttctgtaactaaaaatctaaaacctcaaactgttaaaaattatacgaaaaataatagtaaccaattagtatgggaaaactttttagcacatatgagatctaaaaaacgtgtaactatggtagaggattatggacacgagtatgtttttgtagatgagaggttttctacttgctcattagaagtataaaaaaatagttccgtaattaaatggctaagcgagtaagccttccagatgtggttatttcagcacctaaagcagtctttaagcccgctaaagaagaagcactcgcttgtatactaccaaagtattataaatctatggcagatgtgtctattaagacaaatagtgtaattgataagtgttggttttgtaatcaagatttggtttttagacctattagtattgagacattcaagggtggtgaagttgggtatttctgttctaaaatatgtagggattcgttggcttctatggttaagtctcacgtagctcttagagaagaaccaaaaatttctttgttgcctttagtattctatgaagataaggaaaaggtcataaatacaataaacctactaagagataaagacggcgtttacggaagctgttactttaaggaaaactcacaaattatagatatttctctacggagtttattgtaagctttttccattttaaatagaaaatgaataatactatcattaattctttgatcggtggggatgactctattaaacggtctaatgtcttcgcagtcgatagtcaaattccaactttgtatatgccgcaatatatttctctatccggagttatgacaaacgatggtccagacaatcaggctatcgctagcttcgaaattagggatcagtatattactgcgcttaatcatttggttctgagtttggaacttccagaagttaaaggtatgggaagattcggttacgtaccatatgttggatataaatgtattaatcacgtatctatctcttcgtgtaacggtgttatttgggaaattgagggcgaagaattatataataattgtatcaataatacaattgctttgaaacactctggatattctagtgaacttaatgatatttctattggcctaactcctaatgacactattaaagaaccatctacagtatacgtttatattaaaactccgtttgatgtggaagatacattcagcagtcttaaactatccgattcaaaaattaccgtaacggtaaccttcaatccagtatccgatatcgttattcgtgactcttcgttcgactttgaaacgttcaacaaagaatttgtttatgttcctgaattgagctttattggatatatggttaagaatgtacaaattaaaccatcgtttatagagaaacctaggagagtaataggtcaaataaaccaaccaacggcgactgtaactgaagttcatgcggcaacatcgctctctgtttatactaaaccttattatggaaatacggataataaatttatttcgtatccagggtactcacaagatgaaaaagattatatagatgcatatgtgagtagattgttggatgatctagttattgttagtgatggcccaccgactggttatccggagtctgccgagattgttgaggttccagaagatggtatcgtttctattcaagatgctgatgtgtatgtaaaaattgataatgttcctgataatatgagtgtttatcttcatactaatctgctaatgtttggaacacgaaaaaattcttttatatataacatttctaaaaagttttccgccattactggaacatatagtgatgccactaagagaacaatctttgctcacatatcacatagtatcaacatcatcgatacatctattcctgtaagtctttggactagtcaacgtaacgtctataacggagataatagatcagccgaatcaaaggccaaggatttgttcattaacgatcccttcatcaagggaatagattttaagaataagaccgatattatttctagactagaagttagatttggaaatgatgttctatattcagagaacggacccatctcgagaatttataatgaactactgacaaaaagcaataatggaacaagaaccctaacttttaactttacaccaaagatattctttaggccgacaactattacggccaatgtatctagggggaaagataaactatctgttcgagtagtttattccaccatggatgtcaaccatccaatctattatgtacaaaaacaattggtagttgtatgtaatgacctgtataaggtatcttacgatcaaggggtaagtattaccaagattatgggagataataactaataataatgaaaacaaactatagagttgtaaatggatgaaattgtaaaaaatatccgggagggaacgcatgtccttcttccattttatgaaacattgccagaacttaacttgtctttaggtaaaagcccattacctagtctggaatacggagctaattactttcttcagatttctagagttaatgatctaaatagaatgccgaccgacatgttaaaactttttacacatgatatcatgttaccagaaagcgatctagataaagtctatgaaattttaaagattaatagcgtaaagtattatgggaggagtactaaagcggacgccgtagttgccgacctcagcgcacacaataaactgttcaaacgtgaacgagatgctattaaatctaataatcatctcactgaaaacaatctatacattagcgattataagatgttaaccttcgacgtgtttcgaccattatttgattttgtaaacgaaaaatattgtattattaaacttccaactttattcggtagaggtgtaatcgatactatgagaatatattgtagtctctttaaaaatgttagactgctaaaatgcgtaagcgatagctggttgaaagatagcgccattatggtggctagtgatgtttgtaaaaaaaatttggatttatttatgtctcatgttaagtccgtcactaagtcttcttcttggaaggatgtgaacagtgttcaatttagtattttaaacaatccagtggatacggaattcattaataagttcttagagttttcgaatagagtatacgaagctctctattacgttcactcgttgctttattctagtatgacttctgattcaaaaagtatcgaaaacaaacatcagagaagactagttaaactactgctgtgatttttaaaacatagttattacttatcactcataaatgagtaaatcacacgcggcctatatcgattatgcattgcgcagaactactaatatgcctgttgaaatgatgggaacagacgtagtacgcctcaaggattatcaacattttgtagcaagagttttcttaggattagacagtatgcattctcttttattgttccatgaaacgggtgttggtaaaacaatgactactgtatatattctcaaacatcttaaggatatttatacgaattgggctattatcttattggtgaaaaaggctttgatagaagatccttggatgaacactatactcagatacgctccagagataacgaaggattgtatttttattaattacgatgatcaaaattttagaaataaattttttactaatatcaaaactattaattccaagagtagaatatgcgtcattattgatgaatgtcataacttcatttctaaatcattaatcaaagaagatggtaagatccgtcctactcgttcagtatataattttttatctaagaccatcgcattaaaaaaccataagatgatttgtttatcggctacacctatcgttaatagtgtgcaagaattcaccatgttggttaacttactacgaccaggatccttacaacaccaatcgctatttgagaataaacgtctagttgatgagaaagaattagtctccaaactaggaggcctatgttcgtacatagttaataacgagttttctatttttgatgacgtagaagggtctgcatcattcgctaagaaaacagtattaatgcgatacgttaatatgtcgaaaaagcaagaagaaatttatcaaaaggctaaactcgctgaaataaaaacaggtatatcatcatttagaattctgagacgtatggctactacgtttacgttcgatagctttcctgaaagacaaaatcgtgatccgggcgaatacgcgcaagagatagcaacactatataatgattttaaaaattcattaagagatagagagttttctaaatccgcattagatacctttaaaaagggagaactattgaaaggggatgctagtgcggctgatatctctctatttactgaattaaaagagaaaagcgtcaaatttatagatgtatgtttgggaatattagcatcccatggtaaatgtctagtctttgaaccatttgttaatcagtcaggaatagaaatcttattactatatttcaaagtctttggtatctctaatatagagttctcatctagaacaaaagatactagaatcaaggcggtggctgagtttaaccaagaatcaaacactaacggagaatgcattaaaacatgcgtattctcttctagtggaggcgagggtattagttttttctcaattaatgatatcttcattttagatatgacatggaacgaggcgtctcttcgtcagatagtaggaagagccattcgtctcaatagtcacgttcttactcctccagaacgtagatatgtaaacgtgcactttataatggctagattatctaatggtatgcctactgtagacgaagacctatttgaaatcattcaaagcaaatcaaaagaatttgtccaattgtttagagtgtttaaacatacatcattagaatggattcatgctaatgaaaaagacttctcaccgatcgacaatgagtccggttggaaaaccttggtttcaagagccatcgatctatcgtctaaaaaaaatattaccaataaactaattgagggtactaatatttggtattccaattctaatagattaatgtcaataaatagaggatttaaaggcgtagatggtcgagtatacgatgtagacggtaactatctacatgatatgccggacaatcccgttataaaaatacacgatggtaaattaatttatattttctaatcaatcatcctcagttaatttttttaatgattcgtaataacatcctctatggccataacatttgagttttgcagtatctagagcatattttgcaatttcgtattggagtccattacctgaattcggatctaaaaatattagatccttgatttcatgattacttttaaatgtatcaatgatttgatcactcgttaaagatattcttccgacaaagaagattacctcaaaaaatttattaataatggtatcttctatgatgccatgaataaaaaaccgagtgtctatgaatacaaaagaattgtctatattaacttcttctttaatttccctggataaacactctggaacattctcaccccttttgggtatgccacccggataaatagcatctattctatcattatcagtagtagctggatctagagaaaaaaatgacgatagtatacttctttcctgtttggacaaataattggaataattcagaaataatcgtttctttctagacatgtttctagttctaattatttcagaatagagaaaactatctcgtctgttacatactaatattttgttatcagtagttaataaaatagcggacacggatactcgtttatgacaccataggcattggttaacgaatgccgtgagtgtaataatttgagagtcatcctcgcaaataatagacttagctagtcgtctattatacttaataatctgactaattatactagatctgtaaaagttcatttactattaactagcatattataaatataagacagatattcgtatttatcgtcgttaatacaatcatttaatgatttaatctttctaatttctacattgtaaatactagtatgcgataaatcctccaacgattgattaataaaacacgccatgcaataacttatatatactttattaaataatttatcccgtgttgtaagttttagaattacatttccaaattcttttactgttatacgttcatcactttcctcttttaattctcttcttaaacaatctttaatagattcctttttatctagtttaccacccaataatattaactcttcaaagtgaggatcgatatcgttgatagaaccttttctcaaccgtctaaagatttctcttagttcattgtagtacataaaccgtaatagtttagtggatactctaaagatagaatctgaattttgttgagataatatcgcttggaatgcgaatgaagttcttctagctcctattaacggatatccgtcacttgttatacacgcagcaaacacatgcgtgtcttttgatcttggaatatcttttattcgtttaatagatattaattctctaggagtttcaaatatcacttcctcatccattgtaattcccatactaagagctatttttaaacagttatcatttcatttttactatgccgcaacaactatctcctattaatatagaaactaaaaaagcaatttctaacgcgcgattgaagccgttagacatacattataatgagtcgaaaccaaccactatccagaacactggaaaactagtaaggattaattttaaaggaggatatataagtggagggtttctccccaatgaatatgtgttatcatcactacgtatatattggggaaaggaagacgattatggatccaatcacttgatagatgtgtacaaatactctggagagattaatcttgttcattggaataagaaaaaatatagttcttatgaagaggcaaaaaaacacgatgatggacttatcattatttctatattcttacaagtatcggatcataaaaatgtatattttcaaaagatagttaatcaattggattccattagatccaccaatacgtctgcaccgtttgattcagtattttatctagacaatttgctgcctagtaagttggattattttacatatctaggaacaactatcaaccactctgcagacgctgtatggataatttttccaacgccaataaacattcattctgatcaactatctaaatttagaacactattgtcgtcgtctaatcatgatggaaaaccgcattatataacagagaactatagaaatccgtataaattgaacgacgacacgcaagtatattattctggggagattatacgagcagcaactacctctccagcgcgcgagaactattttatgagatggttgtccgatttgagagagacatgtttttcatattatcaaaaatatatcgaagggaataaaacattcgcaattattgccatagtattcgtgtttatacttaccgctattctcttttttatgagtcaacgatattcgcgagaaaaacaaaactagattcgataccttgttgagcctccattagaacggcagtgacttcgctgccattgtcatacgcattaccatttcgaaaaaagcagtactttgaatcgctaaatgatacagtacccgaatctctacttagtttacagattaaatctccacattgaatagttacatttgattcatcttcgatgtttaatgttcctctgactatatccccaacgtgataatacgcgtaggttattacacacggaacgtttataactacagaattattaactatttcgccgagaggtaattccacgtcttcacgaatttctattttcttggccattttaccactaatttctctatggagatacgtcttatatacggcattcctaatattagtttttatgtctaacgtcagctcgtgtggctccaatgtaactggaaggtatccattggtaacaaagctcgacatttatttctttatatatctcatcagttttatggagaagataccacgttatattctttacgaaagttaactccccaaacaaatggttcgtaggaaagtctaaacggtactgtaataatatcgttttcgtatttgtataaaagagatttatcaaatactttattcgatacaaagaagtgaccgtttacaatatcatctatgtaattcctagcgtcttctttattttttataactgatgtaaccattttaagtaacttggtatcgttatacttaatacgagaattattataaaaaaattgtctgaccaattctcccaataaaacttttacaattgatggatgtggtggaagagaatacgtttcagacatctcttgaagaatagagtatattctattcgtttctttagtcttaaattttagatacaacagctttttgatgtcaaatggtaaaacattaattaattcatcctgtgtgtaatcgtttaatgacgtcacttcgtcattgaaatcggaatatacggcggctaaaagatatacattaactggttcagaaatatcggcgtaagagaattttctaatagatcgtccaagaatttggttgtattgagaaaaagtatctgggatagtcataaaccaaatatgccttacctctttcagagtataggattcggacataatgtttgacgaaaacaaaaacatcaattgactgccatcatcgttttcaggagaattatacacatctaatagatcctctaaagacgatttcattttactagtaacgatagcaaatgtttttggtttgccgtttatcatatgtggattagttccctgagaaccattatattcagaatatccattactgagcatgatatatttaattaccaatccgccatatgtagaattagaaaagtatataaaatgttttccgttgagtgtctgtatccgattaataaagtatttaaatttggaactaatgtttaacgttaccaattcttctccgtataacacgccattatttattttcagatttgggtacaattccttatcctgttcctgaaataaagtatctaaattattcatcagattaagttgtcccaatactgccattgacacgttatacatatttttatcaaacatttcatgataacatagctgtcgtctagtaatcatataatctctctcttgaagtttagacatgtgacaatatactactctagtatctagaaacttacgtccgtgatatcttatcgttggtagatctttatcaggcatttcgtaatatgatattcttcctttaagcaaatccttaagtacattcacacctcgttcgttaagaagtgtctgaattactttcttaccacgactaataatttcaccaaaatctatcgtctcttcggacattaaatctataatatgacccagagtattaggtgtgttagtaatgggagatccagacaatagtaaaaaaggaatcttgtttttattttttatcacggtcataagttctccagtattattcccaaagatattatgtgcctcatcaacgataaaaatagagttattgtagcgagataatccgttataattaatgacgttatcgttataattaagagaataaaaacttgttgtggaatgaataaagatattctcagctatgaattcgtcattaaacaagttcatagctacacccatattataattaaaaattttcaaaatgttaatattaggcactagaatgtaaacctttttaaatctggaagctaccaaggcgaacaacaaagcgattattgttttacctgatcccataatatggaacaataacacgcttctgttctcatctatgatagttctaactagataatctagagtagctaactgatgaggtaatatagttggtatactatcaacatgattatcaaataaatctatgattccggtattcatttagatatgtgatatcttctataaatatatgagcatatatttacggagatgaaatatcctctatgaatatatattgcagatattcatcactaacattggcaaatttcttatgtctattgatgaagctctctatatcgtgtccaaatagtctagaattaaaatacttggatattttctgttggaaagtagtcaacggaagagtaaccacattatcgtacaatatatatccctttttggagaggtccgtcattaatggaatatgctttacagagctagatactaacagagtacctattttgagatagaatgcaaagtccggaatctcttccggtgtaggatatagtttcataataggaacatgatattttttgtaccatttcaccaacaagtatagaaatgcgaatctatatctattattttgtattttaccatctaacccctcgtctaatagtttgactttatcgtacgcgtcattattttcagcagcctctctaccagaaggttgagaaaagtgtgttctgaatcgcacgacggcaattcttctcattaatgcgttatctatcctatcaaagacaggtttgtaattagtatcgataatgattgtcgcatggtttctattattaattttattggagaaacacggtcttccaatgacacaaggttctgtcaactttttaatattgtcagatctaattttctttgatccactacaggcaaaatcaggtagttcgctacagaatacagatcttttcaaatgcatgttagcgataaatggattaggtcctttatccaatacatctgttaaaattgtttgacccgtctcaacaaacaggtcaccgatagcagactttaacaaacgtttggttgtcgactttccagttgcagtttctccaaaaaagaatgttaaacatcctttggtagcaccgcataaacaactagataaagttttttcgtacaactctctatttttcttattttcatccgttaatggttggatatcgttaatgatattcattaactcttccatttctggactgtcttcgacgaactttgtatcgtcaaatttaaatccggttgatacagtacacgtatattttttagcatcatctccagagtaaaacattccgtctaccaggtccaatacaccatttttaaacggaagtttatccggataggtatcggtctccactgaatctattaacatgtctcgtatgttagcttctacagtctttcgtttcctcggacagagtaattcgcttgaatattccttaggtagttgatgtcttattgacagaattagttttgttatcaagggttcttcgctgttaaatttccatgaattattaatccaaactatatggtctcctcgttcggttaataaaacagagttagtgtctaaaattctttgtgcaatattaaacagtttattaccatccaacggaacaattttaactttacaactatgtggattaccagttttgtaaattctaatagcaccattttccaacgatagttgatgcggatgtttatgcgatcgttttttacataatgcacaaggtgttacgtaatctatgaccagtggtaccgttgtaaaattattttcatcgagatcattaaagtttattatagaattaatgaatatttttgaaactctttttatagcgtcttcgaatgaaataaaccctggttcccataacttatcaggaactaaatcctccaatcgtcgttgtagagaaaagtaataactattgttgttcatatccacgtaagtgaataggtaatcttctatattatcatgcggtggttgcattacatgaatagtgtcgcaatttggatttttcctagtacctacaacccgaagagttgtttttctcctatatacggcagtgtctatcgatctggttagtggattttcagatgatctacttaattctaatagtgttcgtttcatagctatcaatgtatccatagtggtatacgtgtctaaaaagataatatgaaaacttgttttatctctatttgtagacttagtcaatgaaaaattagatctcatggattttattacattttcatgaatggcaccgcattctgtaaacgcgaatctagctacacagtttgacacctcgataataaaatcttgaatagccgttaaataatctatttcgtctagacacgcgtctaaatctacatccatgaaaattctgactatagagtatgcttcctcatccctaagactttcgaatagtgtacattctggattattctcaatatatctttctaactcgtcgcatttaaatgcttctacaaatcttggatcttcattttgtctgcacgctgacgggacacctatagtcttaagaacaaagataacatcattacctctaatagttgcatccatttagaacacaagttaaaatttcactaaagcattaataaataaacccttgagcccaatttataggtgccttgttgtctaattccagtaaaacgttgataatttcaaatgatctatctttctcgaattggcggtctctagccgctggatgatatccgactatggtagttaccggggattctaactttgcccgtatattcgagaaatctgttttacccaaacaataaagaacactaacgtgtttagttatatgctgcagcagtaacttggaaatcttatcccagtagatcgcgtgactttttgtttctcctaatttacaacttaagtaataattccagggtataaccccgtctattatattaaggttataacctttataatcaattactccggttaatctagatatagatgaagctatctccttaattgatttttttgtaaaatttggtgattcgaacggtacaccagttccatctttcggatacggatctataccacacacacatactcgtttatttctaagcggttgtttcaactgtataaagaacttatcaggaataggcgacgtctcgtctcgtagcagccaactggctacttcgttataaaactctaccaattgactcattactggttcccaatcatcgtgataagtaatagtatatggcgcgtgtgatacagtcactgaattcattatatcaaattagatacctttttatacgtatctagtaccttttcattttccgaaacgccgttaaaccaagcgaatacaaacgccttaaacttatctctattataacatattctcgttagatttaattcttcggtatccttgtatctaactattgttatatgtttagaaactctatagtggcttctaatcagatgttctaataaatattttaaaaatgaatcttgattaaaaatcatatcattgaccatttgtctggctactgaattacgataatacggagacaataacggattagtcaaatagctatcatctcctaatgatggaggcacctgtacaaataccaatcgtttattcttgaacgaatcaaacgtataaatatcataaatgacaaatttatcagtgtgcatatcttcagatatatgattggatgtagaacaccatctaacttgtcttccagcatctataaattcctccacctctatatatctaccaccgtgctgaaaactagacccatttaattcgacgcataaagatccatatgtatctggagtcacaatatattcagaaaaaaatacatgctcctttaattttgttaattttgatctgataaagtcattgtgatttcctaaaagtataaatacttcgttatcgtcgttatcgactttgggatacttattatccttaactataaaaatgtccatcaatatcgatataaaaaaaataactgatttactcaacagtagtattttattcccagatgatgtgcaagaacttcttcgagagaaatatatagtattagaaagaaaatcaaatggtacacctacagtagctcacatctataagacaatggctagatttgataataagagtatatatagaatcgccaagtttttatttatgaacaggccagatgttatcaaacttttatttttagaagacgtagaacctctgttacccgacaaaagtattaatatatctattaacaatacagagtatccacagttggaaggtcctataggaacaaaaatcgctctattggaattatttaatgcatttagaacggggatatcagaacccataccatattattatttaccgcttagaaaagacataaacaacatagtaactaagtaagtcttcgacatctaaaccttcacatttaatggctcctctatttagttcgaaaaagttttttgtagacggtctatcttccattgtagatgcgccattaataaactttttacttcgttctataattgtagcgaaatcaacgttatctacaagaacaaatccgtattcgttaaacactctgactatatcgttctttttgataatgtattcagtcattggagtagacattgttgatggattatataccactattctatcatcagctattttttctacagacatatagttttcgctactaggtaaattcttatgaattataaaagtctttttatctgttaattttgataatttgtctccgtccatggtagtgattaataccttgcctccagaagcagttagttcggataagttattcatgacggtagcataatgtctcggatgaaaagaataatggatagcaaactgccagtcgatgatattaaactttccaaaatagaatacttctctgacactagagacaaatgtatcggatcgaatagtttcctgaatgtagtcaaatttgtagtacttggttttaattccagagtttaatttgttgtatctttcatttcctctagctatagcatcagcatccggatccgtcgctaccaataacgcaatctctccataaaagtatttttccaggtccgcaccgtttccaaaatcaatcgccaatacctttcgtttgttggaatcgtctaaaaatgttttggaacaatacatagaaataagaagagtcttgacgtagtttgataaaattcccaacggtcctctagttcgtttattcgtaaaataactaacttctggatttaatctaaatttatcattattggcgtattgatgtcccacatccgatagtttatcctcgttaaagatatctcctattttgatgctttgatctcttaaatgttcgactatgatattatgttgatttccataataatcttctgagttaatatatttcatggttttatcaattctaggtttaagtatttctccattaactaagaattctgctataaacttaataggtacaaccacggacttaatacccacttcattatgtgtattaatatattccaaacaatagatattatttagataattaacgccgttatataacacaatcttaccagaaccatattctttaggaaagcctttatcgttgctaaatttcttatactctacaaagatagacgattctccaaagataattggttcactggacatgtacctaaatactacatttgcagtttggtctatagtattttcctttttaattttaaaatcaatgttagatttaggtccctttgaatagaacagaataacaccttctggttgctttggtaaatatgtagataacatatcgacgacttcactagttgtagtaaacggaccttcgtatttctttgacttgaatactatccgatcacaaatatccactagtttagattcaacatacttactttcttctagtctatcattgattgcattcacaggctctattagcttaatgagatatacggtccagttcttatccttaactgcctcaccaaagactactacttcggaatctattattctcttaacaggatatctaataatataaccaagatgtgtaaaataacaatacaacccttttgatgtaactctgatagttataggaattccgtcagtcttagttacggcatatagattttccagatccaaacctactatatcttgtttaggcaacataaaggtttttataggcgcgttaataggcggagaaagaattacattctctggagaagccataaatatatgacgtgatagagtcgtcaattcctttattagttcatcatatggaactttttcattgtctctaggtgtgaattctatttccagagatgtattaggccttgactttggatgattaatagcgtgtaataaagaacttttagactgggcaccggatcctagaaaatattttagcttaaagtctatcgtaaagttttttgtttttgcttgaataagattgactaattctagtcggatagaggatccatatttcttgtaatccaaaaatatatgacgttcctctgtcgatagtctcaacaagcattctttgtgaagacgattttccgtcactaatgatttcttttcccaaactatgttatctatagcatctactaactgtacattttttacatctagaccatgtaccttagataatggaatcttagttctaattttaacaccttccttattagtaacggtaaatcgaataaacgattcctgaatcgtagagatattcactacatttgtcaaagtaattaatggcggcttaataaatactagttccaattcattatttatttcgtatgcggtagacctctgttctaattctgaagcattcttcgctaaagcgtctatatacgttgcaatagtagaagatgatactatgttggcatccatattgtgttttattataaacgactagttttttttcatttacttattaacaagcgtcttttatatattcgtaatctatgcctttagctagagctattttaagcttttctgtatctcgactgatacgagtatctgatctattgcggtatttttttataaatcgtttaagacggggagtagttttgatatattcgctaatatcctctttaataatatccacacacgccaattgttcggctatactcgatgcgtgagtcttgcattcatctccagatatcggagtgagggtcagatccaaatattgagaagccttataattatcgtattcgaaatcaatcataaactgaccatccttatccactgaaaaaatggtattgtttggatggcgtttaaatatagacattatcaatgccataatatctaatgtgtttagctctccgaagaaagctgtcattgcgagagatttcatacgcttatccatttccatttatcggtcttgtaattatttgtgtaaagatctatatcatccatccgtagatgatttaacgtgatctacgactatagataggaattcatcgaacgtagttttagatactacatctaaaaaatttttatcctttaccatttctaaaatagtcgttgccatataagctctttttgaaattgatggagtatgacctaccacttcagcagtttgtttgatagttaacgctattaacttttttggtgacggaagaggagatatggactttacatttgtccaaaaattatataaaaacgtataattgactccatacgttcggagatccttgattctaataccaaactgtttgatacattcgtatacctttcgttcacttagtttgttgaacagaaattcttcgggactagaatcatccgtcagtttcaatagcggtttatatagtctattagacttatgaacaacaaattcatgtgaaactttgtcctttcctacaaacttgataactatttcatcgggacttatttctatgtgtttattttttagtgttaataaccctactgtttcattctccttaagatatttcattttaccaaatctaataaaaaacatagtttccattaacataaaaaccgccaactgataattggaatctgtggacgatttctttatatttttgtttataaaacaattaattcgtttcataacgttatatactctaacaaaaatacgatctcttttagcgttgcgattctgtacatgcatttttccgtaaaagtattgtctacgtccttttgaatcacttcccacaaaaattaatctagttaacgcctcctcccacgtttgttcatatactactacatctgttaaatgagtaggaattttaacatgttgcaaaacctcatacgctggattatcgtctgatacaggatttaaaaaattattatcggtaaagagtttaccatctttataaaaaagtgcacgcatttacttcttacaagttttaacttttttacgaacaactttagattttccctcggtgactagatcagatagtgttgtaatagctttagtcatagaagtaaattgtctagagataccagctgcttgaacatcctctagaaccgtcgatactgcagagattctagtaataattttcttaagatctttaacgatattgtcggtagccacctttaggtcagaaagatcgcttctagcactatgatttactttaccagcttcaacttgtaccataggttcatcatccccgtcgctatcatcgagctctacagcagccacgctttctacaatgtcgctgactccaggagatggagaatttttttcagttgtttgatgatattcctctataactacttcttcttccacttcctcctttttggttgatcttttagtagccgctggtttacgaggagtagtggctcgtttggttttgggcttagtagatggaattattacatcttccgggaaaatatcctcgtttttatctttattttcagcgctattttttagatgagctctgatttcagccatctttgtgaagctactagtatccgctttatttgtaattgaccacgccattacgatacaaacttaacggatatcgcgataatgaaataatttatgattatttctcgctttcaatttaacacaaccctcaagaacctttgtatttattttcattttttaagtatagaataaagaatctataaaaactaaaaaaattatacatcataaaccaatttcctagttgtttgtaactttaaatggactctaaagagactattctaattgagatcattccaaaaataaaagcatatctactagacgcgaatataagtccaaaatcctacgatgactttatctcacgaaataaaaatattttcgttatcaacctttataacgtatcgactatcacagaagaagatatacgattgttatacactacgatagaacagaatattgacgcggatgatcaaacactggttgctattttttcgtatataggatataaatttgaacaggctgttaaagaagagattagtacgagtttatccttcaatgacaagaataccacagatgaaatgacgtataacttgtatgatcttttttttaacacattagacatgtatttacgacaaaagaagatcagtattctggtaaatgatgatgttagaggtgatgtaatcgttagttataaaaatagtgacttagtttcatcatttaatgcggaactagaaccagagattaagaagataccgttcaatatgaaaaatctattaccgtacttggaaaagaatttggaccaactaagattctctaaaaaatatttagactttgcatatttatgtagacacatcggtattcccatttccaaaaaaaagtataatgtgcgatatgtatttctttataaaatagacggattatccattcctattatcattaaggattttttagatgttaagtacgtatatttggaaaatactggaaaaatttataaaaattctttttccgaagaccataacaacagtctatctgattggggtaaagtcatcatacctctcttaaaggatcgtcatctatatagctacatctttctatctagttatcatttacatagttactatacagatctcatcgcgagagacgagcctgtgtttgtgaaacgcaaaaaactagatattatagagatcgatgaacctgaggcatggaaaagggatgttagagtggaattcgcaccgtgtgagcatcaaattagattgaaggaagctatgaaagttgacgctaactatttcactaaaattaataattttgctaacgaatttatttattatgaagatggtgtggcatattgtagagtgtgtggaataaatatacctatatttaatttagatgccgctgacgtgattaaaaatacagttatcgtttccacgtttaacaagactatattcttgagcgaaccatatagctatttcgttcatagtcagcgctttatctttaatattatcatgtcttttgataatattatgaaatctcaaacttgggtaatgaaatacaacattaaccgactaattcttaactttcttattgatataaactctagacgtcaggaatacgaaaaaaagttttcttctgaaattaagagaggtctgttctttcttcgtttgtctgcaaacttattcgaaagtcaagtatcgtctacagagttattttatgtttccaagatgcttaatttaaactatatagttgcgttagtaatcattcttaacagtagtgcggactttatagtttcttatatgaaatccaagaacaaaacggtagaagaatccactcttaaatacgccatctccgtggttatatacgattttttggttaagactagaatttgcgagaagggatcgttggatactatagttttatttaccgatgtatacacatctataatgccggaggaattggatttacattttcagagaatcacattagaacttagaaaactagtatccattcagagatcggcgttagaacccaattacgatgtagaaagtcgcggcgaagagcttccattatctgcattaaagtttttcgatacaagcaccattatagttaagacaatggctccagtacatacatacatcgaacaaaaaattgttgcacctactccatcggtcgaaccaactgatgcatctcttaaaaacttcaaagaactaacgtgtgacgaagatattaagatctcgattagagttcatgatactaatgctacaaaattagtcatttttccatcacatctaaaaatagaaatcgagagaaaaaaactaattataccgctaaagagtttatatattaccaatactctcaaatattattattctaactcctatttatacgttttcagattcggagatcctatgccattcgaagaagaactcatagatcacgaacatgtgcaatacaaaataaattgttacaatattctaagatatcatttattgccagacagtgacgtgtttgtatattttagtaattcattaaacagagaagcattggaatacgcattttatatctttttgtcgaaatatgtaaatgtgaaacaatggatagacgaaaatataactcgtattaaagagttgtatatgattaatttcaataactaaatggcggcggtgaaaactcctgttattgttgtgccagttattgatagacctccatcagaaacatttcctaatgttcatgagcatattaatgatcagaagttcgatgatgtaaaggacaatgaagttatgccagaaaaaagaaatgttgtggtagtcaaggatgatccagatcattacaaggattatgcgtttatacagtggactggaggaaacattagaaatgatgacaagtatactcacttcttttcagggttttgtaacactatgtgtacagaggaaacgaaaagaaatatcgctagacatttagccctatgggattctaatttttttaccgagttagaaaataaaaaggtagaatatgtagttattgtagaaaacgataacgttattgaggatattacgtttcttcgtcccgtcttgaaggcaatgcatgacaaaaaaatagatatcctacagatgagagaaattattacaggcaataaagttaaaaccgagcttgtaatggacaaaaatcatgccatattcacatatacaggagggtatgatgttagcttatcagcctatattattagagttactacggcgctgaacatcgtagatgaaattataaagtctggaggtctatcatcgggattttattttgaaatagccagaatcgaaaacgaaatgaagatcaataggcagatactggataacgccgccaaatatgtagaacacgatcctcgacttgttgcagaataccgtttcgaaaacatgaaaccgaatttttggtctagaataggaacggcagctgctaaacgttatccaggagttatgtacgcgtttactactccactgatttcattttttggattgtttgatattaatgttataggtttgattgtaattttgtttattatgtttatgctcatctttaacgttaaatctaagctgttatggttccttacaggaacattcgttaccgcatttatctaacactattccatattactaaaatcggaacaccaatgcggtgacataaaataaccgctataacctaattcatttaacatctcattaccacaagtaataacattattagacttgtgttttatcaaatactgacaaaattgttgagcagatggatcgacctttgccgcctttttaaccatccacgcgtctccagtacctcgcctaatagcttgcggcagatatgttttcttatccaatcgcatagctataaaataggcgccgaaatccacacatttgaattcgaatatatcatccttaccagcggctagaagtctacctctatcactttctaattttgttttgctatccgttaatgatttccaatcgttaaccgtatttttaattcgcatatatctcgttaattcattaaagactggattatcagacgtctgaaaccagagtaatagcgcactaattgccaatataataacaaagaatataagtgttgatgttttggctgcttgtacgcctactatagccttttctctaacgtattctaaattacacgcgtttaccgataaagtagttttatccatttgtacgttataaatggataagaaaagtttgtataaatacttactactacgttcaactggagatatgcacaaagccaaatctcccactataatgacaagagtaaccaataatgtgtatttgggaaattataaaaatgctatggatgcaccatcatctgaagttaagttcaaatatgttttaaatttgacgatggataaatatacattacctaactctaatattaatattattcatataccgttggtagatgatacaactaccgatattagtaaatattttgacgacgtaaccgcctttttatctaaatgtgatcaacgaaacgagcccgtgttggttcattgtgctgcgggagtaaatagaagcggggctatgattttggcatatctaatgtctaaaaataaggagtcattgcctatgctatattttttatacgtgtatcattctatgagggacttgagaggcgcatttgtggaaaatccatcgtttaaaagacagatcatagaaaaatatgttattgataagaattaaaagtcttctgtttcttccatttccttgatcttttgagaagatatcttatcagatagtttcctttccattctcatgagaagacccaagtcgataaagtatttgtaatatccagttcctatattagggaccttgctaaaaaagtggcagctactattatcgttaataggttctgactttttatgaagagccgcgtttaacaatgctttattgtctccgaacgtagctctcttaagagtactagctgcgccgaacttgaatttattcactgattctggttcgtaactagcacatagtaaactagcgagaagatcacaaggctgatacagataatcgaacccttctccataggtgtttaacatggcttcgcacaagtattcacgagcggcctcgataccgaagatatcgtatgtattccatactccaggatacacgttgacgttttccaaatcgaaagaacccaattcttttagattcatgagttctacagtcatcttattgagcttttttgtttgattgaagtcgtcatatcccgtataatcagagataggaatcttgaatttactaatcttgcccttgttggcggcacccggaagaaccatcataaacttactaagattcaattcttccggttcaacgaaatttaggtagacagtaaatctaatattatcctcatcctcaatgaatgtttccataccccattcctttacaatgacgctaaaggagatgaatcgttcaatcatatattcgacgactaattcggtaatttctgctctcttgatgtataatctattgactattatatctactacatacctatctgtttcttttcgaagagtgatgtttggatttaattctcccaaacatacaaattcgaaattaatcttaacagattgaagtttagagatatcatcggataccagagtgataatttcggtcttattcttactcaaattagtcaagttattaaactcgttgaaaccaagtttttgtttgacggcaccacttttttcagtagtgtgaaaactggacagggcttgttgtgtaaacttctcagacaatacctgtgcagaaataattccaataggagttccaccacctagagtataattgagtttttcatagaacttttcaaagatagtgatagccgtttcttttgtaattctaattctagaaggattaagatgcgtcaagaatatatactccataaaatctatattagataccgtaaagaagtatttctctctcacatcatcaatgacgttatgaatcatatcgtacagatccttaaccttaatagcattatcctcagtggtgggtttgacgaatactaggaaattaaacggcgccaatgtcttttttgcaagtttctgtttctgagagtaaacgaatccctgttttattttattccacagagcactaatttccaaataccaagtcatggactcatctggatagataagatctacaggtttacatactgagcctagaattttggtataattggcggcgtacttgatgagcgtattacctataactacttgtccgtatccgtcgaccaccatatcctccatctttttaatgatttttctagccagtgttccggtacgtgatgtttcacagacgatatcagtagattgagatcttgcaaccagcatcgaaaagtaatattgagaacccgttaatccttttgttaaagaattaagaatgtaacctcttccttctggatccttagagtctggaagatagtaaggtaagactctacccaatactcgagtctctgctggttcaccatcaatcctctgttgtccataagtacctagaatatacattagttctgtgggatttactttataacccgctttggccattttcaggaggttattatctggatcatctatcagcgtttgtctcatatgttcttctatctctcggatattaagatttgtcaagttggataacatggattccacatagtccgcctctaaagctttagataatggaactattttcccgtctcttacatcgttgagatatttggcgtatgcttctttgataagttctattttttctacgttgatggcctccaatttattagtgaacgtcgaatttggtctcagatctttgaatgtcaccccaaaaccgtaaatagatagatatctcttaaaaacataagatgacttgataataaagttgataccttccacgttcgacttatgatcggataagagtccagccaatgacagatgcctcatagccacaacaaaattactatctacgtcgtttgcaataatttcaccatcctttaagagacccggataattaatcttttcacctatcaagtaagtatagatatctttaccgctgaatttacatttacctttaggatcgaactctcttccatatttccccaagatgttcaatacttcatctaaacaaagatcttgtatcctaaacaatgaatacgctgctacgatttcatcttgaatagatccataaacgggggctccatgaatatcgtgtttgagtaacgtcgtcggatacataagaatactttgttcaattacggctttaggattttgctccaatatcatccattcttcatctccgtcgaaatcagcattttgagagttgacaattccgggagatattttgatagtatctccttcggtagctctgatagatgaagcgatgacgttgtatctatgtagagacggctgtcttccaaaaataatacttgtatattcttgaacagctacttctacccaatcaccaggcaataaatgtattttatttttgataaactttccttggcgtattcttgttaattggtttaatcgtttattaaagtaaaatttaacttggtttgacgctaatagttgtttaactttatccactgtaaaggcattaacaaatatcttttctgtaagtgtatttctaatatatgcgggcattcctacctcattaacggtgatagatgtactgggaccaattacagatctagcggtctgatcttttcgccgggcgacgatataacttctaatcatattatttttgccggatgtgatatatgataaattgatactggaagtgttattagaaataattttaatatcatcgtattctattaccgccttctggataacctgttcatcagcattcaagttacaattcttaacgatcatacctaataagtaagttaattcattggtttctttgggtatactatctatccaaaaactaatagccggtctaataatcaacggagggatgggaaagtagtctgtataaaataagttagctggatattgatgaatttctaataatggccaaaacttttcatgaatagaaattaacttttgatagatgagagaattaggaacgttaatatcatccaacttgttgacgaaacaaacctttttctttgaaaaagtaattttttgatacggttgcatacattcactgttccaacatgactttttcttggataatattttatcctttaatctcctaagagcgtgtcccgataactcttttaggttaatatcgtcggaatacggttctcgtgaacgcaataatccgcagtgaatacatatataattcagtaaacgaataatttctgaaataaattcaggcttaactatatgagttttataaatacttactttaccccagtgaccgaaacattccaattccgttttcccacaagttttacataatgccccatccatagcacctagtctaccatctttaacggtaccgatatcgtcgtcatttttaacatgactaatgataatatctgtagcattaatctctttttgatcatatagactatacgtaaccttagagattacagccatttttatcaagtcagtttcttttaaagaaccgaaagtatacaatcaaatttccctttttattacaactataaaataatagttatatttacactttaaatttttatcatgacggacgaacaaatttatgcattctgtgatgctaacaaagacgatatacgatgtaaatgtatttatcctgataaaagcatagtacggataggaatagatacaagattaccctattattgttggtacgagccatgtaaacgaagcgatgcgttgttaccagcctctttaaaaaaaaatataacaaaatgcaatgtatcggattgtaccatttcattgggaaacgtttccattacagatagtaaattagatgtaaataatgtttgtgattccaaacgagtagctaccgagaatatagctgtccgctatctgaatcaggaaattagataccctattatagatatcaaatggcttccgattggattactagcgttagctattttaatattagcatttttctaaacaagatataagatataaaatatattattgattattataatgttcttatctcatctctactaattgattaatcagcgactgaaataacagatctatcggctatctctactccagttaccatgttattgcggaaaaatctaacaatttttaatggtatattaggacggtagagaatcttgacaactatttccgtctctaacattttgggaagacgaagagtctttttaccatcgcctgtttgtagtacactattaactatattagtttctgtagtatctagtctaacaaagctaggaaataagtcagatattaccacatctatgtatagaaaatttacaggaaacttgttatgacctttttgaagatacgatgttataatgattggttctccataatcatcatcaaccggagtataatttatggatgattcgttaaatacctgaaacacagttttcatagaatcaattttatgtctaacggttattaaagtaactaaatcattatactctatatcggtagtatatctcagtagtacgtttctatttattactgcgtacggatctcttgctatttctgtttttagacatagaatttttgctagatattttacgttgtattggttcatgactaactttttcagttgatgttgttggaatatttaagaaacgaaatatagattgttgtagaaatagtacctttgctttagtagtaggaaatgttttattgcagtacacggtcctcagcataaagtacatgtgaaaatagtcatattcctgattaggataatcaaagttaacaactactttgttacggacgatcttattaaggtagtacatctttttttcataatttacagcgtctgatttggtaactcgagtcagtctcatgttctcaccggtataaatacttaataatctcatttcagctgaatatgaaggagcaaaaggttgtaacattttattaccgtgtgggatataaaagtccttgatccattgatctggaaacgggcatctccatttaagactagacgccacggggtttaaaatactaatcatgacattttgtagagcgtaattacttagtaaatccgccgtactaggttcatttcctcctcgtttggatctcacatcagaaattaaaataatcttagaaggatgcagttgttttttgatggatcgtagatattcctcatcaacgaaccgagtcactagagtcacatcacgcaatccatttaaaataggatcatgatggcggccgtcaattagcatccatttgatgatcactcctaaattatagaaatgatctctcaaataacgtatatgtgtaccgggagcagatcctatatacactacggtggcaccatctaatataccgtgtcgctgtaacttactaagaaaaaataattctcctagtaatagttttaactgtccttgatacggcagtttttttgcgacctcatttgcactttctggttcgtaatctaactcattatcaatttcctcaaaatacataaacggtttatctaacgacacaacatccatttttaagtattatattaaaatttaatcaatgtttatttttagttttttagataaaaaatataatattatgagccgacgtaacactttctacacaccgattgatacatatcattacctcctattatctctatctcggtttcctcacccaatcgtttagaaaaggaagcctccttaaagcatttcatacacacagcagttagttttaccaccatttcagataatggaataagattcaaaatattattaaacggtttacgttgaaatgtcccatcgagtgcggctactataactatttttccttcgtttgccatacgctcacagaattcaacaatgtctggaaagaactgtccttcatcgatacctatcacggagaaatctgtaattgattccaagacatcacatagtttagttgcttccaatgcttcaaaattattcttatcatgcgtccatagtcccgttccgtatctattatcgttagaatattttatagtcacgcatttatattgagctatttgataacgtctaactcgtctaattaattctgtacttttacctgaaaacatggggccgattatcaactgaatatgtccgccgttcatgatgacaataaagaattaattattgttcactttattcgactttaatatatccatcacgttagaaaatgcgatatcgcgacgaggatctatgtatctaataggatctattgcggtggtagctagagaggattcttttttgaatcgcatcaaactaatcacaaagtcgaacaaatatcctttattaagtttgacccttccatctgtaacaatagggaccttgttaaacagttttttaaaatcttgaaagtctgtgaattttgtcaattgtctgtattcctctgaaagagattcataacaatgacccacggcttctaatttattttttgattggatcaataataataacagaaagtctagatattgagtgatttgcaatatatcagataatgaagattcatcatcttgactagccaaatacttaaaaaatgaatcatcatctgcgaagaacatcgttaagagatactggttgtgatccatttattgatcgcaaaagctttgcacaatctttatacactatcggtttactatttattgataacgcagatgtttgagttgtcatccatggtaatccatagatcattaatttatcgtcttcacacgctagattagcacgtcgtaatctatcaataggatcgggtatttttcgtttaggcatgaagaacatatttaattcagatctaaaaaatacatatattagaatgaatacaatgaatacttcaaataaaactattaatctgtgtttataaacacttaataaagaatgtttaaacgtgggctctataaacacaggattaaagtatacattaggaacattctccatttatagtaatcaatcctttgtcggaatatctgttagaggaatattctttttaacacattccaatagtcccagaaaatcatttaacctaatgggttcttgaagagggctagaactatacgattctagttccttattagtactaagtttctctagtttatttttattggtgaatccgtaaatggcattcaatctcatggatgtggaaggagaatacacattcaggacattaattcccgcatatttagatatcaaagccttgttgtctccgataggtgtatactcagtcgacgcggattccatattttctttataaatattaatctttttacgagtttcgaaaatacacaagataggtgatgatccttgacggaaaataaccatgttcttattctttctcctaccactttttttaggaatgacggcagtcatatccgtattattaatgataagaggcataacctcattgttaggatccaagaaatagttttcaatggcttcaatagttagtttatcatctcctagacccaaattgtttcttatattatacattgtggtaaacattgcccctgtgatattctcgttatgaatcaactccaactcatattttgacatcgttctaggtactatattaaaaatagaaagcatagatcttggaaatttggattttgcgccggcaataaccatttgtaaatcatcatactcagatatacttcctgcaaaaaatatggtatcttcttcgatgaggttttctagcagtagactcatttagagaagttttttttgtgataaatgaatacccgtaccgatgttacaaacgataatatagacaaaaatccaaccaaacgaggtgatagaaatataccaggaagaaatgaaagatttaatgaccaaaatagattcaacaacgatataccaaagcctaaaccaagactacagcctaatcagccaccgaaacaagataataaatgcagagaagagaatggagattttatcaatattagattgtgtgcctacgaaaaggaatattgcaatgacggatatctatctcctgcctattatatgttaaaacaggtggatgatgaagaaataagttgctggtcagaactatcgtcgttggtgagatccagaaaggcggtgggatttcctctattaaaggcggctaaacgtatttctcatggatcgatgctatattttgaacagtttaaaaacagtaaagttgtgaaattaaccccgcaagttaaatgtttaaatgatactgttatttttcaaactgtagttattttatattccatgtataaacgtggcatatattctaacgaattttgttttgatctggtttctattcccagaacgaacattgttttttctgttaatcaattaatgtttaacatttgtacagacatattggtagttctatctatttgcggcaaccggctctatagaacaaatctaccacagtcgtgttacttaaatttcatacacggccatgagacaatagcccgtagaggatatgaacactccaattacttttttgagtggttgataaaaaatcacatatcgctattgaccaagcaaacgatggatattctcaaggtaaagaaaaagtatgctacaggagcaccagtaaataggttgttagaacctggtacactggtatatgtgcccaaagaagattattactttataggcatatcactcaccgatgtgtcaattagcgataatgtcagagtattattttccacagatggaatagtgttagaaatagaagactttaatatcaagcatttatttatggcaggtgagatgtttgttagaagtcagtctagtactattatagtataaagtaataaaaaatagttaatgtgatgactagcgccaccaacgccaacaacatttgataatttctacttactagacgtaccgtaaaaatataaattactataacaaataatagtatatcaataaacaacctaattaatggtcgaagtatagcaggacattgatgctctagaccgtgtataacaaaatctacaaatttttcatccgctatattttgtttcactatatcgtctagacgatcagcgataacttccatgttaatctattaaaatattatcaatatattttcagttttgcatatccgtggtagcaataaccatcggagaagttctaaagaatgtgtccatgtaagtagtccaatggacgttttccttattggctaaaataagtttgattttatcattggtggatgtgaacaacatacgcttggcatagtacataaacaacgctgccaatattataacaccgataacaatcatataaaactgaactcctgtaccagcaacttgtctaggtgctatttgagtagtggccttagtcgtcaattgcatcaacgctttaatggcacaatttcctttgctagatcctgtattaataaattccaaatttgttggagatcctggggctccgtaacattcatctatgattacgttttgtatctttaatttgttatcgacgaccgcgctagaattacaagtctgtttcacataattttcaaaatctctaacaacagtgtttacactcgtctgaatgtttaacgcagcagtaaacatagctggtacgtatgctttttgttccggtgttaatccactatatgtttctgtagcggctgataacacagcatccaactgagcatccgcgtccgcagagcacatatttttaacagtgaggttacatccatggttttgtcggatataaaaatttccgatttctatatcacattttgtttgagcactagcgttcgcttcttgttctaatttagacgagatacgttcgctgagtgtattcaccgtcgtctgtatgcttgccgcggcacccatttaaatagctacaattagtatccatattaccaagagagataataaactgatcaaatgcaattttaggtcgaacgaatgtttaatattatgttgaactacttttgctctattgacgggaacagtagaaaatctatcactattgcttaatccacatgacaatcttaatgaggaagttttatccatctgtaagttgttcacgctagtattacatcgtacaatattgcaaagtcctaaattattataattacgtgttagtaagaaattaacattggcattcgaacactctggatcccaacattctcgaggttccgcatattttaatgactcttctaacttatctctagtgggataactacatctcatatatttctgtttaaagtccgcagactgttgtcttagaatataatcgatcatctctttgctatcttctgtattgtgtgcgcgtaaatgatgcaaaaatgattcacatattggtacactagcatctttactacataatgtttgcatcttattatacagatttattaacgattgttgaccctctacagttctatcactcctattaaaggctgaaccgatccactgatggcatatgtttctatcgaacgtatccccctgacaccagtcgaataaatcaacatcgcattttccagtgtcgtgaacgtctggccaacacgattctaatactgcaccctcttcatacttatctgatatctttccatcctttttccaataatgagtacgattaaaagtgcgacagcaattgggtgcggttccattatacgattgtcttaacaatgccgaaagaccaccgggtcctgtgttgactaatctaaactctggatatcttttctttactttatcctctttatcttttgctagtggtcctatatgcacatattctaaaagcttagcgggagctatcacgtcatgcattttatccacgtttaataacatctcatcagtgggtactcccggaggcggatcccgtttagggagctcaacacttactctgccacccatatttatctcattgaaagtattaatctaaaaacgtcataaagatgttgatcttaaaggattgaactctatccgaaaacaacattcctagaatgttatcgtcattatccattacgattctagtttcaaaaacattgactctctttttgaatcctcgtagtttgttgagagacgagatagctattttgaaagaaaacttttgtagttcttgagaacattcagtcatagaatattccctggaaaacgcatcagtattaccaggagtcttcataataatattgtcatctttaaacataatagccaaatgctgatgctgactaatacacttgataaagcccaacaaccattctaaatgaatgacggttctaccacaacatttttcttcatacttgtgaaaattaaacacgtaagatttcttttgatctatacttagacaaatagtagtatctgtcctaataggcatcagttccttgttacaatcgacacttactacatgataactagaaagttttactagattattttccagatcaggttctatatctatgatggcatcattgtgaaaactacacaaacacgattttaccttggacacaggaagattaaacacaatgttttcggctccgcggtagaacactgatgcactgagaggtataatggcccaaatgtttacagatccgcccaaggcggcaaaaatatacattaactcatccgtcgagtctacatttatagatacttcttcactgaactctgaaaaatatgccacaatttggcgcagtttatcgatttttatacggatgctcattttaaatttttgtaaattatttaaagttaaatggctgcagaacagcgtcgttctacaatttttgacatagtttcaaaatgtatagtgcaatctgtattgagagatatatctattaattctgaatacatagagtccaaagctaaacaattgtgctattgtccggcatcgaaaaaggaatcagtgattaatggtatctacaattgttgcgagtcaaatatagaaataatggacaaagagcagctattaaaaatattggacaatcttcgatgtcattcggctcatgtatgtaacgccacagatttctggagactatataattcgttaaaacggtttactcatactaccgcattctttaatacatgcaagcccactattctagccacgctaaacactttgataaccctgattttatctaacaagttattgtatgcggcagaaatggtagagtatctagagaaccaactagattcatcaaataaatcaatgtctcaagaactagcagaattattggaaatgaaatatgctctcattaatctggtacaatataggattttgccaatgatcatcggtgagcctattatagtagctggattttctggtaaagaaccaatttctgattattctgcagaagtggaaaggctaatggaactaccagttaaaactgatatagtgaataccacatatgacttcttagccagaaaaggtattgatactagcaacaatatagcagaatatatagccggcttgaaaatagaagagattgaaaaggtagaaaaatatttaccagaagttatatctacaattgccaatagtaatataataaaaaataaaaaatctatctttccggccaatatcaacgataaacagatcatggaatgctctagaatgttagacacgagtgagaaatactctaaaggatataaaactgatggagctgtgactagtccattgacgggaaataatacaattacaacatttataccaatttctgcgtccgatatgcaaaagtttaccattttagaatatctttacattatgagagtgatggcaaacaacgttaagaaaaagaacgagggaaaaaacaacggaggagtagttatgcatattaactcaccctttaaggtaatcaatttgccaaaatgttaaattattagaatcatatatctttatccatctattatcatctgtgaagttttggctcagaaaaatatctttacctaatattcgtttagacgatgtatcaataccggcatttttataacattcagctaccaatttaaaacaatacattctattatgaccaaatccatacggaatacccaataaagttaatgacgtatcagctgctattttcataacttgaatattctctaacatgtatacttttgcactaagatatccttcaaaaaaactatccaatgggactattcgaactccttttctataggtagattcaactaccaagtgttccgataataacgtaccataataaatacccacatgtttttctatcgatggattcaaatgacttgttaatgacatggtataattaataaaaataatagaacctctaggcgcatatgtcttgataaaattaaccggatccatattagtttatatccaatagaggttgcactgttaagttacgttgaggttctatttgtgtagataattttaacctacaacattcgttctttacactgacgagtacatcctttaatgatttttttcgtataatcaatttatatcgttcgtgagaaatatctttgccgcacgtagagcacacgagttggaacaccatttttaatctagtaacctatatttattatattatcacttttagtattggttattaacagactatcgttaaagtaaaatgaactatttatactcagtagtaaattatccttatagatgccgaacactatagtgttacagttttcgttagaatatccgaagatcgaagatatagattttataacatcgatatctgatttagtattttcgaaatctatataagatgataaagttttaacttctgaaacatcaatctccttgcgtggttctaatgcatacattaattggcagggtaacggaacggtctctaaataagatgatttaaaattgttccatttttcatctaatgcggaaaatataaattcttgaacagtgcattgacaaggaggaacatacgagtagacatcgtctaaattagcgtaatcgtttataaacgtaacaatattacgcacgtctacggtagagttagtctttctataataatttttaattgccaaactagtgactatattatcgattgtaaaatcactaaacaattgtattttgtttaagttggtgggtgttatagatgccccatagagtccaggaaagaaatcacatccattcactaacgccgttaattttgctaaatagttatcctctgccgagggaacaaatttaaacagttgagttaagttttttatcatcttaggatgattatcagtagatgcaaatagcatagtatcctgatcggtacttattatcaacggccattctcctgtggtagagaaatgtgttttagcctcgagacacataacgaattctgcgtctcgttcatcacaataaaataatgtaacattctcattattgttgaaatgtgttagtatctcattcaatgatatttttatgttatcagaatcagataaatatatttgaaatgtgagtttatcgatttttaattgcatttctgcctttatttcttcttccataaatccggtaacattttgtatttcggatgtacacttttctaattctagcatttttctgtccttggttaatttagaatatttacgtctcttgtctctaacgtcttgtttaatttttatacttcctctatcgataaaaagggttacatgccctcccttttttacccatccgtttacgtattttatgaataccgtagttaactcttctaagtttctgacacaattggcgacggctatataaatactcattgtatccacaaatattccattgtatactttgtataaattatcatctaatatcgtcagtgatttattttccagcagtaacgattttaagtttttgatacccataaatgaagaacgtactgattattttcggtaaaccgtattgtagtatttgtgaaaatgtcagtgatgcagtagaagaattaaaatccgagtatgatatactccatgttgatatcttatcattttttttaaaggatggtgattcaagtatgctgggtgacgtaaagcgcggaaccctaataggaaactttgcagcgcatctatctaactacatcgtttccattttcaaatacaatccacagacaaaacaaatggcatttgtggacattaataaatccttggatttcaccaaaaccgataaatcgctggtgaatttggaaattctaaaatccgaaatagaaaaagcaaattatggagtttggccaccggttaccgaataaaatatcttcctgagtattttgtttattgttccgcttcctttatcaaataaattttgaagtatttgataatttattggatcgactagttcatctatacattttttggaaacgagaacatctacctttttatttgttgttatatgcgacgttgtatacgccataaatgtaacttgagaaaatccacaaagaattaattttgaaccagttatggggacgcgctcattagaatatgtataaatctgaggagtgtattccttgaggagatattctatcaagtcatcatttgcttctctaatataaatattcctgtcctttataacaactatgccttttagattatctcctaatttataacacggccgtatacacttggaatacttgtacaatctaagatagactaaatctctcagagttgtatcatggaattccatacttaattcctccgataaatctacatgttcaaatactttagaaatatatttatggtatttcctatctggaaagtaagttatcaagtcatcataagatatttcaaatcctctacataaggaagacactatctccaacgattcctcatcttctgtaagtaggaatttggacaagttaaacaaaattagatctctaaatggcatctttattatatcttattttatttttgttattcgtatgtatttcttattattttacatattatccgaccaataaacttcaggcagctgtaatggaaacagatagagaaaacgctattattagacagcgaaatgatgaaataccgactagaacattagatacagctatatttaccgatgcatcaaccgtcgcgagtgcgcaaatacacctatattataattccaatattggtaaaattataatgtcacttaatggtaaaaaacacacctttaatttatacgatgataacgacatacgaacattacttcctattttactccttagtaaatgattgtcttaccgaataaagttcgtattttcatcaacgatcggatgaaaaaggatatctacttgggaatttctaatttcggattcgagaatgatatagatgaaatcttgggaattgctcacttgttggaacatctacttatatcctttgattctactaattttttagcgaatgcttctacatctagaagttatatgagtttttggtgtaaatccattaattcagcaacggaatcggacgcaatcagaacattagtttcgtggttcttttctaacggaaaactcaaagataatttttccctttctagtatacgatttcacattaaagaattagaaaacgaatactattttagaaatgaagtattccattgtatggatatactaacgtttcttagcggaggcgatttatataacggtgggagaatagacatgatagataatcttaatatagttcgtgatatgctggtaaatagaatgcaaaggatatcgggatcgaatatcgtaatttttgttaagagattaggacctggaacattggatttcttcaaacagacatttgggtctttaccagcatgtccggagattattccttcgtctattccagtaagtacaaacggtaaaatagttatgactccgtctccattttatacagttatggtaaagattaatccaacattagataatattttagggattctgtatttgtacgaaacttaccacttaatagactatgagactatcggcaaccagttatatttaacggtatcctttatcgatgaaactgaatacgagagctttcttcgtggcgaggctatattacaaattagtcaatgtcaacgtattaatatgaattatagcgacgattatatgatgaacatctatttaaattttccttggctatcgcatgatttatatgattacattacacgtattaatgacgatagcaagtcgatactaatatccttgacaaatgaaatatatgcatctataattaatagagatatcatagttatttacccaaactttagtaaggccatgtgtaacactagagatacccaacaacatccgatagtagttcttgacgcaaccaatgatggactgattaagaaaccttatagaagtatacccctaatgaagcgtctaacatctaatgaaatatttatacgatacggagacgcgtctctcatggacatgataactttatcattgtctaaacaagatatatcattaaaaagaaatgccgaaggaatacgtgtaaaacatagtttttcagctgatgatatacaggcaattatggaatctgattcgtttttaaagtatagtagatcaaaaccagctgcgatgtatcaatatatatttctatcattttttgctagtggtaattccatagatgacatattggcaaatagagattctaccttagaattttctaaaagaactaaaagtaaaattttgtttggtaggaataccagatacgacgtcactgcaaaatctagttttgtatgtggtatagtacgaggtaaatcattggataaaacgtctctggttgaaatgatgtgggatctcaagaagaaaggattaatatattctatggaatttaccaatctattgagcaagaataccttttatctgttcacatttactatctacactgatgaagtatacgattatctaaacactaataaacttttttctgcaaaatgtttagtcgtgtctacaaaaggagatgtggaaaatttttcatctctaaaaaaagatgtggtcattagagtttgatttttagttattatctacaggaacaaatatagtatctgaaatcatattcatatatcccgttagaggtctatgataatatatagtagcgtttgttccgttatagacaccgaataatattttacaaaagtgtatatacgtatcatcatctttatgtttaaaatttaaaatcttaattcgtaaatttagagataaaatggcttcttgtacaatactagttaattctcccgtcctctcaaaattatccaactcctcagcgagaataggacttagtacataaagtttagcatactctatcatcttcatataataattggataataatttattccatgtttctgtactaatatcgaacgagtctatatattcctttgtacgccatagaatatccaaatttgtagggattataaacaaatcttcggggagtgttagattaaacttattagcgtacaatatataattatgtagaaattctgaatctattcgctgtatagacttcatataagacagatcatagaaatatacgtatgtcccaggattaactcttcctactcttccttttcgttgatctctcatagatttagaaataaattcttgcgatcctccaaaaggagcggggacaaaaactctacccatatcataaatgtgtgtaacattgcgtatagtaacgctggattccaaataaggagtagaaataattatcgatacattaggtgatgaatacactttttctaatatttcgtctatatctaagaccttaccatgaataatatacatatcatacggtaatcttttttctaaatatgatttatattcgtgacactgtgcaacggatgccacaaagactataccggatgatccatcaggaggagtatacatctgtatagcagtaactaaatttctcttttcttcttctatgtatgccattctggaagatggatttatcttattatgaataaatacctcgctaattttaaacagtgtatctccaggaatatgtataaatgcgggattaggtaaaaatacttttagccgttccctgtcatcctctaacgtggcagtcattaaaaacatagaatctattttcgtatgatgctttctcgctactgctataataatatctcctatttgatcatgctcatgaacttcgtctataataagagtgccataactaaatagttttgttagagataacttatgggtagaaaatacaattccatatttttttggttgtttgtttattaattcttccggtatagatccgtaccgtaaagaaataggagatccatctagtaccttaaatcccaatgattttaaaatggtattgctatgcaatctaactaaagctatcctaggaagagatagaatgactggtctttcgtgaaagtcagtgattttatctagagtagagaatccaccaaataaataattaaaccaaagcaataacttgggtacctgtgacgtcttacccactccagttcctccagttaaaactacaggtctatgagaaatccacgctgaaaatatctctcgttgtgctttaggagttaatgaggccaatggaattttgctaaatggatacttattccctagtacacctatagtatctgttcgtttacctacggaattggaaaatatctgtaaactattaccagcttctaatagtcctcttaatatatactcatcgaacgaattgatttctgtatttgttatacattttagaaaactataacattcaaagcttattgtgtgttcactgtatatatattgcttcgcggttaggtttatgtgtgcgggttttcctagtgatggaggagatactttttttccacgtgcttctgtactaactttgtatattcctttatgttttacaacgtgtgcgttatgccatctatgttttataactggaaacaccgccaatgagaaactatcacgttccgttttactcatattactgaattcatcttgtgaatatttgtaagagaatacattaacgcagtttggaaaaaagaagatatctggtaaattcttttccatgataaatggaaagatatacagatttagtaattagtaaaataccagaactaggatttaccaatttattatgtcatatatattcactagctggattatgtagcaatatagatgtatctaaatttttaacaaattgtaacggatatgtagtggagaaatatgataaatctacaaccgccggcaaagtgtcttgtattcctatcggtatgatgttggaactagtagagtcggggcacctgagcagacccaatagtagcgacgaactcgatcaaaagaaagagttaaccgacgagttaaagacgcgttaccattctatatatgatgtctttgagttacctactagtataccgttagcgtatttctttaaacctcgactacgggaaaaagtatctaaggcgatagacttctcacaaatggatttgaaaatcgatgatttatcacgtaaaggaatacatactggtgaaaatccaaaggtcgtcaagatgaaaatagagcctgagagaggagcctggatgagcaatcgaagtattaagaacttagtctctcagtttgcttatggatccgaagtggattatataggacaatttgacatgagattcttaaactccttagcgattcatgaaaaatttgacgcgtttatgaataaacatatcttatcgtatatacttaaagacaaaattaaaagttctacctctagatttgtaatgtttggattttgttatttgtctcattggaaatgtgtaatttatgataaaaaacaatgtttagtatccttttatgactccggaggcaatattccaactgaattccaccactataataatttttatttttattccttctccgatggttttaacacgaatcacaaacattctgtattggataatacaaattgtgacatcgatgttttattcagatttttcgaatgtacatttggagcgaaaataggctgtattaatgtagaagttaatcagctgttggaatctgaatgtggaatgtttattagtttgtttatgatattgtgtactaggacaccacctaagagtttcaaatctctgaaaaaggtttatacattctttaaatttttagcggataagaaaatgacattatttaagagcattctatttaacttgcaagatctatccctggatataacggaaacggataacgcaggattaaaagaatataaacgtatggaaaaatggaccaaaaagtcaattaatgtgatatgtgataaattaactacaaaattaaatagaatagtaaacgacgatgaataactttgttaaacaagtagcttcaaagtctctaaaacctaccaaaaaattgtctccgtcagatgaggtgatatctttaaacgaatgcataatatcctttaacttggataacttttattattgcaacgatggactgtttactaagcccattaatactccggaggatgttcttaaatcactcttgatcatggaatcattcgcctacgagaagatgataatcaaaggattgataaaaatactaatatctagagcatatattaatgatatttattttactccattcggttggttgacgggcgtcgacgatgatcctgaaacacacgtggtgataaaaataattttcaattcatcactaatatctatcaagtctcaagttatagaatatttaaaaccatacaatgtcaataacctatcggtacttaccacagaaaaagaattaagtattaatacgttcaatgttccagattctatacctatgtcgataatttcgtttttcccattcgatacagattttatactagttattttgttttttggagtatataatgactcgtattgtggaataagctatataagtccgaaagagagactaccgtatatcatcgaaatattaaaaccgttggtgtcggaaattaacatgttatcggatgaaataggtagaacatcatccattagaatcttcaattccactagcgtcaaaaaatttcctactaatacattaacatccatttgtgaaattgtttattcgtttgatgaatcatcctttccgacgcctaagacgttcactcctctaaacgcgagtccatacattcctaaaaagatagtttcactattggatttaccatctaatgtggaaataaaagcgatatctagaggcggtgtggatttcatcactcatattaataataagcgtctaaacacaatcttggtaatagcaaaagataactttttaaaaaattctacattttctggaacttttatcaaagagaatattatttggaagggtatctatacttatagaataatcaagtctagttttccagttcctactattaagtcggttactaataaaaaaaaaatatgtaagaaacattgtttcgtcaattctcagtatacaactaggactttgtcacatattctttgatctaatttttagatataaatggtggatgctataaccgttctaactgcgatcggcataactgtattaatgcttttgatggtaatttctggcgccgccatgatagtcaaggagttaaatcctaatgatatattcactatgcaatcattaaagtttaatcgagccgtaacgattttcaaatatataggactctttatctatataccaggaacgataattttgtacgctacatacgtcaaatccctattaatgaaaagttaaataattttttttattacaccaacaaaaatgtttgtcattaaacgaaatggatacaaggaaaatgtcatgtttgataaaatcacgtctcgtattagaaaattatgttatggcttaaacacggatcatatagatcctattaaaatagctatgaaggttattcaaggaatatataatggagtaacaacggtagaattggacactctggcagccgaaatagcagccacttgtactacacaacatccggattatgccattctagccgccagaatagccgtatcaaatctacacaaggaaacaaaaaaactatttagtgaagtgatggaggatttattcaactatgttaatcctaaaaatgggaaacattctccgattatttcaagtatcaccatggatatagttaacaaatataaggataaactcaactcggttattatttacgaacgagacttttcatacaactattttggttttaaaactttggaaaaatcctacttgttgaaaataaacaacaagatcgttgaaagacctcagcacatgttaatgcgtgtcgcagtaggaattcatcaatgggatatagactcagctattgagacgtacaatctactttctgaaaaatggtttacgcacgcttctcctaccttatttaatgcgggaactagtcgtcaccaaatgtctagctgttttctacttaacatgatcgatgatagcatagagggtatctatgacacgttaaaacgatgcgcattaatctctaaaatggcagggggaataggtctatcaattagtaatattcgtgccagtggaagctatatctccggtaccaatggtatatcaaacggtattattccaatgttgagagtttataataacaccgctagatacatagatcagggaggaaacaaacggcctggagttatggccatatacttggaaccgtggcattctgatattatggcgttcctcgatcttaaaaagaatacaggaaacgaggaacatagaaccagagatctatttatagctctttggattcctgatctctttatgaaacgagtgaaggatgacggagagtggtcgttgatgtgtccggatgaatgtcctggattggacaatgtttggggagacgagttcgaacgattgtatacactatacgaaagagaaaggagatacaaatctataataaaggctcgagtcgtctggaaagcgattatagaatctcagattgaaactggtactccattcattctttataaggatgcgtgtaacaaaaagagtaatcaacaaaatttaggaactatcaagtgtagtaatctttgcactgagataatacaatatgcggatgctaatgaggtagccgtttgtaatctggcatctgttgccttgaacatgtttgtaatagatgggcgatttgattttctcaaactgaaggatgtggtcaaagtaattgtcagaaatctcaataaaattatagatattaattattatcctattccagaagctgaaatctctaataagagacatagacctatcggtattggtgttcaaggattagcggacgcgtttattctcttaaattatccatttgatagcctggaagcacaagatctaaataagaagatcttcgaaaccatttattacggtgcattagaggcgagttgtgaactagctgagaaggaaggaccatacgatacatatgtaggatcgtacgccagtaacggtattctacaatatgatctttggaatgttgtaccgtcggatctttggaattgggaacctctaaaagataaaatcagaacatacggtcttagaaatagtttattggtggcacctatgccgactgcatcaactgctcaaattttgggaaataatgagtcggtggaaccgtataccagtaatatttacactcggagagtattgtctggagaatttcaagtagttaatccgcatctccttagagttttaaccgagagaaaattatggaatgatgagatcaagaataggattatggcagatggtggatccattcagaatacaaaccttccagaagatattaagcgagtttataaaactatttgggaaattccacaaaagacgatcataaaaatggctgcagacaggggagccttcatcgatcaaagtcaatctatgaatatccatatagcagatccgagttattccaaactaacgagtatgcatttttacggatggagtctcggtctaaaaacgggaatgtactatctacgtacgaaacccgcatccgctcccattcaattcacattggacaaggataaaataaaaccactggtggtttgcgattccgaaatctgtacatcatgcagtggttaaacaaaaacatttttattctcaaatgagataaagtgaaaatatatatcattatattacaaagtacaattatttaggtttaatcatgagtaaggtaatcaagaagagagttgaaacttcaccaagacctactgcatctagcgattctctacagacttgtgcgggtgttatagagtatgcaaaatcgattagtaaatctaatgcaaaatgtatcgaatacgttacactaaatgcttctcaatacgctaattgttcgtctatctctataaaacttactgatagtttatctagtcaaatgacttccacttttattatgttggaaggagagactaaactttataaaaataaatctaaacaagatagaagcgatggatactttctaaaaataaaagttaccgcggctagtcctatgttgtatcaacttctagaagccgtctatggaaacattaagcacaaggaacgcattccaaattctttgcatagtctttcggtggaaactattacagagaaaacatttaaggatgaatccatcttcatcaacaaattaaacggatccatggtagaatatgtttcgactggagaatcatccattctcagatctatagaaggtgaactagaatcactcagtaaaagagaaagacaattggccaaggcaattatcacacctatcgtcttctatagatccggaacggaaacaaaaattacattcgcactcaagaaactaatcattgatagagaagtggtggctaacgttatcggactctctggagatagtgaacgtgtatcaatgactgaaaatgtagaagaagatctggctcgtaatctgggacttgttgatattgatgatgaatatgatgaagatagcgataaagaaaagccaatattcaatgtataaatggataagttgtacgccgctatatttggtgtatttatggggtctccggaagatgatttgacagactttatagaaattgttaaatctgttctaagtgatgagaaaacagtcacatcaactaataataccggttgttggggttggtattggttaattattattttttttatagttcttattctactactattgatatatttgtatttaaaagttgtttggtgaacttaaatggcggaatttgaagatcaactcgttttcaatagtatcagtgcccgtgcattgaaagcttatttcactgctaaaatcaatgaaatggtagatgagttggtcacaagaaaatgtccacaaaagaaaaaatcacaagctaagaaacctgaattacgcattcctgtagatcttgtaaagtctagttttgttaaaaagtttggattgtgcaattatggaggaatccttatcagtcttattaatagtctagtagaaaataatttctttacaaaggatggaaaactggatgatacaggcaaaaaggaattggttttgacagatgtcgaaaaacgaattcttaataccatagataaatcatctcctttgtatatcgatattagtgatgttaaagtattggctgctagactaaaaagaagcgctacacaatttaactttaatggacatacatatcatctggaaaatgataaaatagaagatctcattaatcagttggttaaggacgaatccattcaactggatgaaaagagttctattaaagatagtatgtatgtcattcccgatgaacttatcgatgttctcaaaactagattgtttagatctcctcaagtcaaggataatattatttcgcgtactagattgtatgattattttactagagttactaagagagacgaatcgtcaatctatgtgattctaaaggatcctaggatcgctagcattttgtcactagaaactgttaaaatgggcgcctttatgtatacaaaacatagtatgttgacgaacgctatttcatctagagtcgatagatattctaaaaagtttcaagaatctttttacgaagatattgtagaatttgttaaagaaaatgagagagttaatgtatcgagagtggttgaatgtttgactgtgcctaatattactatatcaagtaatgctgaataaaaatatttataaatatgctcgtcgtaattatgttttttatagcgtttgccttctgtagttggctatcatatagctatctgcgtccatatatctcgactaaagagttaaataagtcgagatagttttatatcacttaaatattaaaatggccgaggaatttgtacaacaaaggttggccaataacaaagtgacaatttttgtcaagtatacatgtcctttttgtagaaatgcactggatattctaaataagtttagtttcaaaagaggagcgtatgaaattgtcgatattaaagaatttaaacccgaaaatgaattgcgtgactattttgaacaaattactggtggtagaactgttcctagaatcttttttggaaaaacttctattggtggatatagcgacctgttggaaatagacaacatggacgcattgggtgatattctatcatctattggggtattgagaacttgttgagaaaataatgaaaaaacaatacttaatcatgtcgccgacatgttcatgtatccggaatttgcgaggaaggctttatcaaagcttatttcaaaaaaattaaacattgaaaaggtgtctagcaagcaccagctcgtgttactggattatggattacacggactattgccaaaatcactgtatctggaagctattaattccgatattctcaatgttagattctttcctcctgaaataataaacgtcactgatatcgttaaggctctccaaaattcttgtagagtagatgagtacctaaaatctgtttccttatatcataagaattctttaatggtatcgggaccaaatgtagtcaagcttatgatagaatataatcttcttacacacagtgacttggaatggttaattaatgagaatgtagtcaaggctacataccttttaaaaatcaatgcctatatgattaactttaaaatagatctaacggttgatgaaatcattgacttagttaaagatattcctgtaggagctacgctacatctatataatatattaaacaatatagatttggacattgttcttcgtatatctgatgaatataatataccacctgttcacgatattctgtctaaacttaccgatgaagagatgtgtataaaactagttacaaagtatcctatggacaatgttataaattttattaatcaagatgttagatatagtcccaccttcatcaagacaattaaagattttgtcaacaagcatcttccaaccatgtacgatggattaaatgattatctacattctgttattatcgacgaggacttaatagaggaatataaaattaaatccgttgccatgtttaatttggaatacaaaactgatgtagatactctaacattggacgaacagatatttgtagaggtaaacatctcatattatgattttagatatagacaatttgccgatgaatttagagattacattatgataaaagaaagaagacaaatcaccatgcaatctggtgatagaataagaaggtttagacgtcccatgtcattgagatccactatcatcaaaaaggatactgattctctagaggatattctcgcacatatagataatgccagaaaaaatagcaaggtatccattgaagatgttgagagaatcatttcatctttccgtcttaatccttgtgttgtcagacgcaccatgctgtctgatatagatatcaaaacaaagattggaaatcttgtgctctgacactatcagccatcaaaggaattatggtaacagataccatcaataccgtgttatccaaaattctgcatcatcataggaatgtcttcaagtatcttacatctgtagagaataaagaaattgctgtctgtcgctcttctatagagaattaaaaagtgtacgatgtgatctacgcacagatgatggattattggataggctatacgatctgactagatacgccttacacggaaaaatcaatcaaaacttaatcggtcaacgatgttggggtccgttgacagaaatgctgtttaacgagaataaaaagaaaaaactaaataatttaatggaatacatcaaaatatcagacatgttggtatacggacactctatcgagaagacgcttattccaattactgattctctttcattcaagctatctgttgataccatgtctgtgttaaatgatcaatatgccaagattgtcatcttcttcaataccatcatagaatatattatagctactatctattatagattgacagtcttggacaattatactaatgtcaaacattttgtatccaaagtgttacacactgtcatggaagcatgtggcgtactgttttcatacattaaagttaatgacaaaatagagcatgaattggaggagatggtggacaaaggtaccgtaccttcttatttgtatcatctgtccatcaacgtcatttcaataatattggatgatataaatggaactcgttaatatttttttagaaacggatgctggaagagtaaagtttgccataaaaaataccgacgatgtatgtgcctcggagttaataaataaatttgtggaactgttaagtgaatacattcacattgaccaatcagaattttatttggtggtaaaggataaggatattttttattttaagtgtgatagggggtctatttcgattgtaaacaatgagttttatgtctttgacgaacccttgctgtttgttaaagatttcactaatgtaacgggggttgaattcatagttacagaaaccatgccgtgtagaattataccaaaaaataatcacgcggttatttcagtcgtgactaatcataagttttataatgggttaagtttataaagggttaacctttgtcacatcgatcgcgtatttgggatcagatgccaaattgttaaataatctgatgaaaaaataataaatataattcagatcatcgctagacatgacattattgtcctctatagcgatagtcgcgtgccgtctacatgcaggacatggaagagtgctgactatagtatatagttttcgtttacacgcttctatgttgccgtctaaacccgcttgcgaaagtactataaaaataatggtccatacggctcttccccaatgtttgggattcatttaaatgaaaatatatttctaaattctataaatggatgttcggtgcattaattggtttgaaagtcacggtgaaaacagatttttatatctgaaatccagatgtcgaaatggcgagaccgtatttatacgatttcctcattacttttattacgtagttacggacgaaatatatcagtcattgtctcctcctccatttaatgcgaggccgttgggaaagatgagaactatagacattgacgagacaataagttataatctagatattaaagatagaaaatgctccgtcgcagatatgtggttgatagaagagccaaagaaacgcagcatacaaaatgccaccatggatgaatttctcaatattagttggttttatatttctaacgggatatctccagacggatgttactcgttggacgagcaatatttgacaaagattaacaatggatgttatcattgtgacgatccacgtaactgtttcgctaaaaaaatacctagattcgatatcccaagatcgtacttatttctagatatagagtgtcacttcgataagaagtttccttctgtatttattaacccaatctcgcatacaagttactgttatatcgatttaagtggtaaacgattattgtttacgctcattaatgaagagatgttaacggaacaggaaatacaagaagccgtcgatagaggatgtttgaggatacagtcactaatggaaatggattacgaacgagaactagttctatgttctgaaatagttttgttacgaatagctaaacaattgttggaactaacgttcgactacgtcgttacctttaacggacataactttgatctgagatatattactaatcgtctagagttattaacaggagagaagattatctttagatctccggacaaaaaggaagctgtacatctctgtatttatgagagaaatcagtctagtcataagggagtaggcggcatggccaatactacgtttcacgttaataacaataatggaactatatttttcgatctatattcattcattcaaaaatctgaaaaattggattcgtacaaattggattctatatccaagaacgcgttcagttgcatgggtaaagtattaaatagaggagttagagaaatgacgttcatcggtgacgatactacggacgcgaaaggcaaagccgctgcatttgcaaaggttttaaccacaggtaattatgtgactgttgatgaggatattatatgtaaagtaattcgtaaagatatttgggaaaatggatttaaagtcgtactatcatgtcctactttacctaatgatacatataaattatctttcggaaaggatgacgttgatttagctcagatgtataaggattataatctaaacatagctttagatatggctagatactgtattcatgatgcttgtttgtgtcagtatttgtgggagtattatggagtagaaacaaaaacagacgcgggtgcgtcaacatatgtgcttcctcaatccatggtattcgaatatagagcgagtacagtcatcaagggtccactgttaaagctattgttggaaactaaaactatcttagttagatcagaaacaaaacaaaagtttccttatgaaggcggtaaggtatttgctccaaaacaaaaaatgtttagtaataatgtattaatctttgattataacagtctgtatcctaatgtgtgtatctttggaaatctatctccggaaacattagtcggtgtcgttgttagtaccaatagattggaagaagaaataaataatcagctcttgcttcagaaatatccacctcctagatatattacggttcattgtgaacctagactaccgaacctcatctctgaaatagcaattttcgatagatcgatagaaggaaccattcctagactattaagaacatttttggcagagagagccagatataaaaagatgctaaaacaggctaccagttcaactgaaaaggccatctatgattccatgcaatatacgtacaagatagtagccaactcagtatatggtctgatgggatttagaaatagtgctctatactcatacgcttcggctaagagttgcacatccataggacgtagaatgatcttgtatctagaatcggtactaaatggagcagagttatctaacggtatgttacggtttgccaatccattaagtaatccattttatatggacgatagagatattaatccgattgtgaaaacatcgttgcctatagattacagatttcgttttcgtagcgtgtatggagataccgactccgtgtttacagagatagacagtcaagatgtcgataagtctatagaaatagcgaaagagttagaacgactgattaataatagagtattgtttaataattttaaaatagagtttgaggcggtatataagaatctgattatgcaatcgaagaagaaatatacaacgatgaaatactcagcatcgtcgaattcaaaatctgtacctgagagaattaataaaggtactagtgaaactagaagagatgtttccaagtttcataagaatatgattaagacatacaagaccagactgtctgagatgttgtctgaaggacggatgaattctaatcaggtatgtatagatattctccgttctttagaaacagatttacgatccgaatttgatagtagatcgtctcctctagaattatttatgttgagtcgaatgcatcactcaaattataaatccgcagataaccctaatatgtatttggttactgaatataataaaaataatccagaaactatagaacttggagaacgatattattttgcatatatttgtccggctaatgtaccatggaccaaaaaacttgtaaatattaaaacatatgaaacaattatcgatagaagttttaaactcggcagtgatcaaagaatattttacgaagtttactttaaacgattgacgtccgaaatagtcaatctattggataataaagttttatgcatctcattctttgaaagaatgtttggttcaaaacctacattttacgaagcataaaataatttacaacagttgtacgtcgctctttgttagattcagtttatccattagatattctacggctggagtaatttttgtagtaattgaatacccaggtacgtaaaacaataaaaacattaataccggatcatacttggtaaagtacaatatagtactaatattagctaatgtctcataatcggatggacttcttgctctactaagattagggtgatctatatctttgatacttattggaacgggaccggtaaatatataattctctttgagacgactaaacaaaggcttatagtctcctgtttgatgcatatacctagccaacgccaggtgtttggcattatctgtatagacgtatctgaataatgtgtgtttagtaatatcaaactgagtatttttaaaccagaacaagatattatagattggaatggaagtcagtaacaaaaaactaataaactcgtttgttggaagggcagaaaactttcgaagatatgatacgtatctaccagagaacgatccattagcaatgaattcagtatttactttttcttcatatacgggaataggacctttatatagagacatgagaaactcgaactttttaagtaatcccaacgaaatgggataatagtcacttgcatctaagtttgcgtatctactactccaaattgcagtttctggtatccatccatacgcgtaattatcaaatagatatgtgtttttcattaacggtctacttagacctctactaaaaaatgtttcttgatctagaattcttctttgtgcatttttgtacacgtcgtcaaaacgcggaacggtggcagccataatatttatatcctaccgctttttatcaactatgttatgtcttttagttaggttaatattctaataagatgcaggtaatacatcaggttaaagtattagaatgggattatacttatatatttagtttatctttcatcagataactaaaaaatgtataaaacagacgcgttacattgcctatgctacatgagttccatgtgcggagattgttaaagtttagggtagagagttgttctagcatccattcattgtctttacatccgttatatgtaattatgtcacccatggacattataggttttaatagtacttcagatactggaataagatattgttcgtatatgtgtttgataatcatttgttgcgtttctggagaacttttctctgcatgatttttacatagctttacaaactcgtgatcactttttataatgagagatctatagtcttcgtatctgttacgaaaatcaatatattcaggattattttctgaatcacttgattcgtcactaatatacataactatatgatcaacaattttttccaaaatcatttctgcattttctttattcattaatttggtgggagtctgaattgttgcagcagttcccatttatcaaaaatgtgtgataattatatctatgtatatacaaattattaataatatatttggtctgttctatgatctaccgtgtcttatcaattgaagtatatatttcttatccgtcttggttagatggatgcttttatccaagaattcttctacatgatatagattatctctcaaaaacctttgaaataagacgatgatggaaatattataactagctaaaatagttttgattagcgatggaagaatactagggtttttatctgtagaaaatcgaacgaatgcggatacatcctccacagacttaagatggtttatcattttaactaacaagtctcctttataatcatctaacacaagacctagacgagccaattgtccgtctaaataatattttaaaatgtttttgctaaacaagttcagccgtctactcttggattttatagatgacagtttctttagtattccgttctctatgaccatgggatccgactcgtgcaaaaacaggtatgtattgtattccttaacataatacgacgtatcctcgttaaagatttgacacttcttcatatcatccattgtatctaatgtgatatcgttcacctcataaattatattagatatataactgttattaatatctaactcatcaaatcgtttaatgtccgcagcactcgtagagaataaagacataattcgttcattatcgaaatattcgggatctatttgaacaacaactctgaataagtcctcgttaaacgttctatcactaataaatgtattaccgttaataaaattagctttataaaaactaataaaatattttttccaatcgtcgttttttgcatcaataatagataggatatctccaggaatatcatatatgatttctagcagaagttctctgatatctttttcaggctcaaagatataaactaacttaagcatgtttttattaacgcgtccttgttttatctcagagtatatataactaagaattttatcatacatggtaatggcgtgtctattaagaacaatatccgcatcattagctatactttcgtttaaccacacccaatagctagtcttatctccgggaaagatataatctctaaacgtttctgttagattgctatggactattctaccagtttgttgaaatagagtgtatatatcttgtctatcattatccgcaaactctatgcctgtatctttcttgcttaggtaagcccaaataagtaatcttataacacttaattttttaacaactttgaagtctgttttcttaaacagttttaaaaaaatgatttccatatctttgatgggttcccctccgtgaaataatggattaagaactaagtcataatttacgcctataagttctcctaattcgtccatgtattcggcaacctctaaaaatttttcatcttgtgttagtttatgcacggtttccttgtacgattgtattgcgttactaaacgataaataattcttagtcaatgctttaacgtacaacgtgggtgcatcaaatgcgtgtcgtaaaaccgcttcgtacactttacaacatcgtaccatatgtatgaaaacaaaaaaattggtattagctaatacatccttagtaataacgtgttgaggaaaattgctaactagatacttgagagctagtacatgattgagggtaaaattgtttactttacttaatgtatcatcctttaaaaaatctatattattttctactgtgtatataagatactttctacgaataaaatccatttttatagaagagagttatctatgatactacactcttattctaataattatttttactattttatcactaataactatttatactatccattattaatttaacaatttgaattagtattggagaaagatgaaatgcctattcactaacaaagtcaatgcactcttgaagatttttaccaataatatgtagcggattctcaaagtatttatttaccatttctttacacagtatcttagccactggcctacgaacaggcatacatggttcaactatatcatgaacaatgtttaattttagtgtatccaatccaataaatccattttctacatcatcatcgtatctgtacgctttgtattctccactcttgaacaaatctctaaacagtatcttcaacactttttccaccaattcctgggatgtcacacattgttctaccatctgtttgataaagagggtatcttcttcagattttcccttgattctatacatgggttttaggttataccttgcatctattgtatatttatcagattcagctatcaacattttatcaacttgtgttccaattattctccatttattagatctgcattcattattatagaatctttcttccactaatattctaataagattaagtttgaaaggagagaagatcttgtgtttgattttactcattggatgcattctataacgaatgaatctatgatcatctccaaacaaattgggaaacatgtaaagcagcagagcatataccatcttactcggattctgtgatgctttgccgatggctgaaatgtccgagaatagtttataagttttctgattatttggaatagatggtgctatatcttctagagtagtagtcctaatcattctcttaaattttatgtatcctagtttcaatgtctcgtaatgagtttgtgctgctcttattgtctgatttatttcttttaccattttggctctattctgaaactttatcctcttcttatccatttttattgttgactccgcactatcgatttgatactttcttttcagagtaaagctatcgtcattgatcatcggacactgacttccactcattatgaaattgtagccgtataaccacaatacaattattaacatatatattcacttttgttaatatcaacataataatgaaaaaatataaatgaacaaagttaatacataagtgttataaatggaaaatgtatacattagtagttactcatccaatgaacaaacatcaatggcggtagccgctactgatatccgagaattactatcacaatatgtggatgatgccaacttggaagacttaatagaatgggccatggaaaaatcatcaaagtactacatcaagaatataggtaatacaaaatctaatatcgaagaaactaaattcgaatcaaagaataatattggtatagaatactcaaaggattccagaaacaaactatcgtatagaaataaaccgtctattgccacaaatttggaatataaaacactatgtgatatgattaagggtactagcggcaccgaaaaagaattccttcgctatctcttattcggtataaaatgcattaagaaaggagtagaatacaatatagataaaataaaggatgtgagttacaacgattattttaacgttctcgacgagaaatacaatacaccgtgtcctaactgtaaaagtaggaatactacgccgatgatgattcaaactagagccgctgacgaacctccactagttagacatgcgtgtagagattgcaaacaacactttaagcctcccaaatttagagcatttcgcaatcttaatgttacaacgcaatcgatacatgaaaacaaggaaataacagagattcttccagataataatccatctcctccagaatctccagaaccagcatcacctatagatgacgggttaatcagatccacattcgatagaaacgacgaaccaccagaggatgatgaataaaaaaatgataaaataaattagttttattgctggttgtgttagttctctctaaaaatgtctaagatctatattgacgagcgttctgacgcagagattgtgtgtgcggctattaaaaacattggaatcgaaggagctactgctgcacaactaactagacaacttaatatggagaagcgagaagttaataaagctctgtacgatcttcaacgtagtgctatggtgtacagctccgacgatattcctcctcgttggtttatgacaacggaggcggataagccggatgctgatgttatggctgacgccataatagatgatgtatcccgcgaaaaatcaatgagagaggatcataagtcttttgatgatgttattccggctaaaaaaattattgattggaaagatgctaaccctgtcaccattattaatgagtactgccaaataactaagagagattggtcttttcgtattgaatcagtggggcctagtaactctcctacattttatgcctgtgtagatatcgacggaagagtattcgataaggcagatggaaaatctaaacgagatgctaaaaataatgcagctaaattggcagtagataaacttcttggttacgtcatcattagattctgattctagttatcaataacagttagtagtttagttatacattgaatcatacatattaatttttttattgagatagattaaaaaatacaaattgtagtactattaacgcgactagtatattctctaaagatgatatctgtcacagatattcgtagagcgtttctagacaatgaatgccatactatcacaaaagcgtttggatatctgcacgaggacaaggctatcgcattaattaaaataggatttcatcccacttatctacccaaagtcctttataataatgttgtagaattcgttccagaaaaactatatctgtttaagccaagaactgtagctccattggatttgatatctactataacaaaattaaagaacgtggacaaatttgcctcacacataaattatcacaagaatagtatattgataacaggagacaagtctctaattgttaaatgtatgccttacatgattatttcagatgatgatatacgattcataagagaacagtttgttggtacaaattctattgagtatattctatccttcatcaacaaggaaagcatatatagaatgagttaccaattttcagagaatgaaatagtcactatcatcaatagagatcatttcatgtatgaaccaatatatgaacatcaggtcttagattctgactttcttaaaactatgttagatagatacggaatagttcccattaattctggtataatagatgaattatgtccagaagctataatagagatattaatggcagtagttcgtcctagggacgctatccgtttcttagatatagtgaataagaatcaattgacagaagatagtgtcaaaaactatatcattaatgatatcagaagaggtaaaatagattattatattccatacgttgaagattttttagaagatagaactgaagacttgggaatatatgcgaatatattttttgaggatgctatagatattacgaaactagacatcacaaagacagagttggaacatatatcgaaatacatgaattattacactacttatatagatcacatagttaacatcatcttacaaaataattatatagatatcttggcatctataatagattacgtgcaagacgtattaacagaagaattatgtattagaatagtttgcgaatcaacaaaccctgttcccgttacatctcttcctatacattctacgttagtaatggttatgtgtatacaaatgaaatatgtcgatatagttgaattcttagacgagatcgacatcgatactttaatagaaaaaggagcagatccgataaccgaatacacatttacaacaagatggtacaataaacacaatgatttgatcactctttacattaagaaatatggattctgtccaatgatgatgaaacggttaatgttcgaatatccattgactaaagaagccagtgatcatttacttaaaaccatggatgaaaacaggggagctattatgttttttccgcgtactatttgcacacttccttatctattatgttgtaattataaactaattcaaaaacctattccattcaaagaagaaaatcgtaacatcgtatataagaaaaccaacagagtattatgctttgactcgttggagaactctgcgtttaaaagcctcattaaaatagattctattccaggattaaaaacttataacatgaaagacattacatacgaaaagtctaataatataatttgtgttaggtttatacctcaagaatcaattcataatgaagagcgaagaataaaattacagttattcgacattgctagattggcatcctatggactatattatattccctctagatatttatcatcgtggacaccagtagtgaacatgatagagggaagagagtacactaatccacaaaaaatagaatgtctagttattttggatttattttcagaggaattcatagaatatcaaaatctgggcaatgcggtatctaataaatatgaactggaatatactatatctaattatcaagctgccataaactgcctaatgagcacgttattaatatatctagttctaggatcaatcagatcgatatcaaaaactgaagattttgtattatctatattaaatatcttctataaaggactgaaaattaatgaattactttctgaaccagtatcaggagtttgtatcgaattaaataaaataaaagatagagcgagctctggagacagtagttttatatttcttaagaaaaacgagttatcaaaaactctatcgctctgtgaaaaagtttgtgttgagaccatattagacaataatcagagttttaaatcctcaaaatgaataggaatcctgatcagaatacttttcctaatattacattaaagattatagaaacctatttaggcagagtacctagtgtgaacgaatatcatatgttaaaattacaagctagaaatattcagaaaataactgtttttaacaaagacatatttgtatctttagtaaaaaagaataaaaaaagatttttttccgatgttgatacatctgcatcagaaataaaagatcgtatacttagctacttttctaaacagactcaaacatataatataggtaaattatttacgattatagaactacaatctgtattagtgaccacatacacggacatattaggagttcttactattaaagctccaaatgtaatttcatctaaaatttcttataatgtaacatcaatggaagaattggcaagagatatgctaaattctatgaacgtcgcagtaatagacaaggcaaaagtaatgggacgtcataatgtatcttccctagtcaaaaatgttaataagttgatggaagaatatcttagacgccataataaaagttgtatatgttacggatcatattctctatatctaattaatccaaatatacggtacggcgatatagatattcttcaaactaattcgaggacttttcttatagatttggcatttctaataaaatttattacgggaaataatattatattaagtaaaatcccatatcttagaaactatatggtgataaaagatgaaaacgataatcatatcattgatagttttaatattcgccaggataccatgaacgtagttcctaaaatctttatagataatatctatatagtggatccgacgtttcaactattgaacatgataaaaatgttttctcaaatagatagattggaagatctatccaaagatcctgaaaagtttaatgcgcgtatggcaaccatgctagaatacgttagatatacacatggtatagtctttgatggtaagcgtaataatatgccgatgaaatgtatcatcgatgaaaataatcgcatagttactgttactactaaagactattttagctttaaaaaatgtctagtgtatctagatgaaaatgtgttatcgagtgatatattagatcttaacgccgacacatcgtgtgatttcgagagtgttacaaattctgtatatctaattcatgataatatcatgtatacatatttctcaaatactattctccttagtgataaggggaaggtacatgaaataagtgccagaggtttatgtgcacatatattgttgtatcagatgctgacatctggagaatacaaacaatgtttatcggatctcttaaattcgatgatgaatagagataaaatacctatctattcacatactgaaagagataaaaaacctggacgacacggatttattaatatcgaaaaggatataattgtattttaggacaaaagtctagaagctacattatcgcgattagccgcgaacatattttgtagcatgtccgtcctcataaacggaacttgttttcctgggttattcattctctcgcatctggaaggggacgaaggtcttctctcacacgcgggtgatgagggtttatctactttgagtacgcaggaagctttacttccttcgcattctacagttctaacatcgcatactttaacggcttttagaaccagatatcttccttctttggtattgatataaaacggagtatgagcagatgcaaaatgagaattcatttatagcatagaaaaaaaacaaaatgaaattctactatatttttacatatatatattctaaatatgaaagtggtgattgtgactagcgtagcatcgcttctagacgcatctattcagtttcaaaaaacggcatgtaggcatcactgtaattacctatctatgcaagtagttaaagagatagaagaatttggtactatcaatgaaaaaaatttggaatttgacacttggaaagacgttatacaaaacgatgaaatagatgcattagtattttatagagtaaaacaaattagtatttctacgggtgttctatataaatctatgatgcgcaatagaacaaaacctatttccatgtactttgtacgtgattgtctggcatttgatggagatcctccgtcttttagaatgacgtcttgcaatatcaacgcatacaatcgtagtaagattaaagatttaataatcctaatgaatatgaaaacatgtaataaaaaaattatcggtgagtttataatagacaattttggaagcgtcgatgcattactatcgatagttaattccaatgttacgtggattacatcagttataaataatagtaacggcaggggtattaatatcagggtatcaaataataaaatgttaactataactagttttcgacgattcgtcaataaacttaaaatgtacaaaactactaaatgcgcttctcaattggataatctatgtaccgagatgaacaaaatggatattatagacaaaaaatgaaacgtaatgaggagtattgcggggctacataaattaaaaatggaaatttttaatgtagaagaattgataaatatgaaaccttttaagaatatgaataaaataacaattaatcaaaatgataattgtatattagcaaatcgatgctttgttaaaatagatactcctagatacataccatcgacatccattagcagttctaatatcatcagaatacggaatcatgattttacattatctgaattattgtattcaccgtttcattttcaacagcctcagtttcaatatctccttcctgggtttgtattaacgtgtattgataaagtttcgaaacagcaaaaaaaatgtaaatattgtatctctaatcgtggagatgatgatagtttaagcattaatctatttattccgactattaacaagtctatatatattattatcggtttacggatgaaaaatttttggaagcctaaattcgaaatagaataatgtttttatattatacatgttctaaaagaataatcgatacagtttaagtgaaagctagagaggggtttttaaatggtcatcggtttagtcatattcgtgtctgtggcggccgccatcgtcggtgtgttgtctaacgtattggacatgtttatgtacgtagaagaaaataatgaagaggatgctagaatcaaggaggagcaagaactactgttgctatattgatacataattgaaaatctaccaacttaaatacaccgcctataaatttacaatgaaacacagattgtattctgaaggattgagtattagtaatgatttaaactcgataatcggtcaacaatctacaatggatacggatatagaaatagacgaagatgacatcatggaacttcttaatatattgactgagttaggttgtgatgtcgactttgatgaaaattttagcgatatagccgatgatattctagaatcgttgatagaacaggatgtataagtttttatgttaactaaatgtggccatttgcaccggtacctgcgggagcaaaatgtaggctggtagaaacactaccagaaaatatggattttagatccgatcatttaacaacatttgaatgttttaacgaaattatcactctagctaagaaatatatatacatagcatctttttgttgtaatcctctgagtacgactaggggagcgcttatttttgataaactaaaagaggcatctgaaaaagggattaaaataatagttttgctagatgaacgagggaaaagaaatctgggagagctacaaagtcactgcccggatataaattttataaccgttaatatagataaaaaaaataatgtgggactactactcggttgtttttgggtgtcagataatgaaagatgttatgtaggaaacgcgtcatttactggaggatctatacatacgattaaaacgttaggtgtatattctgattatcccccgctggccacagatcttcgtagaagatttgatacttttaaagcctttaatagcgcaaaaaattcatggttgaatttatgctctgcggcttgttgtctgccagttagcactgcgtatcatattaagaatcctataggtggagtgttctttactgattctccggaacacctattgggatattctagagatctagacactgatgtagttattgataaactcaagtcggctaagactagtatagatattgaacatttggccatagttcccactacacgtgtcgacggtaatagctactattggcccgacatttacaactccattatagaagcagccattaatagaggagttaagatcagacttctagttggtaattgggataagaacgacgtatattctatggcaaccgccagaagtctagacgcgttgtgtgttcaaaatgatctatctgtgaaggttttcactattcagaataatacaaaattgttgatagtcgacgacgaatatgttcatatcacttcggcaaatttcgacggaacccattaccaaaatcacggattcgtcagttttaatagtatagataaacagcttgtaagcgaggctaaaaaaatatttgagagagattgggtatctagccacagtaaatcgttaaaaatttaaaaaatagaaacgtatagaacgccatcatgttaaacaggatacaaaccttgatgaaaacagctaacaattatgaaactattgagatattgcgtaactatttaagactgtatatcattttggcacgaaatgaagaaggtcatggtatactaatatacgatgataacatagatagtgttatgtcgatgatgaatattacaatattagaagttataggattgacgactcattgcacaaaattaagatcatcgcctccaattcctatgtctagattgtttatggacgaaatagatcatgagtcatattattctccaaaaacttcagattatccgttgatcgatattatacgaaagcgttctcacgaacagggagatatagcactggctttagaacgatacggtattgagaatacagattccatatcagaaattaatgaatggttgtcgtcaaaaggtttagcatgttatagatttgtaaaatttaacgattataggaaacagatgtatcgtaagttctctaggtgtactatagttgacagtatgataatagggcatataggtcatcattatatttggattaaaaatttagaaacatatacgcgtcccgaaattgatgtgttaccgtttgatattaaatacatatctagagatgaattgtgggcgcgaatttcttcctcgttagatcaaacacatataaaaaccatcgccgtatcagtttatggagctattactgataatggaccaataccatatatgatatccacgtatccgggtaatacctttgttaactttaacagtgtaaaaaatctaattttaaatttcttagattggattaaagatattatgactagtacacgaactatcattctagtaggttacatga

SEQ ID NO: 45 - DNA, 31690 bp, synthetic construct
  misc_feature 1..31690: note = Vector
  source 1..31690: mol_type = other DNA
tggctggaatcattaacaagaaaataaaaaacaatttcaaagttaaacataactggtattacgatttccatttctttgttcatactttattaaaaacatatccagaaatcgaaaaagatatcgaatttagtacggcattggaagaattcatcatgtgtaccaaaacagactgtgataaatatagattaaaggtttccattcttcacccaattagtttcttggaaaaatttattatgagagacattttctcagactggataaatggcggaaactaaagagtttaaaactttgtataatctttttatagatagttatttacaaaaattagctcaacattctatccctactaatgtcacttgtgctattcatataggagaggttataggacagtttaaaaattgcgcgctccgaataactaacaaatgcatgagtaattctcgacttagtttcacactcatggttgaatcatttattgaagtgatttcattgcttccggaaaaggatagaagagctatcgctgaggaaataggaatagatctagacgatgtacctagtgcggtatccaagctagaaaagaactgtaatgcgtatgcggaggttaataatattatagatatacagaaattggatatcggagaatgttcggctccgcccggtcaacatatgcttttacagatagttaatacaggatccgcggaagcaaattgtggtttacagacaattgttaagtccttaaataaaatatacgttccacctattatcgaaaaccgattgccgtattacgatccgtggtttctagtgggtgtagcaattattctagttatttttactgtagctatttgttctattagacgaaatctggctcttaaatacagatacggaacgtttttatacgtttaattaataaaaaaatttaattacaaggtataaaatagtactccatctacgcaatcgcgataatggagggatctaaacgcagacacgacagtcggcgactacaacaagaacaggagcagcctcgtccacgtacaccgccatcatatgaagaaattgcaaaatatggacactcatttaacgtgaaaagatttacgaatgaagaaatgtgtcttaagaatgattatccacgaattatatcatataatcctccaccaaaatagagtatatatatatcatcatttcatgatgtatactactgacatagtttcaatgtgaacttttcactttcttgccggttatgaagaatatttttattttaatggtcattactaatcgtatattataattgaaaatggattagtttaatatgacgctcgtcatgggatcctgctgtggtagattctgtgacgctaagaataagaataagaaggaagatgtagaagagggaagagaaggatgttacaattataagaaccttaatgatctggatgaatccgaagcacgtgtagaatttggaccattatatatgataaatgaagaaaaatcagacataaatacattggatataaaaagaagatatagacacacgatagagtctgtatatttctaaaagtttttataaaaaatgagtaaaatactcacgtttgttaaaaataagataattgacttgattaataatgaccaaattaaatattctagagttataatgatagaagagtccgatagtcttttaccggttgatgaggtgcatgctaaccacggatttgactgtgtggagatgatagatgaaaatataagcaatgagaatatcgaacagtataaaaccgaatctttttttacaataaattgaaatcaaaacatttattaaaccgcatcaagatgggtacgaacggcgttagagtatttgtcattttatatttgttggctgtatgcggatgtatcgaatacgacgtagacgataatgtacatatttgtacccacactaacgtgtcacatattaatcacactagttggtattataatgataaggttatagcgctagccaccgaggataaaacttctggttatatatcatcattcataaaacgcgttaatatctcattaacttgtttaaatatatcgagtttgaggtacgaagattctggtacatacaaaggagtatcgcatctaaaagatggagtcatcgttacgactactatgaatatatctgtaaaggctaatatcattgacttgactggtagagtgcgttatctaaccagaaattattgcgaagttaaaatacgatgcgaaataacatctttcgcgcttaatggttctactacaccaccacatatgatattaggaacagtagataaatggaaatatcttccatttcctacagatgattatagatacgtaggggaactgaaaagatatatatctggaaacccatatccaacagagtcgctagcgttagaaattagctcgacgtttaatcggtttactatcgttaaaaatttgaacgatgacgagttttcttgttatctgtttccataaaatgttgaacgcgcgccatatttgtgaatccgaatgggaggctttaaataataataacgataattcatcctccatgcccgcttcccacaacaatctcgcaaacgatttatctagtatgatgtcacaattacaaaatgataataacgataatttaattatgatagtactaataacaatgctatcaataatacttgtaattattgtagtgattgcggcgatatcgatgtacaaaagatccaagtacaggcatatagataactgaaaaaaaatttattgttattgttaatttagttatggaacccatccttgcaccaaatccaaatagatttgttattttcccaatccaatatcatgacatctggaacatgtataaaaaggcagaggcatcattttggacagtggaagaagtagatatatctaaagatatcaatgattggaataaactaacaccagacgaaaaatattttataaaacatgtattggcgttttttgcagccagtgacggaatagtgaatgaaaatttggcggaacgattttgtacagaagtacagattaccgaggctagatgtttctacggatttcagatggccattgaaaacattcattcggaaatgtatagtcttttgatcgatacttatgttaaagatagtaatgaaaaaaactatctctttaatgccatagaaacgatgccttgtgtaaaaaagaaggccgattgggctcaaaagtggatacatgacagcgccggttatggagagagacttattgcctttgctgcagtagaaggaatcttcttttccggatcattcgcttccatattttggcttaaaaagcgtggcctaatgcccggactcacgttttccaacgaattgattagtagagacgagggtctacactgcgacttcgcatgtttgatgtttaaacatttattgcatccaccgagtgaagaaaccgttagatctattataacagatgcggtatccattgaacaagaatttcttactgcggctcttccagttaaacttataggaatgaattgtgaaatgatgaaaacatatatagaattcgtcgcggatagattgatttctgaattgggatttaaaaaaatttataatgttaccaatccgtttgatttcatggaaaatatatcattagaaggtaaaactaattttttcgaaaaacgtgtgggtgaataccaaaaaatgggagttatgtctcaagaagataatcatttttctttagatgttgacttttaaagaaacataaatgccgatatttgtcaatactgtgtactgtaagaatatattagcattgtctatgactaagaaattcaaaacaattattgatgccataggaggcaatataatagtcaattctacgatattgaaaaagttatctccttactttcgcacacatttacgtcaaaaatacacgaaaaataaagatccagttacgagggtttgtctagaccttgacattcacagtctaacttctatagttatttactcatatactggaaaggtatatatagatagtcataacgtcgtcaatttattacgtgcttctatattaacctctgtagaatttatcatctacacttgtataaactttatcctacgagattttagaaaggaatattgtgtcgagtgttacatgatgggtatagaatacggactatccaatctcttatgtcatactaaaaactttattgccaaacactttttggaactggaagatgacatcatagacaattttgattatctatctatgaaacttattctagaaagcgatgaactaaatgttccagatgaggattttgtcattaagtggtatataaagcgaagaaataaattaggaaatctgctactccttatcaaaaatgtaatcaggtcaaattatctttctcccagaggtataaataatgtaaaatggatactagactgtaccaaaatatttcattgtgataaacaaccacgcaaatcatacaagtatccattcatagagtatcctatgaacatggatcaaattatagatatattccatatgtgtacaagtactcatgttggagaagtagtatatctcatcggtggatggatgaacaatgaaatacataacaatgctatagctgtaaattatatatcaaacaattggattccaattcctccgatgaatagccccagactgtatgctagcgggatacccgctaacaataaattatacgtagtaggaggtctaccaaatcccacatctgttgagcgttggttccacggggatgctgcttgggttaatatgccgagtcttctgaaacctagatgtaatccagcagtggcatccataaacaatgttatatacgtaatgggaggacattctgaaactgatacaactacagaatatttgctacccaatcatgatcagtggcagtttggaccatccacttattatcctcattataaatcatgcgcgttagtgttcggtagaaggttattcttggttggtagaaatgcggaattttattgtgaatccagcaatacatggactctgatagatgatcctatttatccgagggataatccagaattgatcatagtggataataaactgctattgataggaggattttatcgtgaatcgtatatagatactatagaagtgtacaatcatcacacttattcatggaatatatgggatggtaaataattttgaaataaaatattagttttatgttcaacatgaatattaactcaccagttagatttgttaaggaaactaacagagctaaatctcctactaggcaatcgccgggtgctgccggatatgatttgtatagcgcttacgattatactatccctccaggagaaagacagttaattaagacagatattagtatgtccatgcctaagatttgctatggtagaatagctcctaggtctggtctgtcactaaaaggcattgatataggaggtggtgtaatagacgaagattataggggaaacataggagtcattcttattaataatggaaaatgtacgtttaatgtaaatactggagatagaatagctcagctaatctatcaacgtatatattatccagaactggaagaagtacaatctctagatagtacaaatagaggagatcaagggtttggatcaacaggacttagataataaacaatagtatgttgtcgatgtttatgtgtaataatatcgtagattatgtagatggtatagtacaggatatagaagatgaggctagcaataatgttgatcacgactatgtatatccacttccagaaaatatggtatatagatttgacaagtccactaacatactcgattatctatcaacggaacgggaccatgtaatgatggctgttcgatactatatgagtaaacaacgtttagacgacttgtatagacagttgcccacaaagactagatcatatatagatattatcaacatatattgtgataaagttagtaatgattataatagggacatgaatatcatgtatgatatggcatctacaaaatcatttacagtttatgacataaataacgaagttaatactatactaatggataacaaggggttgggtgtaagattggcgacaatttcattcataaccgaattgggtagacgatgtatgaatccagtaaaaactataaaaatgtttactctactatcgcatactatatgcgatgattgttttgtagattatataacggacatttcaccaccagataataccatccctaacactagcacgcgtgaatatctaaagcttattggcatcacagctatcatgtttgctacatataaaactctcaaatacatgataggataattttttttaacacggatatagaatgctaacgtaataattatgcgttatgaagacccctatatcatcaattcaattttttttctagaaaaagtatcaagatgtttatattatcgtcggtcatcagatctgtaatgtttcccaatgattggaatctagattctgaaatctttttgtatccccaatgttcagtgattttagcacatattcctatggtagcgaataaactttcctgatcatggacttttgtaaaattgatgtagtggtctccttcgctcatagcttcgacaatctcattaattttatcaataccatagtaccgtatagcgacatcatcgaacttcatcaattccttgtacagtcttccacaactggtaatatctttgttaaacactataacatgatttctccacgtaatatattcatctattagatcgatgatggagtcgcgactacatattttatcatcatccacaaagtaaaaaacagcatcctcataatctaatttagtcgccatgactatctcacaaaagacagtagccgtctccttcctctatattgatttaattgtatgtttttacaattatcaataaaacataaaaataatatgatcatcaaacgaactgttaatattgatagttatataacgtgaatcatgagtgcaaactgtatgttcaatctggacaatgattacatatcctaaggcattagtattcataagtcatggagctggtaaacattctggacgttatgacgaactagctgaaaacatatcatcgttaggaattttagtattctcacatgatcatattggacatggaagaagtaatggtgaaaaaatgatgattgatgactttggtacagcacgtggtaactattaaatctacttattgggtcattccatgggagcaacaatttctatactagcctcttacgataatccaaacttgtttacagcaatgattctaatgtctcctctagttaatgcagatgctgtttcaagactgaatctgctagctgccaaacttatgggaaccatcacaccaaatgcgccagtcggaaagctatgtccagaatcagtatctagagatatggataaagtttataaataccaatacgacccattaatcaatcatgaaaaaattaaggctggatttgctagtcaggtcttgaaggctaccaacaaggttagaaaaataatttccaagattaacaccccgactctcatactccagggaacaaacaatgagattagcgatgttttaggtgcatattatttcatgcaacatgcaaattgtaatagagaaataaaaatttatgaaggtgccaaacatcatcttcataaggaaacggatgaagttaaaaaatcagtcatgaaagaaatagaaacttggatttttaatagagtgaagtgatataggattattcttttaacaaataaaatgaatccggataatacaatcgcagtgattacagagactattcctataggtatgcaatttgataaagtatatttgtctacatttaacatgtggagggaaattctatccaataccacaaaaacactagatatatcatctttttattggagtttatcggatgaagtgggtacgaatttcggcacgataatattaaacgagattgtacaattacccaaaagaggagtacgagttagagtagccgtcaataaatctaacaaaccattaaaggatgttgaaagactacaaatggccggagttgaagtacgatacatagatattacaaatatcctaggaggagttcttcatacaaaattttggatatctgataatacacatatttatttaggaagcgctaacatggattggagatcactaactcaggtcaaagaattgggtattgcgatcttcaataataggaacttggcagcggatctcactcaaatttttgaggtatactggtatcttggagttaacaatctaccatataattggaaaaacttttatccgtcgtattataatacagatcatcctcttagtattaacgtaagtggtgttccacactctgtatttattgcttctgcaccgcaacaactatgtactatggaaagaaccaatgatttaaccgctttattgtcatgtattagaaatgcgagtaaattcgtttatgtatctgttatgaactttatccctattatttattcgaaggcgggtaaaattttgttttggccttatatagaagatgaattaagaagatccgctatagacagacaagtatccgttaagctattgattagttgctggcaacgatcttcgtttatcatgagaaactttttaagatctatcgctatgctaaaatctaaaaacataaatatagaagtaaagctatttattgtaccagatgctgatcctcccattccgtattctagggtaaaccatgccaaatatatggtaaccgataaaacggcctatataggtacctcaaattggacaggaaattactttacggatacatgtggagcatctattaatattacaccggatgatggattaggtcttcgtcaacaattagaagatatttttatgcgtgattggaattcaaaatacagctatgaattgtacgatactagtcctactaaaaggtgtaaactattaaaaaatatgaaacaatgtacaaatgatatatactgcgatgagatacaaccggaaaaagaaattcctgaatattctcttgaataaaatagatataaaaacataatttttatcccaatttacgagcccgttaacaagatgcttgcattttgttattcgttgcccaatgcgggcgatgtaataaagggcagagtatacgagaatgattatgctctatatatttatctttttgactatcctcactctgaagctatcttggcagagagtgttaagatgcatatggatagatatgttgaatatagggataaactggtagggaaaactgtaaaagttaaagtgattagagttgattatacaaaaggatatatagatgtcaattacaaaaggatgtgtagacatcaataatttttataccgaacataaaaataaggttaattattaataccataaaatcatgattgcgttattgatactatcgttaacgtgttcagcgtctacctatcgtctacaaggatttaccaatgccggtatagtagcgtataaaaatattcaagatgataatattgtcttctcaccgtttggttattcgttttctatgtttatgtcgctattgcctgcatcaggtaatactagaatagaattattgaagactatggatttgagaaaaagagatctgggtccagcatttacagaattaatatcaggattagctaagctgaaaacatctaaatatacgtacactgatctaacttatcaaagtttcgtagataatactgtgtgtattaaaccgtcgtattatcaacaatatcatagattcggcctatatagattaaactttagacgagatgcggttaataaaattaattctatagtagaacgtagatccggtatgtctaatgtagtagattctaatatgctcgacaataatactctatgggcaatcattaatactatatattttaaaggtatatggcaatatccgtttgatatcactaaaacacgcaatgctagttttactaataagtacggtacgaaaacggttcccatgatgaacgtagttactaaattgcaaggaaatacaatcacaatcgatgacaaagaatatgacatggtacgccttccgtataaggatgctaatattagtatgtacctggcaataggtgataatatgacccatttcacagattctattacggctgcaaaattagactattggtcgtttcaattagggaataaagtgtacaatcttaaactccctaaattttctatcgaaaataagagggatattaagtcgatagccgaaatgatggctcctagtatgtttaatccagataatgcgtcgtttaaacatatgactagggacccattatatatttataaaatgtttcagaatgcaaagatagatgtcgacgaacaaggaactgtagcagaggcatctactattatggtagctacggcgagatcatctcctgaaaaactggaatttaatacaccatttgtgttcatcatcagacatgatattactggatttatattgtttatgggtaaggtagaatctccttaatatgggtacggtgtaaggaatcattattttatttatattgatgggtacgtgaaatctgaattttcttaataaatattatttttattaaatgtgtatatgttgttttgcgatagccatgtatctactaatcagatctattagagatattattaattctggtgcaatatgacaaaaattatacactaattagcgtctcgtttcagacatggatctgtcacgaattaatacttggaagtctaagcagctgaaaagctttctctctagcaaagatgcatttaaggcggatgtccatggacatagtgccttgtattatgcaatagctgataataacgtgcgtctagtatgtacgttgttgaacgctggagcattgaaaaatcttctagagaatgaatttccattacatcaggcagccacattggaagataccaaaatagtaaagattttgctattcagtggactggatgattcgtattcgatgattatttttaacaaaataacataaaaataatatatttttttaggatgcgatcatgacgtcctctgcaatggataacaatgaacctaaagtactagaaatggtatatgatgctacaattttacccgaaggtagtagcatggattgtataaacagacacatcaatatgtgtatacaacgcacctatagttctagtataattgccatattggatagattcctaatgatgaacaaggatgaactaaataatacacagtgtcatataattaaagaatttatgacatacgaacaaatggcgattgaccattatggagaatatgtaaacgctattctatatcaaattcgtaaaagacctaatcaacatcacaccattaatctgtttaaaaaaataaaaagaacccggtatgacacttttaaagtggatcccgtagaattcgtaaaaaaagttatcggatttgtatctatcttgaacaaatataaaccggtttatagttacgtcctgtacgagaacgtcctgtacgatgagttcaaatgtttcattgactacgtggaaactaagtatttctaaaattaatgatgcattaatttttgtattgattctcaatcctaaaaactaaaatatgaataagtattaaacatagcggtgtactaattgatttaacataaaaaatagttgttaactaatcatgaggactctacttattagatatattctttggagaaatgacaacgatcaaacctattataatgatgattttaaaaagcttatgttgttggatgaattggtagatgacggcgatgtatgtacattgattaagaacatgagaatgacgctgtccgacggtccattgctagatagattgaatcaaccagttaataatatagaagacgctaagcgaatgatcgctattagtgccaaagtggctagagacattggtgaacgttcagaaattagatatagagaaaatagctccagaatacagacaatgcttacaggatctataccatatgaaaattacgcgtcctagacactttgataactagttatgtcattctcgtttttatagagtgtactcgttactaaatattaattaatgaaataataacaattattaagacatactattcatccaacttaaacaacgaaaaacatttttcattaagtttatcatgaatgcgtataataaagccgattcgttttctttagagtctgattctatcaaagatgttatacacgattatatttgttggctcagtatgactgatgaaatgagaccatctatcggaaacgtctttaaagcgatggaaacgtttaagatagacgcggttagatattacgatggtaacatatacgatttagctaaagatataaatgcgatgtcattcgacagttttataagatctctacaaaatatctcttcaaagaaagataaactcactgtttatggaaccatgggactgctgtctattgtcgtagatattaacaaaggttgtgatatatccaatatcaagttcgctgccggaataatcattttaatggagtatatttttgatgacacggatatgtctcatcttaaagtagcactctatcgtagaatacagagacgtgatgatgtagatagatatttttttttcctaaactgatttctctgtttaaattcgtagcgatatataaaacaacatgtaattaattaataaactttaagacatgtgtgttatactaagatggttggcttattccatagtagcttgtggaatttataaacttatgatagtaaaactagtacccaatatgtaaagatgaaaaagtaaattactattaacgccgtcggtattcgttcatccattcagtatgggtatacagcacgaattcgacatcattattaatggagatatcgcgttgagaaatttacagttacataaaggggataactacggatgcaaactaaaaattatttcgaatgattacaagaaattaaagtttagattcattatacgcccagattggtcggaaatcgacgaggtcaaaggattaaccgtatttgcaaacaactatgcggtgaaagttaataaggtagatgacacgttctattacgtaatatatgaggctgtaatacatctgtataacaaaaaaacagagatattgatttattctgatgatgagaacgaactctttaaacactattacccatacatcagtctaaatatgattagtaaaaagtataaagttaaagaagaaaactactcatccccgtatatagaacatccgttaatcccgtatagagattatgagtccatggattaatatgagtatagtgttaaatgacacttactaaatagccaaggtgattattcgtatttttttaaggagtaaccatgtccgcaattagatttattgcatgtctatatctcatttccatcttcggaaattgtcatgaggatccatattatcaaccatttgataaattaaacattactctagatatatacacttatgaggatctagtaccatacaccgtagacaatgacacaacttctttcgttaagatatactttaaaaatttttggattacggttatgactaaatggtgtgctccgtttattgataccgttagcgtatacacatctcatgataatctgaatatacaattttatagtagggacgaatatgatacacaaagcgaggataaaatttgtaccattgatgttaaagcacgatgcaaacatctaacaaaacgagaagttacagtacaacaagaagcctacagatattcattatcttctgacctatcgtgttttgattctatagatctagagattgatcttattgaaactaatagtactgacactacagtactgaaatcatatgagctcatgcttcccaaacgtgctaaatccatacataactgaaatgaaagaaaccaaaaaatgcgatagcatcaacaaccaatcatggttaacgataagatactctatgatagttgtaaaacatttaacatcgatgccagcagtgcacaatcattgatagaaagtggtgcaaatccattatatgagtatgatggtgaaactccattaaaggcatacgttaccaagaaaaataataatatcaaaaacgatgttgtgattttgttattgtcgtcagtcgactataaaaatatcaatgattttgatatactcgaatatctatgttctgataacatcgatatagacttattgaaattactaatttcgaaaggtatagaaataaatagtatcaaaaatggtattaatattgtagaaaaatacgctacaacatcaaatcccaatgtagatgtgtttaaactattattggataaaggaatacctacatgtagcaacatacagtatggatacaagatcaaaatagaacagattagacgtgctggtgaatattataattgggatgatgaattagacaattacgattacgactacaccactgattatgatgatagaatgggtaaaacagttctctattattatattattactaggtcacaagatggttatgctacatctttggacgtaataaactatttaatttcacacgaaaaagagatgcgttattatacttatcgtgaacataccacactctattattatcttgacaaatgcgatattaaacgggaaatatttgacgcgttattcgatagtaactatagtggtcatgaactaatgaatattctatctaactatttacgtaaacagtttaggaagaaaaatcacaaaatcgataattatatagttgatcaactattattcgaccgtgatacgttttatattttagaattgtgtaatagtttacgtaataatatccacaattcttaaaagatatacagattctatacaagatctattgttagaatatgtatcttatcatacagtatacatcaatgttattaaatgtatgattgatgaaggagctacattatatagatttaagcatataaataaatattttcaaaaatttggcaatagagatcctaaagttgtcgagtatattttaaaaaatggaaacttagttgtagataatgacaatgatgataacctaataaatattatgccattattccctaccttctctatgcgtgagttggatgtgttatcgatactaaaactttgtaagccgtatattgatgatataaacatggatgtagtatactttatcattgtattaagtcgcatagtgtcagcctagtagaatggttaatagataatggcgcagacattaatataataacaaaatatgggtttacatgtattactatttgtgttatactggcagataaatatatcccagaaatagcagaattatatattaagatattggaaattattctgagtaaattaccaaccatcgaatgtattaagaaaacagttgattacctagacgatcacaggtacttattcataggtggtaataataaatcgttactgaaaatatgtatcaagtacttcatattagtcgattataagtacacatgtagcatgtatccatcatatatagaatttataaccgactgcgaaaaagaaattgcggatatgcgtcaaattaaaataaatggtacggacatgcttacagtgatgtacatgttaaataaacctacaaagaaacgatatgttaataatccgatatttacagattgggctaataagcaatataagttttataatcaaataatatataatgctaataagttaatagaacaaagtaagaaaatagacgacatgatagaggaggtatccattgacgataatcgtttatcaacactaccgttagaaattagacatttgattttctcgtacgcgttcctataaaaatagaaactataatcatataatagtgtaggttggtagtattgctcttgtgactagagactttagttaaggtactgtaaaaatagaaactataatcatataatagtgtaggttggtagtagggtactcgtgattaattttattgttaaacttgtccttaagtcttattaatatgtcttctaaagggggtagtggcggcatgtggagtgtctttatccatggacatgatggtagtaataaaggatctaaaacttatacatctggtggcggtggaatgtggggaggaggatcgtccagtggtgtaaaaagtggggttaacggaggtgtaaaatctggaactggtaaaatttaaacactaaattatttttattaataattgtacaagtttttgacatgatatttaatgacattagttgtgtgggtgtatagagttcacagtagctcattcagtcaaaatgtttgactatttggaaaatgaggaggtggctctcgatgaacttaaacagatgttgagagacagagatcctaatgataccaggaaccaattcaagaataatgctttacacgcatacctttttaatgagcattgtaataatgtcgaggtcgtcaaactactactagacagtggcactaatccattacgcaaaaattggagacagctaccccattagaagaatacacaaatagtagacatgttaaagttaaaggatatagcgatggctctactagaagccactggatttagcaacataaatgactttaatatattcagctatatgaaatccaaaaatgtagacgttgacttgataaaggtgttggtagaacatggatttgacttgagtgttaaatgtgaaaaccatcgttcagttatagaaaattatgtaatgacagatgatcctgttcctgaaattattgatttgttcatagaaaatggctgcagtgttctttatgaggacgagtactgatacgcgtatgatgattatcaactacgaaattgcggtaccgtattgcatctgtatatcatctctcatctgtattcagagtcggatacgagagcatatgtgcgtccggaagttgttaaatgtctaattaatcacggaatcaagccgtcttttatagataaaaactattgtacagctcttcaatattatattaagtcatctcatatagatatagacatcgttaaattgttaatgaaaggaatagataacacggcttattcatatatagacgatctaacatgttgcactcgagtaattatggctgattatctaaatagtgattatagatacaataaagatgtagatttggtcaaattgtttttggaaaatggaaagccgcacggaataatgtgtagtattgtaccactatggagaaatgataaggaaaccatctttttgatattgaaaacaatgaactcggatgtcctccaacatatactaattgagtatatgacattcggcgatatccctctagtggaatatggaactgtggtaaataaagaggctatacacggatactttagaaatattaatattgattcttacacgatgaaatatctactaaaaaaggaagggagatgccatcaattatctcgatgatggagagatcccgattggacacctatgtaaatccaactatgaatgttataatttttacacttatacatacaaaaagggtctttgtgacatgtcttatgcttgcccaattcttagtactataaacatttgcctaccttatcttaaagacattaacatgattgacaaacgaggagaaacacttcttcacaaggctgttagatataataaacaatctctagtatctttactgctagaatccggttcagatgtcaacattagatcaaataacggatatacatgtatagccattgccatcaacgaatctaaaaacattgaactgctgaaaatgctattatgtcataaacctacattagattgtgtgattgattcattgagagaaatatctaacatcgtagataacgactatgctataaaacaatgtattaaatatgccatgattatagatgactgtacatcgtctaagattccagagtccataagtcaacgctataatgattatatagatctttgcaattaagaattgaatgagatgaaaaaaataatggtaggtggtaatactatgttctcattaatatttactgatcatggagctaaaattattcatagatatgtcaataatccagaattacgtgagtattatgagttaaaacaaaataaaatatatgtggaagcatatgatattatttccaacgcaatagtgaaacatgatagaatacataaaaccatagaatcagttgatgataatacctacatttctaatcttccgtataccatcaaatacaaaatattcgagcaacaataagtattttttatacctttaaaattgataaataaattttttctagtgatattttggcaagatgaaaatcctatttctcatcgctttcatgtatgggtgtgttcactcatatgttaacgcggttgaaaccaaatgtccaaatctagacattgtaacatcttctggagaatttcattgttcaggatgtgtggaacatatgcctgagtttagctatatgtattggttggcaaaggataccaagtttatagaacatctgggtgatggcatcaaagaagatgaaaccgttcgtaccacagatagtggaatcaccactctacgtaaagtccttcatgtaaccgatactaataaatttgctcattataggttcacttgtgtcctcactacgatagatggcgtttcaaaaaagaatatttggctgaagtagtgcgtgctactatttttatttatgatataatctaatggaattaatttgaattgatatttatccaatactaaagattatattagaatcaaattaatcttttatacgagaaaaaataacgacatacgtcgtcaacaaattaaactttttatttattagttaacttgctcattgttatgtttctaaaacgggtacgacatataggacaattatccgacgcaccggtttctcttcgtgttctatgccatatattgatgcatgttatgcaaaatatatgattacacgaatccaataaaccaaagtatctatcgttttgagtaaacaacttcatagcaaatttcacattctttttctttacttactctatacacgtcctcgtatttatccagtattttgatgatatccaactcagaaatggttgttgtattattgggtgtataggtattattagctatgtaccaatttaccaaccttcttaatattgattgataatcacatcggttatccaattaataactaaattgtagtgtatatatagaccatatatgtttctatttttttgacagtttcagtaagttttgattgttgtattcctgtatctctagataagttagtcatatagtcccttccggcgatacgttttttccaagcccgaaattgattagccaaatgtggatttatttttgtgataatgcatactgttagtcttatatcatttggttcatctatgtattgtaatattgttacatgatctatagatgatgtattgattttggcaggatcgaattccatatccgcgactaaacagtgaaaaaaatgtaaataattttaaattagtaaaacttttttttattttttatgattccaaaaaaactgaatacaaagtcctaaattataaatatggagatcatactaccacaacttattattatgtatacaaggccggtgtaatagatagatatatataattctattacaccggcagacaattaccgatcggtatttgtcgttaccaacataccgtataatatgtaatatacaattccataacccattgacagttgttatacatcaaaattgcaattcttttgattacgatgttataagaatgtagttaattgatgtatgatgttaatgtgtcctctttcctcttataacatcgtaatcaaaaacttttttataatatatacctaataatgtgtcttaatagttctcgtgattcgtcaaacaatcattcttataaaatataataaagcaacgtaaaaacacataaaaataagcgtaactaataagacaatggatatttacgacgataaaggtctacagactattaaactgtttaataatgaatttgattgtataaggaatgacatcagagaattatttaaacatgtaactgattccgatagtatacaacttccgatggaagacaattctgatattatagaaaatatcagaaaaatactatatagacgattaaaaaatgtagaatgtgttgacatcgatagtacaataacttttatgaaatacgatccaaatgatgataataagcgtacgtgttctaattgggtacccttaactaataactatatggaatattgtctagtaatatatttggaaacaccgatatgtggaggcaaaataaaattataccaccctacaggaaatataaagtcggataaggatattatgtttgcaaagactctagactttaaatcaacgaaagtgttaactggacgtaaaacaattgccgttctagacatatccgtttcatataatagatcaatgactactattcactacaacgacgacgttgatatagatatacatactgataaaaacggaaaagagttatgttattgttatataacaatagatgatcattacttggttgatgtggaaactataggagttatagtcaatagatctggaaaatgtctgttagtaaataaccatctaggtataggtatcgttaaagataaacgtataagcgatagttttggagatgtatgtatggatacaatatttgacttttctgaagcacgagagttattttcattaactaatgatgataacaggaatatagcatgggacgatgatacagatatatggactcccgtcacagaagatgattacaaatttctttctagactagtattgtatgcaaaatctcaatcggatactgtattcgactattatgttcttactggtgatacggaaccacccactgtattcattttcaaggtaactagattttactttaatatgccgaaataaaaaatttttgtataatatctagaggtagaggtattgtttagataaatacaaataacatagatacatcgcatatttagcatttttataaatatacataagacatacactttatacatttttgtaaaaatactcataaaaaaaatttataaaaattatggcacaaccatatcttgtataggtagtttagttcgtcgagtgaacctataaacagataatagacaacacgtaataataataatgcctactaatacaagcataataccgggagatgggatatatgacgttgtagtgtttggtttttctgaacgttgatagtctactaatactacatgctgacatctaatgcctgtataaccatgagagcatctacaatacataccgtcgatatctctagcgtggatacagtcaccgtgtaaacaatatccatctccctctggaccgcataatctgatagctggaatatctgttgtagcgtttgtaatttctggcgatgtcgtttcgatagcgttaccactatcggcgaatgatctgattatcatagcagcgaacaacaacatcagataatttatcaacatttttgatggattttgtgtttatgctgtttctcagtgtgtgtttatgacaagattgggaattttatattattaattcagtaatataaactaataatatattgttaattgtgtaaataatataaaaataacaatacaatattgaatgtgttgctgttaaaaatgtatgtgttaatataatagaataaaataaatgagtatgatcattttagataacgattgattttatcattaccgcttcattcttatattctttgcttacggaacctatatttagaaacatctactaacgattttttatgcttgcattattaatggtatgtaataccaatttgttaagtatgaatacggggtacaaacataaactgaaatttagatcattaaatgtttcatcagaaatgactccatgaaaaccgccgaagaacttcgtgcaatcattggactttgtactcaatcagctatcgtctctggaagagtcttcaacgataagtatatcgacatactacttatgctgcgaaagattctgaacgagaacgactatctcaccctcttggatcatatccgcactgctaaatactaaatctccttcatgctctctcactacactttttatcatcttatgaggaataattagcaccagaatagctatggattgcacatgtattctatgtcgtctactggatgaagatgtgacgtacaaaaaaataaaactagaaattgaaacgtgtcacaacttatcaaaacatatagatagacgaggaaacaatgcgctacattgttacgtctccaataaatgcgatacagacattaagattgttcgactgttactctctcgcggagtcgagagactttgtagaaacaacgaaggattaactccgctaggagcatacagtaagcatagatacgtaaaatctcagattgtgcatctactgatatccagctattcgaattcctctaacgaactcaagtcgaatataaatgatttcgacttacgtctgctaaaatacctaattgtggataaacggatacgtccgtccaagaatacgaattatgcaatcaatggtctcggattggtggatatatacgtaacgacgcctaatccgagaccagaagtattgctatggcttcttaaatcagaatgttacagcaccggttacgtatttcgtacctgtatgtacaacagtgatatgtgtaagaactctcttcattactatatatcgtctcatagagaatctctatccaaggatgtaattaaatgtttgatcgataacaatgtttccatccaatactactggtcttgctcaaccatagatatagagattattaataaaggatgtggacacgtgtagagtatacgacgtcagccctatattagaggcgtattatctaaacaagcgatttagagtaaccccatataatgtagacatggaaatcgttaatcttcttattgagagacgtcatactcttgtcgacgtaatgcgtagtattacttcgtacgattccagagaatataaccactacatcatcgataacattctaaagagatttagacaacaggatgaatccatcgtacaagccatactgataaactacttacattacggcgatatggtaagtatacctatcattcaatgcatgttggataagacgacggacaacaactttgttaataataatctcgtcgatgtaaacgtcgtaaggtttatcgtggaaaatatggacacgcggctgtaaatcacatatctaacaatggccgtctatgtatgtacggtctgatattatcgagatttaataattgcgggtatcactgttatgaagatgtatttgatatactaagcaagtacatggatgatatagatatgatcgataactctactatattacgcggtcgatgtcaataatatacaatttgcaaagcggttattggaatatggagcgagtgtcacgctcgataatcaatacggccatccagaaaagcagttaccaaagagaagctagttgatttattactgagttaccatcccactctagagactatgattgacgcatttaatagagatatacgctatctatatcctgaaccattattcgcctgtatcagatacgccttaatcctagatgatgattttccttctaaagtaagtatgatatcgccggtcgtcataaggaactaaagcgctatagagcagacattaatagaatgaagaatgcctacatatcaggcgtctccatgtttgatatattatttaaacgaagcaaacgccacagattgagatacgcaaagaacaatgagaggatcgactccattaaataatttatcatggagtgataatgtcctgtttccatggcatattacaaaatcgattccgtccaagatgataaaaacatttaccggcatcataaacacggagtttattttatatgtctcgcataaacattactaaaaaaatatattgttcggttttctttcacatctttaattatgaaaaagtaaatcattatgagatggacgcatcgttcgcgacagtatgtggtacatacctaacgtatttatggacgacggtaagaatgaaggtcacgtttctgtcaacaatgtcgacgcgatcgtgtaacacgactcacaatagaatctgtgaatgctctcccgatcatggatgcaaggcatgtgtttcccaaacaaaatgtggaataggatacggagtatccggagacgtcatctgttctccgtgtggtctcggaacatattctcacaccgtctcttccgcagataaatgcgaacccgtacccagaaatacctttaactatatcgatgtggaaattaacctgtatccagttaacgacacatcgtgtactcggacgaccactaccggtctcagcgaatccatctcaacgtcggaactaactattactatgaatcataaagactgcgatcccgtcttcttaataaggtagcgacttcaggtttctttacaggagaaaggtgtgcactctgaatttcgagattaaatgcaataacaaagattcttcctccaaacagttaacgaaagcaaagaatgatactatcatgccgcattcggagacagtaactctagcgtcgacatctatatactatatagtaataccaatactcaagactacgaaactgatacaatctcttatcatgtgggtaatgtagccatatgcccggtagttgcgatatacataaactgatcactaattccaaacccacccgctttttatagtaagtttttcacccataaatacaataattaatttctcgtaaaagtagaaaatatattctaatttattgcacggtaaggaagtagaatcataaagaacagtactcaatcaatagcaatcatgaaacaatatatcgtactggcatgcatgtgcctgccagtcttcagcaatcatcctcatcgtgtacggaagaagaaaacaaacatcatatgggaatcgatgttattatcaaagtcacaaagcaagaccaaacaccgaccgatgataagatttgccaatccgtaacggaaattacagagtccgagtcagatccagatcccgaggtggaatcagtcgaggatgtagatcctcctaccacttattactccatcatcggtggaggtctgagaatgaactttggattcaccaaatgtcctcagattaaatccatctcagaatccgctgatggaaagactgtgaggtgtctatcgacatcagatgtagcgaagaagagaaagacagcgacatcaagacccatccagtactcgggtctaacatctctcataagaaagtgagttacgaagatatcatcggttcaacgatcgtcgatacaaaatgtgtcaagaatctagagtttagcgttcgtatcggagacatgtgcaaggaatcatctgaacttgaggtcaagtatgtcgacggatcggcatctgaaggtgcaaccgatgatacttcactcatcgattcaacaaaactcaaagcgtgtgtctgaatcgataactctattcatctgaaattggatgagtagggttaatcgaacgattcaggcacaccacgaattaaaaaagtgtaccggacactatattccggtttgcaaaacaaaaagttacctctcgcgacttcttctttttctgtctcaatagtgtgatacgattatgacactattcctatttcctttcagggtatcacaaaaatattaaacctctttctgatggtctcatacaaaaatatttttattctctttctctctttgatggtctcataaaaaatatttttattctctttctctctttgatggtctcataaaatatttttattctctttctctctttgatggtctcataaaaaatatttttattctctttctctctttgatggtctcataaaatatttttattctctttctctctttgatggtctcataaaaaatatttttattctctttctctctttgatggtctcataaaaaatattaaacctctttctgatggtgtcactaaaatatttttattctctttctctcttcaatggagtcataaaatatttttattctctttctctcttcgatggtctcacaaaaatattaaacctctttctgatggtgtcactaaaatatttttattctctttctctcttcaatggagtcataaaatatttttattctctttctctctttgatggtctcacaaaaatatttttattctctttctctctttgatggtctcacaaaaatatttttattctctttctctctttgatggtctcacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaatattaaacctctttctgatggtgtcactaaaatatttttattctcattctctcttcaatggagtcataaaatatttttattctctttctctcttcgatggtctcacaaaaatattaaacctctttctgatggtgtcactaaaatatttttattctcattctctcttcaatggagtcataaaatatttttattctctttctctcttcgatggtctcacaaaaatattaaacctctttctgatggtgtcactaaaatatttttattctcattctctcttcaatggagtcataaaatatttttattctctttctctctttgatggtctcataaaaaaagttttacaaaaatatttttattctctttctctctttgatggtctcataaaaaatattaaacctctttctgatggtgtcactaaaatatttttattctctttctctcttcaatggagtcataaaatatttttattctctttctctcttcgatggtctcacaaaaatattaaacctctttctgatggtgtcactaaaatatttttattctcattctctcttcaatggagtcataaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaatattaaacctctttctgatggtgtcactaaaatatttttattctcattctctcttcaatggagtcataaaatatttttattctctttctctcttcgatggtctcacaaaaatattaaacctctttctgatggtgtcactaaaatatttttattctcattctctcttcaatggagtcataaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcacaaaaatattaaacctctttctgatggagtcgtaaaaaagttttatctctttctccttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcactaaaatattttttattctctttctgatgcatcaactatttcttaaacaataacgtccaacaacatatactcgtcgagcttatcaacatcccctatgcccatctaggttaccagacaattgtatatcataaaataatgtttataatttttacaaaaatatttttattctctttctctctttgatggtctcataaaaaaagttttacaaaaatatttttattctctttctctctttgatggtctcataaaaaatattaaacctctttctgatggtgtcactaaaatatttttattctcattctctcttcaatggagtcataaaatatttttattctctttctctcttcgatggtctcacaaaaatattaaacctctttctgatggtgtcactaaaatatttttattctcattctctcttcaatggagtcataaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaatattaaacctctttctgatggtgtcactaaaatatttttattctctttctctcttcaatggagtcataaaatatttttattctctttctctcttcgatggtctcacaaaaatattaaacctctttctgatggtgtcactaaaatatttttattctcattctctcttcaatggagtcataaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaatattaaacctctttctgatggtgtcactaaaatatttttattctctttctctcttcaatggagtcataaaatatttttattctctttctctcttcgatggtctcacaaaaatattaaacctctttctgatggtgtcactaaaatatttttattctctttctctcttcaatggagtcataaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaatattaaacctctttctgatggtgtcactaaaatatttttattctcattttctctttctctcttcaatggagtcataaaatatttttattctctttctctctttgatggtctcataaaaaatattaaacctctttctgatggtgtcactaaaatatttttattctcattctctcttcaatggagtcataaaaaagttttatctctttctctcttcgatggtctcacaaaaatattaaacctctttctgatggagtcgtaaaaaagttttatctctttctctcttcgatggtctcacaaaaatattaaacctctttctgatgcatcaactatttcttaaacaataacgtccaacaacatatactcatcccctatgcccatctaggttaccagacaattgtatatcataaaataatgtttataatttacacgttaaaatcatataataaaacgtagatcgtataatattttttggtatataaatgatctagtaaaatccatgtaggggatactgctcacattttttctttggtacaaaatttcacacaagtttttatacagacaaattcttgtccatatattttaaaacattgacttttgtactaagaaaaatatctagactaactatctctttctctttctctcttcgatggtctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctccttcgatggtctcacaaaaatattaaacctctttctgatggagtcgtaaaaaagttttatctctttctctcttcgatggtctcacaaaaatattaaacctctttctgatggtgtcactaaaatatttttattctctttctctcttcgatggtctcacaaaaatattaaacctctttctgatggagtcgtaaaaaagttttatctctttctccttcgatggtctcacaaaaatattaaacctctttctgatggagtcgtaaaaaagttttatctctttctctcttcgatggtctcacaaaaatattaaacctctttctgatggagtcgtaaaaaagttttatctctttctccttcgatggtctcacaaaaatattaaacctctttctgatggagtcgtaaaaaagttttatctctttctccttcgatggtctcacaaaaatattaaacctctttctgatggagtcgtaaaaaagttttatctctttctccttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctccttcgatggtctcacaaaaatattaaacctctttctgatggtctctataaagcgattgatttttcttaccctctagagtttcctacggtcgttggtcacacatttttttctagacactaaataaatatttaaaatataatattaatatactaaaatttatgtattattaatttataatattaatatactaaaatttatgtattattaatttatctaactaaagttagtaaatcatatacataattttataattaatattatatacataattttataattaatttaatcttac

SEQ ID NO: 46 - DNA, 4590 bp, synthetic construct
  misc_feature 1..4590: note = Vaccine construct
  source 1..4590: mol_type = other DNA
cccgccgccaccatgtctctgctgtggggcggagtgacagtgctggccgccatgctgattgctggccaggtggcctccgtggtgttcctcgtgaaaggcggaggacctggcggcggaatgcagctgtttcacctgtgcctgatcatcagctgtagctgccccaccgtgcaggccagcaagctgtgtctgggatggctgtggggaatggacatcgacccctacaaagaattcggcgccagcgtggaactgctgagcttcctgcccagcgacttcttcccctccatccgggacctgctggatacagccagcgccctgtatagagaggccctggaaagccccgagcactgcagccctcatcacaccgctctgagacaggccatcctgtgctggggcgagctgatgaacctggccacttgggtgggaagcaacctggaagatcccgccagccgggaactggtggtgtcttacgtgaacgtgaacatgggcctgaagatccggcagctgctgtggttccacatctcctgcctgaccttcggccgggaaaccgtgctggaatacctggtgtccttcggcgtgtggatcagaaccccccctgcctacagaccccccaacgcccctatcctgagcaccctgcctgagacaacagtcgtgcggcggagaggcagaagccccagaagaagaacccccagccccagacgcagaagaagccagtcccctcggcggagaagatcccagagcagggaaagccagtgtggcggaggctctgggggaggcatgccactgagctaccagcacttcagaaaactgctgctgctggacgacgaggccggacccctggaagaggaactgcctagactggccgacgagggcctgaacagaagagtggccgaggacctgaacctgggcaacctgaacgtgtccatcccctggacccacaaagtgggcaacttcaccggcctggcctccagcaccgtgcccgtgtttaatcccgagtggcagacccccagcttcccccacatccatctgcaggaagatatcatcaaccgctgccagcagtacgtgggccccctgaccgtgaacgagaagcggagactgaagctgatcatgcccgccagattctaccccaacctgaccaagtacctgcccctggacaagggcatcaagccctactaccctgagcacgccgtgaaccactacttcaagacccggcactacctgcacaccctgtggaaggccggcatcctgtacaagagagagacaaccagaagcgccagcttctgcggcagcccctacagctgggagcaggaactgcagcacggccggctggtgtttcagaccagcacaagacacggcgacgagtctttctgcagccagagcagcgggatcctgtccagatctcctgtgggcccttgtgtgcggagccagctgaagcagagcagactgggactgcagcctcagcagggaagcctggccagaggcaagtctggcagaagcggcagcatccgggccagagtgcaccctaccaccagacggtcctttggcgtggaacctagcggcagcggccacatcgataacagcgccagcagcacctccagctgcctgcatcagtctgccgtgcgcaagaccgcctacagccacctgtccaccagcaagagacagagcagctctggccacgctgtggaactgcacaacatcccccctagcagcgccagaagccagagcgagggccctatctttagcgcctggtggctgcagtttcggaacagcaagcccgccagcgactacgccctgacccacatcgtgaacctgctggaagattggggccctgccacagagcacggcgagcacaatatccggatccccagaacccctgccagagtgacaggcggcgtgttcctggtggataagaatccccacaacaccaccgagagcaggctggtggtggacttcagccagttcagcagaggcagcacccacgtgtcctggcccaagttcgccgtgcccaatctgcagagcctgaccaacctgctgagcagcaacctgagctggctgagcctggacgtgtccgccgccttctaccacatcccactgcatcctgccgctatgccccatctgctcgtgggcagttctggcctgcctagatatgtggcccggctgtctagcaccagcagaaacatcaattaccagcacggcaccatgcaggacctgcacgacagctgcagccggaatctgtacgtgtccctcctgctgctgtacaaaaccttcggcagaaagctgcacctgtactcccaccccatcatcctgggcttcagaaagatccccatgggcgtgggcctgagcccattcctgctggcccagtttaccagcgccatctgtagcgtcgtgcggagagccttccctcactgcctggcctttagctacatggacgacgtggtgctgggcgccaagagcgtgcagcacctggaatccctgtttacctccatcaccaactttctgctgtccctgggcatccacctgaaccccaacaagaccaagaggtggggctacagcctgaacttcatgggctacgtgatcggcagctggggcacactgcctcaggaacacatcgtgctgaagatcaagcagtgctttcggaagctgcccgtgaaccggcccatcgactggaaagtgtgccagcggatcgtggggctgctgggatttgccgccccttttacccagtgcggctaccccgctctgatgcccctgtacgcctgcattcagagcaagcaggccttcaccttcagccctacctacaaggcctttctgtgcaagcagtatctgaacctgtaccccgtggctcggcagagaagcggcctgtgtcaggtgttcgccgatgccacacctacaggatggggcctggccatcggacacagagccatgagaggcaccttcgtggcccctctgcctattcacacagccgagctgctggctgcctgtttcgccagatctagaagcggcgccaagctgatcggcaccgacaactctgtggtgctgagccggaagtacacctccttcccttggctgctgggctgcgccgccaactggatcctgagaggaaccagcttcgtgtacgtgccaagcgccctgaaccctgccgccgatcctagtgctggaaggctgggcctgtaccggccactgctgcatctgcctttcagacccaccaccggccggaccagtctgtatgccgtgtctcctagcgtgcccagccatctgcccgacagagtgcatttcgccagccctctgcatgtggcctggcggcctccaagaaaaagacgggcccctgtgaagcagaccctgaatttcgacctgctgaagctggccggcgacgtggaaagcaaccctggccctatgggcggctggtccagcaagcctagacagggcatgggcaccaacctgtccgtgcctaaccccctgggattcttcccagaccaccagctggaccctgccttcggagccaacagcaacaaccccgactgggatttcaaccctaacaaggaccactggcccgaggccaaccaagtgggagctggcgcatttggccctggcttcacacctcctcatggcggactgctggggtggtcacctcaggctcagggcatcctgactacagtgcctgccgctcctcctcccgcctccaccaatagacagagcggcagacagcccacccccatcagccctccactgagagatagccacccccaggccatgcagtggaacagcaccacctttcaccaggccctgctggacccaagagtgcgcggcctgtattttcctgccggcggaagctctagcggcaccgtgaatcctgtgcctaccaccgccagcccaatcagcagcatcttctccagaaccggcgaccctgcccccaacatggagaacaccacctccggctttctgggcccactgctggtgctgcaggccggcttttttctgctgacccggatcctgaccatcccccagtccctggactcttggtggacctccctgaactttctgggaggcgcccctacatgtcccggccagaatagccagagccccacctccaatcacagcccaaccagctgcccccctatctgccccggctatcggtggatgtgcctgcggcggttcatcatctttctgttcattctgctgctgtgtctgatcttcctgctggtgctgctggactaccagggcatgctgcctgtgtgccctctgctgcctggcacctctaccacaagcaccggcccttgcaagacttgcaccatcccagcccagggcaccagcatgttccccagctgttgctgcaccaagccctccgacggcaactgtacctgcatccccatcccaagcagctgggccttcgcccggtttctgtgggaatgggccagcgtgcggttctcctggctgagtctgctggtgcctttcgtgcagtggttcgtgggactgtcccctaccgtgtggctgagcgtgatctggatgatgtggtactggggcccctccctgtacaatatcctgtcccccttcctgcccctgctgcccatcttcttttgcctgtgggtgtacatctga

SEQ ID NO: 47 - DNA, 4578 bp, synthetic construct
  misc_feature 1..4578: note = Vaccine construct
  source 1..4578: mol_type = other DNA
cccgccgccaccatgtctctgctgtggggcggagtgacagtgctggccgccatgctgattgctggccaggtggcctccgtggtgttcctcgtgaagggcggatctatgggcggctggtccagcaagcctagacagggcatgggcaccaacctgagcgtgcccaatcccctgggattcttcccagaccaccagctggatcctgccttcggcgccaacagcaacaaccccgactgggacttcaaccccaacaaggaccactggcccgaggccaaccaagtgggagctggcgcttttggccctggcttcacacctcctcatggcggactgctgggatggtcacctcaggcccagggcatcctgacaacagtgcctgctgctccacctcccgccagcaccaatagacagagcggcagacagcccacccccatcagccctccactgagagatagccacccccaggccatgcagtggaacagcaccacctttcaccaggccctgctggaccctagagtgcggggcctgtattttcctgccggcggaagcagcagcggcaccgtgaatcctgtgcctaccaccgccagccccatctccagcatcttcagcagaaccggcgaccctgcccccaacatggagaacaccaccagcggctttctgggccccctgctggtgctgcaggccggcttttttctgctgacccggatcctgaccattccccagagcctggactcctggtggacctccctgaacttcctgggcggagcccctacatgccctggccagaatagccagagccccaccagcaaccacagccctaccagctgccctcctatctgccccggctacaggtggatgtgcctgcggcggttcatcatctttctgttcatcctgctgctgtgcctgatctttctgctggtgctgctggactatcagggcatgctgcccgtgtgccctctgctgcctggcaccagcacaacaagcaccggcccttgcaagacctgcaccatccctgcccagggaaccagcatgttccccagctgctgctgcaccaagcccagcgacggcaactgcacctgtatccccatcccttccagctgggccttcgccagattcctgtgggagtgggccagcgtgcggttcagctggctgtcactgctggtgcccttcgtgcagtggttcgtgggcctgtctcctaccgtgtggctgagcgtgatctggatgatgtggtactggggcccctccctgtacaatatcctgagccccttcctgcccctgctgcccatcttcttttgcctgtgggtgtacatccggaagcggagagcccccgtgaagcagaccctgaatttcgacctgctgaagctggccggcgacgtggaaagcaaccctggccctatgcagctgtttcacctgtgtctgatcatcagctgtagctgccccaccgtgcaggccagcaagctgtgtctgggatggctgtgggggatggacatcgacccctacaaagaattcggcgcctctgtggaactgctgagcttcctgcctagcgacttcttcccctccatccgggacctgctggatacagccagcgccctgtatagagaggccctggaaagccccgagcactgcagccctcatcacaccgctctgaggcaggccatcctgtgttggggcgagctgatgaacctggccacttgggtgggaagcaacctggaagatcccgcctcccgggaactggtggtgtcttacgtgaacgtgaacatgggcctgaagatccggcagctgctgtggttccacatctcctgcctgaccttcggccgggaaaccgtgctggaatacctggtgtccttcggcgtgtggatcagaaccccccctgcctacagaccccccaacgcccctatcctgtccaccctgcctgagacaactgtcgtgcggcggagaggcagaagccccagaagaagaacccccagccccagacgcagaagaagccagtcccctcggcggagaagatcccagagcagggaaagccagtgtggcggaggatctggcggcggaatgcctctgagctaccagcacttccggaaactgctgctgctggatgacgaggccggacccctggaagaggaactgcctagactggccgacgagggcctgaacagaagagtggccgaggacctgaacctgggcaacctgaatgtgtctatcccctggacccacaaagtgggcaacttcaccggcctggcctccagcaccgtgcccgtgtttaatcccgagtggcagacccccagcttcccccacatccatctgcaggaagatatcatcaaccgctgccagcagtacgtgggccctctgaccgtgaacgagaagcgcagactgaagctgatcatgcccgccaggttctaccccaacctgaccaagtacctgcctctggacaagggcatcaagccctactaccctgagcacgccgtgaaccactacttcaagacccggcactacctgcacaccctgtggaaggccggcatcctgtataagcgggaaacaaccagatccgccagcttctgcggctccccctacagctgggagcaggaactgcagcacggccggctggtgtttcagacctctaccagacacggcgacgagtctttctgcagccagagcagcgggatcctgagcagatctccagtgggcccttgtgtgcggtcccagctgaagcagagtagactgggcctgcagcctcagcagggctctctggctagaggcaagtctggcagaagcggcagcatccgggccagagtgcaccctaccaccagacggtcctttggcgtggaacctagcggcagcggccacatcgataacagcgcctctagcacctccagctgcctgcatcagtccgccgtgcggaaaaccgcctacagccacctgagcacaagcaagagacagagcagctccggccacgccgtggaactgcacaatatcccccctagcagcgccagaagccagagcgagggccctatctttagcgcctggtggctgcagtttcggaacagcaagcccgccagcgactacgccctgacccacatcgtgaacctgctggaagattggggccctgccacagagcacggcgagcacaacatccggatccccagaacccctgccagagtgacaggcggcgtgttcctggtggataagaaccctcacaacaccaccgagagcagactggtggtggacttcagccagttcagccggggctctacccacgtgtcctggcctaagttcgccgtgcccaacctgcagagcctgaccaacctgctgtccagcaacctgtcctggctgtccctggatgtgtccgccgccttctaccacatcccactgcatcccgccgctatgccccatctgctcgtgggaagtagcggcctgcctagatacgtggccagactgagcagcacctcccggaacatcaattaccagcacggcaccatgcaggatctgcacgacagctgcagccggaacctgtacgtgtcccttctgctgctgtacaagaccttcggcagaaagctgcatctgtactcccaccccatcatcctgggctttagaaagatccccatgggcgtgggactgtccccattcctgctggcccagttcaccagcgccatctgttccgtcgtgcgcagagccttccctcactgcctggcctttagctacatggacgacgtggtgctgggcgccaagagcgtgcagcacctggaatccctgtttaccagcatcaccaactttctgctgagcctgggcatccacctgaaccctaacaagaccaagcgctggggctacagcctgaactttatgggctacgtgatcggctcctggggcacactgcctcaggaacacatcgtgctgaaaatcaagcagtgctttcggaagctgcctgtgaaccggcccatcgactggaaagtgtgccagcggattgtgggcctgctgggctttgccgccccttttacacagtgcggctaccccgctctgatgcccctgtacgcctgcattcagagcaagcaggccttcaccttcagcccaacctacaaggccttcctgtgcaagcagtacctgaatctgtaccccgtggcccggcagagaagcggcctgtgtcaggtgttcgccgatgccacacctacaggatggggcctggccatcggacacagagccatgagaggcaccttcgtggccccactgcctattcacacagccgagctgctggctgcctgtttcgccaggtctagaagcggcgccaagctgatcggcaccgacaactctgtggtgctgagccggaagtacacctccttcccttggctgctgggctgcgccgccaactggatcctgagaggcacaagcttcgtgtacgtgccctccgccctgaatcctgccgccgatcctagtgctggacggctgggactgtaccggcctctgctgcatctgcccttcagacccaccaccggcagaaccagcctgtatgccgtgtcccctagcgtgccaagccatctgcccgacagagtgcatttcgcctcccccctgcatgtggcttggaggcccccttaa

SEQ ID NO: 48 - DNA, 4704 bp, synthetic construct
  misc_feature 1..4704: note = Vaccine construct
  source 1..4704: mol_type = other DNA
cccgccgccaccatgtctttgctttggggcggagtgacagtgctggccgccatgcttattgctggacaggtggccagcgtggtgttcctggtcaaaggcggaggacctggcggaggcatgcagctgtttcacctgtgcctgatcatcagctgtagctgccctacagtgcaggccagcaagctgtgtcttggatggctgtggggcatggacatcgacccctacaaagaattcggcgcctccgtggaactgctgagcttcctgcctagcgacttcttcccctccatccgggatctgctggatacagccagcgcactgtatagagaggccctggaaagccccgagcactgttctccacatcacacagccctgagacaggccatcctgtgttggggcgagctgatgaatctggccacatgggtcggaagcaacctggaagatcccgccagcagagaactggtggtgtcctacgtgaacgtgaacatgggcctgaagatccggcagctgctgtggttccacatctcctgcctgaccttcggcagagaaaccgtgctggaatacctggtgtccttcggcgtgtggatcagaacccctccagcctacagacctcctaacgctcccatcctgagcaccctgcctgagacaacagtcgtgcggagaagaggcagaagccccagaagaagaaccccatctcctcggaggcggagatctcagtcccctcggagaagaagatcccagagcagggaaagccagtgcggtggtggaagtggtggcggaatgcctctgagctaccagcacttcagaaaactgctgctgctggacgacgaggccggacctcttgaggaagaactgcctagactggccgacgagggcctgaatagaagagtggccgaggacctgaacctgggcaacctgaatgtgtctatcccctggacacacaaagtgggcaacttcacaggcctggccagctctaccgtgcctgtgttcaatcccgagtggcagacccctagcttccctcacatccatctgcaagaggacatcatcaaccggtgccagcagtacgtgggccctctgaccgtgaacgagaagcggagactgaagctgatcatgcccgccagattctaccccaacctgaccaagtacctgcctctggacaagggcatcaagccttactaccctgagcacgccgtgaaccactacttcaagacccggcactacctgcacaccctgtggaaggccggcatcctgtacaagagagagacaaccagaagcgccagcttctgcggcagcccttactcttgggagcaagagctgcagcacggcagactggtgttccagacctctacaagacacggcgacgagtctttctgcagccagagcagcgggatcctgtctagatctcctgtgggaccttgtgtgcggagccagctgaaacagagcagactgggactgcagcctcagcagggatctctggctagaggcaagtctggcagaagcggcagcatcagagccagagtgcaccctaccaccagacggtcctttggcgtggaacctagcggcagcggccacatcgataatagcgccagcagcaccagcagctgtctgcatcagtctgctgtgcgcaagaccgcctacagccacctgagcacaagcaagagacagagcagctctggccacgccgtcgagctgcacaatatccctccaagcagcgccagaagccagagcgagggccctattttttctgcctggtggctgcagttcagaaacagcaagcccgccagcgactacgccctgacacacatcgtgaacctgctggaagattggggccctgccacagaacacggcgagcacaacatcagaatccccaggactcccgccagagtgaccggcggagttttcctggtggacaagaaccctcacaacaccaccgagagcaggctggtggtggacttcagccagtttagcagaggcagcacccacgtgtcctggcctaaattcgccgtgcctaacctgcagagcctgaccaatctgctgagcagcaacctgagctggctgagcctggatgtgtccgccgccttctatcacatcccactgcatcctgccgctatgccccatctgcttgtgggaagttctggcctgcctagatatgtggcccggctgtcctctaccagccggaacatcaattaccagcacgggaccatgcaggacctgcacgatagctgctcccggaacctgtacgttagcctgctcctgctgtacaaaaccttcggccggaagctgcacctgtactctcaccctatcatcctgggcttcagaaagatccccatgggcgtgggactgagcccatttctgctggcccagtttaccagcgccatctgcagtgttgtgcggagagcctttcctcactgcctggccttcagctacatggacgatgtggtgctgggcgccaagtctgtgcagcacctggaatctctgtttacctccatcaccaactttctgctgtccctgggaatccatctgaaccccaacaagacaaagagatggggctacagcctgaacttcatgggctacgtgatcggcagctggggcacactgcctcaagagcacatcgtgctgaagatcaagcagtgctttagaaagctgcccgtgaacagacccatcgactggaaagtgtgccagcggattgtgggcctgctgggatttgccgctcctttcacacagtgcggctaccccgctctcatgcctctgtacgcctgcatccagtccaagcaggccttcacattcagccctacctacaaggccttcctgtgcaagcagtacctgaatctgtaccccgtggctagacagagaagcggcctgtgtcaggtgttcgccgatgccacacctacaggatggggactcgccattggacacagggccatgagaggaacctttgtggcccctctgcctattcacaccgccgaattgctggctgcctgtttcgccagatctagaagcggcgccaagctgatcggcaccgacaattcagtggtgctgagccggaagtacaccagctttccatggctgctgggctgtgccgccaactggattctgagaggcaccagcttcgtgtacgtgcccagcgctcttaaccctgccgctgatcctagtgctggacggctgggactctacagaccactgctgcatctgcccttcagacccaccacaggcagaaccagcctgtatgccgtgtctcctagcgtgccaagccatctgcctgatcgggtgcactttgcctctccactgcatgtggcttggcggcctccaaagagccccaacagcaacaaccccgactgggatttcaaccctaacaaggaccactggcctgaggccaatcaagttggagccggcgcttttggccctggctttacacctcctcatggcggactgcttggatggtcacctcaggctcagggcatcctgactacagtgcctgctgctccacctcctgccagcaccaatagacagtcaggcagacagcccacacctatcagccctccactgcgggatagtcacccacaggccatgcaatggaacagcaccaccttccatcaggccctgctggatcctagagtgcggggcctgtattttcctgccggcggatctagctccggcaccgtgaatcctgtgcctacaaccgcctctcctatcagcagcatcttcagccggacaggcgaccctgctcctaatggctctaagggcaagagaaagcggagggcccctgtgaaacagaccctgaatttcgacctgctgaagctggccggcgacgtggaatctaaccctggacctatgggcggctggtccagcaaacctagacaaggcatgggcaccaatctgagcgtgcccaatccactgggattcttcccagaccaccagctggaccctgccttcggcgccaatagcaacaatcctgattgggactttaatcccaacaaagatcattggcccgaagctaatcaagtcggcctggaaggcggaagcggcggcatggagaataccacatctggctttctgggcccactgctggttctccaggccggcttttttctcctgacacggatcctgacaatccctcagagcctggactcttggtggacctctctgaacttccttggcggagcccctacctgtcctggacagaatagccagtctcctaccagcaatcacagccccacaagctgccctccaatctgccctggctatcggtggatgtgcctgcggcggttcatcatctttctgttcatcctgctgctgtgtctgatcttcctgctggtcctcctggactaccagggcatgctgccagtttgtcctctgctgcctggcacctccacaacatctacaggcccttgcaagacctgtacaatccccgctcagggaaccagcatgttccccagctgttgctgcaccaagccaagcgacggcaactgcacatgcatccccattcctagcagctgggcctttgccagatttctgtgggagtgggcctccgtgcggtttagctggctgtctctgctcgtgcctttcgtgcagtggttcgtcggactgtctccaaccgtgtggctgagcgtgatctggatgatgtggtactggggccctagcctgtacaatattctgagccccttcctgcctctcctgccaatcttcttttgtctgtgggtgtacatctga

SEQ ID NO: 49 - DNA, 4800 bp, synthetic construct
  misc_feature 1..4800: note = Vaccine construct
  source 1..4800: mol_type = other DNA
cccgccgccaccatgtctttgctttggggcggagtgacagtgctggccgccatgcttattgctggacaggtggccagcgtggtgttcctggtcaaaggcggaggacctggcggaggcatgcagctgtttcacctgtgcctgatcatcagctgtagctgccctacagtgcaggccagcaagctgtgtcttggatggctgtggggcatggacatcgacccctacaaagaattcggcgcctccgtggaactgctgagcttcctgcctagcgacttcttcccctccatccgggatctgctggatacagccagcgcactgtatagagaggccctggaaagccccgagcactgttctccacatcacacagccctgagacaggccatcctgtgttggggcgagctgatgaatctggccacatgggtcggaagcaacctggaagatcccgccagcagagaactggtggtgtcctacgtgaacgtgaacatgggcctgaagatccggcagctgctgtggttccacatctcctgcctgaccttcggcagagaaaccgtgctggaatacctggtgtccttcggcgtgtggatcagaacccctccagcctacagacctcctaacgctcccatcctgagcaccctgcctgagacaacagtcgtgcggagaagaggcagaagccccagaagaagaaccccatctcctcggaggcggagatctcagtcccctcggagaagaagatcccagagcagggaaagccagtgcggtggtggaagtggtggcggaatgcctctgagctaccagcacttcagaaaactgctgctgctggacgacgaggccggacctcttgaggaagaactgcctagactggccgacgagggcctgaatagaagagtggccgaggacctgaacctgggcaacctgaatgtgtctatcccctggacacacaaagtgggcaacttcacaggcctggccagctctaccgtgcctgtgttcaatcccgagtggcagacccctagcttccctcacatccatctgcaagaggacatcatcaaccggtgccagcagtacgtgggccctctgaccgtgaacgagaagcggagactgaagctgatcatgcccgccagattctaccccaacctgaccaagtacctgcctctggacaagggcatcaagccttactaccctgagcacgccgtgaaccactacttcaagacccggcactacctgcacaccctgtggaaggccggcatcctgtacaagagagagacaaccagaagcgccagcttctgcggcagcccttactcttgggagcaagagctgcagcacggcagactggtgttccagacctctacaagacacggcgacgagtctttctgcagccagagcagcgggatcctgtctagatctcctgtgggaccttgtgtgcggagccagctgaaacagagcagactgggactgcagcctcagcagggatctctggctagaggcaagtctggcagaagcggcagcatcagagccagagtgcaccctaccaccagacggtcctttggcgtggaacctagcggcagcggccacatcgataatagcgccagcagcaccagcagctgtctgcatcagtctgctgtgcgcaagaccgcctacagccacctgagcacaagcaagagacagagcagctctggccacgccgtcgagctgcacaatatccctccaagcagcgccagaagccagagcgagggccctattttttctgcctggtggctgcagttcagaaacagcaagcccgccagcgactacgccctgacacacatcgtgaacctgctggaagattggggccctgccacagaacacggcgagcacaacatcagaatccccaggactcccgccagagtgaccggcggagttttcctggtggacaagaaccctcacaacaccaccgagagcaggctggtggtggacttcagccagtttagcagaggcagcacccacgtgtcctggcctaaattcgccgtgcctaacctgcagagcctgaccaatctgctgagcagcaacctgagctggctgagcctggatgtgtccgccgccttctatcacatcccactgcatcctgccgctatgccccatctgcttgtgggaagttctggcctgcctagatatgtggcccggctgtcctctaccagccggaacatcaattaccagcacgggaccatgcaggacctgcacgatagctgctcccggaacctgtacgttagcctgctcctgctgtacaaaaccttcggccggaagctgcacctgtactctcaccctatcatcctgggcttcagaaagatccccatgggcgtgggactgagcccatttctgctggcccagtttaccagcgccatctgcagtgttgtgcggagagcctttcctcactgcctggccttcagctacatggacgatgtggtgctgggcgccaagtctgtgcagcacctggaatctctgtttacctccatcaccaactttctgctgtccctgggaatccatctgaaccccaacaagacaaagagatggggctacagcctgaacttcatgggctacgtgatcggcagctggggcacactgcctcaagagcacatcgtgctgaagatcaagcagtgctttagaaagctgcccgtgaacagacccatcgactggaaagtgtgccagcggattgtgggcctgctgggatttgccgctcctttcacacagtgcggctaccccgctctcatgcctctgtacgcctgcatccagtccaagcaggccttcacattcagccctacctacaaggccttcctgtgcaagcagtacctgaatctgtaccccgtggctagacagagaagcggcctgtgtcaggtgttcgccgatgccacacctacaggatggggactcgccattggacacagggccatgagaggaacctttgtggcccctctgcctattcacaccgccgaattgctggctgcctgtttcgccagatctagaagcggcgccaagctgatcggcaccgacaattcagtggtgctgagccggaagtacaccagctttccatggctgctgggctgtgccgccaactggattctgagaggcaccagcttcgtgtacgtgcccagcgctcttaaccctgccgctgatcctagtgctggacggctgggactctacagaccactgctgcatctgcccttcagacccaccacaggcagaaccagcctgtatgccgtgtctcctagcgtgccaagccatctgcctgatcgggtgcactttgcctctccactgcatgtggcttggcggcctccaaagagccccaacagcaacaaccccgactgggatttcaaccctaacaaggaccactggcctgaggccaatcaagttggagccggcgcttttggccctggctttacacctcctcatggcggactgcttggatggtcacctcaggctcagggcatcctgactacagtgcctgctgctccacctcctgccagcaccaatagacagtcaggcagacagcccacacctatcagccctccactgcgggatagtcacccacaggccatgcaatggaacagcaccaccttccatcaggccctgctggatcctagagtgcggggcctgtattttcctgccggcggatctagctccggcaccgtgaatcctgtgcctacaaccgcctctcctatcagcagcatcttcagccggacaggcgaccctgctcctaatggctctaagggcaagagaaagcggagggcccctgtgaaacagaccctgaatttcgacctgctgaagctggccggcgacgtggaatctaaccccggacctatggacgccatgaagaggggcctgtgttgtgtcctgctgctgtgtggcgctgtgttcgtgtctcccagccaagagatccacgccagatttcggagaatgggcggctggtccagcaagcctagacaaggcatgggcaccaatctgagcgtgcccaatccactgggattcttcccagaccaccagctggaccctgccttcggcgccaatagcaacaatcctgattgggactttaatcccaacaaagatcattggcccgaagctaatcaagtcggcctggaaggcggaagcggcggcatggagaataccacatctggctttctgggcccactgctggttctccaggccggcttttttctcctgacacggatcctgacaatccctcagagcctggacagttggtggacctctctgaacttccttggcggagcccctacctgtcctggacagaatagccagtctcctaccagcaatcacagccccacaagctgccctccaatctgccctggctatcggtggatgtgcctgcggcggttcatcatctttctgttcattctgctgctttgcctgatcttcctgctggtcctcctggactaccagggcatgctgccagtttgtcctctgctgcctggcacctccacaacctctacaggcccttgcaagacctgtacaatccccgctcagggaaccagcatgttccccagctgttgctgcaccaagccaagcgacggcaactgcacatgcatccccattcctagcagctgggcctttgccagattcctctgggaatgggcctccgtgcggtttagctggctgtctctgctcgtgcctttcgtgcagtggttcgtcggactgtctccaaccgtgtggctgagcgtgatctggatgatgtggtactggggccctagcctgtacaatattctgagccccttcctgcctctcctgccaatcttcttttgtctgtgggtgtacatctga

SEQ ID NO: 50 - DNA, 2100 bp, synthetic construct
  misc_feature 1..2100: note = Promoter
  source 1..2100: mol_type = other DNA
tgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgca

SEQ ID NO: 51 - DNA, 673 bp, synthetic construct
  misc_feature 1..673: note = Promoter
  source 1..673: mol_type = other DNA
cctttcactcattagatgcatgtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggt

SEQ ID NO: 52 - protein, 119 aa, Hepatitis B virus
MGGWSSKPRQGMGTNLSVPNPLGFFPDHQLDPAFGANSNNPDWDFNPNKDHWPEANQVGAGAFGPGFTPPHGGLLGWSPQAQGILTTVPAAPPPASTNRQSGRQPTPISPPLRDSHPQA

SEQ ID NO: 53 - protein, 55 aa, Hepatitis B virus
MQWNSTTFHQALLDPRVRGLYFPAGGSSSGTVNPVPTTASPISSIFSRTGDPAPN

SEQ ID NO: 54 - protein, 25 aa, Hepatitis B virus
FLGPLLVLQAGFFLLTRILTIPQSL

SEQ ID NO: 55 - protein, 19 aa, Hepatitis B virus
FIIFLFILLLCLIFLLVLL

SEQ ID NO: 56 - protein, 25 aa, Hepatitis B virus
RFLWEWASVRFSWLSLLVPFVQWFV

SEQ ID NO: 57 - protein, 22 aa, Hepatitis B virus
TVWLSVIWMMWYWGPSLYNILS

SEQ ID NO: 58 - DNA, 7239 bp, synthetic construct
  misc_feature 1..7239: note = Vaccine construct
  source 1..7239: mol_type = other DNA
gtaatctattcgatataccgttgctaacagtatactggcccaataactgtggatggaaaatctataataatacattaatatcatccgatggtgctagggttatttggatggatgcgtataaattttcttgcggtttatctttacaagactattgttatcattggggtagcaaaccagagagccgaccattcgatttaataaaaaaatcagatgctaaacgcaattctaaatcgttggtcaaagaatctatggcatccttgaaatccttgtacgaggcattcgagacacaatcaggagcgttagaagttttaatgagtccatgtaggatgttttcgttttctagaatagaagacatgttcttaactagtgtcattaatagagtatccgagaatactggaatggggatgtattatcctaccaacgatataccttctctatttatcgaatcatctatctgtctagattatattatagtaaataatcaggaatccaacaaatatcgtatcaaatctgttctcgatatcatttcttcaaaacaataccctgcaggacgtcccaactacgttaaaaatggtacaaaaggaaagttatatatcgcgttgtgtaaagttaccgtacctactaacgaccatattccagtagtttatcacgatgatgacaatactaccacctttattacagtattgacgtccgtcgatattgaaactgctatcagagcaggatattcgatagtcgaattaggggctttacaatgggataataatattccagaacttaaaaacggtttactggatagtatcaagatgatttatgacttgaacgcagttacaacaaataatttattggaacagctcatagaaaatattaactttaacaactctagtataatttcgttgttttatacatttgccattagttattgccgagcattcatttactcaattatggaaaccatagatccggtgtatatatctcagttcagttataaagaattatacgttagtagctcttataaagatattaatgaatccatgagtcagatggtaaaattataaaaagtgaaaaacaatattatttttatcgttggttgttacactatgtcacttctttggggcggagttacagttcttgctgctatgcttattgctggacaagttgcttctgttgtgtttcttgtttctaaatctggacctccttctggaaaatctaattctaacaatcctgattgggatttcaatcctaacaaagatcattggcctgaagctaatcaagttggagctggtgcttttggacctggttttacacctcctcatggtggattgcttggatggtcacctcaagctcagggaattcttacaacagttccagctgctcctcctcctgcttctacaaatagacaatctggtagacaacctacacctatttctccacctcttagagattctcatcctcaagctatgcaatggaattctactacatttcatcaagctttgcttgatcctagagttagaggactttattttcctgctggtggaagttcttctggaacagttaatcctgttcctacaacagcttctccaatttcttctatattttctagaacaggcgatcctgctcctaatggatcaaaatctggatcaaaaatgcctctgtcttatcaacactttagaaaattgctgcttcttgatgatgaagctggacctcttgaagaagaattgcctagacttgctgatgaaggacttaatagaagagttgctgaagatcttaatctgggaaatcttaatgtttctattccttggacacacaaagttggaaatttcacaggacttgcatcttctacagtgcctgtttttaatcctgaatggcaaacaccttcttttccacatattcatctgcaagaggatatcatcaatagatgtcaacaatatgttggaccactgacagttaatgagaagagaaggcttaaacttattatgcctgctagattctatcctaatcttacaaagtatttgcctctggataagggaatcaaaccttattatcctgaacatgctgtgaatcactactttaaaacaagacattatctgcatacactgtggaaagctggtattctttacaaaagagaaacaacaagatctgcttcattttgtggatctccatattcttgggaacaagaacttcaacatggtagacttgtttttcaaacatctacaagacatggggatgaatcattttgttctcaaagttctggaattctttctagatctcctgttggaccttgtgttagatctcaacttaaacaatctagacttggacttcaacctcaacaaggatctcttgctagaggaaaaagtggaagatctggatctattagagctagagttcatcctacaactagaagatcttttggagttgaaccttctggatctggacatattgataattctgcctcttctacatcttcttgtctgcatcaatctgctgttagaaagacagcttattctcatttgtctacttctaagagacaatcatcttctggacatgctgttgaacttcataatattcctccaagtagtgctagaagtcaatctgaaggaccaatattttcagcttggtggcttcaattcagaaattctaaacctgcttctgattatgctctgacacatatagttaatttgcttgaagattggggacctgctacagaacatggcgaacacaatattagaatacctagaactcctgctagagttacaggcggagtctttttggttgataagaatcctcataataccacagaatcaagacttgttgttgatttttcacagttttctagaggatctacacatgtttcttggcctaaatttgctgttccaaatcttcaatctcttacaaatttgctttcatctaatctttcttggctgtctcttgatgtttctgctgccttttatcatattcctcttcatcctgctgcaatgcctcatttgcttgttggatcatctggacttccaagatatgttgctagacttagctctacatctagaaatatcaattatcagcatggaacaatgcaggatcttcacgattcttgtagtaggaatctgtatgtttctttgcttctgctgtataagacatttggaagaaaacttcatctgtattctcaccctattattctgggttttagaaagattcctatgggagttggactttctccttttttgcttgctcaattcacatctgctatttgttctgttgttagaagggcttttcctcattgtcttgcattttcttatatggatgatgttgttcttggagctaaatctgttcaacatcttgaaagtctgtttacctctattactaattttctgctttctctgggaattcatctgaatccaaacaaaacaaagagatggggatattctcttaatttcatgggatatgttattggatcttggggaacacttcctcaagaacatatcgttttgaaaatcaagcaatgtttcagaaaactgcctgtgaatagacctattgattggaaagtttgtcaaagaattgtgggacttcttggatttgctgctccttttacacaatgtggatatcctgctcttatgccactttatgcttgtattcaatctaaacaggcttttacattttctccaacatacaaagcttttctgtgtaaacagtatctgaatctttatcctgtggctagacaaagatctggtctttgtcaagtttttgctgatgctacaccaacaggatggggacttgctattggacatagagctatgagaggaacatttgttgctccattgcctattcatacagctgaattgcttgctgcttgttttgctagatctagaagcggagcaaaacttattggtacagataatagtgttgtcctgagtagaaagtacacatcttttccatggttgttgggatgtgctgctaattggattcttagaggaacttcttttgtttatgttccttctgctcttaatcctgcagctgatccatctgctggtagattgggactgtatagaccacttcttcatttgccttttagaccaacaactggaagaacatctctttatgctgtttctccttctgttccatctcatttgcctgatagagttcattttgcttctccacttcatgttgcttggaggccaccatctaaatctccaggttctggaccacctatgcaactttttcatttgtgtttgatcattagctgttcttgtcctacagttcaagcttctaaactttgtcttggatggctttggggaatggatattgatccatacaaagaatttggagctagtgttgaattgctgtcatttcttccatctgattttttcccttctattcgtgatcttcttgatacagcatctgctctgtatagagaagctcttgaatctcctgaacactgttctccacatcatacagcacttagacaagctattctttgttggggagaacttatgaatcttgctacatgggttggatctaatttggaagatccagcttctagagaattggtggtttcttatgttaatgtgaatatgggactgaaaattagacaactgctttggtttcatatctcttgtcttacatttggtagagaaacagttttggaatatttggtttcttttggcgtttggattagaacacctccagcttatagacctcctaatgctcctattttgtctacacttcctgaaacaacagtcgttagaagaagaggaagatctccaagaagaagaacaccaagtcctagaagaagaagatctcaatcaccaagaagaagaagaagtcaatctagagaatctcaatgttgatttttgttcgacattaaaaattgaaaataaatacaaaggttcttgagggttgtgttaaattgaaagcgagaaataatcataaatatgattcaggtgacggatccatggatgctatgaagcgaggactttgttgtgttttgcttctttgtggtgctgtgtttgtttctccatctcaagaaattcatgccagattcagaagaatgggaggctggtcatctaaacctagacaaggcatgggaacaaatctttctgttcctaatcctttgggattctttcctgatcatcaattggatccagcatttggagcaaatagtaacaatccagattgggactttaacccaaacaaagatcattggccagaagctaatcaagttggagcatctaaaggtggaaaaagtggaatggaaaacactacatctggatttcttggacctttgcttgttcttcaagctggatttttcctgttgacaagaatacttacaattcctcaatcactggattcttggtggacaagtcttaattttcttggaggtgctcctacatgtcctggacaaaattctcaatctccaacttctaatcattctcctacatcttgtcctccaatttgtcctggatatagatggatgtgtcttagaagattcattatctttcttttcatactgctgctgtgtctgattttccttcttgttttgttggattatcagggaatgcttcctgtttgtcctttgcttcctggaacttctacaacaagtacaggaccttgtaaaacatgtacaattccagcacagggaacatctatgtttccaagttgttgttgtacaaaaccttctgatggaaattgcacatgtattcctattccaagttcttgggcatttgctagatttctttgggaatgggcttctgttagattcagttggttgtctcttttggttccatttgttcagtggtttgttggattgtctcctacagtttggctttctgttatttggatgatgtggtattggggaccttctctttacaatattttgagtccttttctccctttgctgccaattttcttttgtctttgggtttacatttgattaaccgagtttctgcattattgtaattcgtatgctggcaccatcaaagaatcacttctaaaagatatcaatatcacacatacaaatattactaccctattgaatgagacagccaaggttatcaagttagtaaaatctctggtagataaagaagatactgatattgtgaataatttcattaccaaagaaattaaaaacagagacaaaatagttaatagtttgtctctatcaaacctggactttcgtttgtaaattggggctttttgtacaataaatgggtgttgccaatgattcatcccctgaatatcaatggatgtctccccatagattatcagatactgttatattaggagactgtttgtattttaacaatataatgtcccaattagatttacaccaaaattgggctccatcagttagattgttaaattattttaagaattttaataaggaaacactactaaagatagaagagaatgattacattaattcatcctttttccaacaaaaggataaacgattttatcctataaacgacgatttttatcacatatctacaggaggatatggtatagtctttaagatagataactatgtagtaaaatttgtattcgaggccacaaaattatatagtcccatggaaactacggcggagttcacagtacccaaatttctatacaacaatctaaagggagatgaaaaaaaattaatcgtgtgtgcgtgggccatgggattaaactataaattaacatttttacatactctgtataaacgtgttcttcatatgttgctattattgatacaaactatggatggtcaggaactatcattgagatattcttctaaagtttttttaaaggcgtttaacgagagaaaggacagtatcaaattcgtgaaattactatcccacttttatccggcagttattaacagtaatattaatgttataaactattttaaccgcatgtttcactttttcgaacatgaaaagagaactaactacgaatacgaaagaggaaatattataatttttcccctagcactgtattcggcagataaagtagataccgagctagctatcaaattaggatttaaatctttggtacaatacataaagtttatctttttacagatggctctgttatacattaaaatttacgaactaccatgctgcgacaactttttacacgcagatcttaaacccgataatatcttactttttgattccaatgaaccaataataattcatctaaaggataaaaagtttgtttttaatgaacgtattaaatcggcattaaacgactttgacttttcccaag

SEQ ID NO: 59 - DNA, 3741 bp, synthetic construct
  misc_feature 1..3741: note = Vaccine construct
  source 1..3741: mol_type = other DNA
atgtcacttctttggggcggagttacagttcttgctgctatgcttattgctggacaagttgcttctgttgtgtttcttgtttctaaatctggacctccttctggaaaatctaattctaacaatcctgattgggatttcaatcctaacaaagatcattggcctgaagctaatcaagttggagctggtgcttttggacctggttttacacctcctcatggtggattgcttggatggtcacctcaagctcagggaattcttacaacagttccagctgctcctcctcctgcttctacaaatagacaatctggtagacaacctacacctatttctccacctcttagagattctcatcctcaagctatgcaatggaattctactacatttcatcaagctttgcttgatcctagagttagaggactttattttcctgctggtggaagttcttctggaacagttaatcctgttcctacaacagcttctccaatttcttctatattttctagaacaggcgatcctgctcctaatggatcaaaatctggatcaaaaatgcctctgtcttatcaacactttagaaaattgctgcttcttgatgatgaagctggacctcttgaagaagaattgcctagacttgctgatgaaggacttaatagaagagttgctgaagatcttaatctgggaaatcttaatgtttctattccttggacacacaaagttggaaatttcacaggacttgcatcttctacagtgcctgtttttaatcctgaatggcaaacaccttcttttccacatattcatctgcaagaggatatcatcaatagatgtcaacaatatgttggaccactgacagttaatgagaagagaaggcttaaacttattatgcctgctagattctatcctaatcttacaaagtatttgcctctggataagggaatcaaaccttattatcctgaacatgctgtgaatcactactttaaaacaagacattatctgcatacactgtggaaagctggtattctttacaaaagagaaacaacaagatctgcttcattttgtggatctccatattcttgggaacaagaacttcaacatggtagacttgtttttcaaacatctacaagacatggggatgaatcattttgttctcaaagttctggaattctttctagatctcctgttggaccttgtgttagatctcaacttaaacaatctagacttggacttcaacctcaacaaggatctcttgctagaggaaaaagtggaagatctggatctattagagctagagttcatcctacaactagaagatcttttggagttgaaccttctggatctggacatattgataattctgcctcttctacatcttcttgtctgcatcaatctgctgttagaaagacagcttattctcatttgtctacttctaagagacaatcatcttctggacatgctgttgaacttcataatattcctccaagtagtgctagaagtcaatctgaaggaccaatattttcagcttggtggcttcaattcagaaattctaaacctgcttctgattatgctctgacacatatagttaatttgcttgaagattggggacctgctacagaacatggcgaacacaatattagaatacctagaactcctgctagagttacaggcggagtctttttggttgataagaatcctcataataccacagaatcaagacttgttgttgatttttcacagttttctagaggatctacacatgtttcttggcctaaatttgctgttccaaatcttcaatctcttacaaatttgctttcatctaatctttcttggctgtctcttgatgtttctgctgccttttatcatattcctcttcatcctgctgcaatgcctcatttgcttgttggatcatctggacttccaagatatgttgctagacttagctctacatctagaaatatcaattatcagcatggaacaatgcaggatcttcacgattcttgtagtaggaatctgtatgtttctttgcttctgctgtataagacatttggaagaaaacttcatctgtattctcaccctattattctgggttttagaaagattcctatgggagttggactttctccttttttgcttgctcaattcacatctgctatttgttctgttgttagaagggcttttcctcattgtcttgcattttcttatatggatgatgttgttcttggagctaaatctgttcaacatcttgaaagtctgtttacctctattactaattttctgctttctctgggaattcatctgaatccaaacaaaacaaagagatggggatattctcttaatttcatgggatatgttattggatcttggggaacacttcctcaagaacatatcgttttgaaaatcaagcaatgtttcagaaaactgcctgtgaatagacctattgattggaaagtttgtcaaagaattgtgggacttcttggatttgctgctccttttacacaatgtggatatcctgctcttatgccactttatgcttgtattcaatctaaacaggcttttacattttctccaacatacaaagcttttctgtgtaaacagtatctgaatctttatcctgtggctagacaaagatctggtctttgtcaagtttttgctgatgctacaccaacaggatggggacttgctattggacatagagctatgagaggaacatttgttgctccattgcctattcatacagctgaattgcttgctgcttgttttgctagatctagaagcggagcaaaacttattggtacagataatagtgttgtcctgagtagaaagtacacatcttttccatggttgttgggatgtgctgctaattggattcttagaggaacttcttttgtttatgttccttctgctcttaatcctgcagctgatccatctgctggtagattgggactgtatagaccacttcttcatttgccttttagaccaacaactggaagaacatctctttatgctgtttctccttctgttccatctcatttgcctgatagagttcattttgcttctccacttcatgttgcttggaggccaccatctaaatctccaggttctggaccacctatgcaactttttcatttgtgtttgatcattagctgttcttgtcctacagttcaagcttctaaactttgtcttggatggctttggggaatggatattgatccatacaaagaatttggagctagtgttgaattgctgtcatttcttccatctgattttttcccttctattcgtgatcttcttgatacagcatctgctctgtatagagaagctcttgaatctcctgaacactgttctccacatcatacagcacttagacaagctattctttgttggggagaacttatgaatcttgctacatgggttggatctaatttggaagatccagcttctagagaattggtggtttcttatgttaatgtgaatatgggactgaaaattagacaactgctttggtttcatatctcttgtcttacatttggtagagaaacagttttggaatatttggtttcttttggcgtttggattagaacacctccagcttatagacctcctaatgctcctattttgtctacacttcctgaaacaacagtcgttagaagaagaggaagatctccaagaagaagaacaccaagtcctagaagaagaagatctcaatcaccaagaagaagaagaagtcaatctagagaatctcaatgttga

SEQ ID NO: 60 - DNA, 96 bp, synthetic construct
  misc_feature 1..96: note = TPA encoded
  source 1..96: mol_type = other DNA
atggatgctatgaagcgaggactttgttgtgttttgcttctttgtggtgctgtgtttgtttctccatctcaagaaattcatgccagattcagaaga

SEQ ID NO: 61 - DNA, 882 bp, synthetic construct
  misc_feature 1..882: note = Vaccine construct
  source 1..882: mol_type = other DNA
atgggaggctggtcatctaaacctagacaaggcatgggaacaaatctttctgttcctaatcctttgggattctttcctgatcatcaattggatccagcatttggagcaaatagtaacaatccagattgggactttaacccaaacaaagatcattggccagaagctaatcaagttggagcatctaaaggtggaaaaagtggaatggaaaacactacatctggatttcttggacctttgcttgttcttcaagctggatttttcctgttgacaagaatacttacaattcctcaatcactggattcttggtggacaagtcttaattttcttggaggtgctcctacatgtcctggacaaaattctcaatctccaacttctaatcattctcctacatcttgtcctccaatttgtcctggatatagatggatgtgtcttagaagattcattatctttcttttcatactgctgctgtgtctgattttccttcttgttttgttggattatcagggaatgcttcctgtttgtcctttgcttcctggaacttctacaacaagtacaggaccttgtaaaacatgtacaattccagcacagggaacatctatgtttccaagttgttgttgtacaaaaccttctgatggaaattgcacatgtattcctattccaagttcttgggcatttgctagatttctttgggaatgggcttctgttagattcagttggttgtctcttttggttccatttgttcagtggtttgttggattgtctcctacagtttggctttctgttatttggatgatgtggtattggggaccttctctttacaatattttgagtccttttctccctttgctgccaattttcttttgtctttgggtttacatttga

(19) United States
(12) Patent Application Publication
(10) Pub. No.: US 2024/0115694 A1
(43) Pub. Date: Apr. 11, 2024

(54) HBV VACCINE

(71) Applicant: Oxford University Innovation Limited, Oxford (GB)

(72) Inventors: Eleanor BARNES, Oxford (Oxfordshire) (GB); Senthil CHINNAKANNAN, Oxford (Oxfordshire) (GB)

(21) Appl. No.: 18/206,249

(22) Filed: Jun. 6, 2023

Related U.S. Application Data
(63) Continuation of application No. 16/604,221, filed on Oct. 10, 2019, now abandoned.

(30) Foreign Application Priority Data
Apr. 10, 2017 (GB) 1705765.4

(51) Int. Cl. A61K 39/29; A61K 39/39; C07K 14/005; C12N 7/00

(52) U.S. Cl. CPC A61K 39/292; A61K 39/39; C07K 14/005; C12N 7/00; A61K 2039/5256

(57) ABSTRACT

The invention relates to a multi-HBV immunogen viral vector vaccine comprising: a viral vector comprising an immunogen expression cassette, wherein the expression of a protein encoded by the expression cassette is arranged to be driven by a promoter, wherein the immunogen expression cassette encodes: a) HBV Core; b) a modified HBV polymerase (Pₘᵤₜ), wherein the modification is a mutation to wild-type HBV polymerase to substantially remove polymerase function; c) HBV surface antigen (HbsAg); and d) an intergenic sequence that is arranged to cause expression of at least the HBV surface antigen (HbsAg) as a separate protein from the HBV core and the modified HBV polymerase (Pₘᵤₜ), wherein the intergenic sequence is downstream (3′) of the sequences encoding the HBV core and the modified HBV polymerase (Pₘᵤₜ) and upstream (5′) of the sequence encoding the HBV surface antigen (HbsAg); and related compositions, vaccination methods and methods of treatment or prophylaxis of HBV infection.

### CROSS-REFERENCE TO RELATED APPLICATIONS

This application is a Continuation of U.S. patent application Ser. No. 16/604,221, filed Oct. 10, 2019, which is the National Stage of International Application No. PCT/GB2018/050948, filed Apr. 10, 2018, which claims the priority to GB 1705765.4, filed Apr. 10, 2017, which are entirely incorporated herein by reference.

### SEQUENCE LISTING

This application contains a sequence listing filed in electronic form as a Sequence Listing XML file entitled “JDM86936P.USPC1_ST26.XML”, created on Aug. 30, 2023, and having a size of 391 KB. The content of the sequence listing is incorporated herein in its entirety.

### FIELD OF THE DISCLOSURE

This invention relates to multi-antigen HBV immunogen for viral vectored vaccines for therapeutic vaccination of chronic hepatitis B.

### BACKGROUND

Hepatitis B is a viral infection that attacks the liver and can cause both acute and chronic disease. The Hepatitis B virus (HBV) is transmitted through contact with the blood or other body fluids of an infected person. There is no specific treatment for acute hepatitis B. Therefore, care is aimed at maintaining comfort and adequate nutritional balance, including replacement of fluids lost from vomiting and diarrhoea. However, the mainstay of tackling acute Hepatitis B is by prevention using vaccination. Once established, chronic hepatitis B infection can be treated with drugs, including oral antiviral agents. Treatment can slow the progression of cirrhosis, reduce incidence of liver cancer and improve long-term survival. However, once chronic infection is established spontaneous viral control is rare. A major goal therefore is to develop immunotherapeutic HBV vaccines to induce viral control or cure.

Currently, there are few different T-cell inducing vaccines in Phase I and Phase II clinical trials, all of which are based on either one or two full length HBV-antigens or multiple truncated or full length chimeric HBV-antigens or synthetic peptides based on the HBV-proteome. GS-4774, by GlobeImmune is currently in Phase II clinical trial (GS-4774 has heat-inactivated yeast cells expressing a chimera of HBx HBsAg and HBcAg. TG1050, by Transgene is currently in Phase I clinical trial (TG1050 is a human adenovirus serotype 5 based vaccine encoding a chimera of truncated version of three HBV antigens, core-Polymerase-Envelope). HepTcell™, by Altimmune is currently in Phase I clinical trial (HepTcell is a completely synthetic peptide product, based on 9 synthetic 32-40 mer peptides derived from conserved regions of HBV protein). INO-1800, by Inovio is currently in Phase I clinical trial (INO-1800 is a multi-antigen SynCon® DNA immunotherapy targeting hepatitis B virus clades A & C surface antigens & HBV core antigens). Currently available prophylactic HBV vaccines (generally based on HBV protein vaccines) have no therapeutic effect on chronically infected HBV individuals and hence they fail to control chronic HBV infection. Therefore, an aim of the present invention is to provide an improved vaccine for HBV infection.

### SUMMARY OF THE DISCLOSURE

According to a first aspect of the invention, there is provided a multi-HBV immunogen viral vector vaccine comprising:
a viral vector comprising an immunogen expression cassette, wherein the expression of a protein encoded by the expression cassette is arranged to be driven by a promoter, wherein the immunogen expression cassette encodes:
a) HBV Core;
b) a modified HBV polymerase (Pₘᵤₜ), wherein the modification is a mutation to wild-type HBV polymerase to substantially remove polymerase function;
c) HBV surface antigen (HbsAg); and
d) an intergenic sequence that is arranged to cause expression of at least the HBV surface antigen (HbsAg) as a separate protein from the HBV core and the modified HBV polymerase (Pₘᵤₜ),
wherein the intergenic sequence is downstream (3′) of the sequences encoding the HBV core and the modified HBV polymerase (Pₘᵤₜ) and upstream (5′) of the sequence encoding the HBV surface antigen (HbsAg).

The intergenic sequence may comprise a cleavage domain, an IRES (Internal Ribosomal Entry Site), a splicing signal, or a secondary promoter. In one embodiment, the intergenic sequence comprises a cleavage domain comprising a sequence arranged to cause ribosome skipping. In another embodiment, the intergenic sequence comprises a secondary promoter to promote expression of at least the surface antigen (HbsAg).

Induction of a strong, multi-antigen specific T cell response against different HBV proteins is thought to play a major role in viral clearance of a resolving chronic HBV infection. For induction of a multi-antigen T-cell response, it can be important to have maximal number of T-cell epitopes, which requires encoding full-length HBV proteins within a T-cell inducing HBV vaccine. Some of the current HBV vaccines under development do not encode full-length HBV antigens. The present invention vaccine can encode 3 full-length proteins of HBV (namely the Core, optionally with Pre-Core region, Polymerase and Surface proteins). In addition, the vaccine design utilizes an at least two-protein expression, for example using an F2A peptide cleavage strategy to encode a separate surface protein, which in addition to the induction of T-cell response could also induce an antibody response that could possibly have a role in potentiating the therapeutic effect of T-cell vaccine, with possible help in clearance of the virus within a chronically infected individual. Inducing antibodies based on a mammalian system (compared to other systems, for e.g. Yeast) has a selective advantage of providing the mammalian-type glycosylation that could induce antibodies appropriate for the natural host.

The invention advantageously provides a single vaccine encoding full-length multiple-HBV antigens that could induce broad T-cell response and in-addition could also induce antibodies to the surface protein. The new HBV immunogen at least encompasses three full length HBV-antigens (namely the Core, Polymerase and Surface) and encoded them into a viral vector, such as the Chimpanzee adeno and MVA viral vectors. In addition, the HBV-polymerase protein in the immunogen is provided with mutations that abolish its function, which avoids its participation in HBV genome replication and improves safety for the vaccine.

By using a peptide cleavage strategy, for example by using a Furin-2A cleavage sequence, or a secondary promoter, the transgene cassette can generate at least two proteins, a fused core and polymerase protein and a separate surface protein, both of which generates a T-cell immune response. The encoded surface protein, in addition to the generation of T-cell immune response can also generate an antibody response.

Immunogen Expression Cassette

The skilled person will recognize that the cleavage domain may not lead to cleavage on all occasions, for example F2A based ribosomal skipping events may occur in a small proportion of expressions, such that a small proportion of proteins made will be a fusion of all the HBV proteins/antigens that would otherwise be separated by the cleavage. Therefore, in one embodiment, the immunogen expression cassette may further encode a fusion protein comprising at least the HBV Core, modified HBV polymerase (Pₘᵤₜ) and HBV surface antigen.

In one embodiment, the immunogen expression cassette further encodes HBV Pre-Core (PreC). In another embodiment, the immunogen expression cassette further encodes HBV PreS1, and/or a truncated form thereof. In another embodiment, the immunogen expression cassette further encodes HBV PreS2.

In one embodiment, the immunogen expression cassette further encodes HBV Pre-Core (PreC) and HBV PreS1, or a truncated form of HBV PreS1. In another embodiment, the immunogen expression cassette further encodes HBV Pre-Core (PreC) and HBV PreS2. In one embodiment, the immunogen expression cassette further encodes HBV Pre-Core (PreC) and HBV PreS1, and a truncated form of PreS1. In one embodiment, the immunogen expression cassette further encodes HBV Pre-Core (PreC), HBV PreS1, and/or a truncated form thereof, and HBV PreS2.

In an embodiment wherein the HBV Pre-core and Core are encoded, the immunogen expression cassette may be capable of expressing the HBV e-Antigen. The skilled person in the art will recognise that HBV e-Antigen comprises or consists of the 10 C-terminal amino acids of Pre-Core and the 149 N-terminal amino acids of the Core. For the expression of HBV e-Antigen, the Pre-Core and Core are expressed together and this whole expressed protein undergoes N-terminal and C-terminal cleavage.

In one embodiment, the HBV Core and modified polymerase (Pₘᵤₜ) are arranged to be expressed as a fusion protein. In an embodiment wherein the immunogen expression cassette encodes HBV Pre-core, the HBV Pre-core, HBV Core and modified polymerase (Pₘᵤₜ) are arranged to be expressed as a fusion protein.

In one embodiment the immunogen expression cassette encodes at least two proteins, comprising a first fusion protein comprising at least the HBV Core and the modified HBV polymerase (Pₘᵤₜ), and a second protein comprising at least the HBV surface antigen (HbsAg). In another embodiment the immunogen expression cassette encodes only two proteins, comprising a first fusion protein comprising at least the HBV Core and the modified HBV polymerase (Pₘᵤₜ), and a second protein comprising at least the HBV surface antigen (HbsAg).

In one embodiment, the immunogen expression cassette does not encode HBV X protein.

In one embodiment, the immunogen expression cassette may comprise a nucleic acid sequence encoding any one of:
SIi-HBV-CPmutS;
SIi-HBV-SCPmut;
HBV-CPmutS
SIi-HBV-CPmutPreS-S(sh);
SIi-HBV-CPmutPreS-TPA-S(sh);
SIi-HBV-PreS-Pmut-C;
MVA-SIi-HBV-PreS-Pmut-C-S(sh); or
MVA-SIi-HBV-PreS-Pmut-C-TPA-S(sh), as described herein.

In one embodiment, the immunogen expression cassette comprises SEQ ID NO: 46 (SIi-HBV-CPmutS) or a variant thereof. A variant of SEQ ID NO: 46 may comprise a sequence having at least 70%, 75%, 80%, 85%, 90%, 95%, 98%, 99%, or 99.5% identity with SEQ ID NO: 46. The variant of SEQ ID NO: 46 may encode protein that substantially retains the immunogenicity of equivalent protein encoded by SEQ ID NO: 46.

In one embodiment, the immunogen expression cassette comprises SEQ ID NO: 47 (SIi-HBV-SCPmut) or a variant thereof. A variant of SEQ ID NO: 47 may comprise a sequence having at least 70%, 75%, 80%, 85%, 90%, 95%, 98%, 99%, or 99.5% identity with SEQ ID NO: 47. The variant of SEQ ID NO: 47 may encode protein that substantially retains the immunogenicity of equivalent protein encoded by SEQ ID NO: 47.

In another embodiment, the immunogen expression cassette comprises SEQ ID NO: 48 (SIi-HBV-CPmutPreS-S(sh)) or a variant thereof. A variant of SEQ ID NO: 48 may comprise a sequence having at least 70%, 75%, 80%, 85%, 90%, 95%, 98%, 99%, or 99.5% identity with SEQ ID NO: 48. The variant of SEQ ID NO: 48 may encode protein that substantially retains the immunogenicity of equivalent protein encoded by SEQ ID NO: 48.

In another embodiment, the immunogen expression cassette comprises SEQ ID NO: 49 (SIi-HBV-CPmutPreS-TPA-S(sh)) or a variant thereof. A variant of SEQ ID NO: 49 may comprise a sequence having at least 70%, 75%, 80%, 85%, 90%, 95%, 98%, 99%, or 99.5% identity with SEQ ID NO: 49. The variant of SEQ ID NO: 49 may encode protein that substantially retains the immunogenicity of equivalent protein encoded by SEQ ID NO: 49.

In one embodiment, the immunogen expression cassette comprises SEQ ID NO: 59 (SIi-HBV-PreS-Pmut-C) or a variant thereof. A variant of SEQ ID NO: 59 may comprise a sequence having at least 70%, 75%, 80%, 85%, 90%, 95%, 98%, 99%, or 99.5% identity with SEQ ID NO: 59. The variant of SEQ ID NO: 59 may encode protein that substantially retains the immunogenicity of equivalent protein encoded by SEQ ID NO: 59.

In one embodiment, the immunogen expression cassette comprises SEQ ID NO: 24 (MVA-SIi-HBV-PreS-Pmut-C-S(sh)) or a variant thereof. A variant of SEQ ID NO: 24 may comprise a sequence having at least 70%, 75%, 80%, 85%, 90%, 95%, 98%, 99%, or 99.5% identity with SEQ ID NO: 24. The variant of SEQ ID NO: 24 may encode protein that substantially retains the immunogenicity of equivalent protein encoded by SEQ ID NO: 24.

In one embodiment, the immunogen expression cassette comprises SEQ ID NO: 27 (MVA-SIi-HBV-PreS-Pmut-C-TPA-S(sh)) or a variant thereof. A variant of SEQ ID NO: 27 may comprise a sequence having at least 70%, 75%, 80%, 85%, 90%, 95%, 98%, 99%, or 99.5% identity with SEQ ID NO: 27. The variant of SEQ ID NO: 27 may encode protein that substantially retains the immunogenicity of equivalent protein encoded by SEQ ID NO: 27. In one embodiment, the immunogen expression cassette comprises SEQ ID NO: 58 (MVA-SIi-HBV-PreS-Pmut-C-TPA-S(sh)) or a variant thereof. A variant of SEQ ID NO: 58 may comprise a sequence having at least 70%, 75%, 80%, 85%, 90%, 95%, 98%, 99%, or 99.5% identity with SEQ ID NO: 58. The variant of SEQ ID NO: 58 may encode protein that substantially retains the immunogenicity of equivalent protein encoded by SEQ ID NO: 58.

Viral Vector

The viral vector may comprise a virus. The immunogen expression cassette sequence of the invention may be cloned into any suitable viral vector that is known to elicit good immune response. Suitable viral vectors have been described in Dicks et al (Vaccine. 2015 Feb. 25; 33(9):1121-8. doi: 10.1016/j.vaccine.2015.01.042. Epub 2015 Jan. 25), Antrobus et al (Mol Ther. 2014 March; 22(3):668-74. doi: 10.1038/mt.2013.284. Epub 2013 Dec. 30.), and (Warimwe et al. (Virol J. 2013 Dec. 5; 10:349. doi: 10.1186/1743-422X-10-349), which are incorporated herein by reference.

The viral vector may be an attenuated viral vector. The viral vector may comprise an adenovirus, such as a human or simian adenovirus. In one embodiment, the viral vector comprises an adenovirus, such as a group E simian adenovirus, when used in a prime vaccine of a prime boost regime. The viral vector may comprise a group E simian adenovirus. The viral vector may comprise ChAdOx1 (a group E simian adenovirus, like the AdCh63 vector used safely in malaria trials) or ChAdOx2. The skilled person will be familiar with ChAdOx1 based viral vectors, for example from patent publication WO2012172277, which is herein incorporated by reference. The viral vector may comprise AdCh63. The viral vector may comprise AdC3 or AdH6. In one embodiment, the viral vector is a human serotype. In another embodiment, the viral vector comprises Modified Vaccinia Ankara (MVA). The viral vector may comprise MVA when used as a vaccine boost in a prime boost regime. In one embodiment, the viral vector comprises an adenovirus, such as a group E simian adenovirus, when used in a prime vaccine of a prime boost regime, and may comprise MVA when used as a vaccine boost in a prime boost regime. The skilled person will be familiar with MVA based viral vectors, for example from US patent publication U.S. Pat. No. 9,273,327, which is herein incorporated by reference.

MVA advantageously allows expression of more than one protein using different pox viral promoters.

In an alternative embodiment, the viral vector may comprise Adeno-associated virus (AAV) or Lentivirus. In another embodiment, the viral vector may comprise any of Vaccinia virus, fowlpox virus or canarypox virus (e.g. members of Poxviridae and the genus Avipoxvirus), or New York attenuated vaccinia virus (Tartaglia et al. Virology. 1992 May; 188(1):217-32, which is herein incorporated by reference). In another embodiment, the viral vector may comprise any of Herpes simplex virus, Human Cytomegalo virus, Measles virus (MeV), Sendai virus (SeV), Flavivirus (e.g. Yellow Fever Virus—17D), or alphavirus vectors, such as Sindibis virus (SINV), Venezuelan equine encephalitis virus, or Semliki forest virus.

In one embodiment, the viral vector may comprise nucleic acid comprising the sequence of SEQ ID NO: 39 and 40 (ChAdOx1) or a variant thereof. A variant of SEQ ID NO: 39 and 40 may comprise a sequence having at least 70%, 75%, 80%, 85%, 90%, 95%, 98%, 99%, or 99.5% identity with SEQ ID NO: 39 and 40. The variant of SEQ ID NO: 39 and 40 may encode a viral vector that substantially retains the function of the viral vector of SEQ ID NO: 39 and 40 (ChAdOx1).

In one embodiment, the viral vector may comprise nucleic acid comprising the sequence of SEQ ID NO: 41 and 42 (ChAdOx2) or a variant thereof. A variant of SEQ ID NO: 41 and 42 may comprise a sequence having at least 70%, 75%, 80%, 85%, 90%, 95%, 98%, 99%, or 99.5% identity with SEQ ID NO: 41 and 42. The variant of SEQ ID NO: 41 and 42 may encode a viral vector that substantially retains the function of the viral vector of SEQ ID NO: 41 and 42 (ChAdOx2).

In one embodiment, the viral vector may comprise nucleic acid comprising the sequence of SEQ ID NO: 44 and 45 (MVA) or a variant thereof. A variant of SEQ ID NO: 44 and 45 may comprise a sequence having at least 70%, 75%, 80%, 85%, 90%, 95%, 98%, 99%, or 99.5% identity with SEQ ID NO: 44 and 45. The variant of SEQ ID NO: 44 and 45 may encode a viral vector that substantially retains the function of the viral vector of SEQ ID NO: 44 and 45 (MVA).

In one embodiment, the viral vector encodes any one of:
SIi-HBV-CPmutS;
SIi-HBV-SCPmut;
SIi-HBV-CPmutPreS-S(sh);
SIi-HBV-CPmutPreS-TPA-S(sh);
MVA-SIi-HBV-PreS-Pmut-C-S(sh); or
MVA-SIi-HBV-PreS-Pmut-C-TPA-S(sh), as described herein.

In one embodiment, the viral vector encodes SEQ ID NO: 3 (SIi-HBV-CPmutS) or a variant thereof. A variant of SEQ ID NO: 3 may comprise a sequence having at least 70%, 75%, 80%, 85%, 90%, 95%, 98%, 99%, or 99.5% identity with SEQ ID NO: 3. The variant of SEQ ID NO: 3 may substantially retain the immunogenicity of SEQ ID NO: 3.

In one embodiment, the viral vector encodes SEQ ID NO: 11 (SIi-HBV-SCPmut) or a variant thereof. A variant of SEQ ID NO: 11 may comprise a sequence having at least 70%, 75%, 80%, 85%, 90%, 95%, 98%, 99%, or 99.5% identity with SEQ ID NO: 11. The variant of SEQ ID NO: 11 may substantially retain the immunogenicity of SEQ ID NO: 11.

In one embodiment, the viral vector encodes SEQ ID NO: 13 (SIi-HBV-CPmutPreS-S(sh)) or a variant thereof. A variant of SEQ ID NO: 13 may comprise a sequence having at least 70%, 75%, 80%, 85%, 90%, 95%, 98%, 99%, or 99.5% identity with SEQ ID NO: 13. The variant of SEQ ID NO: 13 may substantially retain the immunogenicity of SEQ ID NO: 13.

In one embodiment, the viral vector encodes SEQ ID NO: 25 (SIi-HBV-CPmutPreS-TPA-S(sh)) or a variant thereof. A variant of SEQ ID NO: 25 may comprise a sequence having at least 70%, 75%, 80%, 85%, 90%, 95%, 98%, 99%, or 99.5% identity with SEQ ID NO: 25. The variant of SEQ ID NO: 25 may substantially retain the immunogenicity of SEQ ID NO: 25.

In one embodiment, the viral vector encodes SEQ ID NO: 23 (MVA-SIi-HBV-PreS-Pmut-C-S(sh)) or a variant thereof. A variant of SEQ ID NO: 23 may comprise a sequence having at least 70%, 75%, 80%, 85%, 90%, 95%, 98%, 99%, or 99.5% identity with SEQ ID NO: 23. The variant of SEQ ID NO: 23 may substantially retain the immunogenicity of SEQ ID NO: 23.

In one embodiment, the viral vector encodes SEQ ID NO: 26 (MVA-SIi-HBV-PreS-Pmut-C-TPA-S(sh)) or a variant thereof. A variant of SEQ ID NO: 26 may comprise a sequence having at least 70%, 75%, 80%, 85%, 90%, 95%, 98%, 99%, or 99.5% identity with SEQ ID NO: 26. The variant of SEQ ID NO: 26 may substantially retain the immunogenicity of SEQ ID NO: 26.

In one embodiment, the viral vector comprises the nucleic acid sequence of SEQ ID NO: 46 (SIi-HBV-CPmutS) or a variant thereof. A variant of SEQ ID NO: 46 may comprise a sequence having at least 70%, 75%, 80%, 85%, 90%, 95%, 98%, 99%, or 99.5% identity with SEQ ID NO: 46.

In one embodiment, the viral vector comprises the nucleic acid sequence of SEQ ID NO: 47 (SIi-HBV-SCPmut) or a variant thereof. A variant of SEQ ID NO: 47 may comprise a sequence having at least 70%, 75%, 80%, 85%, 90%, 95%, 98%, 99%, or 99.5% identity with SEQ ID NO: 47.

In one embodiment, the viral vector comprises the nucleic acid sequence of SEQ ID NO: 48 (SIi-HBV-CPmutPreS-S(sh)) or a variant thereof. A variant of SEQ ID NO: 48 may comprise a sequence having at least 70%, 75%, 80%, 85%, 90%, 95%, 98%, 99%, or 99.5% identity with SEQ ID NO: 48.

In one embodiment, the viral vector comprises the nucleic acid sequence of SEQ ID NO: 49 (SIi-HBV-CPmutPreS-TPA-S(sh)) or a variant thereof. A variant of SEQ ID NO: 49 may comprise a sequence having at least 70%, 75%, 80%, 85%, 90%, 95%, 98%, 99%, or 99.5% identity with SEQ ID NO: 49.

In one embodiment, the viral vector comprises the nucleic acid sequence of SEQ ID NO: 24 (MVA-SIi-HBV-PreS-Pmut-C-S(sh)) or a variant thereof. A variant of SEQ ID NO: 24 may comprise a sequence having at least 70%, 75%, 80%, 85%, 90%, 95%, 98%, 99%, or 99.5% identity with SEQ ID NO: 24.

In one embodiment, the viral vector comprises the nucleic acid sequence of SEQ ID NO: 27 (MVA-SIi-HBV-PreS-Pmut-C-TPA-S(sh)) or a variant thereof. A variant of SEQ ID NO: 27 may comprise a sequence having at least 70%, 75%, 80%, 85%, 90%, 95%, 98%, 99%, or 99.5% identity with SEQ ID NO: 27. In one embodiment, the viral vector comprises the nucleic acid sequence of SEQ ID NO: 58 (MVA-SIi-HBV-PreS-Pmut-C-TPA-S(sh)) or a variant thereof. A variant of SEQ ID NO: 58 may comprise a sequence having at least 70%, 75%, 80%, 85%, 90%, 95%, 98%, 99%, or 99.5% identity with SEQ ID NO: 58.

Promoter

In one embodiment, the promoter is encoded in the immunogen expression cassette, for example the promoter may be encoded at, or adjacent to, the 5′ end of the immunogen expression cassette. Alternatively, the promoter may be encoded as part of the viral vector nucleic acid outside of the immunogen expression cassette. For example the promoter may be encoded upstream (5′) of the immunogen expression cassette.

The promoter may promote the expression of all the encoded protein of the immunogen expression cassette. In an alternative embodiment wherein the immunogen expression cassette comprises a secondary promoter, the promoter may be a primary promoter that is arranged to promote expression of at least the HBV core and modified polymerase (Pₘᵤₜ), and optionally not the HBV surface antigen (HbsAg) which may be arranged to be promoted separately by the secondary promoter.

The skilled person will recognise that any suitable promoter may be used as the primary and/or secondary promoter as appropriate for the host for expression. In one embodiment, the promoter comprises a CMV promoter. The CMV promoter may comprise the long or short CMV promoter. In an embodiment wherein the viral vector comprises an Adenoviral vector, such as ChAdOx1 or 2, the promoter may comprise a CMV promoter, SV40 promoter, or EF1a promoter. In an embodiment wherein the viral vector comprises an Adenoviral vector, such as ChAdOx1 or 2, the promoter may comprise a CMV promoter.

The promoter element(s) used in the vector, for example adenoviral vector, (such as ChAdOx1 or 2) may comprise a tetracycline operator (tetO) sequence. A tetracycline operator (tetO) sequence is helpful for generation of viral vectors that can express foreign proteins that are toxic to cells within which they are generated.

In another embodiment, the promoter comprises a pox viral promoter. In an embodiment wherein the viral vector comprises MVA, the promoter may comprise a pox viral promoter, such as F11. In one embodiment, the promoter comprises early Flt promoter. Alternatively, the pox viral promotor may comprise an early promoter, for example selected from any of B8R, K6L, A44L, C11R, and B2R, a promoter with early and late activity, for example selected from mH5, p7.5, and SSP, or a late promoter, for example FP4b.

Early promoter based transgene expression is can be useful for an immunogen intended primarily for T-cell response (there is a higher magnitude of T-cell induction upon usage of these early promoters). However promoters with both early and late activity can be used for an immunogen intended for inducing antibody response. However, these early and late activity promoters can also be used for an immunogen intended for T-cell induction.

In one embodiment, the promoter comprises or consists of the nucleic acid sequence of SEQ ID NO: 50 or 52 (CMV long or short promoter) or variants thereof. A variant of SEQ ID NO: 50 or 52 may comprise a sequence having at least 70%, 75%, 80%, 85%, 90%, 95%, 98%, 99%, or 99.5% identity with SEQ ID NO: 50 or 52 respectively. The variant may substantially retain the promoter function of SEQ ID NO: 50 or 52).

In one embodiment, the promoter comprises or consists of a sequence located in the F11 Left flank sequence (SEQ ID NO: 35) or a variant thereof. A variant may comprise a sequence having at least 70%, 75%, 80%, 85%, 90%, 95%, 98%, 99%, or 99.5% identity with the promoter sequence located in the F11 Left flank sequence (SEQ ID NO: 35). The variant may substantially retain the promoter function of the promoter located in the F11 Left flank sequence (SEQ ID NO: 35).

Secondary Promoter

In one embodiment, the immunogen expression cassette encodes a secondary promoter. In an embodiment wherein a secondary promoter is provided in addition to the first/primary promoter, the secondary promoter may be encoded by/within the immunogen expression cassette. The secondary promoter may be encoded 3′ of the first/primary promoter. The secondary promoter may be encoded 3′ of the first protein/antigen to be expressed, for example downstream of the HBV core and HBV modified polymerase and upstream of the HBV surface antigen. The secondary promoter may promote the expression of at least the HBV surface antigen.

The secondary promoter may comprise a pox viral promoter. The secondary promoter may comprise pox viral early promoters, such as any of B8R, K6L, A44L, C11R, and B2R, or pox viral promoters with early and late activity, such as mH5, p7.5 or SSP, or a late promoter such as FP4b.

In one embodiment, the secondary promoter comprises early/late promoter mH5. In one embodiment, the secondary promoter comprises or consists of the nucleic acid sequence of SEQ ID NO: 28 (mH5 promoter) or a variant thereof. A variant of SEQ ID NO: 28 may comprise a sequence having at least 70%, 75%, 80%, 85%, 90%, 95%, 98%, 99%, or 99.5% identity with SEQ ID NO: 28. The variant may substantially retain the secondary promoter function of SEQ ID NO: 28).

In another embodiment, the secondary promoter may comprise a SV40 promoter or EF1a promoter.

Cleavage Domain

In one embodiment, the immunogen expression cassette encodes a cleavage domain. In one embodiment, the cleavage domain is a post-translation proteolytic cleavage domain, which allows for the cleavage of a translated protein, for example by a proteolytic cleavage enzyme. The cleavage enzyme may be provided by the host, for example the host being vaccinated, such as a human. In another embodiment, the cleavage enzyme may be encoded in the immunogen expression cassette or viral vector. In one embodiment, the cleavage domain comprises a non-HBV sequence, for example, a mammalian sequence. In one embodiment, the cleavage domain comprises a human derived sequence.

In one embodiment, the cleavage domain comprises a ribosome skipping cleavage domain. In one embodiment, the cleavage domain comprises a Furin recognition site comprising or consisting of the sequence RXRR, where X could be any amino acid. In one embodiment, the cleavage domain comprises a Furin recognition site and a 2A peptide sequence. The 2A peptide sequence may comprise FMDV (Foot and Mouth Disease Virus) 2A peptide sequence (APVKQTLNFDLLKLAGDVESNPGP—SEQ ID NO: 43). Alternatively, 2A peptides may be selected from P2A (porcine teschovirus-1 2A), T2A (Thoseaasigna virus 2A), and E2A (equine rhinitis A virus [ERAV] 2A). Any picorna virus 2A peptide sequence may be provided for, and function as, a peptide cleavage site. Therefore, the cleavage domain may comprise a sequence encoding picorna virus 2A peptide sequence.

In one embodiment, the cleavage domain comprises or consists of Furin-2A (F2A) peptide sequence or a functional variant thereof. The cleavage domain may comprise or consists of the sequence of SEQ ID NO: 9 or a variant thereof. A variant of SEQ ID NO: 9 may comprise a sequence having at least 70%, 75%, 80%, 85%, 90%, 95%, 98%, 99%, or 99.5% identity with SEQ ID NO: 9. The variant of SEQ ID NO: 9 may substantially retain the cleavage function of SEQ ID NO: 9.

F2A is advantageously short (only 28 amino acids), which helps to provide appropriate inserts and express more than one protein from vectors that can have a size limit, such as Adenovirus based vectors, including ChAdOx1/2.

In an embodiment where the viral vector is MVA, or otherwise a viral vector is used as a boost vaccination following a prime vaccination, a different cleavage domain may be provided to avoid any potential boosting of T-cell responses to the cleavage domain of the prime vaccination. Alternatively a secondary promoter may be used instead of a cleavage domain in order to avoid any potential boosting of T-cell responses to a cleavage domain of a prime vaccination.

IRES (Internal Ribosomal Entry Site)

In one embodiment, the immunogen expression cassette encodes an Internal Ribosome Entry Site (IRES). The skilled person will recognise that an IRES is an RNA structures that allows cap independent initiation of translation, and is able to initiate translation in the middle of a messenger RNA.

Splicing Signal

In one embodiment, the immunogen expression cassette encodes an mRNA splicing signal. The skilled person will be familiar with commonly used splicing signals that are appropriate for this use. One such example of an mRNA splicing signal that may be used is a chimeric intron composed of the 5′ donor site from the first intron of the human β-globin gene and the branch and 3′ acceptor site from the intron of an immunoglobulin gene heavy chain variable region in the expression vector pCI-neo Mammalian Expression Vector (Promega).

HBV Pre-Core (PreC)

The HBV PreC may comprise or consist of a full length wild-type HBV PreC sequence, or a variant thereof. The HBV PreC variant may comprise or consist of a truncated HBV PreC sequence.

The HBV PreC may comprise or consist of the sequence of SEQ ID NO: 16 or a variant thereof. A variant of SEQ ID NO: 16 may comprise a sequence having at least 70%, 75%, 80%, 85%, 90%, 95%, 98%, 99%, or 99.5% identity with SEQ ID NO: 16. The variant of SEQ ID NO: 16 may substantially retain the immunogenicity of SEQ ID NO: 16. The variant of SEQ ID NO: 16 may substantially retain the tertiary structure of SEQ ID NO: 16.

HBV Core

The HBV Core may comprise or consist of a full length wild-type HBV Core sequence, or a variant thereof. The HBV Core variant may comprise or consist of a truncated HBV Core sequence. The HBV Core may not comprise HBV Pre-Core.

The HBV Core may comprise or consist of the sequence of SEQ ID NO: 6 or a variant thereof. A variant of SEQ ID NO: 6 may comprise a sequence having at least 70%, 75%, 80%, 85%, 90%, 95%, 98%, 99%, or 99.5% identity with SEQ ID NO: 6. The variant of SEQ ID NO: 6 may substantially retain the immunogenicity of SEQ ID NO: 6. The variant of SEQ ID NO: 6 may substantially retain the tertiary structure of SEQ ID NO: 6.

HBV e-Antigen (HBeAg)

The HBV e-Antigen (HBeAg) may comprise or consist of a full length wild-type HBV e-Antigen sequence, or a variant thereof. The HBV e-Antigen variant may comprise or consist of a truncated HBV e-Antigen sequence.

The HBV e-Antigen (HBeAg) may comprise or consist of the sequence:

(SEQ ID NO: 17)
SKLCLGWLWGMDIDPYKEFGASVELLSFLPSDFFPSIRDLLDTASALYRE
ALESPEHCSPHHTALRQAILCWGELMNLATWVGSNLEDPASRELVVSYVN
VNMGLKIRQLLWFHISCLTFGRETVLEYLVSFGVWIRTPPAYRPPNAPIL
STLPETTVV.

The HBV e-Antigen (HBeAg) may comprise or consist of the sequence of SEQ ID NO: 17 or a variant thereof. A variant of SEQ ID NO: 17 may comprise a sequence having at least 70%, 75%, 80%, 85%, 90%, 95%, 98%, 99%, or 99.5% identity with SEQ ID NO: 17. The variant of SEQ ID NO: 17 may substantially retain the immunogenicity of SEQ ID NO: 17. The variant of SEQ ID NO: 17 may substantially retain the tertiary structure of SEQ ID NO: 17.

Modified HBV Polymerase

The modified HBV polymerase (Pₘᵤₜ) may comprise or consist of a truncated HBV polymerase. In particular, the mutation to wild-type HBV polymerase to substantially remove polymerase function may comprise a sequence encoding a truncated HBV polymerase. Alternatively or additionally, the mutation comprises one or more point mutations to the encoded HBV polymerase sequence. The modification may comprise one or more amino acid substitutions, deletions or additions in the encoded HBV polymerase sequence. In one embodiment, the modified HBV polymerase (Pₘᵤₜ) is not a truncated form of HBV polymerase (i.e. it is full length relative to wildtype HBV polymerase).

The modified HBV polymerase (Pₘᵤₜ) may comprise or consist of the sequence of SEQ ID NO: 8 or a variant thereof. A variant of SEQ ID NO: 8 may comprise a sequence having at least 70%, 75%, 80%, 85%, 90%, 95%, 98%, 99%, or 99.5% identity with SEQ ID NO: 8. The variant of SEQ ID NO: 8 may substantially retain the immunogenicity of SEQ ID NO: 8. The variant of SEQ ID NO: 8 may substantially retain the tertiary structure of SEQ ID NO: 8.

The modification may be a mutation that prevents protein priming. Additionally or alternatively mutations may be provided in the reverse transcriptase and/or RNAase domains to prevent their function. Further additionally or alternatively the mutation may be structural such that is disrupts the correct protein folding of the polymerase. The modifications may comprise one or more, or all of Y63, C323, C334, C338, C352, R714, D788, R792, or equivalent residues thereof, by reference to wild-type HBV polymerase consensus sequence herein (i.e. SEQ ID NO: 19). In one embodiment the modifications may comprise one or more, or all of Y63A, C323A, C334A, C338A, C352A, R714A, D788A, R792A, or equivalent residues thereof, by reference to wild-type HBV polymerase consensus sequence herein (i.e. SEQ ID NO: 19). In another embodiment, the modifications may comprise one or more, or all of R714, D788, and R792, or equivalent residues thereof, by reference to wild-type HBV polymerase consensus sequence herein (i.e. SEQ ID NO: 19). In one embodiment the modifications may comprise one or more, or all of R714A, D788A, and R792A, or equivalent residues thereof, by reference to wild-type HBV polymerase consensus sequence herein (i.e. SEQ ID NO: 19). Reference to equivalent residues is understood to mean that the HBV polymerase may be based on an alternative sequence than the HBV polymerase sequence provided herein, and that numbering or identity of amino acid residues may differ between the sequences. Such differences and equivalency may be readily determined by an alignment of the HBV polymerase sequence with the wild-type HBV polymerase consensus sequence herein (i.e. SEQ ID NO: 19).

Mutations R714A, D788A, R792A advantageously stop polymerase function. However, one or more, or all of, additional mutations of Y63 and other cysteine mutations of C323, C334, C338, and C352 may be added as an extra measure in the event of a reversion within those functional mutations. The additional mutations may comprise Y63A, C323A, C334A, C338A, and/or C352A.

The mutations advantageously provide that the HBV polymerase function is disrupted by (a) RNAase H functional mutations, to stop its enzyme activity, and/or (b) Y63A mutation to stop the first step of replication (Priming of DNA synthesis), and/or (c) cysteine mutations to disrupt its native conformation and stop the HBV polymerase from participating in the initial steps of viral replication (protein priming, RNA binding and RNA packaging).

The skilled person will be familiar with various HBV polymerase modifications that can be provided which significantly reduce or ablate function, and can be provided in the modified polymerase according to the invention. Such modifications are for example described in WO2016020538, WO2013007772, and WO2011015656, which are incorporated herein by reference.

In one embodiment, the modified polymerase is modified so as to exhibit a reduced reverse-transcriptase (RTase) enzymatic activity with respect to a wild-type HBV polymerase. The reduction of RTase activity may be provided by one or more mutation(s) in the domain responsible for RTase enzymatic activity. Four residues have been found to be involved in RTase activity, forming a motif “YMDD” (for Tyr, Met, Asp and Asp residues) at approximately position 538 to approximately position 541 of a wild-type HBV polymerase. The present invention encompasses any mutation(s) in this motif, or elsewhere in the RTase domain, that provides a significant reduction (e.g. at least a 10 fold reduction) or ablation of the RTase activity. Representative examples of suitable RTase-deficient polymerase mutants are described in Radziwill et al. (1990, J. Virol. 64:613), in Bartenschlager et al. (1990, J. Virol. 64:5324) and in Jeong et al. (1996, Biochem Bioph Res Commun. 223(2):264), which are incorporated herein by reference. In one embodiment, the modified polymerase may comprise the substitution of the first Asp residue of the YMDD motif or of the amino acid residue located in an equivalent position in a native HBV polymerase to any amino acid residue other than Asp, with an optional substitution to a His residue (D540H mutation).

Additionally or alternatively, the modified polymerase may be modified to provide a reduced RNase H enzymatic activity with respect to wild-type HBV polymerase. The reduction of RNase H activity may be provided by one or more mutation(s) in the domain responsible for RNase H enzymatic activity. The functional domain involved in RNase H activity is within the C-terminal portion of HBV polymerase, approximately from position 680 to the C-terminal position 832 of wild-type polymerase, and the modified polymerase of the present invention may encompasses any mutation(s) in this domain that provides a significant reduction (e.g. at least a 10 fold reduction) or ablation of the RNase H activity. Representative examples of suitable RNase H-deftcient polymerase mutants are described in Radziwill et ai. (1990, J. Virol. 64:613), in Bartenschlager at al. (1990, J. Virol. 64:5324).

HBV Surface Antigen (HbsAg)

The skilled person will understand that PreS1 and PreS2 are components of the Large (L) form of RBA/surface protein (e.g. L form=PreS1+PreS2+S). The medium (M) form of HBV surface protein has PreS2-'S. Having such sequences together means that T-Cell epitopes are included in those sequential order.

The HbsAg may comprise or consist of a full length wild-type HbsAg sequence, or a variant thereof. The HbsAg variant may comprise or consist of a truncated HbsAg sequence.

The HbsAg including the PreS1 and PreS2 sequences may comprise or consist of the sequence of SEQ ID NO: 10 or a variant thereof. A variant of SEQ ID NO: 10 may comprise a sequence having at least 70%, 75%, 80%, 85%, 90%, 95%, 98%, 99%, or 99.5% identity with SEQ ID NO: 10. The variant of SEQ ID NO: 10 may substantially retain the immunogenicity of SEQ ID NO: 10. The variant of SEQ ID NO: 10 may substantially retain the tertiary structure of SEQ ID NO: 10.

In another embodiment, the HbsAg may comprise the surface antigen without PreS1 and/or PreS2. The HbsAg may comprise or consist of the sequence of SEQ ID NO: 18 or a variant thereof. A variant of SEQ ID NO: 18 may comprise a sequence having at least 70%, 75%, 80%, 85%, 90%, 95%, 98%, 99%, or 99.5% identity with SEQ ID NO: 18. The variant of SEQ ID NO: 18 may substantially retain the immunogenicity of SEQ ID NO: 18. The variant of SEQ ID NO: 18 may substantially retain the tertiary structure of SEQ ID NO: 18.

In one embodiment, the HbsAg may comprise or consist of any one or all of the four known transmembrane regions in HbsAg (amino acids 1-226), which are amino acids (8-32) FLGPLLVLQAGFFLLTRILTIPQSL (SEQ ID NO: 54), amino acids (80-98) FIIFLFILLLCLIFLLVLL (SEQ ID NO: 55), amino acids (160-184) RFLWEWASVRFSWLSLLVPFVQWFV (SEQ ID NO: 56), and amino acids (189-210) TVWLSVIWMMWYWGPSLYNILS (SEQ ID NO: 57) respectively. In one embodiment, the HbsAg may at least comprise or consist of the HbsAg transmembrane region of amino acids (8-32) FLGPLLVLQAGFFLLTRILTIPQSL (SEQ ID NO: 54).

HBV PreS1

The HBV PreS1 may comprise or consist of a full length wild-type HBV PreS1 sequence, or a variant thereof. The HBV PreS1 may comprise or consist of the sequence of SEQ ID NO: 52 or a variant thereof. A variant of SEQ ID NO: 52 may comprise a sequence having at least 70%, 75%, 80%, 85%, 90%, 95%, 98%, 99%, or 99.5% identity with SEQ ID NO: 52. The variant of SEQ ID NO: 52 may substantially retain the immunogenicity of SEQ ID NO: 52. The variant of SEQ ID NO: 52 may substantially retain the tertiary structure of SEQ ID NO: 52.

The HBV PreS1 variant may comprise or consist of a truncated HBV PreS1 sequence, for example CΔPreS1 or NΔPreS1 described herein (CΔPreS1 refers to C-terminal truncated PreS1 and NΔPreS1 refers to N-terminal truncated PreS1). In one embodiment, the immunogen expression cassette may encode both NΔPreS1 and CΔPreS1. In an embodiment wherein the immunogen expression cassette encodes both NΔPreS1 and CΔPreS1, the NΔPreS1 may be encoded upstream (5′) of intergenic sequence and the CΔPreS1 may be encoded downstream (3′) of intergenic sequence. In an embodiment wherein the immunogen expression cassette encodes NΔPreS1 upstream (5′) of intergenic sequence, it may be fused with PreS2 and/or the modified polymerase (Pₘᵤₜ). In an embodiment wherein the immunogen expression cassette encodes NΔPreS1 upstream (5′) of intergenic sequence, it may be fused with PreS2 and/or the modified polymerase (Pₘᵤₜ) and the CΔPreS1 encoded downstream (3′) of intergenic sequence may be fused with the surface antigen (HbsAg).

CΔPreS1

In one embodiment, the immunogen expression cassette encodes a truncated form of HBV PreS1. The truncation may comprise a C-terminal truncation. In one embodiment, the truncated HBV PreS1 comprises CΔPreS1 (SEQ ID NO: 21) described herein. In one embodiment, the truncated PreS1, such as CΔPreS1 described herein, is arranged to be expressed a fusion protein with the HBV surface antigen (S/HbsAg). A linker sequence, such as a linker described herein, may be provided between the truncated PreS1 and surface antigen (S/HbsAg). For example, CΔPreS1+linker+S (described herein as S(sh)). The nucleotide sequence encoding S(sh) may comprise or consist of SEQ ID NO: 61 or SEQ ID NO: 38.

The CΔPreS1 may comprise or consist of the sequence of SEQ ID NO: 21 or a variant thereof. A variant of SEQ ID NO: 21 may comprise a sequence having at least 70%, 75%, 80%, 85%, 90%, 95%, 98%, 99%, or 99.5% identity with SEQ ID NO: 21. The variant of SEQ ID NO: 21 may substantially retain the immunogenicity of SEQ ID NO: 21. The variant of SEQ ID NO: 21 may substantially retain the tertiary structure of SEQ ID NO: 21.

In one embodiment, the truncated PreS1, such as CΔPreS1 described herein, optionally with a fused surface antigen, is encoded downstream (3′) of the intergenic sequence.

The PreS1 truncation advantageously favours antibody generation for both T-cell and antibody response for the HEW antigens provided in the immunogen expression cassette. Without being bound by theory, antibody generation is possibly due to proper folding of PreS1 and Surface antigens.

NΔPreS1

In one embodiment, the truncated PreS1 comprises NΔPreS1 as described herein. The NΔPreS1 and PreS2 fusion may comprise or consist of SEQ ID NO: 15, or a variant thereof. A variant of SEQ ID NO: 15 may comprise a sequence having at least 70%, 75%, 80%, 85%, 90%, 95%, 98%, 99%, or 99.5% identity with SEQ ID NO: 15. The variant of SEQ ID NO: 15 may substantially retain the immunogenicity of SEQ ID NO: 15.

In one embodiment, the truncated PreS1 comprises NΔPreS1 as described herein. The NΔPreS1 and PreS2 fusion may be encoded by nucleic acid comprising or consisting of SEQ ID NO: 38 or 61, or a variant thereof. A variant of SEQ ID NO: 38 or 61 may comprise a sequence having at least 70%, 75%, 80%, 85%, 90%, 95%, 98%, 99%, or 99.5% identity with SEQ ID NO: 38 or 61. The encoded variant of SEQ ID NO: 38 or 61 may substantially retain the immunogenicity of peptide encoded by SEQ ID NO: 38 or 61.

The NΔPreS1 and PreS2 fusion sequence advantageously provides a good T-cell response. NΔPreS1 advantageously includes amino acids in a sequence that would still preserve T-cell epitopes (8-11 amino acids for CD8 T-cell epitopes and slightly longer (12-16) for CD4 T-cell epitopes.

In an embodiment wherein the immunogen expression cassette encodes NΔPreS1 fused with PreS2 upstream (5′) of intergenic sequence, it may be further fused with the modified polymerase (Pₘᵤₜ). A linker sequence, such as a linker described herein, may be provided between the PreS2 and modified polymerase (Pₘᵤₜ). For example, NΔPreS1+PreS2+linker+Pmut.

HBV PreS2

The HBV PreS2 may comprise or consist of a full length wild-type HBV PreS2 sequence, or a variant thereof. The HBV PreS2 variant may comprise or consist of a truncated HBV PreS2 sequence.

The HBV PreS2 may comprise or consist of the sequence of SEQ ID NO: 53 or a variant thereof. A variant of SEQ ID NO: 53 may comprise a sequence having at least 70%, 75%, 80%, 85%, 90%, 95%, 98%, 99%, or 99.5% identity with SEQ ID NO: 53. The variant of SEQ ID NO: 53 may substantially retain the immunogenicity of SEQ ID NO: 53. The variant of SEQ ID NO: 53 may substantially retain the tertiary structure of SEQ ID NO: 53.

Peptide Adjuvant

The immunogen expression cassette may further encode a peptide adjuvant. The peptide adjuvant may comprise TPA (tissue plasminogen activator). In one embodiment, the peptide adjuvant may comprise a human or non-human invariant chain (Ii), or a fragment thereof. A fragment of the long isoform (isoform (b)) of the human CD74 molecule, is also known as the invariant chain (Nucleic Acids Res. 1985 Dec. 20; 13(24): 8827-8841). It is known that N-terminal fragments of the invariant chain (1i) which comprise at least the transmembrane domain thereof, provide a surprisingly effective adjuvant function when expressed as a fusion protein with an antigen of interest. Fragments encompassing the transmembrane domain and the cytoplasmic domain, and preferably including the N-terminal 16 amino acids of the long isoform of the protein are particularly efficacious.

The invariant chain may comprise or consist of a shark invariant chain (SIi), or fragment or a functional variant thereof. The variant shark invariant chain (SIi) may comprise a truncated invariant shark invariant chain. Other non-human animal sources of an invariant chain, or fragment thereof, include chicken, quail, trout, zebrafish, carp, frog, grouper, shark, mandarin fish or mallard. The skilled person will be familiar with appropriate invariant chains, or fragments thereof, for use as peptide adjuvants encoded in an expression cassette/vector, for example the invariant chain may be any invariant chain as provided in WO2015082922, which is incorporated herein by reference.

The peptide adjuvant may comprise or consist of the sequence of SEQ ID NO: 4 (SIi) or a variant thereof. A variant of SEQ ID NO: 4 may comprise a sequence having at least 70%, 75%, 80%, 85%, 90%, 95%, 98%, 99%, or 99.5% identity with SEQ ID NO: 4. The variant of SEQ ID NO: 4 may substantially retain the adjuvant function of SEQ ID NO: 4.

The peptide adjuvant may be encoded by a sequence comprising or consists of the sequence of SEQ ID NO: 29 (TPA nucleic acid sequence) or a variant thereof. A variant of SEQ ID NO: 29 may comprise a sequence having at least 70%, 75%, 80%, 85%, 90%, 95%, 98%, 99%, or 99.5% identity with SEQ ID NO: 29. The variant of SEQ ID NO: 29 may encode a peptide adjuvant that substantially retains the adjuvant function of the TPA encoded by SEQ ID NO: 29. The peptide adjuvant may be encoded by a sequence comprising or consists of the sequence of SEQ ID NO: 60 (TPA nucleic acid sequence) or a variant thereof. A variant of SEQ ID NO: 60 may comprise a sequence having at least 70%, 75%, 80%, 85%, 90%, 95%, 98%, 99%, or 99.5% identity with SEQ ID NO: 60. The variant of SEQ ID NO: 60 may encode a peptide adjuvant that substantially retains the adjuvant function of the TPA encoded by SEQ ID NO: 60.

The peptide adjuvant may comprise or consist of the sequence of SEQ ID NO: 30 (TPA) or a variant thereof. A variant of SEQ ID NO: 30 may comprise a sequence having at least 70%, 75%, 80%, 85%, 90%, 95%, 98%, 99%, or 99.5% identity with SEQ ID NO: 30. The variant of SEQ ID NO: 30 may substantially retain the adjuvant function of SEQ ID NO: 30.

The peptide adjuvant may be encoded at an N-terminal position of a protein/antigen to be expressed from the immunogen expression cassette. A first peptide adjuvant may be encoded at an N-terminal position of a first protein/antigen (such as the Core and Polymerase fusion) to be expressed from the immunogen expression cassette, and a second peptide adjuvant may be encoded at an N-terminal position of a second protein/antigen (such as surface antigen) to be expressed from the immunogen expression cassette.

HBV Genotype

The HBV may be HBV genotype C. In another embodiment, the HBV may be of any one of the 10 genotypes (A-J). All of the encoded HBV proteins/antigens may be derived from one genotype, for example HBV genotype C.

Linkers

In one embodiment, linker residues may be encoded between one or more, or all, of the protein/antigen sequences that are provided in a fusion protein (e.g. providing junctions between the sequences in the protein). In an embodiment comprising a peptide adjuvant, a linker may be encoded between the peptide adjuvant and the downstream encoded protein/antigen. In one embodiment, a linker is encoded between the sequences of the HBV core and modified HBV polymerase.

The linker residues may comprise random amino acid sequences, or amino-acids that have been selected to be non-immunogenic based on epitope prediction computer programs or experiments in animal models. For example, a linker may not be considered if it is predicted or known to be an epitope (i.e. in order to avoid an immune response to epitopes, e.g. artificial epitopes, not found in HBV. The linker may be flexible. The linker may comprise or consist of K, G, P or S amino acid residues, or combinations thereof. In one embodiment, the linker may comprise or consist of G and/or P amino acid residues. The linker residues may be between 1 and 10 amino acids in length. In another embodiment, the linker residues may be between 2 and 8 residues in length. In another embodiment, the linker residues may be between 1 and 6 residues in length.

A linker may comprise or consist of any of the sequences KGGGPGGG (SEQ ID NO: 5), GGGSGGG (SEQ ID NO: 7), KGGS (SEQ ID NO: 14), KSP, GSKGK (SEQ ID NO: 20), LEGGSGG (SEQ ID NO: 22), SKSGPPSGKS (SEQ ID NO: 31), GSKSGSK (SEQ ID NO: 32), SKSPGSGPP (SEQ ID NO: 33), or ASKGGKSG (SEQ ID NO: 34).

In one embodiment, a linker may comprise the sequence KGGGPGGG (SEQ ID NO: 5). In another embodiment, a linker may comprise the sequence GGGSGGG (SEQ ID NO: 7). In another embodiment, a linker may comprise the sequence KGGS (SEQ ID NO: 14). In another embodiment, a linker may comprise the sequence KSP. In another embodiment, a linker may comprise the sequence GSKGK (SEQ ID NO: 20). In another embodiment, a linker may comprise the sequence LEGGSGG (SEQ ID NO: 22). In another embodiment, a linker may comprise the sequence SKSGPPSGKS (SEQ ID NO: 31). In another embodiment, a linker may comprise the sequence GSKSGSK (SEQ ID NO: 32). In another embodiment, a linker may comprise the sequence SKSPGSGPP (SEQ ID NO: 33). In another embodiment, a linker may comprise the sequence ASKGGKSG (SEQ ID NO: 34).

Advantageously, the use of linkers can avoid generation of peptides with homology to human proteome (which could potentially generate immune response to self-antigen) and they avoid immune-dominant artificial epitopes. Additionally, linkers can provide a flexible hinge between segments of protein, so that they can fold into their native conformation. For example, a linker between CΔPreS1 and S allows independent folding of CΔPreS1 and S, so that they could generate antibodies for conformational epitopes for the respective proteins.

Linkers may be varied between different immunogen expression cassettes. Using different linkers avoids boosting of any T-cell response created to any potential artificial epitopes (i.e. by changing linkers or changing junctions). For example, when vaccines are used in prime boost vaccination strategies, changing the linkers or the order of proteins within the immunogen layout, helps to overcome boosting of artificial epitope response.

Other Elements of the Immunogen Expression Cassette(s)

In an embodiment wherein the immunogen expression cassette is for use in an MVA vector, the immunogen expression cassette may further comprise F11 left and right flanking sequences to allow insertion into the MVA F11 locus by homologous recombination.

The F11-left flank sequence may comprise or consist of the sequence of SEQ ID NO: 35, or a variant thereof. The F11-right flank sequence may comprise or consist of the sequence of SEQ ID NO: 36, or a variant thereof. A variant of SEQ ID NO: 35 or 36 may comprise a sequence having at least 70%, 75%, 80%, 85%, 90%, 95%, 98%, 99%, or 99.5% identity with SEQ ID NO: 35 or 36 respectively. The variant of SEQ ID NO: 35 or 36 may substantially retain the homologous recombination function of SEQ ID NO: 35 or 36 respectively. The skilled person will appreciate that 1, 2, 3, 4, 5 or more amino acid residues may be substituted, added or removed without affecting function. For example, conservative substitutions may be considered.

The immunogen expression cassette may further comprise a transcription terminator sequence. The transcription terminator sequence may be provided in embodiments of the immunogen expression cassette comprising a secondary promoter. For example, the transcription terminator sequence may be provided downstream of a protein expressed by the primary promoter, but before (upstream of) the secondary promoter. The transcription terminator sequence may comprise or consist of the sequence TTTTTGT, or variants thereof.

In one embodiment, the immunogen expression cassette described herein may be isolated or provided in a non-viral vector.

Therefore, according to another aspect of the present invention there is provided a nucleic acid comprising the immunogen expression cassette described herein, optionally wherein the nucleic acid is isolated nucleic acid.

According to another aspect of the invention there is provided a composition comprising the viral vector according to the invention, optionally wherein the composition is a pharmaceutically acceptable composition.

The composition may be immunogenic, for example in a mammal, such as a human. The composition may comprise a pharmaceutically acceptable carrier. The composition may be a pharmaceutical composition comprising a pharmaceutically acceptable carrier. The composition may be for use in the prophylaxis or treatment of HBV infection.

According to another aspect of the invention there is provided a method of treatment or prophylaxis of HBV infection comprising the administration of the viral vector, nucleic acid or composition according to the invention.

The method of treatment or prophylaxis of HBV infection may be a method of vaccination.

According to another aspect of the invention there is provided a viral vector, nucleic acid or composition according to the invention for use in the treatment or prevention of HBV infection, optionally wherein the use is in a vaccine.

According to another aspect of the invention there is provided a vaccine comprising the viral vector, nucleic acid or composition according to the invention.

The vaccine may be a prime vaccine. The vaccine may be a boost vaccine. Where a boost vaccine is provided following a prime vaccine, the viral vector may be a different viral vector according to the invention.

Advantageously, the provision of a different viral vector between prime and boost vaccines can avoid the provision of “false” epitopes formed across junctions of one protein/antigen sequence with another. i.e. the same junction may not occur in a re-ordered protein.

According to another aspect of the invention, there is provided a prime boost vaccination kit comprising
a prime vaccination according to the invention;
a boost vaccination according to the invention.

The prime and boost vaccination may comprise different viral vectors.

The viral vector may be used in a vaccine in combination with another therapeutically or prophylactically active ingredient. The viral vector may be used in a vaccine in combination with an adjuvant.

The viral vector, nucleic acid encoding the viral vector may be provided in a pharmaceutically acceptable carrier.

The viral vector or composition according to the invention may not comprise wild-type HBV, or the nucleic acid according to the invention may not encode wild-type HBV.

Reference to sequence “identity” used herein may refer to the percentage identity between two aligned sequences using standard NCBI BLASTp parameters (http://blast.ncbi.nlm.nih.gov).

The term “immunogenic”, when applied to the viral vector, nucleic acid or composition of the present invention means capable of eliciting an immune response in a human or animal body. The immune response may be protective of HBV. The term “protective” means prevention of Hepatitis B disease, a reduced risk of Hepatitis B disease, reduced risk of HBV infection, transmission and/or progression, reduced severity of Hepatitis B disease, a cure of Hepatitis B, an alleviation of symptoms of Hepatitis B, or a reduction in severity of Hepatitis B disease symptoms.

The term “prophylaxis” means prevention of or protective treatment for Hepatitis B. The prophylaxis may include a reduced risk of HBV infection, transmission and/or Hepatitis B disease progression, or reduced severity of Hepatitis B disease.

The term “treatment”, means a cure of Hepatitis B, an alleviation of symptoms, or a reduction in severity of Hepatitis B disease or Hepatitis B disease symptoms.

With reference to “variant” nucleic acid sequences, the skilled person will appreciate that 1, 2, 3, 4, 5 or more codons may be substituted, added or removed without affecting function. For example, conservative substitutions may be considered.

With reference to “variant” amino acid sequences, the skilled person will appreciate that 1, 2, 3, 4, 5 or more amino acids may be substituted, added or removed without affecting function. For example, conservative substitutions may be considered.

The skilled person will understand that optional features of one embodiment or aspect of the invention may be applicable, where appropriate, to other embodiments or aspects of the invention.

### BRIEF DESCRIPTION OF THE DRAWINGS

Embodiments of the invention will now be described in more detail, by way of example only, with reference to the accompanying drawings.

FIGS. 1A-1B: (A) Phylogenetic relationship of 1447 HBV genotype C sequences used to generate consensus sequence (B) Comparison of HBV genotype C consensus (SEQ ID NO: 1) and KP017269.1 HBV isolate JP-02 (SEQ ID NO: 2). 1447 HBV genotype C nucleotide sequences, downloaded from HBV data base were aligned using MAFFT (a multiple sequence alignment program), to generate the HBV genotype C consensus sequence and a phylogenetic tree and. Alignment of 3215 nucleotide sequences of the consensus and chosen patient's sequence (KP017269.1 HBV isolate JP-02) showed three nucleotide differences (at positions 52, 1053 and 2699), which are highlighted in grey colour. The sequence of the HBV genotype C consensus sequence generated from 1447 genotype c isolate jp-02 is provided below.

FIGS. 2A-2D: (A) HBV viral genome and codon layout. The HBV virion has a partial double stranded DNA (having a full length negative strand DNA and a partially synthesised positive strand DNA attached to the polymerase protein). The genome is approximately 3.2 Kb in length and has four major codons: core (including the precore region), polymerase, surface (including the preS1 and preS2 region) and X. (B) HBV Immunogen Layout. Two immunogens SIi-HBV-CPmutS and SIi-HBV-SCPmut were designed. Both immunogens encodes HBV codons (encompassing precore, core, polymerase [Pmut], preS1, preS2 and surface proteins) and non HBV codons (comprising of a truncated Shark Invariant chain [SIi], two linkers [represented in Turquoise blue colour] and a Furin 2A [F2A] peptide sequence). Within the mammalian expression cassette, the immunogen codon sequences are placed next to CMV promoter. (C) Invitro expression analysis. Plasmids encoding SIi-HBV-CPmutS and SIi-HBV-SCPmut were transfected into HEK293A cells. 24 hours post-transfection, cells were lysed and the lysates were analysed in Western blot experiments using mouse anti-HBV-PreS1 and mouse anti-HBV-Polymerase antibodies. Blots probed with mouse anti-GAPDH served as loading controls. (D) ChAdOX2-SIi-HBV-CPmutS. The mammalian expression cassette with SIi-HBV-CPmutS immunogen codons was inserted into the replication deficient ChAdOx2 vector. Recombinant ChAdOX2-SIi-HBV-CPmutS virus was generated by transfecting the ChAdOX2-SIi-HBV-CPmutS vector into T-REx™-293 cells (Thermo Fisher Scientific) using standard methods as previously described.

FIGS. 3A-3D: Testing ChAdOx2-SIi-HBV-CPmutS vaccine in naive mice models. (A) Splenocyte responses in BALB/c mice (B) Intra Hepatic Lymphocyte responses in BALB/c mice (C) Splenocyte responses in CD1 mice (D) Intra Hepatic Lymphocyte responses in CD1 mice. BALB/c mice (4 mice at the age of 7 weeks) and CD1 (2 mice at age of 18 weeks and 5 mice at age of 13 weeks) were vaccinated by intramuscular injections with 4×10⁷ IU and 5×10⁷ IU per mice respectively of ChAdOx2-SIi-HBV-CPmutS vaccine. 14 days post-vaccination, mice were sacrificed, splenocytes and intra hepatic lymphocytes (IHL) were isolated from spleen and liver according to standard protocol. 2×10⁵ splenocytes and 1×10⁵ IHL's were plated on to ELSIPOT plates (pre-coated with Anti-mouse INFγ monoclonal antibody, by overnight incubation) along with DMSO (1%) or non-HBV peptide pool (A, I, L at a concentration of 3 μg/ml) or HBV peptide pool (Core, Pol-1, Poi-2, Poi-3, Pol-4, PreS1/S2 and Surface at a concentration of 3 μg/ml) or a positive control mitogen (PHA or Concanavalin A at a concentration of 10 μg/ml and 12.5 μg/ml respectively). After an overnight 15 hours incubation at 37° C., the plates were washed 7× with PBS and incubated with Biotin conjugated mouse anti-INFγ for 2 hours at room temperature, followed by 4× wash with PBS and AP conjugated anti-biotin incubation for 2 hours at room temperature. The plates were then washed 4× with PBS and developed with BCIP/NBT substrate until spots appeared on the wells. After a final wash with water and drying the spot forming units (SFU) per million cells from individual wells were counted on a automated ELISpot plate reader. ELISPOT responses for each peptide pool stimulus with splenocytes and IHL's from BALB/c and CD1 mice are represented in separate charts. Total responses from HBV peptide stimulus were also calculated by combining ELISPOT responses to all HBV peptide pools (Core, Pol-1, Poi-2, Poi-3, Poi-4, PreS1/S2 and Surface).

FIGS. 4A-4C: Comparison of the immunogenicity of HBV immunogen SIi-HBV-CPmutS with and without Shark Invariant chain (SIi). (A) HBV-CPmutS immunogen layout. The immunogens HBV-CPmutS was generated by deleting the SIi and first linker present in SIi-HBV-CPmutS. HBV-CPmutS has exactly the same amino acids, except that it lacks the SIi and first linker sequence that is present in the SIi-HBV-CPmutS immunogen. (B) Splenocyte responses in CD1 mice. (C) Intra Hepatic Lymphocyte responses in CD1 mice. ChAdOX2-HBV-CPmutS was generated as described earlier and CD1 mice (10 mice at age of 7 weeks) were vaccinated by intramuscular injections with 5×10⁷ IU per mice ChAdOx2-HBV-CPmutS vaccine and 14 days post-vaccination mice were sacrificed and splenocytes and intra hepatic lymphocytes response data were generated, as described earlier. Data from 5 and 10 CD1 mice vaccinated with ChAdOx2-SIi-HBV-CPmutS and ChAdOx2-HBV-CPmutS respectively were compared and represented in charts for ease of comparison.

FIG. 5A: Schematic layout of SIi-HBV-CPmutPreS-S(sh).

FIG. 5B: Schematic layout of SIi-HBV-CPmutPreS-TPA-S(sh).

FIG. 6A: Schematic layout of MVA-SIi-HBV-PreS-Pₘᵤₜ-C-S(sh).

FIG. 6B: Schematic layout of MVA-SIi-HBV-PreS-Pmut-C-TPA-S(sh).

FIG. 7: Testing ChAdOx1-SIi-HBV-CPmutS vaccine in BALB/c mice, splenocyte response: BALB/c mice (8 mice at the age of 8 weeks) were vaccinated by intramuscular injections with 5×10⁷ IU per mice of ChAdOx1-SIi-HBV-CPmutS vaccine. 14 days post-vaccination, mice were sacrificed; splenocyte response data were generated, as described earlier. Splenocyte T-cell response for each peptide pool stimulus and pooled total response from all HBV-peptide pools are represented in the bar chart.

FIG. 8: Comparison of the immunogenicity of HBV immunogen SIi-HBV-CPmutS encoded via ChAdOx1 and ChAdOx2. HBV-CPmutS immunogen encoding ChAdOx1 and ChAdOx2 viral vectored vaccines were generated, as previously described (FIG. 2D). 10 CD1 mice at age of 7 weeks and 8 CD1 mice at age of 8 weeks were vaccinated by intramuscular injections with 5×10⁷ IU per mice of ChAdOx2-HBV-CPmutS and ChAdOx1-HBV-CPmutS vaccine, respectively, and 14 days post-vaccination mice were sacrificed and splenocyte response data were generated, as described earlier. Data from 10 and 8 CD1 mice vaccinated with ChAdOx2-SIi-HBV-CPmutS and ChAdOx1-HBV-CPmutS were compared and represented in charts for ease of comparison. A statistically significant higher magnitude of total splenocyte T-cell response was observed in mice vaccinated with ChAdOx2-HBV-CPmutS vaccine, compared to the ChAdOx1-HBV-CPmutS vaccine.

FIG. 9: Comparison of the immunogenicity of HBV immunogen SIi-HBV-CPmutPreS-S(sh) (termed as HBV-v2) encoded via ChAdOx1 and ChAdOx2 viral vectors using either a short CMV promoter or long CMV promoter. Two short CMV promoter and one long CMV promoter based SIi-HBV-CPmutPreS-S(sh) immunogen (termed as HBV-v2) encoding ChAdOx1 (ChAdOx1-LP-HBV-v2 and ChAdOx1-SP-HBV-v2) and ChAdOx2 (ChAdOx2-SP-HBV-v2) vaccines were generated as previously described. 15 Balbc mice at age of 8 weeks were split into 3 groups (5 mice per group) and vaccinated by intramuscular injections with 5×10⁷ IU of ChAdOx1-SP-HBV-v2 or ChAdOx2-SP-HBV-v2 or ChAdOx1-LP-HBV-v2 vaccine. 14 days post-vaccination mice were sacrificed and splenocyte response data were generated as previously described. Comparative data from all three HBV-v2 vaccines are represented in the same chart for ease of comparison. A statistically significant higher magnitude of total splenocyte T-cell response was observed in mice vaccinated with ChAdOx1-SP-HBV-v2 vaccine, compared to the other two HBV-v2 vaccines. In addition, the ChAdOx2-SP-HBV-v2 vaccine showed a statistically significant higher magnitude of total splenocyte T-cell response compared to the ChAdOx1-LP-HBV-v2 vaccine.

FIGS. 10A-10G: show immunogen layout for (A) SIi-HBV-CPmutS; (B) SIi-HBV-SCPₘᵤₜ (C) HBV-CPmutS (D) SIi-HBV-CPₘᵤₜ PreS-S(sh) (E) MVA-SIi-HBV-PreS-Pₘᵤₜ-C-S(sh) (F) SIi-HBV-CPₘᵤₜ PreS-TPA-S(sh), and (G) MVA-SIi-HBV-PreS-Pₘᵤₜ-C-TPA-S(sh).

### DETAILED DESCRIPTION

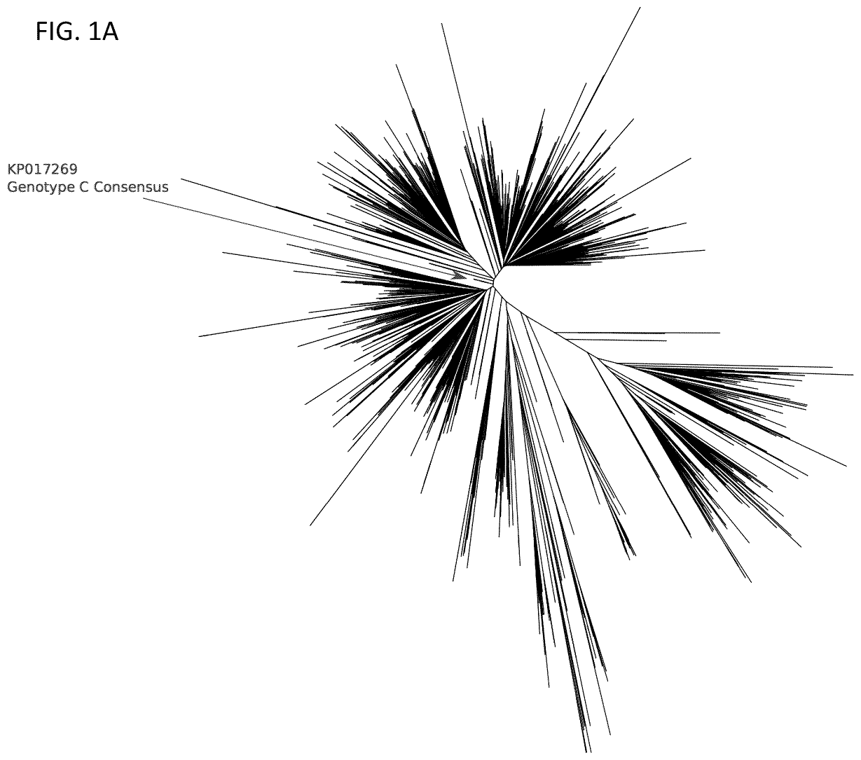

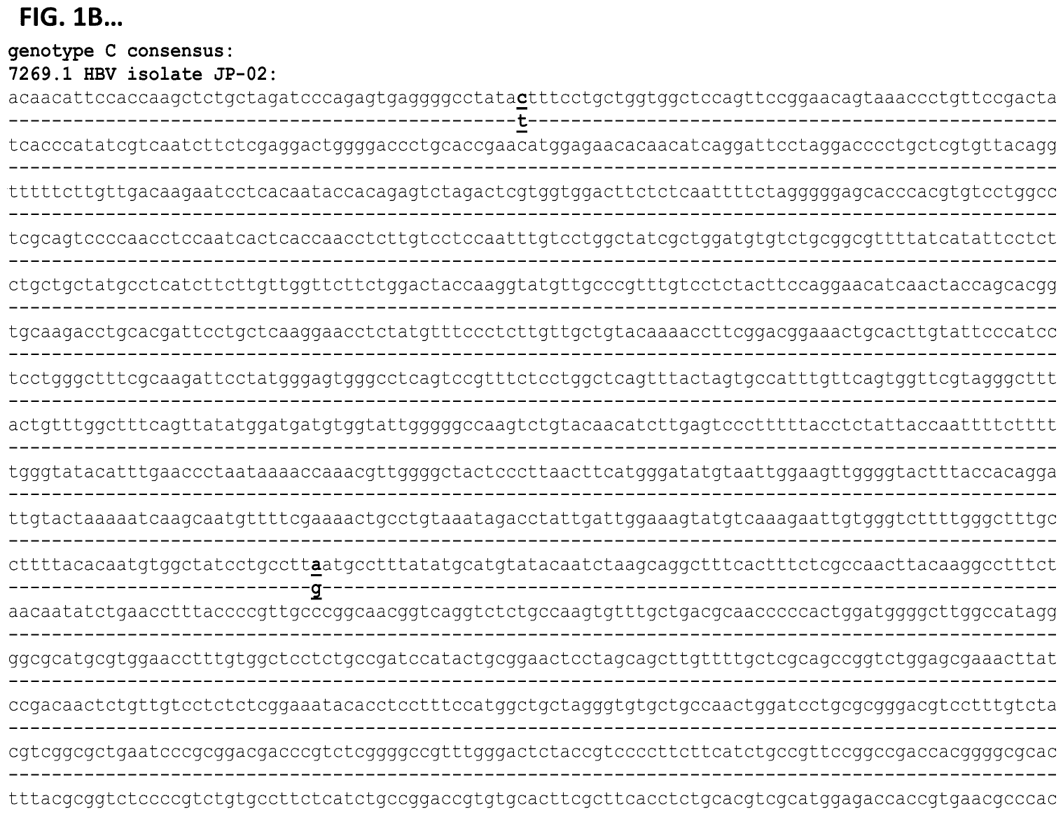

A HBV vaccine was generated based on the HBV genotype C, one of the commonest HBV genotypes in South East Asia that has more frequent association with chronic HBV infection. To design the HBV immunogen, a patient's HBV genotype C sequence (GeneBank: KP017269.1) was selected that was closest to the consensus, this was generated by aligning 1447 HBV-genotype-C sequences from HBVdb, a Hepatitis Virus B database (FIGS. 1A and 1B). The chosen HBV genotype C sequence had only three nucleotide changes compared to the consensus, of which two were silent mutations and one had a mutation in the polymerase protein (FIG. 1C).

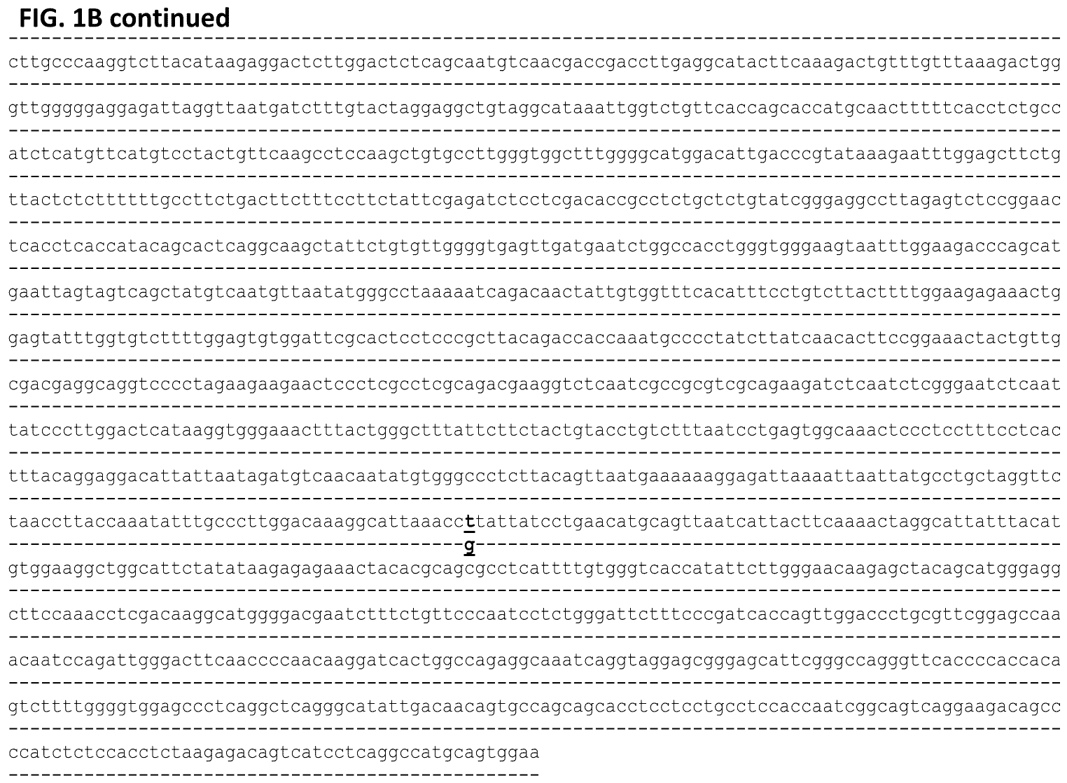

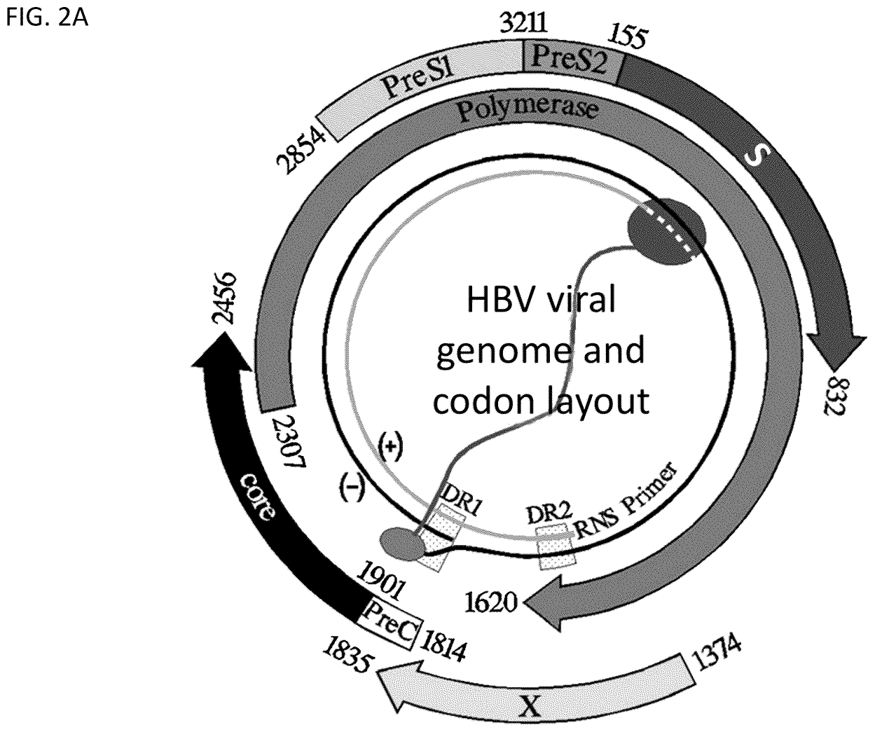

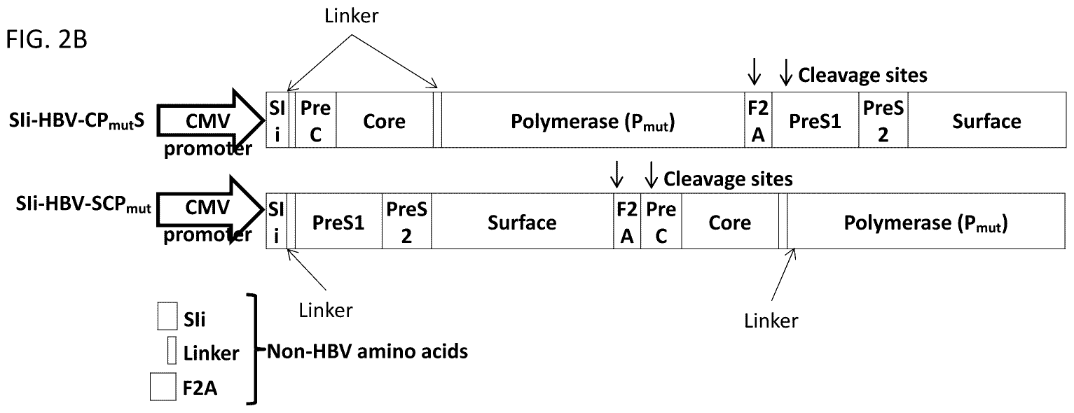

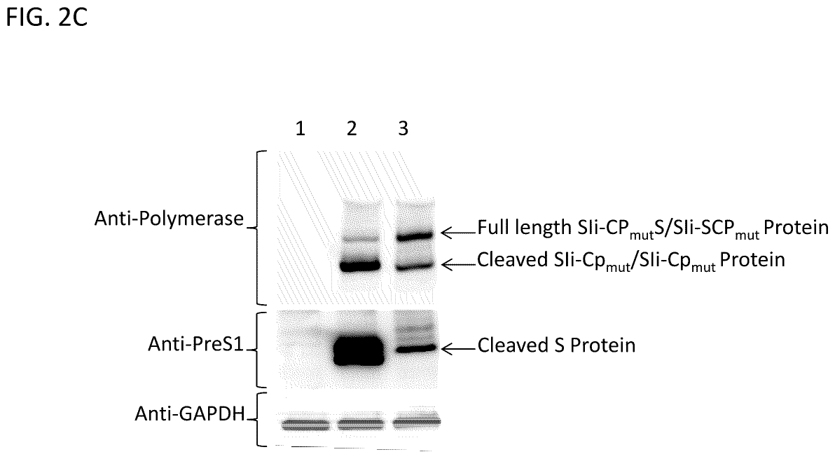

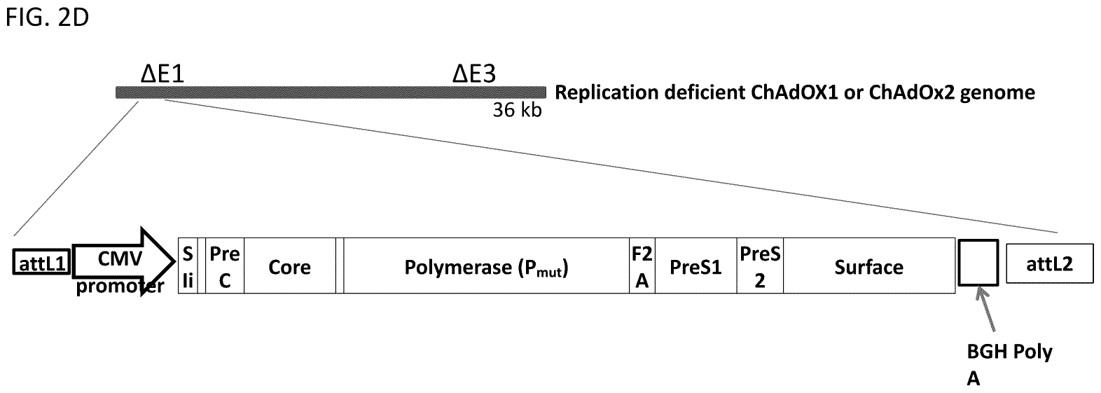

A strong and multi-antigen specific T cell response against different HBV proteins is believed to play a major role in viral clearance of a resolving HBV infection. Based on this HBV immunogens have been designed that encode all major proteins of the virus, namely the core (including the Pre-core region), a non-functional polymerase Pmut (Pmut: HBV polymerase with functional mutations, aiming to discourage the vaccine encoded polymerase's ability to participate in HBV viral replication) and the surface protein (including its PreS1 and PreS2 regions). FIGS. 2A and 2B shows the codon layout of HBV genome and a schematic representation of the first two HBV vaccine immunogen designs respectively. The layout has been designed to encode pre-core, core and Pmut as a fusion protein and a separate surface protein using a furin 2A (F2A) peptide cleavage mechanism, which by causing ribosomal skipping events, helps to encode two proteins from a single open reading frame.

Encoding multiple proteins within a single transgene cassette requires careful design, where the proximity of codon sequence to CMV promoter plays an important role in the level of expression of the encoded proteins. To analyse this two immunogens (FIG. 2B), SIi-HBV-CPmutS and SIi-HBV-SCPmut, we generated which encodes the surface protein either at the end or the beginning of the immunogen cassette respectively and tested them in Western blot expression studies. Western blot expression analysis showed that layout SIi-HBV-CPmutS has the best F2A cleavage possibility, which generates large amounts of the expected CPmut and S proteins when compared to the SIi-HBV-SCPmut (FIG. 2c).

Based on this observation, we decided to take forward SIi-HBV-CPmutS immunogen layout for the generation of chimpanzee adenovirus (ChAdOx2) based T-cell inducing HBV vaccine (FIG. 2D). A small scale batch of ChAdOx2-SIi-HBV-CPmutS was manufactured at the viral vector core facility of the Jenner Institute, University of Oxford, following good manufacturing practices, and used in mice immunogenicity studies.

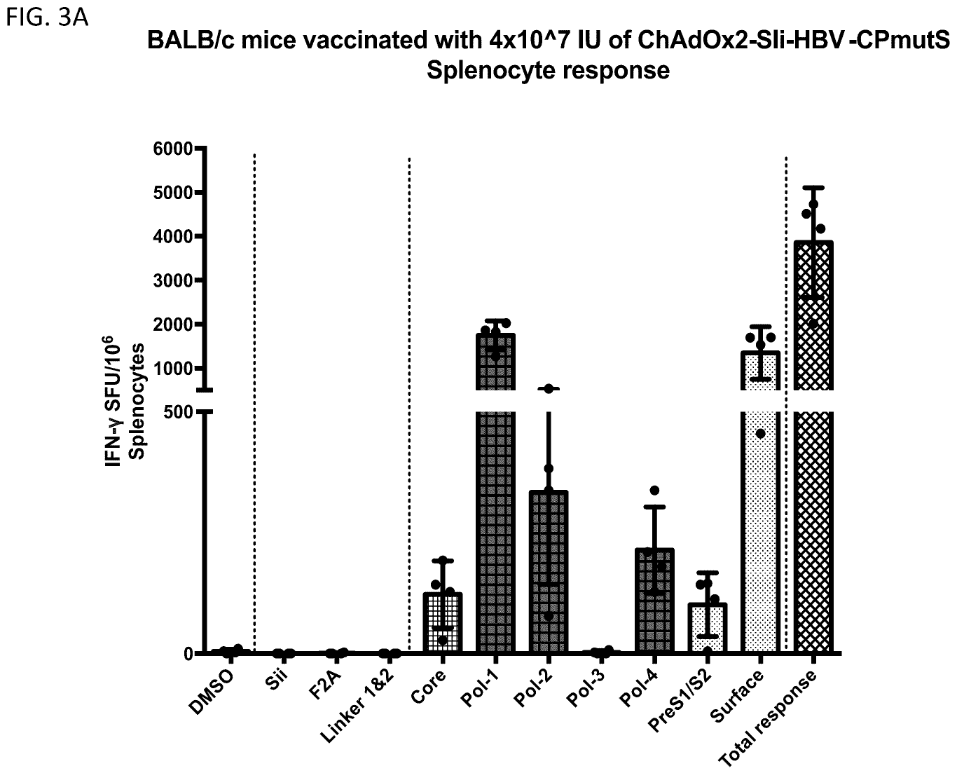

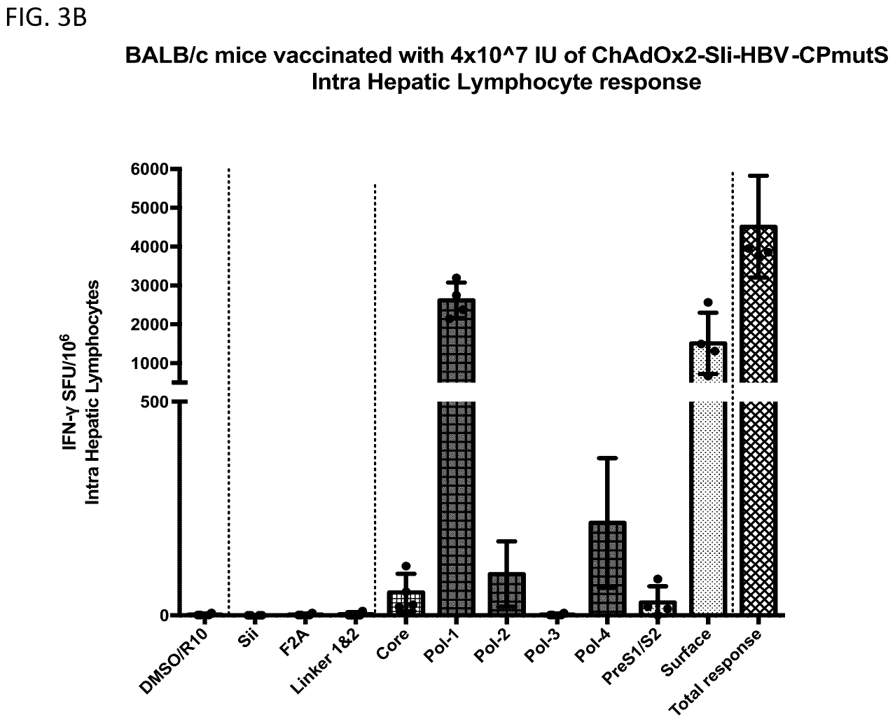

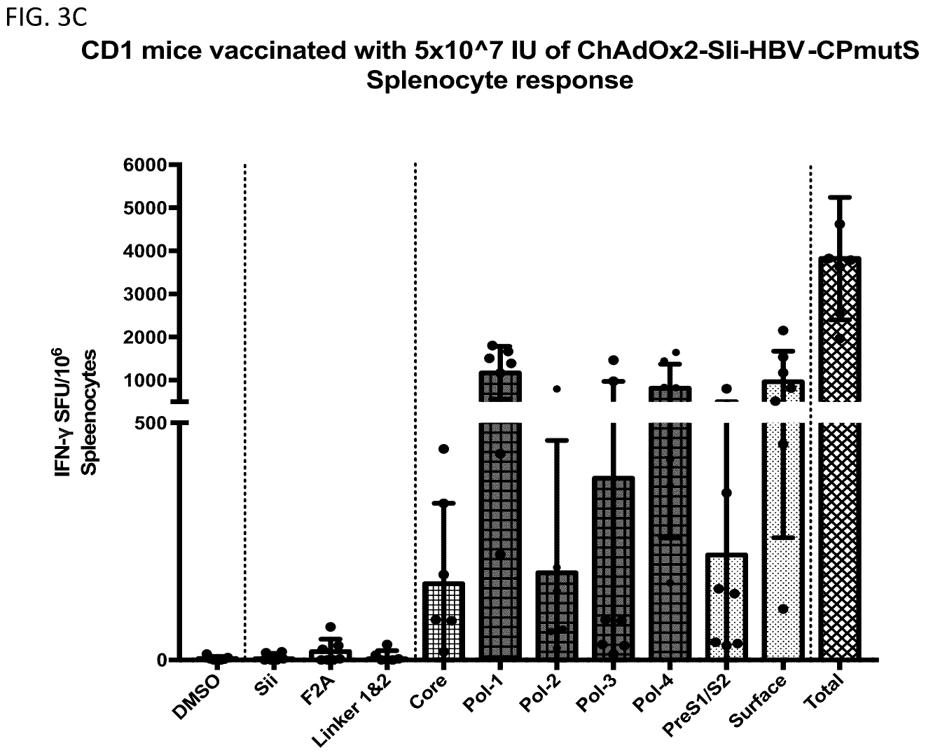

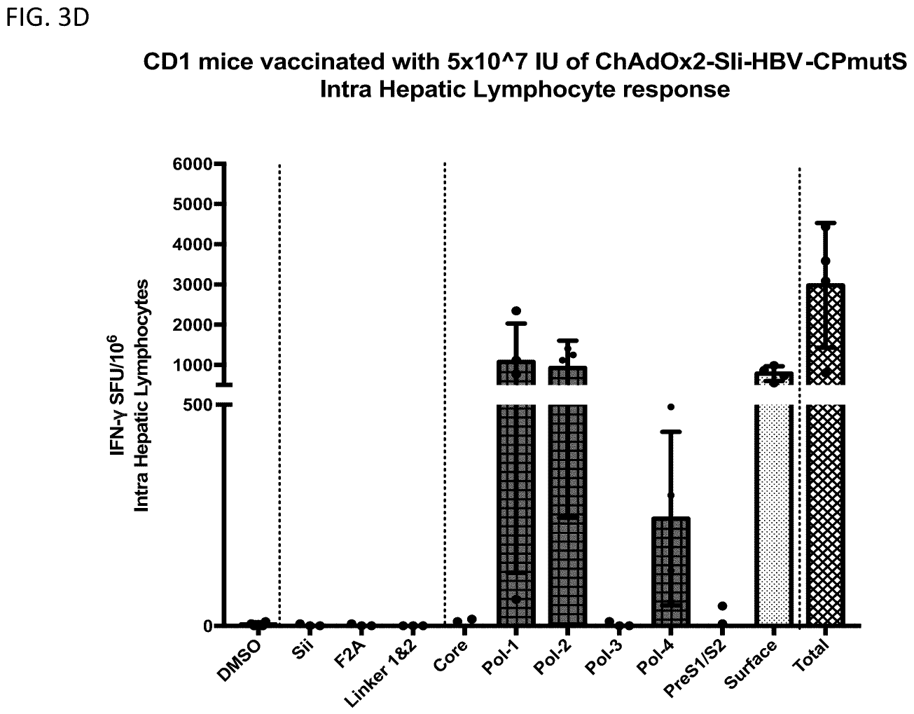

The ability of ChAdOx2-SIi-HBV-CPmutS vaccine to generate a T-cell immune response was tested using naive mice models. Naive BALB/c and CD1 mice were immunized with 4×10⁷ IU and 5×10⁷ IU per mice respectively of ChAdOx2-SIi-HBV-CPmutS vaccine by intra-muscular injections. 14 days post vaccination mice were sacrificed and IFN-γ ELISPOT assays were performed with splenocytes and intra hepatic lymphocytes (IHL) isolated from spleen and liver. Synthetic peptides of 15mers, overlapping by 11 amino acids, generated across the whole of SIi-HBV-CPmutS immunogen, were combined into specific pools (representing different regions of the SIi-HBV-CPmutS immunogen) and used as a stimulant in IFN-γ ELISPOT assays. Results showed that the ChAdOx2-SIi-HBV-CPmutS vaccine generated a good IFN-γ ELISPOT response to SIi-HBV-CPmutS immunogen (FIG. 3). Both BALB/c and CD1 mice showed stronger responses to the polymerase and surface proteins, a weak response to the core protein and negligible or no response to the non-HBV proteins (Shark Invariant chain [SIi], F2A and linkers) in the immunogen.

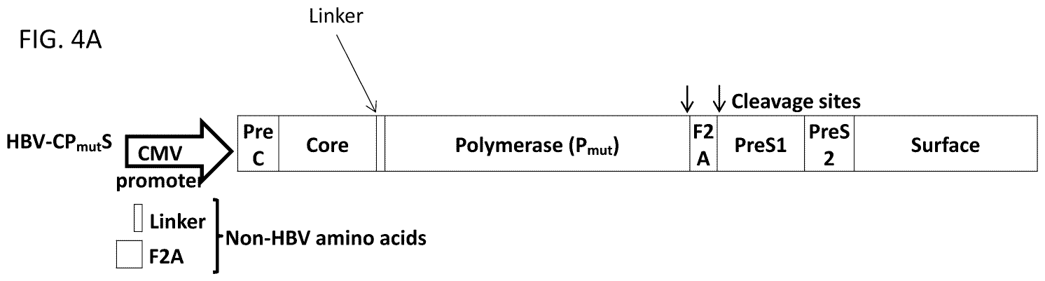

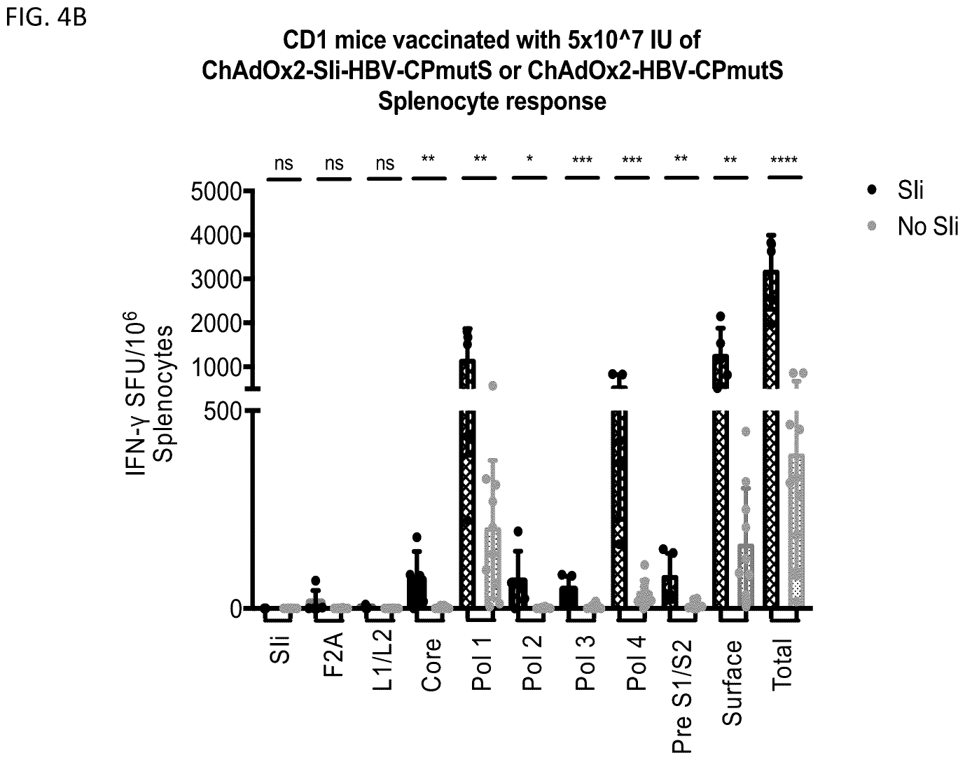

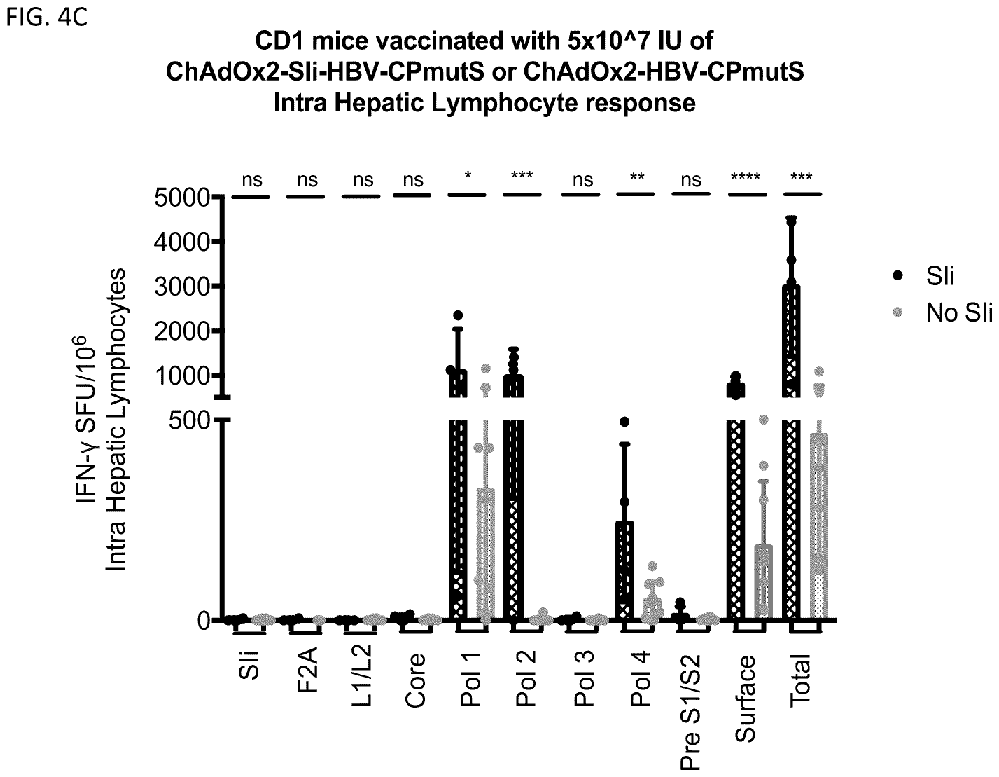

Previous studies have shown that the shark invariant chain (SIi), when placed at the N-terminus of the immunogen, functions as a molecular adjuvant and increases the overall T-cell immune response to the immunogen. To test this, we generated a HBV immunogen without SIi (HBV-CPmutS vaccine) (FIG. 4A), and then generated a ChAdOx2-HBV-CPmutS vaccine and tested them in similar CD1 mice immunogenicity experiments. Results showed that the SIi vaccine (ChAdOx2-SIi-HBV-CPmutS) generates a higher magnitude of IFN-γ ELISPOT response (both Splenocyte and Intra Hepatic Lymphocyte IFN-γ ELISPOT response, represented in FIG. 4B, 4C) compared to the Non-SIi vaccine (ChAdOx2-HBV-CPmutS).

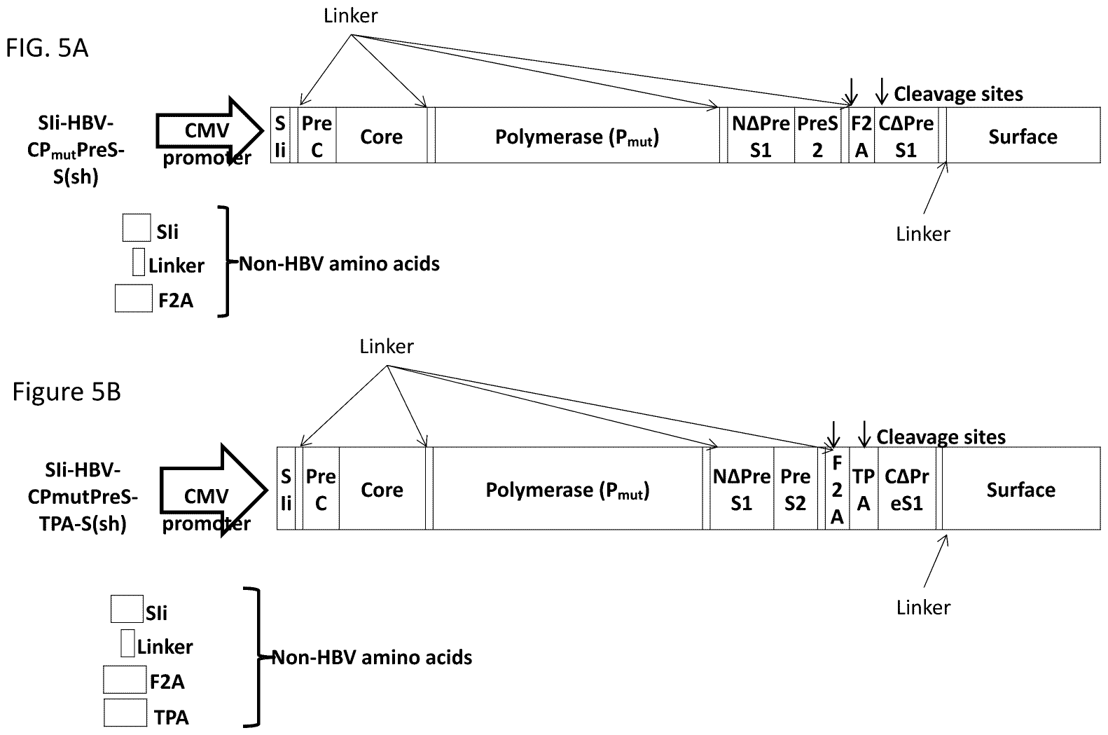

The main aim of the viral vectored HBV vaccine design is to generate both T-cell and antibody response to the HBV immunogen. In order to generate a successful antibody response, the immunogen encoded protein has to fold into its native conformation. To provide this, HBV immunogens, SIi-HBV-CPmutPreS-S(sh) and SIi-HBV-CPmutPreS-TPA-S(sh) (FIGS. 5A and 5B) were designed, which encode the N-terminal half of PreS1 domain fused with a linker to the S domain of surface protein, as the antibody inducing immunogen component, and the remaining peptide sequences of the surface protein (the C-terminal half of PreS1 domain and the whole PreS2 domain) were fused to the PreCore/Core/Pmut, in order to preserve the T-cell epitopes that would be required for generation of T-cell immune response. FIGS. 5A and 5B shows a schematic layout of SIi-HBV-CPmutPreS-S(sh).

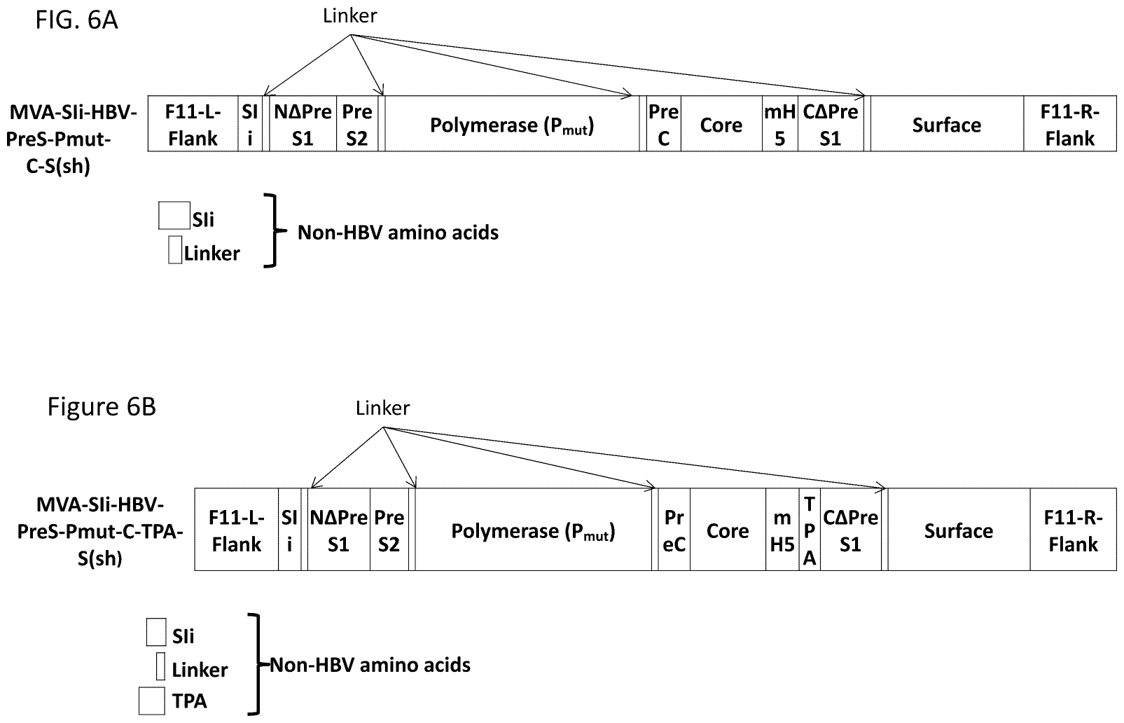

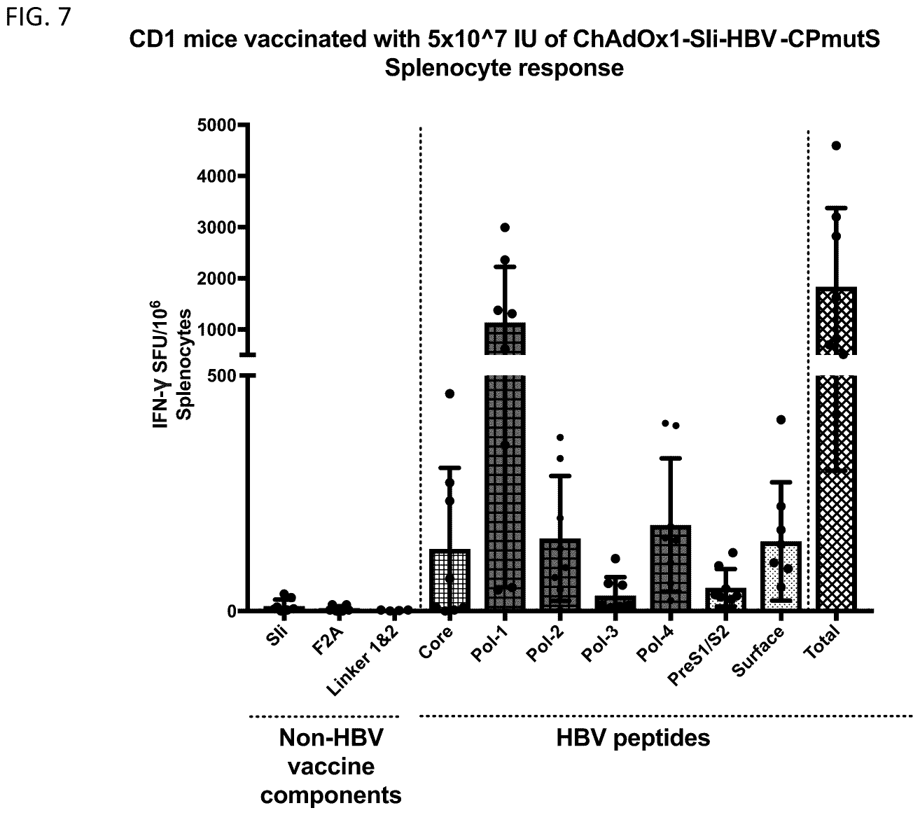

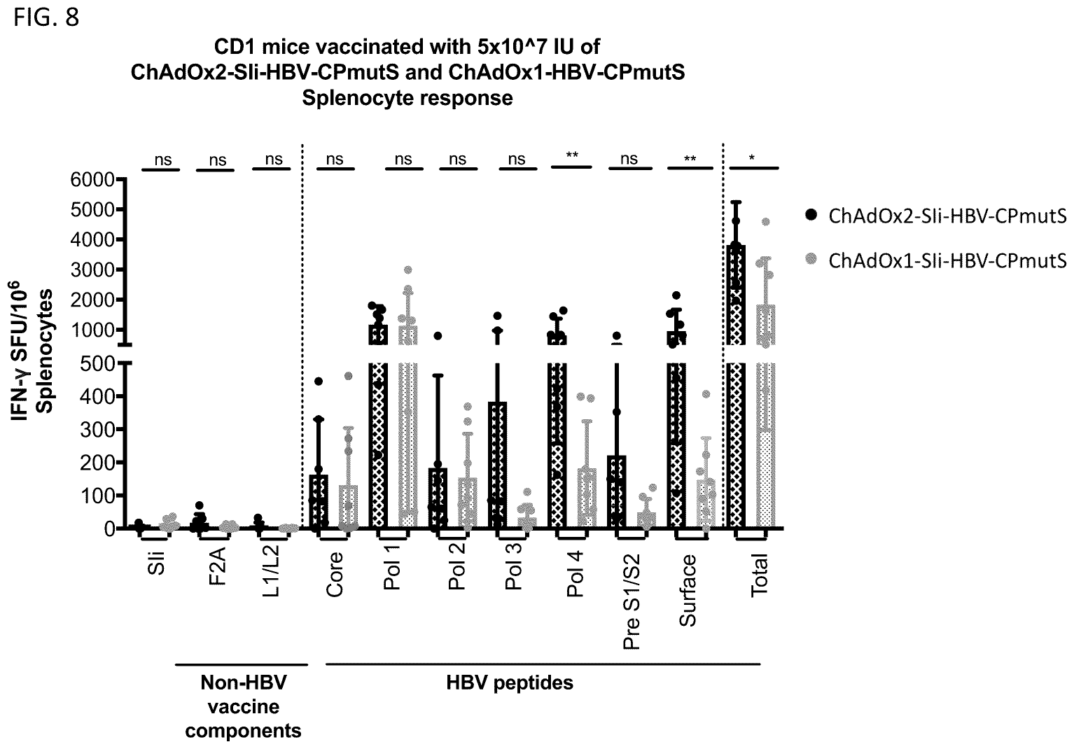

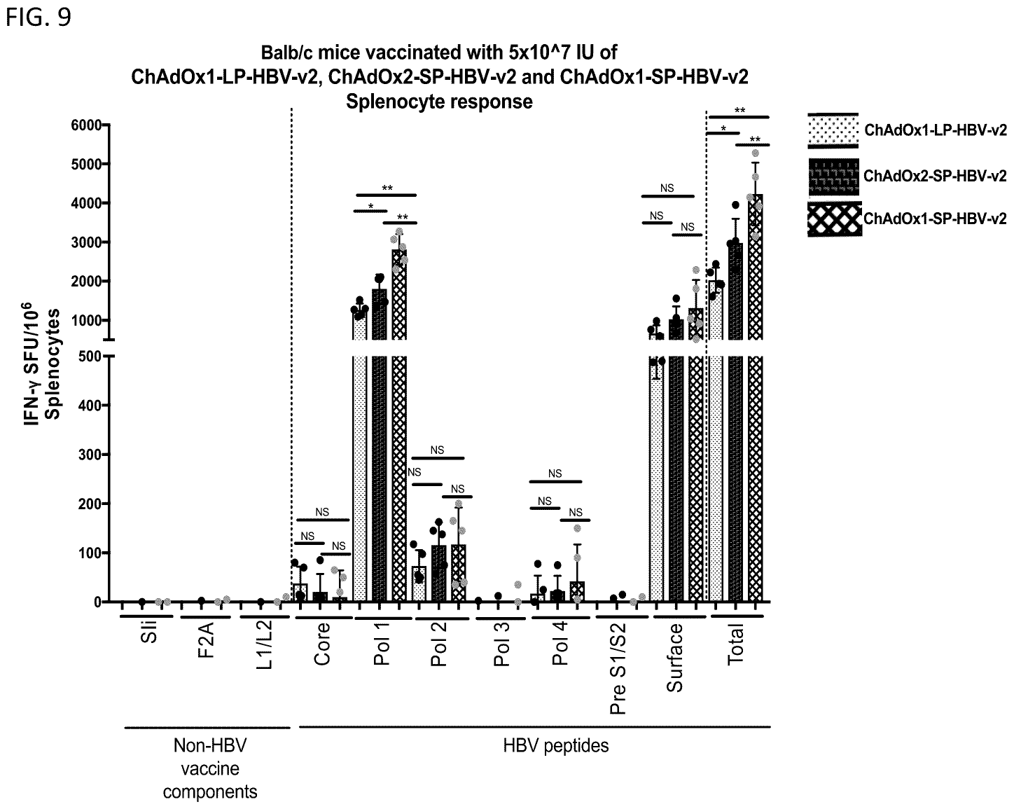

MVA-HBV immunogens were also designed, which followed designs similar to SIi-HBV-CPmutPreS-S(sh) and and SIi-HBV-CPmutPreS-TPA-S(sh). However, the T-cell component of HBV immunogen SIi-HBV-CPmutPreS is encoded by the early promoter F11 and the antibody inducing component S(sh) or TPA-S(sh) is encoded by the early/late promoter mH5. The cloning cassette also has F11-left flank and F11-right flank sequences, to allow insertion into the F11 locus, by homologous recombination. FIGS. 6A and 6B shows schematic layouts of MVA-SIi-HBV-PreS-Pmut-C-S(sh) and MVA-SIi-HBV-CPmutPreS-TPA-S(sh) respectively.

Conclusion: The novel multi-antigen HBV immunogen based ChAdOx2-SIi-HBV-CPmutS are highly immunogenic in naive mice models. These studies pave way for future studies in chronic HBV mice models, to assess their ability to overcome chronic HBV infection through immunotherapeutic approaches.

### Sequences

HBV genotype C consensus sequence generated from 1447 genotype C sequences (3215 base pairs) is provided as SEQ ID NO: 1. The Sequence of KP017269.1 HBV isolate JP-02 is provided as SEQ ID NO: 2.

Example Cassettes

1. SIi-HBV-CPₘᵤₜS

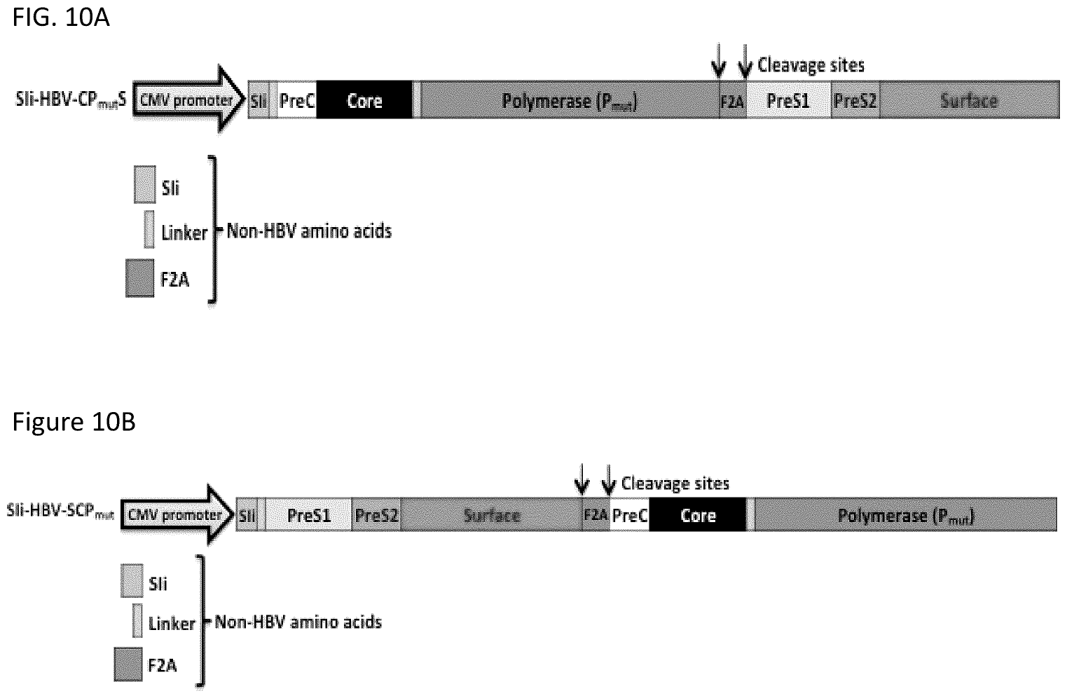

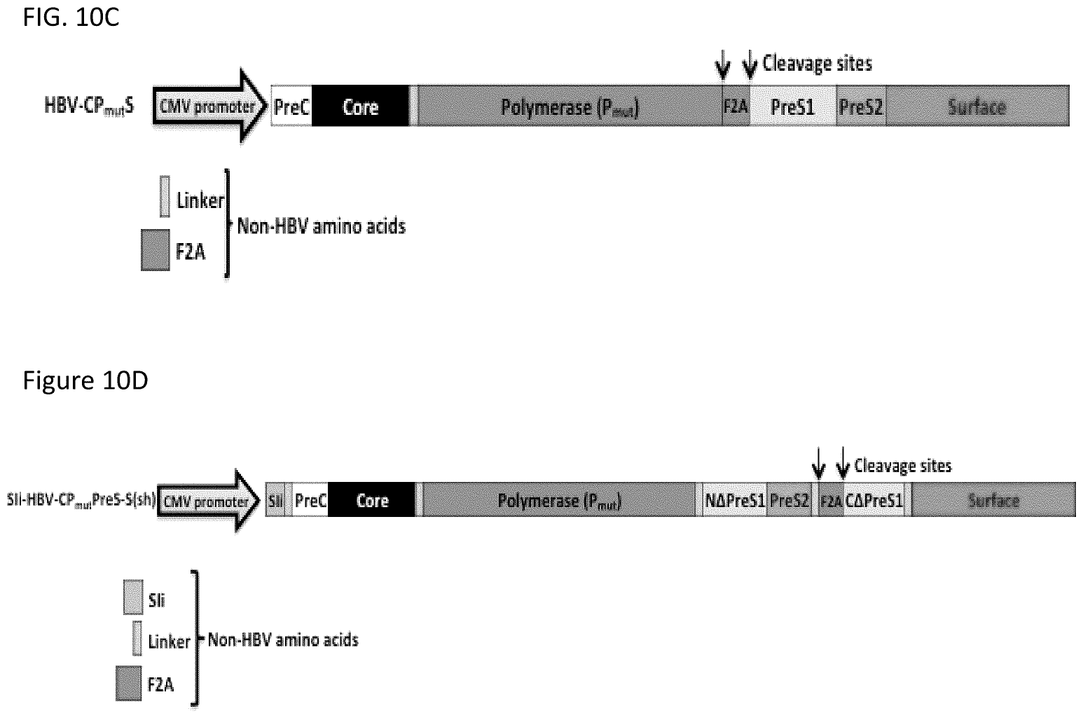

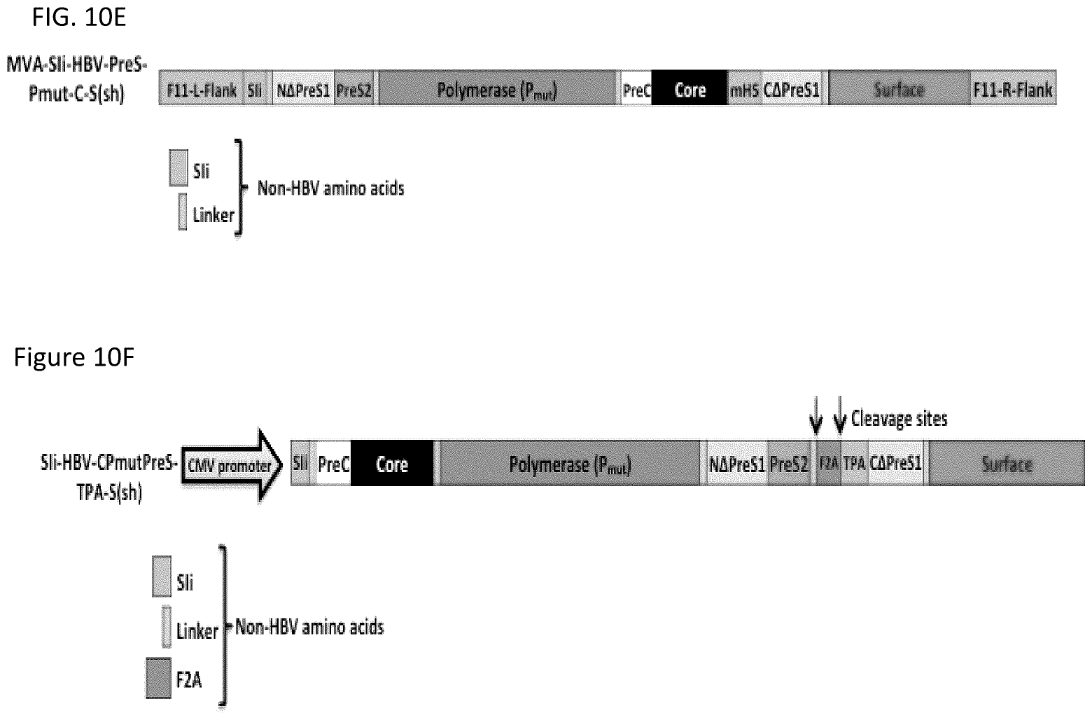

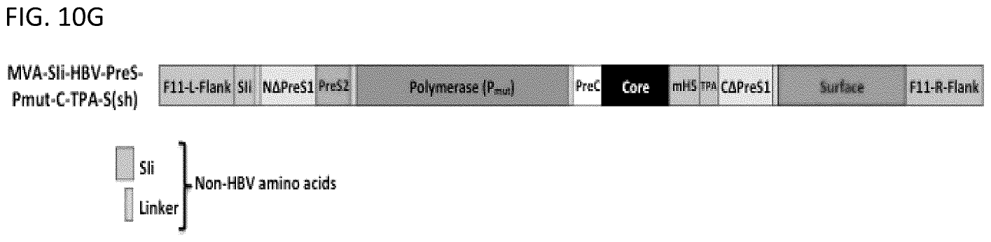

1.1. SIi-HBV-CPₘᵤₜS: Immunogen Layout is Shown in FIG. 10A

1.2. SIi-HBV-CPₘᵤₜS: Amino acid sequence
(SEQ ID NO: 3)
MSLLWGGVTVLAAMLIAGQVASVVFLVKGGGPGGGMQLFHLCLIISCS
CPTVQASKLCLGWLWGMDIDPYKEFGASVELLSFLPSDFFPSIRDLLD
TASALYREALESPEHCSPHHTALRQAILCWGELMNLATWVGSNLEDPA
SRELVVSYVNVNMGLKIRQLLWFHISCLTFGRETVLEYLVSFGVWIRT
PPAYRPPNAPILSTLPETTVVRRRGRSPRRRTPSPRRRRSQSPRRRRS
QSRESQCGGGSGGGMPLSYQHFRKLLLLDDEAGPLEEELPRLADEGLN
RRVAEDLNLGNLNVSIPWTHKVGNFTGLASSTVPVFNPEWQTPSFPHI
HLQEDIINRCQQYVGPLTVNEKRRLKLIMPARFYPNLTKYLPLDKGIK
PYYPEHAVNHYFKTRHYLHTLWKAGILYKRETTRSASFCGSPYSWEQE
LQHGRLVFQTSTRHGDESFCSQSSGILSRSPVGPCVRSQLKQSRLGLQ
PQQGSLARGKSGRSGSIRARVHPTTRRSFGVEPSGSGHIDNSASSTSS
CLHQSAVRKTAYSHLSTSKRQSSSGHAVELHNIPPSSARSQSEGPIFS
AWWLQFRNSKPASDYALTHIVNLLEDWGPATEHGEHNIRIPRTPARVT
GGVFLVDKNPHNTTESRLVVDFSQFSRGSTHVSWPKFAVPNLQSLTNL
LSSNLSWLSLDVSAAFYHIPLHPAAMPHLLVGSSGLPRYVARLSSTSR
NINYQHGTMQDLHDSCSRNLYVSLLLLYKTFGRKLHLYSHPIILGFRK
IPMGVGLSPFLLAQFTSAICSVVRRAFPHCLAFSYMDDVVLGAKSVQH
LESLFTSITNFLLSLGIHLNPNKTKRWGYSLNFMGYVIGSWGTLPQEH
IVLKIKQCFRKLPVNRPIDWKVCQRIVGLLGFAAPFTQCGYPALMPLY
ACIQSKQAFTFSPTYKAFLCKQYLNLYPVARQRSGLCQVFADATPTGW
GLAIGHRAMRGTFVAPLPIHTAELLAACFARSRSGAKLIGTDNSVVLS
RKYTSFPWLLGCAANWILRGTSFVYVPSALNPAADPSAGRLGLYRPLL
HLPFRPTTGRTSLYAVSPSVPSHLPDRVHFASPLHVAWRPPRKRRAPV
KQTLNFDLLKLAGDVESNPGPMGGWSSKPRQGMGTNLSVPNPLGFFPD
HQLDPAFGANSNNPDWDFNPNKDHWPEANQVGAGAFGPGFTPPHGGLL
GWSPQAQGILTTVPAAPPPASTNRQSGRQPTPISPPLRDSHPQAMQWN
STTFHQALLDPRVRGLYFPAGGSSSGTVNPVPTTASPISSIFSRTGDP
APNMENTTSGFLGPLLVLQAGFFLLTRILTIPQSLDSWWTSLNFLGGA
PTCPGQNSQSPTSNHSPTSCPPICPGYRWMCLRRFIIFLFILLLCLIF
LLVLLDYQGMLPVCPLLPGTSTTSTGPCKTCTIPAQGTSMFPSCCCTK
PSDGNCTCIPIPSSWAFARFLWEWASVRFSWLSLLVPFVQWFVGLSPT
VWLSVIWMMWYWGPSLYNILSPFLPLLPIFFCLWVYI
1.3. SIi-HBV-CPₘᵤₜS: Description for amino acid
sequences of HBV immunogen
1.3.1. First amino acid of polypeptide = M
1.3.2. Shark Invariant chain (SIi) =
(SEQ ID NO: 4)
SLLWGGVTVLAAMLIAGQVASVVFLV
1.3.3. Linker =
(SEQ ID NO: 5)
KGGGPGGG
1.3.4. C =
1.3.4.1. PreC =
(SEQ ID NO: 16)
MQLFHLCLIISCSCPTVQASKLCLGWLWG
1.3.4.2. Core =
(SEQ ID NO: 6)
MDIDPYKEFGASVELLSFLPSDFFPSIRDLLDTASALYREALESPEHC
SPHHTALRQAILCWGELMNLATWVGSNLEDPASRELVVSYVNVNMGLK
IRQLLWFHISCLTFGRETVLEYLVSFGVWIRTPPAYRPPNAPILSTLP
ETTVVRRRGRSPRRRTPSPRRRRSQSPRRRRSQSRESQC
1.3.5. Linker =
(SEQ ID NO: 7)
GGGSGGG
1.3.6. Pmut = (mutations: Y63A, C323A, C334A,
C338A, C352A, R714A, D788A, R792A)
(SEQ ID NO: 8)
MPLSYQHFRKLLLLDDEAGPLEEELPRLADEGLNRRVAEDLNLGNLNV
SIPWTHKVGNFTGLASSTVPVFNPEWQTPSFPHIHLQEDIINRCQQYV
GPLTVNEKRRLKLIMPARFYPNLTKYLPLDKGIKPYYPEHAVNHYFKT
RHYLHTLWKAGILYKRETTRSASFCGSPYSWEQELQHGRLVFQTSTRH
GDESFCSQSSGILSRSPVGPCVRSQLKQSRLGLQPQQGSLARGKSGRS
GSIRARVHPTTRRSFGVEPSGSGHIDNSASSTSSCLHQSAVRKTAYSH
LSTSKRQSSSGHAVELHNIPPSSARSQSEGPIFSAWWLQFRNSKPASD
YALTHIVNLLEDWGPATEHGEHNIRIPRTPARVTGGVFLVDKNPHNTT
ESRLVVDFSQFSRGSTHVSWPKFAVPNLQSLTNLLSSNLSTFGRKLHL
YSHPIILGFRKIPMGVGLSPFLLAQFTSAICSVVRRAFPHCLAFSYMD
DVVLGAKSVQHLESLFTSITNFLLSLGIHLNPNKTKRWGYSLNFMGYV
IGSWGTLPQEHIVLKIKQCFRKLPVNRPIDWKVCQRIVGLLGFAAPFT
QCGYPALMPLYACIQSKQAFTFSPTYKAFLCKQYLNLYPVARQRSGLC
QVFADATPTGWGLAIGHRAMRGTFVAPLPIHTAELLAACFARSRSGAK
LIGTDNSVVLSRKYTSFPWLLGCAANWILRGTSFVYVPSALNPAADPS
AGRLGLYRPLLHLPFRPTTGRTSLYAVSPSVPSHLPDRVHFASPLHVA
WRPP
1.3.7. Furin 2A (F2A) =
(SEQ ID NO: 9)
RKRRAPVKQTLNFDLLKLAGDVESNPGP
1.3.8. Surface proteins (S) =
1.3.8.1. PreS1 =
(SEQ ID NO: 52)
MGGWSSKPRQGMGTNLSVPNPLGFFPDHQLDPAFGANSNNPDWDFNPN
KDHWPEANQVGAGAFGPGFTPPHGGLLGWSPQAQGILTTVPAAPPPAS
TNRQSGRQPTPISPPLRDSHPQA
1.3.8.2. PreS2 =
(SEQ ID NO: 53)
MQWNSTTFHQALLDPRVRGLYFPAGGSSSGTVNPVPTTASPISSIFSR
TGDPAPN
1.3.8.3. Surface (S) =
(SEQ ID NO: 10)
MENTTSGFLGPLLVLQAGFFLLTRILTIPQSLDSWWTSLNFLGGAPTC
PGQNSQSPTSNHSPTSCPPICPGYRWMCLRRFIIFLFILLLCLIFLLV
LLDYQGMLPVCPLLPGTSTTSTGPCKTCTIPAQGTSMFPSCCCTKPSD
GNCTCIPIPSSWAFARFLWEWASVRFSWLSLLVPFVQWFVGLSPTVWL
SVIWMMWYWGPSLYNILSPFLPLLPIFFCLWVYI

The SIi-HBV-CPmutS nucleotide sequence is provided as SEQ ID NO: 46.

2. SIi-HBV-SCPₘᵤₜ

2.1. SIi-HBV-SCPₘᵤₜ: Immunogen Layout is Shown in FIG. 10B

2.2. SIi_HBV-SCPₘᵤₜ: Amino acid sequence
(SEQ ID NO: 11)
MSLLWGGVTVLAAMLIAGQVASVVFLVKGGSMGGWSSKPRQGMGTNLS
VPNPLGFFPDHQLDPAFGANSNNPDWDFNPNKDHWPEANQVGAGAFGP
GFTPPHGGLLGWSPQAQGILTTVPAAPPPASTNRQSGRQPTPISPPLR
DSHPQAMQWNSTTFHQALLDPRVRGLYFPAGGSSSGTVNPVPTTASPI
SSIFSRTGDPAPNMENTTSGFLGPLLVLQAGFFLLTRILTIPQSLDSW
WTSLNFLGGAPTCPGQNSQSPTSNHSPTSCPPICPGYRWMCLRRFIIF
LFILLLCLIFLLVLLDYQGMLPVCPLLPGTSTTSTGPCKTCTIPAQGT
SMFPSCCCTKPSDGNCTCIPIPSSWAFARFLWEWASVRFSWLSLLVPF
VQWFVGLSPTVWLSVIWMMWYWGPSLYNILSPFLPLLPIFFCLWVYIR
KRRAPVKQTLNFDLLKLAGDVESNPGPMQLFHLCLIISCSCPTVQASK
LCLGWLWGMDIDPYKEFGASVELLSFLPSDFFPSIRDLLDTASALYRE
ALESPEHCSPHHTALRQAILCWGELMNLATWVGSNLEDPASRELVVSY
VNVNMGLKIRQLLWFHISCLTFGRETVLEYLVSFGVWIRTPPAYRPPN
APILSTLPETTVVRRRGRSPRRRTPSPRRRRSQSPRRRRSQSRESQCG
GGSGGGMPLSYQHFRKLLLLDDEAGPLEEELPRLADEGLNRRVAEDLN
LGNLNVSIPWTHKVGNFTGLASSTVPVFNPEWQTPSFPHIHLQEDIIN
RCQQYVGPLTVNEKRRLKLIMPARFYPNLTKYLPLDKGIKPYYPEHAV
NHYFKTRHYLHTLWKAGILYKRETTRSASFCGSPYSWEQELQHGRLVF
QTSTRHGDESFCSQSSGILSRSPVGPCVRSQLKQSRLGLQPQQGSLAR
GKSGRSGSIRARVHPTTRRSFGVEPSGSGHIDNSASSTSSCLHQSAVR
KTAYSHLSTSKRQSSSGHAVELHNIPPSSARSQSEGPIFSAWWLQFRN
SKPASDYALTHIVNLLEDWGPATEHGEHNIRIPRTPARVTGGVFLVDK
NPHNTTESRLVVDFSQFSRGSTHVSWPKFAVPNLQSLTNLLSSNLSWL
SLDVSAAFYHIPLHPAAMPHLLVGSSGLPRYVARLSSTSRNINYQHGT
MQDLHDSCSRNLYVSLLLLYKTFGRKLHLYSHPIILGFRKIPMGVGLS
PFLLAQFTSAICSVVRRAFPHCLAFSYMDDVVLGAKSVQHLESLFTSI
TNFLLSLGIHLNPNKTKRWGYSLNFMGYVIGSWGTLPQEHIVLKIKQC
FRKLPVNRPIDWKVCQRIVGLLGFAAPFTQCGYPALMPLYACIQSKQA
FTFSPTYKAFLCKQYLNLYPVARQRSGLCQVFADATPTGWGLAIGHRA
MRGTFVAPLPIHTAELLAACFARSRSGAKLIGTDNSVVLSRKYTSFPW
LLGCAANWILRGTSFVYVPSALNPAADPSAGRLGLYRPLLHLPFRPTT
GRTSLYAVSPSVPSHLPDRVHFASPLHVAWRPP
2.3. SIi-HBV-SCPₘᵤₜ: Description for amino acid
sequences of HBV immunogen
2.3.1. First amino acid of polypeptide = M
2.3.2. Shark Invariant chain (SIi) =
(SEQ ID NO: 4)
SLLWGGVTVLAAMLIAGQVASVVFLV
2.3.3. Linker =
(SEQ ID NO: 14)
KGGS
2.3.4. Surface proteins (S) =
2.3.4.1. PreS1 =
(SEQ ID NO: 52)
MGGWSSKPRQGMGTNLSVPNPLGFFPDHQLDPAFGANSNNPDWDFNPN
KDHWPEANQVGAGAFGPGFTPPHGGLLGWSPQAQGILTTVPAAPPPAS
TNRQSGRQPTPISPPLRDSHPQA
2.3.4.2. PreS2 =
(SEQ ID NO: 53)
MQWNSTTFHQALLDPRVRGLYFPAGGSSSGTVNPVPTTASPISSIFSR
TGDPAPN
2.3.4.3. Surface (S) =
(SEQ ID NO: 10)
MENTTSGFLGPLLVLQAGFFLLTRILTIPQSLDSWWTSLNFLGGAPTC
PGQNSQSPTSNHSPTSCPPICPGYRWMCLRRFIIFLFILLLCLIFLLV
LLDYQGMLPVCPLLPGTSTTSTGPCKTCTIPAQGTSMFPSCCCTKPSD
GNCTCIPIPSSWAFARFLWEWASVRFSWLSLLVPFVQWFVGLSPTVWL
SVIWMMWYWGPSLYNILSPFLPLLPIFFCLWVYI
2.3.5. Furin 2A (F2A) =
(SEQ ID NO: 9)
RKRRAPVKQTLNFDLLKLAGDVESNPGP
2.3.6. C =
2.3.6.1. PreCore =
(SEQ ID NO: 16)
MQLFHLCLIISCSCPTVQASKLCLGWLWG
2.3.6.2. Core =
(SEQ ID NO: 6)
MDIDPYKEFGASVELLSFLPSDFFPSIRDLLDTASALYREALESPEHC
SPHHTALRQAILCWGELMNLATWVGSNLEDPASRELVVSYVNVNMGLK
IRQLLWFHISCLTFGRETVLEYLVSFGVWIRTPPAYRPPNAPILSTLP
ETTVVRRRGRSPRRRTPSPRRRRSQSPRRRRSQSRESQC
2.3.7. Linker =
(SEQ ID NO: 7)
GGGSGGG
2.3.8. Pmut = (mutations: Y63A, C323A, C334A,
C338A, C352A, R714A, D788A, R792A)
(SEQ ID NO: 8)
MPLSYQHFRKLLLLDDEAGPLEEELPRLADEGLNRRVAEDLNLGNLNV
SIPWTHKVGNFTGLASSTVPVFNPEWQTPSFPHIHLQEDIINRCQQYV
GPLTVNEKRRLKLIMPARFYPNLTKYLPLDKGIKPYYPEHAVNHYFKT
RHYLHTLWKAGILYKRETTRSASFCGSPYSWEQELQHGRLVFQTSTRH
GDESFCSQSSGILSRSPVGPCVRSQLKQSRLGLQPQQGSLARGKSGRS
GSIRARVHPTTRRSFGVEPSGSGHIDNSASSTSSCLHQSAVRKTAYSH
LSTSKRQSSSGHAVELHNIPPSSARSQSEGPIFSAWWLQFRNSKPASD
YALTHIVNLLEDWGPATEHGEHNIRIPRTPARVTGGVFLVDKNPHNTT
ESRLVVDFSQFSRGSTHVSWPKFAVPNLQSLTNLLSSNLSTFGRKLHL
YSHPIILGFRKIPMGVGLSPFLLAQFTSAICSVVRRAFPHCLAFSYMD
DVVLGAKSVQHLESLFTSITNFLLSLGIHLNPNKTKRWGYSLNFMGYV
IGSWGTLPQEHIVLKIKQCFRKLPVNRPIDWKVCQRIVGLLGFAAPFT
QCGYPALMPLYACIQSKQAFTFSPTYKAFLCKQYLNLYPVARQRSGLC
QVFADATPTGWGLAIGHRAMRGTFVAPLPIHTAELLAACFARSRSGAK
LIGTDNSVVLSRKYTSFPWLLGCAANWILRGTSFVYVPSALNPAADPS
AGRLGLYRPLLHLPFRPTTGRTSLYAVSPSVPSHLPDRVHFASPLHVA
WRPP

The SIi-HBV-SCPmut nucleotide sequence is provided as SEQ ID NO: 47:

3. HBV-CPₘᵤₜS

3.1. HBV-CPₘᵤₜS: Immunogen layout is shown in FIG. 10C

3.2. HBV-CPₘᵤₜS: Amino acid sequence
(SEQ ID NO: 12)
MQLFHLCLIISCSCPTVQASKLCLGWLWGMDIDPYKEFGASVELLSFL
PSDFFPSIRDLLDTASALYREALESPEHCSPHHTALRQAILCWGELMN
LATWVGSNLEDPASRELVVSYVNVNMGLKIRQLLWFHISCLTFGRETV
LEYLVSFGVWIRTPPAYRPPNAPILSTLPETTVVRRRGRSPRRRTPSP
RRRRSQSPRRRRSQSRESQCGGGSGGGMPLSYQHFRKLLLLDDEAGPL
EEELPRLADEGLNRRVAEDLNLGNLNVSIPWTHKVGNFTGLASSTVPV
FNPEWQTPSFPHIHLQEDIINRCQQYVGPLTVNEKRRLKLIMPARFYP
NLTKYLPLDKGIKPYYPEHAVNHYFKTRHYLHTLWKAGILYKRETTRS
ASFCGSPYSWEQELQHGRLVFQTSTRHGDESFCSQSSGILSRSPVGPC
VRSQLKQSRLGLQPQQGSLARGKSGRSGSIRARVHPTTRRSFGVEPSG
SGHIDNSASSTSSCLHQSAVRKTAYSHLSTSKRQSSSGHAVELHNIPP
SSARSQSEGPIFSAWWLQFRNSKPASDYALTHIVNLLEDWGPATEHGE
HNIRIPRTPARVTGGVFLVDKNPHNTTESRLVVDFSQFSRGSTHVSWP
KFAVPNLQSLTNLLSSNLSWLSLDVSAAFYHIPLHPAAMPHLLVGSSG
LPRYVARLSSTSRNINYQHGTMQDLHDSCSRNLYVSLLLLYKTFGRKL
HLYSHPIILGFRKIPMGVGLSPFLLAQFTSAICSVVRRAFPHCLAFSY
MDDVVLGAKSVQHLESLFTSITNFLLSLGIHLNPNKTKRWGYSLNFMG
YVIGSWGTLPQEHIVLKIKQCFRKLPVNRPIDWKVCQRIVGLLGFAAP
FTQCGYPALMPLYACIQSKQAFTFSPTYKAFLCKQYLNLYPVARQRSG
LCQVFADATPTGWGLAIGHRAMRGTFVAPLPIHTAELLAACFARSRSG
AKLIGTDNSVVLSRKYTSFPWLLGCAANWILRGTSFVYVPSALNPAAD
PSAGRLGLYRPLLHLPFRPTTGRTSLYAVSPSVPSHLPDRVHFASPLH
VAWRPPRKRRAPVKQTLNFDLLKLAGDVESNPGPMGGWSSKPRQGMGT
NLSVPNPLGFFPDHQLDPAFGANSNNPDWDFNPNKDHWPEANQVGAGA
FGPGFTPPHGGLLGWSPQAQGILTTVPAAPPPASTNRQSGRQPTPISP
PLRDSHPQAMQWNSTTFHQALLDPRVRGLYFPAGGSSSGTVNPVPTTA
SPISSIFSRTGDPAPNMENTTSGFLGPLLVLQAGFFLLTRILTIPQSL
DSWWTSLNFLGGAPTCPGQNSQSPTSNHSPTSCPPICPGYRWMCLRRF
IIFLFILLLCLIFLLVLLDYQGMLPVCPLLPGTSTTSTGPCKTCTIPA
QGTSMFPSCCCTKPSDGNCTCIPIPSSWAFARFLWEWASVRFSWLSLL
VPFVQWFVGLSPTVWLSVIWMMWYWGPSLYNILSPFLPLLPIFFCLWV
YI
3.3. HBV-CPₘᵤₜS: Description for amino acid
sequences of HBV immunogen
3.3.1. C =
3.3.1.1. PreCore =
(SEQ ID NO: 16)
MQLFHLCLIISCSCPTVQASKLCLGWLWG
3.3.1.2. Core =
(SEQ ID NO: 6)
MDIDPYKEFGASVELLSFLPSDFFPSIRDLLDTASALYREALESPEHC
SPHHTALRQAILCWGELMNLATWVGSNLEDPASRELVVSYVNVNMGLK
IRQLLWFHISCLTFGRETVLEYLVSFGVWIRTPPAYRPPNAPILSTLP
ETTVVRRRGRSPRRRTPSPRRRRSQSPRRRRSQSRESQC
3.3.2. Linker =
(SEQ ID NO: 7)
GGGSGGG
3.3.3. Pmut = (mutations: Y63A, C323A, C334A,
C338A, C352A, R714A, D788A, R792A)
(SEQ ID NO: 8)
MPLSYQHFRKLLLLDDEAGPLEEELPRLADEGLNRRVAEDLNLGNLNV
SIPWTHKVGNFTGLASSTVPVFNPEWQTPSFPHIHLQEDIINRCQQYV
GPLTVNEKRRLKLIMPARFYPNLTKYLPLDKGIKPYYPEHAVNHYFKT
RHYLHTLWKAGILYKRETTRSASFCGSPYSWEQELQHGRLVFQTSTRH
GDESFCSQSSGILSRSPVGPCVRSQLKQSRLGLQPQQGSLARGKSGRS
GSIRARVHPTTRRSFGVEPSGSGHIDNSASSTSSCLHQSAVRKTAYSH
LSTSKRQSSSGHAVELHNIPPSSARSQSEGPIFSAWWLQFRNSKPASD
YALTHIVNLLEDWGPATEHGEHNIRIPRTPARVTGGVFLVDKNPHNTT
ESRLVVDFSQFSRGSTHVSWPKFAVPNLQSLTNLLSSNLSTFGRKLHL
YSHPIILGFRKIPMGVGLSPFLLAQFTSAICSVVRRAFPHCLAFSYMD
DVVLGAKSVQHLESLFTSITNFLLSLGIHLNPNKTKRWGYSLNFMGYV
IGSWGTLPQEHIVLKIKQCFRKLPVNRPIDWKVCQRIVGLLGFAAPFT
QCGYPALMPLYACIQSKQAFTFSPTYKAFLCKQYLNLYPVARQRSGLC
QVFADATPTGWGLAIGHRAMRGTFVAPLPIHTAELLAACFARSRSGAK
LIGTDNSVVLSRKYTSFPWLLGCAANWILRGTSFVYVPSALNPAADPS
AGRLGLYRPLLHLPFRPTTGRTSLYAVSPSVPSHLPDRVHFASPLHVA
WRPP
3.3.4. Furin 2A (F2A) =
(SEQ ID NO: 9)
RKRRAPVKQTLNFDLLKLAGDVESNPGP
3.3.5. Surface proteins (S) =
3.3.5.1. PreS1 =
(SEQ ID NO: 52)
MGGWSSKPRQGMGTNLSVPNPLGFFPDHQLDPAFGANSNNPDWDFNPN
KDHWPEANQVGAGAFGPGFTPPHGGLLGWSPQAQGILTTVPAAPPPAS
TNRQSGRQPTPISPPLRDSHPQA
3.3.5.2. PreS2 =
(SEQ ID NO: 53)
MQWNSTTFHQALLDPRVRGLYFPAGGSSSGTVNPVPTTASPISSIFSR
TGDPAPN
3.3.5.3. Surface (S)
(SEQ ID NO: 10)
MENTTSGFLGPLLVLQAGFFLLTRILTIPQSLDSWWTSLNFLGGAPTC
PGQNSQSPTSNHSPTSCPPICPGYRWMCLRRFIIFLFILLLCLIFLLV
LLDYQGMLPVCPLLPGTSTTSTGPCKTCTIPAQGTSMFPSCCCTKPSD
GNCTCIPIPSSWAFARFLWEWASVRFSWLSLLVPFVQWFVGLSPTVWL
SVIWMMWYWGPSLYNILSPFLPLLPIFFCLWVYI

4. SIi-HBV-CPₘᵤₜPreS-S(sh)

4.1. SIi-HBV-CPₘᵤₜPreS-S(sh): Immunogen layout is shown in FIG. 10D

4.2. SIi-HBV-CPₘᵤₜPreS-S(sh): Amino acid sequence
(SEQ ID NO: 13)
MSLLWGGVTVLAAMLIAGQVASVVFLVKGGGPGGGMQLFHLCLIISCS
CPTVQASKLCLGWLWGMDIDPYKEFGASVELLSFLPSDFFPSIRDLLD
TASALYREALESPEHCSPHHTALRQAILCWGELMNLATWVGSNLEDPA
SRELVVSYVNVNMGLKIRQLLWFHISCLTFGRETVLEYLVSFGVWIRT
PPAYRPPNAPILSTLPETTVVRRRGRSPRRRTPSPRRRRSQSPRRRRS
QSRESQCGGGSGGGMPLSYQHFRKLLLLDDEAGPLEEELPRLADEGLN
RRVAEDLNLGNLNVSIPWTHKVGNFTGLASSTVPVFNPEWQTPSFPHI
HLQEDIINRCQQYVGPLTVNEKRRLKLIMPARFYPNLTKYLPLDKGIK
PYYPEHAVNHYFKTRHYLHTLWKAGILYKRETTRSASFCGSPYSWEQE
LQHGRLVFQTSTRHGDESFCSQSSGILSRSPVGPCVRSQLKQSRLGLQ
PQQGSLARGKSGRSGSIRARVHPTTRRSFGVEPSGSGHIDNSASSTSS
CLHQSAVRKTAYSHLSTSKRQSSSGHAVELHNIPPSSARSQSEGPIFS
AWWLQFRNSKPASDYALTHIVNLLEDWGPATEHGEHNIRIPRTPARVT
GGVFLVDKNPHNTTESRLVVDFSQFSRGSTHVSWPKFAVPNLQSLTNL
LSSNLSTFGRKLHLYSHPIILGFRKIPMGVGLSPFLLAQFTSAICSVV
RRAFPHCLAFSYMDDVVLGAKSVQHLESLFTSITNFLLSLGIHLNPNK
TKRWGYSLNFMGYVIGSWGTLPQEHIVLKIKQCFRKLPVNRPIDWKVC
QRIVGLLGFAAPFTQCGYPALMPLYACIQSKQAFTFSPTYKAFLCKQY
LNLYPVARQRSGLCQVFADATPTGWGLAIGHRAMRGTFVAPLPIHTAE
LLAACFARSRSGAKLIGTDNSVVLSRKYTSFPWLLGCAANWILRGTSF
VYVPSALNPAADPSAGRLGLYRPLLHLPFRPTTGRTSLYAVSPSVPSH
LPDRVHFASPLHVAWRPPKSPNSNNPDWDFNPNKDHWPEANQVGAGAF
GPGFTPPHGGLLGWSPQAQGILTTVPAAPPPASTNRQSGRQPTPISPP
LRDSHPQAMQWNSTTFHQALLDPRVRGLYFPAGGSSSGTVNPVPTTAS
PISSIFSRTGDPAPNGSKGKRKRRAPVKQTLNFDLLKLAGDVESNPGP
MGGWSSKPRQGMGTNLSVPNPLGFFPDHQLDPAFGANSNNPDWDFNPN
KDHWPEANQVGLEGGSGGMENTTSGFLGPLLVLQAGFFLLTRILTIPQ
SLDSWWTSLNFLGGAPTCPGQNSQSPTSNHSPTSCPPICPGYRWMCLR
RFIIFLFILLLCLIFLLVLLDYQGMLPVCPLLPGTSTTSTGPCKTCTI
PAQGTSMFPSCCCTKPSDGNCTCIPIPSSWAFARFLWEWASVRFSWLS
LLVPFVQWFVGLSPTVWLSVIWMMWYWGPSLYNILSPFLPLLPIFFCL
WVYI
4.3. SIi-HBV-CPₘᵤₜPreS-S(sh): Description for
amino acid sequences of HBV immunogen
4.3.1. First amino acid of polypeptide = M
4.3.2. Shark Invariant chain (SIi) =
(SEQ ID NO: 4)
SLLWGGVTVLAAMLIAGQVASVVFLV
4.3.3. Linker =
(SEQ ID NO: 5)
KGGGPGGG
4.3.4. C =
4.3.4.1. PreCore =
(SEQ ID NO: 16)
MQLFHLCLIISCSCPTVQASKLCLGWLWG
4.3.4.2. Core =
(SEQ ID NO: 6)
MDIDPYKEFGASVELLSFLPSDFFPSIRDLLDTASALYREALESPEHC
SPHHTALRQAILCWGELMNLATWVGSNLEDPASRELVVSYVNVNMGLK
IRQLLWFHISCLTFGRETVLEYLVSFGVWIRTPPAYRPPNAPILSTLP
ETTVVRRRGRSPRRRTPSPRRRRSQSPRRRRSQSRESQC
4.3.5. Linker =
(SEQ ID NO: 7)
GGGSGGG
4.3.6. Pmut = (mutations: Y63A, C323A, C334A,
C338A, C352A, R714A, D788A, R792A)
(SEQ ID NO: 8)
MPLSYQHFRKLLLLDDEAGPLEEELPRLADEGLNRRVAEDLNLGNLNV
SIPWTHKVGNFTGLASSTVPVFNPEWQTPSFPHIHLQEDIINRCQQYV
GPLTVNEKRRLKLIMPARFYPNLTKYLPLDKGIKPYYPEHAVNHYFKT
RHYLHTLWKAGILYKRETTRSASFCGSPYSWEQELQHGRLVFQTSTRH
GDESFCSQSSGILSRSPVGPCVRSQLKQSRLGLQPQQGSLARGKSGRS
GSIRARVHPTTRRSFGVEPSGSGHIDNSASSTSSCLHQSAVRKTAYSH
LSTSKRQSSSGHAVELHNIPPSSARSQSEGPIFSAWWLQFRNSKPASD
YALTHIVNLLEDWGPATEHGEHNIRIPRTPARVTGGVFLVDKNPHNTT
ESRLVVDFSQFSRGSTHVSWPKFAVPNLQSLTNLLSSNLSTFGRKLHL
YSHPIILGFRKIPMGVGLSPFLLAQFTSAICSVVRRAFPHCLAFSYMD
DVVLGAKSVQHLESLFTSITNFLLSLGIHLNPNKTKRWGYSLNFMGYV
IGSWGTLPQEHIVLKIKQCFRKLPVNRPIDWKVCQRIVGLLGFAAPFT
QCGYPALMPLYACIQSKQAFTFSPTYKAFLCKQYLNLYPVARQRSGLC
QVFADATPTGWGLAIGHRAMRGTFVAPLPIHTAELLAACFARSRSGAK
LIGTDNSVVLSRKYTSFPWLLGCAANWILRGTSFVYVPSALNPAADPS
AGRLGLYRPLLHLPFRPTTGRTSLYAVSPSVPSHLPDRVHFASPLHVA
WRPP
4.3.7. Linker =
KSP
4.3.8. NΔPreS1 and PreS2 =
(SEQ ID NO: 15)
NSNNPDWDFNPNKDHWPEANQVGAGAFGPGFTPPHGGLLGWSPQAQGI
LTTVPAAPPPASTNRQSGRQPTPISPPLRDSHPQAMQWNSTTFHQALL
DPRVRGLYFPAGGSSSGTVNPVPTTASPISSIFSRTGDPAPN
4.3.9. Linker =
(SEQ ID NO: 20)
GSKGK
4.3.10. Furin 2A (F2A) =
(SEQ ID NO: 9)
RKRRAPVKQTLNFDLLKLAGDVESNPGP
4.3.11. S(sh) =
4.3.11.1. CΔPreS1 =
(SEQ ID NO: 21)
MGGWSSKPRQGMGTNLSVPNPLGFFPDHQLDPAFGANSNNPDWDFNPN
KDHWPEANQVG
4.3.11.2. Linker =
(SEQ ID NO: 22)
LEGGSGG
4.3.11.3. Surface (S) =
(SEQ ID NO: 18)
MENTTSGFLGPLLVLQAGFFLLTRILTIPQSLDSWWTSLNFLGGAPTC
PGQNSQSPTSNHSPTSCPPICPGYRWMCLRRFIIFLFILLLCLIFLLV
LLDYQGMLPVCPLLPGTSTTSTGPCKTCTIPAQGTSMFPSCCCTKPSD
GNCTCIPIPSSWAFARFLWEWASVRFSWLSLLVPFVQWFVGLSPTVWL
SVIWMMWYWGPSLYNILSPFLPLLPIFFCLWVYI

The SIi-HBV-CPmutPreS-S(sh) nucleotide sequence is provided as SEQ ID NO: 48.

5. MVA-SIi-HBV-PreS-Pₘᵤₜ-C-S(sh)

5.1. MVA-SIi-HBV-PreS-Pₘᵤₜ-C-S(Sh): Immunogen Layout is Shown in FIG.

5.2. MVA-SIi-HBV-PreS-Pₘᵤₜ-C-S(sh): Amino acid
sequence
(SEQ ID NO: 23)
MSLLWGGVTVLAAMLIAGQVASVVFLVSKSGPPSGKSNSNNPDWDFNP
NKDHWPEANQVGAGAFGPGFTPPHGGLLGWSPQAQGILTTVPAAPPPA
STNRQSGRQPTPISPPLRDSHPQAMQWNSTTFHQALLDPRVRGLYFPA
GGSSSGTVNPVPTTASPISSIFSRTGDPAPNGSKSGSKMPLSYQHFRK
LLLLDDEAGPLEEELPRLADEGLNRRVAEDLNLGNLNVSIPWTHKVGN
FTGLASSTVPVFNPEWQTPSFPHIHLQEDIINRCQQYVGPLTVNEKRR
LKLIMPARFYPNLTKYLPLDKGIKPYYPEHAVNHYFKTRHYLHTLWKA
GILYKRETTRSASFCGSPYSWEQELQHGRLVFQTSTRHGDESFCSQSS
GILSRSPVGPCVRSQLKQSRLGLQPQQGSLARGKSGRSGSIRARVHPT
TRRSFGVEPSGSGHIDNSASSTSSCLHQSAVRKTAYSHLSTSKRQSSS
GHAVELHNIPPSSARSQSEGPIFSAWWLQFRNSKPASDYALTHIVNLL
EDWGPATEHGEHNIRIPRTPARVTGGVFLVDKNPHNTTESRLVVDFSQ
FSRGSTHVSWPKFAVPNLQSLTNLLSSNLSWLSLDVSAAFYHIPLHPA
AMPHLLVGSSGLPRYVARLSSTSRNINYQHGTMQDLHDSCSRNLYVSL
LLLYKTFGRKLHLYSHPIILGFRKIPMGVGLSPFLLAQFTSAICSVVR
RAFPHCLAFSYMDDVVLGAKSVQHLESLFTSITNFLLSLGIHLNPNKT
KRWGYSLNFMGYVIGSWGTLPQEHIVLKIKQCFRKLPVNRPIDWKVCQ
RIVGLLGFAAPFTQCGYPALMPLYACIQSKQAFTFSPTYKAFLCKQYL
NLYPVARQRSGLCQVFADATPTGWGLAIGHRAMRGTFVAPLPIHTAEL
LAACFARSRSGAKLIGTDNSVVLSRKYTSFPWLLGCAANWILRGTSFV
YVPSALNPAADPSAGRLGLYRPLLHLPFRPTTGRTSLYAVSPSVPSHL
PDRVHFASPLHVAWRPPSKSPGSGPPMQLFHLCLIISCSCPTVQASKL
CLGWLWGMDIDPYKEFGASVELLSFLPSDFFPSIRDLLDTASALYREA
LESPEHCSPHHTALRQAILCWGELMNLATWVGSNLEDPASRELVVSYV
NVNMGLKIRQLLWFHISCLTFGRETVLEYLVSFGVWIRTPPAYRPPNA
PILSTLPETTVVRRRGRSPRRRTPSPRRRRSQSPRRRRSQSRESQC
MGGWSSKPRQGMGTNLSVPNPLGFFPDHQLDPAFGANSNNPDWDFNPN
KDHWPEANQVGASKGGKSGMENTTSGFLGPLLVLQAGFFLLTRILTIP
QSLDSWWTSLNFLGGAPTCPGQNSQSPTSNHSPTSCPPICPGYRWMCL
RRFIIFLFILLLCLIFLLVLLDYQGMLPVCPLLPGTSTTSTGPCKTCT
IPAQGTSMFPSCCCTKPSDGNCTCIPIPSSWAFARFLWEWASVRFSWL
SLLVPFVQWFVGLSPTVWLSVIWMMWYWGPSLYNILSPFLPLLPIFFC
LWVYI
5.3. MVA-SIi-HBV-PreS-Pₘᵤₜ-C-S(sh): Description
for amino acid sequences of HBV immunogen
5.3.1.1. First amino acid of polypeptide = M
5.3.1.2. Shark Invariant chain (SIi) =
(SEQ ID NO: 4)
SLLWGGVTVLAAMLIAGQVASVVFLV
5.3.1.3. Linker =
(SEQ ID NO: 31)
SKSGPPSGKS
5.3.1.4. NΔPreS1 and PreS2 =
(SEQ ID NO: 15)
NSNNPDWDFNPNKDHWPEANQVGAGAFGPGFTPPHGGLLGWSPQAQGI
LTTVPAAPPPASTNRQSGRQPTPISPPLRDSHPQAMQWNSTTFHQALL
DPRVRGLYFPAGGSSSGTVNPVPTTASPISSIFSRTGDPAPN
5.3.1.5. Linker =
(SEQ ID NO: 32)
GSKSGSK
5.3.1.6. Pmut = (mutations: Y63A, C323A, C334A,
C338A, C352A, R714A, D788A, R792A)
(SEQ ID NO: 8)
MPLSYQHFRKLLLLDDEAGPLEEELPRLADEGLNRRVAEDLNLGNLNV
SIPWTHKVGNFTGLASSTVPVFNPEWQTPSFPHIHLQEDIINRCQQYV
GPLTVNEKRRLKLIMPARFYPNLTKYLPLDKGIKPYYPEHAVNHYFKT
RHYLHTLWKAGILYKRETTRSASFCGSPYSWEQELQHGRLVFQTSTRH
GDESFCSQSSGILSRSPVGPCVRSQLKQSRLGLQPQQGSLARGKSGRS
GSIRARVHPTTRRSFGVEPSGSGHIDNSASSTSSCLHQSAVRKTAYSH
LSTSKRQSSSGHAVELHNIPPSSARSQSEGPIFSAWWLQFRNSKPASD
YALTHIVNLLEDWGPATEHGEHNIRIPRTPARVTGGVFLVDKNPHNTT
ESRLVVDFSQFSRGSTHVSWPKFAVPNLQSLTNLLSSNLSTFGRKLHL
YSHPIILGFRKIPMGVGLSPFLLAQFTSAICSVVRRAFPHCLAFSYMD
DVVLGAKSVQHLESLFTSITNFLLSLGIHLNPNKTKRWGYSLNFMGYV
IGSWGTLPQEHIVLKIKQCFRKLPVNRPIDWKVCQRIVGLLGFAAPFT
QCGYPALMPLYACIQSKQAFTFSPTYKAFLCKQYLNLYPVARQRSGLC
QVFADATPTGWGLAIGHRAMRGTFVAPLPIHTAELLAACFARSRSGAK
LIGTDNSVVLSRKYTSFPWLLGCAANWILRGTSFVYVPSALNPAADPS
AGRLGLYRPLLHLPFRPTTGRTSLYAVSPSVPSHLPDRVHFASPLHVA
WRPP
5.3.1.7. Linker =
(SEQ ID NO: 33)
SKSPGSGPP
5.3.1.8. C =
5.3.1.8.1. PreCore
(SEQ ID NO: 16)
MQLFHLCLIISCSCPTVQASKLCLGWLWG
5.3.1.8.2. Core =
(SEQ ID NO: 6)
MDIDPYKEFGASVELLSFLPSDFFPSIRDLLDTASALYREALESPEHC
SPHHTALRQAILCWGELMNLATWVGSNLEDPASRELVVSYVNVNMGLK
IRQLLWFHISCLTFGRETVLEYLVSFGVWIRTPPAYRPPNAPILSTLP
ETTVVRRRGRSPRRRTPSPRRRRSQSPRRRRSQSRESQC
5.3.1.9. S(sh)
5.3.1.9.1. CΔPreS1 =
MGGWSSKPRQGMGTNLSVPNPLGFFPDHQLDPAFGANSNNPDWDFNPN
KDHWPEANQVG
5.3.1.9.2. Linker =
(SEQ ID NO: 34)
ASKGGKSG
5.3.1.9.3. Surface =
(SEQ ID NO: 18)
MENTTSGFLGPLLVLQAGFFLLTRILTIPQSLDSWWTSLNFLGGAPTC
PGQNSQSPTSNHSPTSCPPICPGYRWMCLRRFIIFLFILLLCLIFLLV
LLDYQGMLPVCPLLPGTSTTSTGPCKTCTIPAQGTSMFPSCCCTKPSD
GNCTCIPIPSSWAFARFLWEWASVRFSWLSLLVPFVQWFVGLSPTVWL
SVIWMMWYWGPSLYNILSPFLPLLPIFFCLWVYI
5.4. Nucleotide sequence of MVA-SIi-HBV-PreS-
Pₘᵤₜ-C-S(sh) is provided as SEQ ID NO: 24.
5.5. Description for nucleotide sequences of
MVA-SIi-HBV-PreS-Pₘᵤₜ-C-S(sh):
5.5.1. F11-L-Flank = bases 1-1097
(SEQ ID NO: 35)
5.5.2. SIi-HBV-PreS-Pₘᵤₜ-C = bases 1098-4838
(SEQ ID NO: 37)
5.5.3. Transcription terminator sequence =
bases 4839-4845
TTTTTGT
5.5.4. mH5 promoter = bases 4846-4942
(SEQ ID NO: 28)
5.5.5. S(sh) = bases 4943-5824 (SEQ ID NO: 38)
5.5.6. F11-R-Flank = bases 5825-7143
(SEQ ID NO: 36)

6. SIi-HBV-CPQₘᵤₜPreS-TPA-S(sh)

6.1. SIi-HBV-CPₘᵤₜPreS-TPA-S(Sh): Immunogen Layout is Shown in FIG. 10F

6.2. SIi-HBV-CPₘᵤₜPreS-TPA-S(sh): Amino acid
sequence
(SEQ ID NO: 25)
MSLLWGGVTVLAAMLIAGQVASVVFLVKGGGPGGGMQLFHLCLIISCS
CPTVQASKLCLGWLWGMDIDPYKEFGASVELLSFLPSDFFPSIRDLLD
TASALYREALESPEHCSPHHTALRQAILCWGELMNLATWVGSNLEDPA
SRELVVSYVNVNMGLKIRQLLWFHISCLTFGRETVLEYLVSFGVWIRT
PPAYRPPNAPILSTLPETTVVRRRGRSPRRRTPSPRRRRSQSPRRRRS
QSRESQCGGGSGGGMPLSYQHFRKLLLLDDEAGPLEEELPRLADEGLN
RRVAEDLNLGNLNVSIPWTHKVGNFTGLASSTVPVFNPEWQTPSFPHI
HLQEDIINRCQQYVGPLTVNEKRRLKLIMPARFYPNLTKYLPLDKGIK
PYYPEHAVNHYFKTRHYLHTLWKAGILYKRETTRSASFCGSPYSWEQE
LQHGRLVFQTSTRHGDESFCSQSSGILSRSPVGPCVRSQLKQSRLGLQ
PQQGSLARGKSGRSGSIRARVHPTTRRSFGVEPSGSGHIDNSASSTSS
CLHQSAVRKTAYSHLSTSKRQSSSGHAVELHNIPPSSARSQSEGPIFS
AWWLQFRNSKPASDYALTHIVNLLEDWGPATEHGEHNIRIPRTPARVT
GGVFLVDKNPHNTTESRLVVDFSQFSRGSTHVSWPKFAVPNLQSLTNL
LSSNLSTFGRKLHLYSHPIILGFRKIPMGVGLSPFLLAQFTSAICSVV
RRAFPHCLAFSYMDDVVLGAKSVQHLESLFTSITNFLLSLGIHLNPNK
TKRWGYSLNFMGYVIGSWGTLPQEHIVLKIKQCFRKLPVNRPIDWKVC
QRIVGLLGFAAPFTQCGYPALMPLYACIQSKQAFTFSPTYKAFLCKQY
LNLYPVARQRSGLCQVFADATPTGWGLAIGHRAMRGTFVAPLPIHTAE
LLAACFARSRSGAKLIGTDNSVVLSRKYTSFPWLLGCAANWILRGTSF
VYVPSALNPAADPSAGRLGLYRPLLHLPFRPTTGRTSLYAVSPSVPSH
LPDRVHFASPLHVAWRPPKSPNSNNPDWDFNPNKDHWPEANQVGAGAF
GPGFTPPHGGLLGWSPQAQGILTTVPAAPPPASTNRQSGRQPTPISPP
LRDSHPQAMQWNSTTFHQALLDPRVRGLYFPAGGSSSGTVNPVPTTAS
PISSIFSRTGDPAPNGSKGKRKRRAPVKQTLNFDLLKLAGDVESNPGP
MDAMKRGLCCVLLLCGAVFVSPSQEIHARFRRMGGWSSKPRQGMGTNL
SVPNPLGFFPDHQLDPAFGANSNNPDWDFNPNKDHWPEANQVGLEGGS
GGMENTTSGFLGPLLVLQAGFFLLTRILTIPQSLDSWWTSLNFLGGAP
TCPGQNSQSPTSNHSPTSCPPICPGYRWMCLRRFIIFLFILLLCLIFL
LVLLDYQGMLPVCPLLPGTSTTSTGPCKTCTIPAQGTSMFPSCCCTKP
SDGNCTCIPIPSSWAFARFLWEWASVRFSWLSLLVPFVQWFVGLSPTV
WLSVIWMMWYWGPSLYNILSPFLPLLPIFFCLWVYI
6.3. SIi-HBV-CPₘᵤₜPreS-TPA-S(sh): Description for
amino acid sequences of HBV immunogen
6.3.1. First amino acid of polypeptide = M
6.3.2. Shark Invariant chain (SIi) =
(SEQ ID NO: 4)
SLLWGGVTVLAAMLIAGQVASVVFLV
6.3.3. Linker =
(SEQ ID NO: 5)
KGGGPGGG
6.3.4. C =
6.3.4.1. PreCore
(SEQ ID NO: 16)
MQLFHLCLIISCSCPTVQASKLCLGWLWG
6.3.4.2. Core =
(SEQ ID NO: 6)
MDIDPYKEFGASVELLSFLPSDFFPSIRDLLDTASALYREALESPEHC
SPHHTALRQAILCWGELMNLATWVGSNLEDPASRELVVSYVNVNMGLK
IRQLLWFHISCLTFGRETVLEYLVSFGVWIRTPPAYRPPNAPILSTLP
ETTVVRRRGRSPRRRTPSPRRRRSQSPRRRRSQSRESQC
6.3.5. Linker =
(SEQ ID NO: 7)
GGGSGGG
6.3.6. Pmut = (mutations: Y63A, C323A, C334A,
C338A, C352A, R714A, D788A, R792A)
(SEQ ID NO: 8)
MPLSYQHFRKLLLLDDEAGPLEEELPRLADEGLNRRVAEDLNLGNLNV
SIPWTHKVGNFTGLASSTVPVFNPEWQTPSFPHIHLQEDIINRCQQYV
GPLTVNEKRRLKLIMPARFYPNLTKYLPLDKGIKPYYPEHAVNHYFKT
RHYLHTLWKAGILYKRETTRSASFCGSPYSWEQELQHGRLVFQTSTRH
GDESFCSQSSGILSRSPVGPCVRSQLKQSRLGLQPQQGSLARGKSGRS
GSIRARVHPTTRRSFGVEPSGSGHIDNSASSTSSCLHQSAVRKTAYSH
LSTSKRQSSSGHAVELHNIPPSSARSQSEGPIFSAWWLQFRNSKPASD
YALTHIVNLLEDWGPATEHGEHNIRIPRTPARVTGGVFLVDKNPHNTT
ESRLVVDFSQFSRGSTHVSWPKFAVPNLQSLTNLLSSNLSTFGRKLHL
YSHPIILGFRKIPMGVGLSPFLLAQFTSAICSVVRRAFPHCLAFSYMD
DVVLGAKSVQHLESLFTSITNFLLSLGIHLNPNKTKRWGYSLNFMGYV
IGSWGTLPQEHIVLKIKQCFRKLPVNRPIDWKVCQRIVGLLGFAAPFT
QCGYPALMPLYACIQSKQAFTFSPTYKAFLCKQYLNLYPVARQRSGLC
QVFADATPTGWGLAIGHRAMRGTFVAPLPIHTAELLAACFARSRSGAK
LIGTDNSVVLSRKYTSFPWLLGCAANWILRGTSFVYVPSALNPAADPS
AGRLGLYRPLLHLPFRPTTGRTSLYAVSPSVPSHLPDRVHFASPLHVA
WRPP
6.3.7. Linker =
KSP
6.3.8. NΔPreS1 and PreS2 =
(SEQ ID NO: 15)
NSNNPDWDFNPNKDHWPEANQVGAGAFGPGFTPPHGGLLGWSPQAQGI
LTTVPAAPPPASTNRQSGRQPTPISPPLRDSHPQAMQWNSTTFHQALL
DPRVRGLYFPAGGSSSGTVNPVPTTASPISSIFSRTGDPAPN
6.3.9. Linker =
(SEQ ID NO: 20)
GSKGK
6.3.10. Furin 2A (F2A) =
(SEQ ID NO: 9)
RKRRAPVKQTLNFDLLKLAGDVESNPGP
6.3.11. TPA =
(SEQ ID NO: 30)
MDAMKRGLCCVLLLCGAVFVSPSQEIHARFRR
6.3.12. S(sh) =
6.3.12.1. CΔPreS1 =
(SEQ ID NO: 21)
MGGWSSKPRQGMGTNLSVPNPLGFFPDHQLDPAFGANSNNPDWDFNPN
KDHWPEANQVG
6.3.12.2. Linker =
(SEQ ID NO: 22)
LEGGSGG
6.3.12.3. Surface (S) =
(SEQ ID NO: 18)
MENTTSGFLGPLLVLQAGFFLLTRILTIPQSLDSWWTSLNFLGGAPTC
PGQNSQSPTSNHSPTSCPPICPGYRWMCLRRFIIFLFILLLCLIFLLV
LLDYQGMLPVCPLLPGTSTTSTGPCKTCTIPAQGTSMFPSCCCTKPSD
GNCTCIPIPSSWAFARFLWEWASVRFSWLSLLVPFVQWFVGLSPTVWL
SVIWMMWYWGPSLYNILSPFLPLLPIFFCLWVYI

The SIi-HBV-CPmutPreS-TPA-S(sh) nucleotide sequence is provided as SEQ ID NO: 49.

7. MVA-SIi-HBV-PreS-Pₘᵤₜ-C-TPA-S(sh)

7.1. MVA-SIi-HBV-PreS-Pₘᵤₜ-C-TPA-S(Sh): Immunogen Layout is Shown in FIG. 10G

7.2. MVA-SIi-HBV-PreS-Pₘᵤₜ-C-TPA-S(sh): Amino
acid sequence
(SEQ ID NO: 26)
MSLLWGGVTVLAAMLIAGQVASVVFLVSKSGPPSGKSNSNNPDWDFNP
NKDHWPEANQVGAGAFGPGFTPPHGGLLGWSPQAQGILTTVPAAPPPA
STNRQSGRQPTPISPPLRDSHPQAMQWNSTTFHQALLDPRVRGLYFPA
GGSSSGTVNPVPTTASPISSIFSRTGDPAPNGSKSGSKMPLSYQHFRK
LLLLDDEAGPLEEELPRLADEGLNRRVAEDLNLGNLNVSIPWTHKVGN
FTGLASSTVPVFNPEWQTPSFPHIHLQEDIINRCQQYVGPLTVNEKRR
LKLIMPARFYPNLTKYLPLDKGIKPYYPEHAVNHYFKTRHYLHTLWKA
GILYKRETTRSASFCGSPYSWEQELQHGRLVFQTSTRHGDESFCSQSS
GILSRSPVGPCVRSQLKQSRLGLQPQQGSLARGKSGRSGSIRARVHPT
TRRSFGVEPSGSGHIDNSASSTSSCLHQSAVRKTAYSHLSTSKRQSSS
GHAVELHNIPPSSARSQSEGPIFSAWWLQFRNSKPASDYALTHIVNLL
EDWGPATEHGEHNIRIPRTPARVTGGVFLVDKNPHNTTESRLVVDFSQ
FSRGSTHVSWPKFAVPNLQSLTNLLSSNLSWLSLDVSAAFYHIPLHPA
AMPHLLVGSSGLPRYVARLSSTSRNINYQHGTMQDLHDSCSRNLYVSL
LLLYKTFGRKLHLYSHPIILGFRKIPMGVGLSPFLLAQFTSAICSVVR
RAFPHCLAFSYMDDVVLGAKSVQHLESLFTSITNFLLSLGIHLNPNKT
KRWGYSLNFMGYVIGSWGTLPQEHIVLKIKQCFRKLPVNRPIDWKVCQ
RIVGLLGFAAPFTQCGYPALMPLYACIQSKQAFTFSPTYKAFLCKQYL
NLYPVARQRSGLCQVFADATPTGWGLAIGHRAMRGTFVAPLPIHTAEL
LAACFARSRSGAKLIGTDNSVVLSRKYTSFPWLLGCAANWILRGTSFV
YVPSALNPAADPSAGRLGLYRPLLHLPFRPTTGRTSLYAVSPSVPSHL
PDRVHFASPLHVAWRPPSKSPGSGPPMQLFHLCLIISCSCPTVQASKL
CLGWLWGMDIDPYKEFGASVELLSFLPSDFFPSIRDLLDTASALYREA
LESPEHCSPHHTALRQAILCWGELMNLATWVGSNLEDPASRELVVSYV
NVNMGLKIRQLLWFHISCLTFGRETVLEYLVSFGVWIRTPPAYRPPNA
PILSTLPETTVVRRRGRSPRRRTPSPRRRRSQSPRRRRSQSRESQCMD
AMKRGLCCVLLLCGAVFVSPSQEIHARFRRMGGWSSKPRQGMGTNLSV
PNPLGFFPDHQLDPAFGANSNNPDWDFNPNKDHWPEANQVGASKGGKS
GMENTTSGFLGPLLVLQAGFFLLTRILTIPQSLDSWWTSLNFLGGAPT
CPGQNSQSPTSNHSPTSCPPICPGYRWMCLRRFIIFLFILLLCLIFLL
VLLDYQGMLPVCPLLPGTSTTSTGPCKTCTIPAQGTSMFPSCCCTKPS
DGNCTCIPIPSSWAFARFLWEWASVRFSWLSLLVPFVQWFVGLSPTVW
LSVIWMMWYWGPSLYNILSPFLPLLPIFFCLWVYI
7.3. MVA-SIi-HBV-PreS-Pₘᵤₜ-C-TPA-S(sh):
Description for amino acid sequences of HBV
immunogen
7.3.1.1. First amino acid of polypeptide = M
7.3.1.2. Shark Invariant chain (SIi) =
(SEQ ID NO: 4)
SLLWGGVTVLAAMLIAGQVASVVFLV
7.3.1.3. Linker =
(SEQ ID NO: 31)
SKSGPPSGKS
7.3.1.4. NΔPreS1 and PreS2 =
(SEQ ID NO: 15)
NSNNPDWDFNPNKDHWPEANQVGAGAFGPGFTPPHGGLLGWSPQAQGI
LTTVPAAPPPASTNRQSGRQPTPISPPLRDSHPQAMQWNSTTFHQALL
DPRVRGLYFPAGGSSSGTVNPVPTTASPISSIFSRTGDPAPN
7.3.1.5. Linker =
(SEQ ID NO: 32)
GSKSGSK
7.3.1.6. Pmut = (mutations: Y63A, C323A, C334A,
C338A, C352A, R714A, D788A, R792A)
(SEQ ID NO: 8)
MPLSYQHFRKLLLLDDEAGPLEEELPRLADEGLNRRVAEDLNLGNLNV
SIPWTHKVGNFTGLASSTVPVFNPEWQTPSFPHIHLQEDIINRCQQYV
GPLTVNEKRRLKLIMPARFYPNLTKYLPLDKGIKPYYPEHAVNHYFKT
RHYLHTLWKAGILYKRETTRSASFCGSPYSWEQELQHGRLVFQTSTRH
GDESFCSQSSGILSRSPVGPCVRSQLKQSRLGLQPQQGSLARGKSGRS
GSIRARVHPTTRRSFGVEPSGSGHIDNSASSTSSCLHQSAVRKTAYSH
LSTSKRQSSSGHAVELHNIPPSSARSQSEGPIFSAWWLQFRNSKPASD
YALTHIVNLLEDWGPATEHGEHNIRIPRTPARVTGGVFLVDKNPHNTT
ESRLVVDFSQFSRGSTHVSWPKFAVPNLQSLTNLLSSNLSTFGRKLHL
YSHPIILGFRKIPMGVGLSPFLLAQFTSAICSVVRRAFPHCLAFSYMD
DVVLGAKSVQHLESLFTSITNFLLSLGIHLNPNKTKRWGYSLNFMGYV
IGSWGTLPQEHIVLKIKQCFRKLPVNRPIDWKVCQRIVGLLGFAAPFT
QCGYPALMPLYACIQSKQAFTFSPTYKAFLCKQYLNLYPVARQRSGLC
QVFADATPTGWGLAIGHRAMRGTFVAPLPIHTAELLAACFARSRSGAK
LIGTDNSVVLSRKYTSFPWLLGCAANWILRGTSFVYVPSALNPAADPS
AGRLGLYRPLLHLPFRPTTGRTSLYAVSPSVPSHLPDRVHFASPLHVA
WRPP
7.3.1.7. Linker =
(SEQ ID NO: 33)
SKSPGSGPP
7.3.1.8. C =
7.3.1.8.1. PreCore =
(SEQ ID NO: 16)
MQLFHLCLIISCSCPTVQASKLCLGWLWG
7.3.1.8.2. Core =
(SEQ ID NO: 6)
MDIDPYKEFGASVELLSFLPSDFFPSIRDLLDTASALYREALESPEHC
SPHHTALRQAILCWGELMNLATWVGSNLEDPASRELVVSYVNVNMGLK
IRQLLWFHISCLTFGRETVLEYLVSFGVWIRTPPAYRPPNAPILSTLP
ETTVVRRRGRSPRRRTPSPRRRRSQSPRRRRSQSRESQC
7.3.1.9. TPA =
(SEQ ID NO: 30)
MDAMKRGLCCVLLLCGAVFVSPSQEIHARFRR
7.3.1.10. S(sh)_ =
7.3.1.10.1. CΔPreS1 =
(SEQ ID NO: 21)
MGGWSSKPRQGMGTNLSVPNPLGFFPDHQLDPAFGANSNNPDWDFNPN
KDHWPEANQVG
7.3.1.10.2. Linker =
(SEQ ID NO: 34)
ASKGGKSG
7.3.1.10.3. Surface (S) =
(SEQ ID NO: 18)
MENTTSGFLGPLLVLQAGFFLLTRILTIPQSLDSWWTSLNFLGGAPTC
PGQNSQSPTSNHSPTSCPPICPGYRWMCLRRFIIFLFILLLCLIFLLV
LLDYQGMLPVCPLLPGTSTTSTGPCKTCTIPAQGTSMFPSCCCTKPSD
GNCTCIPIPSSWAFARFLWEWASVRFSWLSLLVPFVQWFVGLSPTVWL
SVIWMMWYWGPSLYNILSPFLPLLPIFFCLWVYI
7.4. Nucleotide sequences of MVA-SIi-HBV-PreS-
Pₘᵤₜ-C-TPA-S(sh) is provided as SEQ ID NO: 27
7.5. Description for nucleotide sequences of
MVA-SIi-HBV-PreS-Pₘᵤₜ-C-TPA-S(sh):
7.5.1. F11-L-Flank = bases 1-1097
(SEQ ID NO: 35)
7.5.2. SIi-HBV-PreS-Pₘᵤₜ-C = bases 1098-4838
(SEQ ID NO: 37)
7.5.3. Transcription terminator sequence = bases
4839-4845
TTTTTGT
7.5.4. mH5 promoter = bases 4846-4942
(SEQ ID NO: 28)
7.5.5. TPA = bases 4943-5038 (SEQ ID NO: 29)
7.5.6. S(sh) = bases 5039-5920 (SEQ ID NO: 38)
7.5.7. F11-R-Flank = bases 5921-7239
(SEQ ID NO: 36)

8. Nucleotide sequences of low GC content
version MVA-SIi-HBV-PreS-Pmut-C-TPA-S(sh)
(SEQ ID NO: 58):
gtaatctattcgatataccgttgctaacagtatactggcccaataact
gtggatggaaaatctataataatacattaatatcatccgatggtgcta
gggttatttggatggatgcgtataaattttcttgcggtttatctttac
aagactattgttatcattggggtagcaaaccagagagccgaccattcg
atttaataaaaaaatcagatgctaaacgcaattctaaatcgttggtca
aagaatctatggcatccttgaaatccttgtacgaggcattcgagacac
aatcaggagcgttagaagttttaatgagtccatgtaggatgttttcgt
tttctagaatagaagacatgttcttaactagtgtcattaatagagtat
ccgagaatactggaatggggatgtattatcctaccaacgatatacctt
ctctatttatcgaatcatctatctgtctagattatattatagtaaata
atcaggaatccaacaaatatcgtatcaaatctgttctcgatatcattt
cttcaaaacaataccctgcaggacgtcccaactacgttaaaaatggta
caaaaggaaagttatatatcgcgttgtgtaaagttaccgtacctacta
acgaccatattccagtagtttatcacgatgatgacaatactaccacct
ttattacagtattgacgtccgtcgatattgaaactgctatcagagcag
gatattcgatagtcgaattaggggctttacaatgggataataatattc
cagaacttaaaaacggtttactggatagtatcaagatgatttatgact
tgaacgcagttacaacaaataatttattggaacagctcatagaaaata
ttaactttaacaactctagtataatttcgttgttttatacatttgcca
ttagttattgccgagcattcatttactcaattatggaaaccatagatc
cggtgtatatatctcagttcagttataaagaattatacgttagtagct
cttataaagatattaatgaatccatgagtcagatggtaaaattataaa
aagtgaaaaacaatattatttttatcgttggttgttacactATGTCAC
TTCTTTGGGGCGGAGTTACAGTTCTTGCTGCTATGCTTATTGCTGGAC
AAGTTGCTTCTGTTGTGTTTCTTGTTTCTAAATCTGGACCTCCTTCTG
GAAAATCTAATTCTAACAATCCTGATTGGGATTTCAATCCTAACAAAG
ATCATTGGCCTGAAGCTAATCAAGTTGGAGCTGGTGCTTTTGGACCTG
GTTTTACACCTCCTCATGGTGGATTGCTTGGATGGTCACCTCAAGCTC
AGGGAATTCTTACAACAGTTCCAGCTGCTCCTCCTCCTGCTTCTACAA
ATAGACAATCTGGTAGACAACCTACACCTATTTCTCCACCTCTTAGAG
ATTCTCATCCTCAAGCTATGCAATGGAATTCTACTACATTTCATCAAG
CTTTGCTTGATCCTAGAGTTAGAGGACTTTATTTTCCTGCTGGTGGAA
GTTCTTCTGGAACAGTTAATCCTGTTCCTACAACAGCTTCTCCAATTT
CTTCTATATTTTCTAGAACAGGCGATCCTGCTCCTAATGGATCAAAAT
CTGGATCAAAAATGCCTCTGTCTTATCAACACTTTAGAAAATTGCTGC
TTCTTGATGATGAAGCTGGACCTCTTGAAGAAGAATTGCCTAGACTTG
CTGATGAAGGACTTAATAGAAGAGTTGCTGAAGATCTTAATCTGGGAA
ATCTTAATGTTTCTATTCCTTGGACACACAAAGTTGGAAATTTCACAG
GACTTGCATCTTCTACAGTGCCTGTTTTTAATCCTGAATGGCAAACAC
CTTCTTTTCCACATATTCATCTGCAAGAGGATATCATCAATAGATGTC
AACAATATGTTGGACCACTGACAGTTAATGAGAAGAGAAGGCTTAAAC
TTATTATGCCTGCTAGATTCTATCCTAATCTTACAAAGTATTTGCCTC
TGGATAAGGGAATCAAACCTTATTATCCTGAACATGCTGTGAATCACT
ACTTTAAAACAAGACATTATCTGCATACACTGTGGAAAGCTGGTATTC
TTTACAAAAGAGAAACAACAAGATCTGCTTCATTTTGTGGATCTCCAT
ATTCTTGGGAACAAGAACTTCAACATGGTAGACTTGTTTTTCAAACAT
CTACAAGACATGGGGATGAATCATTTTGTTCTCAAAGTTCTGGAATTC
TTTCTAGATCTCCTGTTGGACCTTGTGTTAGATCTCAACTTAAACAAT
CTAGACTTGGACTTCAACCTCAACAAGGATCTCTTGCTAGAGGAAAAA
GTGGAAGATCTGGATCTATTAGAGCTAGAGTTCATCCTACAACTAGAA
GATCTTTTGGAGTTGAACCTTCTGGATCTGGACATATTGATAATTCTG
CCTCTTCTACATCTTCTTGTCTGCATCAATCTGCTGTTAGAAAGACAG
CTTATTCTCATTTGTCTACTTCTAAGAGACAATCATCTTCTGGACATG
CTGTTGAACTTCATAATATTCCTCCAAGTAGTGCTAGAAGTCAATCTG
AAGGACCAATATTTTCAGCTTGGTGGCTTCAATTCAGAAATTCTAAAC
CTGCTTCTGATTATGCTCTGACACATATAGTTAATTTGCTTGAAGATT
GGGGACCTGCTACAGAACATGGCGAACACAATATTAGAATACCTAGAA
CTCCTGCTAGAGTTACAGGCGGAGTCTTTTTGGTTGATAAGAATCCTC
ATAATACCACAGAATCAAGACTTGTTGTTGATTTTTCACAGTTTTCTA
GAGGATCTACACATGTTTCTTGGCCTAAATTTGCTGTTCCAAATCTTC
AATCTCTTACAAATTTGCTTTCATCTAATCTTTCTTGGCTGTCTCTTG
ATGTTTCTGCTGCCTTTTATCATATTCCTCTTCATCCTGCTGCAATGC
CTCATTTGCTTGTTGGATCATCTGGACTTCCAAGATATGTTGCTAGAC
TTAGCTCTACATCTAGAAATATCAATTATCAGCATGGAACAATGCAGG
ATCTTCACGATTCTTGTAGTAGGAATCTGTATGTTTCTTTGCTTCTGC
TGTATAAGACATTTGGAAGAAAACTTCATCTGTATTCTCACCCTATTA
TTCTGGGTTTTAGAAAGATTCCTATGGGAGTTGGACTTTCTCCTTTTT
TGCTTGCTCAATTCACATCTGCTATTTGTTCTGTTGTTAGAAGGGCTT
TTCCTCATTGTCTTGCATTTTCTTATATGGATGATGTTGTTCTTGGAG
CTAAATCTGTTCAACATCTTGAAAGTCTGTTTACCTCTATTACTAATT
TTCTGCTTTCTCTGGGAATTCATCTGAATCCAAACAAAACAAAGAGAT
GGGGATATTCTCTTAATTTCATGGGATATGTTATTGGATCTTGGGGAA
CACTTCCTCAAGAACATATCGTTTTGAAAATCAAGCAATGTTTCAGAA
AACTGCCTGTGAATAGACCTATTGATTGGAAAGTTTGTCAAAGAATTG
TGGGACTTCTTGGATTTGCTGCTCCTTTTACACAATGTGGATATCCTG
CTCTTATGCCACTTTATGCTTGTATTCAATCTAAACAGGCTTTTACAT
TTTCTCCAACATACAAAGCTTTTCTGTGTAAACAGTATCTGAATCTTT
ATCCTGTGGCTAGACAAAGATCTGGTCTTTGTCAAGTTTTTGCTGATG
CTACACCAACAGGATGGGGACTTGCTATTGGACATAGAGCTATGAGAG
GAACATTTGTTGCTCCATTGCCTATTCATACAGCTGAATTGCTTGCTG
CTTGTTTTGCTAGATCTAGAAGCGGAGCAAAACTTATTGGTACAGATA
ATAGTGTTGTCCTGAGTAGAAAGTACACATCTTTTCCATGGTTGTTGG
GATGTGCTGCTAATTGGATTCTTAGAGGAACTTCTTTTGTTTATGTTC
CTTCTGCTCTTAATCCTGCAGCTGATCCATCTGCTGGTAGATTGGGAC
TGTATAGACCACTTCTTCATTTGCCTTTTAGACCAACAACTGGAAGAA
CATCTCTTTATGCTGTTTCTCCTTCTGTTCCATCTCATTTGCCTGATA
GAGTTCATTTTGCTTCTCCACTTCATGTTGCTTGGAGGCCACCATCTA
AATCTCCAGGTTCTGGACCACCTATGCAACTTTTTCATTTGTGTTTGA
TCATTAGCTGTTCTTGTCCTACAGTTCAAGCTTCTAAACTTTGTCTTG
GATGGCTTTGGGGAATGGATATTGATCCATACAAAGAATTTGGAGCTA
GTGTTGAATTGCTGTCATTTCTTCCATCTGATTTTTTCCCTTCTATTC
GTGATCTTCTTGATACAGCATCTGCTCTGTATAGAGAAGCTCTTGAAT
CTCCTGAACACTGTTCTCCACATCATACAGCACTTAGACAAGCTATTC
TTTGTTGGGGAGAACTTATGAATCTTGCTACATGGGTTGGATCTAATT
TGGAAGATCCAGCTTCTAGAGAATTGGTGGTTTCTTATGTTAATGTGA
ATATGGGACTGAAAATTAGACAACTGCTTTGGTTTCATATCTCTTGTC
TTACATTTGGTAGAGAAACAGTTTTGGAATATTTGGTTTCTTTTGGCG
TTTGGATTAGAACACCTCCAGCTTATAGACCTCCTAATGCTCCTATTT
TGTCTACACTTCCTGAAACAACAGTCGTTAGAAGAAGAGGAAGATCTC
CAAGAAGAAGAACACCAAGTCCTAGAAGAAGAAGATCTCAATCACCAA
GAAGAAGAAGAAGTCAATCTAGAGAATCTCAATGTTGATTTTTGTTCG
ACATTAAAAATTGAAAATAAATACAAAGGTTCTTGAGGGTTGTGTTAA
ATTGAAAGCGAGAAATAATCATAAATATGATTCAGGTGACGGATCCAT
GGATGCTATGAAGCGAGGACTTTGTTGTGTTTTGCTTCTTTGTGGTGC
TGTGTTTGTTTCTCCATCTCAAGAAATTCATGCCAGATTCAGAAGAAT
GGGAGGCTGGTCATCTAAACCTAGACAAGGCATGGGAACAAATCTTTC
TGTTCCTAATCCTTTGGGATTCTTTCCTGATCATCAATTGGATCCAGC
ATTTGGAGCAAATAGTAACAATCCAGATTGGGACTTTAACCCAAACAA
AGATCATTGGCCAGAAGCTAATCAAGTTGGAGCATCTAAAGGTGGAAA
AAGTGGAATGGAAAACACTACATCTGGATTTCTTGGACCTTTGCTTGT
TCTTCAAGCTGGATTTTTCCTGTTGACAAGAATACTTACAATTCCTCA
ATCACTGGATTCTTGGTGGACAAGTCTTAATTTTCTTGGAGGTGCTCC
TACATGTCCTGGACAAAATTCTCAATCTCCAACTTCTAATCATTCTCC
TACATCTTGTCCTCCAATTTGTCCTGGATATAGATGGATGTGTCTTAG
AAGATTCATTATCTTTCTTTTCATACTGCTGCTGTGTCTGATTTTCCT
TCTTGTTTTGTTGGATTATCAGGGAATGCTTCCTGTTTGTCCTTTGCT
TCCTGGAACTTCTACAACAAGTACAGGACCTTGTAAAACATGTACAAT
TCCAGCACAGGGAACATCTATGTTTCCAAGTTGTTGTTGTACAAAACC
TTCTGATGGAAATTGCACATGTATTCCTATTCCAAGTTCTTGGGCATT
TGCTAGATTTCTTTGGGAATGGGCTTCTGTTAGATTCAGTTGGTTGTC
TCTTTTGGTTCCATTTGTTCAGTGGTTTGTTGGATTGTCTCCTACAGT
TTGGCTTTCTGTTATTTGGATGATGTGGTATTGGGGACCTTCTCTTTA
CAATATTTTGAGTCCTTTTCTCCCTTTGCTGCCAATTTTCTTTTGTCT
TTGGGTTTACATTTGAttaaccgagtttctgcattattgtaattcgta
tgctggcaccatcaaagaatcacttctaaaagatatcaatatcacaca
tacaaatattactaccctattgaatgagacagccaaggttatcaagtt
agtaaaatctctggtagataaagaagatactgatattgtgaataattt
cattaccaaagaaattaaaaacagagacaaaatagttaatagtttgtc
tctatcaaacctggactttcgtttgtaaattggggctTtttgtacaat
aaatgggtgttgccaatgattcatcccctgaatatcaatggatgtctc
cccatagattatcagatactgttatattaggagactgtttgtatttta
acaatataatgtcccaattagatttacaccaaaattgggctccatcag
ttagattgttaaattattttaagaattttaataaggaaacactactaa
agatagaagagaatgattacattaattcatcctttttccaacaaaagg
ataaacgattttatcctataaacgacgatttttatcacatatctacag
gaggatatggtatagtctttaagatagataactatgtagtaaaatttg
tattcgaggccacaaaattatatagtcccatggaaactacggcggagt
tcacagtacccaaatttctatacaacaatctaaagggagatgaaaaaa
aattaatcgtgtgtgcgtgggccatgggattaaactataaattaacat
ttttacatactctgtataaacgtgttcttcatatgttgctattattga
tacaaactatggatggtcaggaactatcattgagatattcttctaaag
tttttttaaaggcgtttaacgagagaaaggacagtatcaaattcgtga
aattactatcccacttttatccggcagttattaacagtaatattaatg
ttataaactattttaaccgcatgtttcactttttcgaacatgaaaaga
gaactaactacgaatacgaaagaggaaatattataatttttcccctag
cactgtattcggcagataaagtagataccgagctagctatcaaattag
gatttaaatctttggtacaatacataaagtttatctttttacagatgg
ctctgttatacattaaaatttacgaactaccatgctgcgacaactttt
tacacgcagatcttaaacccgataatatcttactttttgattccaatg
aaccaataataattcatctaaaggataaaaagtttgtttttaatgaac
gtattaaatcggcattaaacgactttgacttttcccaag

8.1 Description for Nucleotide Sequences of Low GC Content Version of MVA-SIi-HBV-PreS-Pmut-C-S(Sh):
8.1.1 F11-L-Flank=bases 1-1097 (SEQ ID NO: 35)
8.1.2 SIi-HBV-PreS-Pmut-C=bases 1098-4838 (SEQ ID NO: 59)
8.1.3 Transcription terminator sequence=bases 4839-4845 TTTTTGT
8.1.4 mH5 promoter=bases 4846-4942 (SEQ ID NO: 28)
8.1.5 TPA=bases 4943-5038 (SEQ ID NO: 60)
8.1.6 S(sh)=bases 5039-5920 (SEQ ID NO: 61)
8.1.7 F11-R-Flank=bases 5921-7239 (SEQ ID NO: 36)

The Sequence of wild-type HBV Polymerase is provided as SEQ ID NO: 19.

ChAdOx1 Sequence

ChAdOx1 sequence 5′ to the immunogen cassette is provided as SEQ ID NO: 39.

ChAdOx1 sequence 3′ to the immunogen cassette is provided as SEQ ID NO: 40.

ChAdOx2 Sequence

ChAdOx2 sequence 5′ to the immunogen cassette is provided as SEQ ID NO: 41.

ChAdOx2 sequence 3′ to the immunogen cassette is provided as SEQ ID NO: 42.

MVA Sequence

MVA sequence 5′ to the immunogen cassette is provided as SEQ ID NO: 44. MVA sequence 3′ to the immunogen cassette is provided as SEQ ID NO: 45

The CMV long promoter with Tetron Operator sequence is provided as SEQ ID NO: 50. The CMV short promoter with Tetron Operator sequence is provided as SEQ ID NO: 51.

PreS1 sequence (SEQ ID NO: 52):
MGGWSSKPRQGMGTNLSVPNPLGFFPDHQLDPAFGANSNNPDWDFNPN
KDHWPEANQVGAGAFGPGFTPPHGGLLGWSPQAQGILTTVPAAPPPAS
TNRQSGRQPTPISPPLRDSHPQA
PreS2 sequence (SEQ ID NO: 53):
MQWNSTTFHQALLDPRVRGLYFPAGGSSSGTVNPVPTTASPISSIFSR
TGDPAPN

1-45. (canceled)

46. A multi-HBV immunogen viral vector vaccine comprising:
a viral vector comprising an immunogen expression cassette, wherein the expression of a protein encoded by the expression cassette is arranged to be driven by a promoter, wherein the immunogen expression cassette encodes:
a) HBV Core;
b) a modified HBV polymerase (Pₘᵤₜ), wherein the modification is a mutation to wild-type HBV polymerase to substantially remove polymerase function;
c) HBV surface antigen (HbsAg); and
d) an intergenic sequence that is arranged to cause expression of at least the HBV surface antigen (HbsAg) as a separate protein from the HBV core and the modified HBV polymerase (Pₘᵤₜ),
wherein the intergenic sequence is downstream (3′) of the sequences encoding the HBV core and the modified HBV polymerase (Pₘᵤₜ) and upstream (5′) of the sequence encoding the HBV surface antigen (HbsAg).

47. The multi-HBV immunogen viral vector vaccine according to claim 46, wherein
(i) the intergenic sequence comprises a cleavage domain, an IRES (Internal Ribosomal Entry Site), a splicing signal, or a secondary promoter; or
(ii) the intergenic sequence comprises a cleavage domain; or
(iii) the intergenic sequence comprises a secondary promoter to promote expression of at least the surface antigen (HbsAg).

48. The multi-HBV immunogen viral vector vaccine according to claim 46, wherein the immunogen expression cassette further encodes HBV Pre-Core (PreC).

49. The multi-HBV immunogen viral vector vaccine according to claim 46, wherein
(i) the immunogen expression cassette further encodes HBV PreS1, and/or a truncated form thereof; or
(ii) the immunogen expression cassette further encodes HBV PreS2; or
(iii) the immunogen expression cassette encodes HBV Pre-Core (PreC) and HBV PreS1, and a truncated form of PreS1; or
(iv) the immunogen expression cassette is capable of expressing HBV e-Antigen; or
(v) the HBV Core and modified polymerase (Pₘᵤₜ) are arranged to be expressed as a fusion protein.

50. The multi-HBV immunogen viral vector vaccine according to claim 48, wherein the HBV Pre-core, HBV Core and modified polymerase (Pₘᵤₜ) are arranged to be expressed as a fusion protein.

51. The multi-HBV immunogen viral vector vaccine according to claim 46, wherein
(i) the immunogen expression cassette does not encode HBV X protein; or
(ii) the immunogen expression cassette comprises nucleic acid comprising the sequence of:
SEQ ID NO: 46 (SE-HBV-CPmutS) or a variant thereof;
SEQ ID NO: 47 (SE-HBV-SCPmut) or a variant thereof;
SEQ ID NO: 48 (SE-HBV-CPmutPreS-S(sh)) or a variant thereof;
SEQ ID NO: 49 (SE-HBV-CPmutPreS-TPA-S(sh)) or a variant thereof; SEQ ID NO: 24 (MVA-SE-HBV-PreS-Pmut-C-S(sh)) or a variant thereof; or
SEQ ID NO: 27 or SEQ ID NO: 58 (MVA-SE-HBV-PreS-Pmut-C-TPA-S(sh)) or a variant thereof.

52. The multi-HBV immunogen viral vector vaccine according to claim 46, wherein
(i) the viral vector encodes the amino acid sequence of:
SEQ ID NO: 3 (SE-HBV-CPmutS) or a variant thereof.
SEQ ID NO: 11 (SE-HBV-SCPmut) or a variant thereof.
SEQ ID NO: 13 (SE-HBV-CPmutPreS-S(sh)) or a variant thereof.
SEQ ID NO: 25 (SE-HBV-CPmutPreS-TPA-S(sh)) or a variant thereof.
SEQ ID NO: 23 (MVA-SE-HBV-PreS-Pmut-C-S(sh)) or a variant thereof.
SEQ ID NO: 26 (MVA-SE-HBV-PreS-Pmut-C-TPA-S(sh)) or a variant thereof; or
(ii) the viral vector comprises an adenovirus vector or Modified Vaccinia Ankara (MVA) vector; or
(iii) the viral vector comprises a group E simian adenovirus vector; or
(iv) the promoter is encoded in the immunogen expression cassette; or
(v) the promoter may be encoded as part of the viral vector nucleic acid outside of the immunogen expression cassette.

53. The multi-HBV immunogen viral vector vaccine according to claim 46, wherein
(i) the promoter promotes the expression of all the encoded protein of the immunogen expression cassette; or
(ii) the immunogen expression cassette comprises a secondary promoter, wherein the promoter is a primary promoter that is arranged to promote expression of at least the HBV core and modified polymerase (Pₘᵤₜ), and not the HBV surface antigen (HbsAg) which is arranged to be promoted separately by the secondary promoter; or
(iii) the promoter comprises a CMV promoter or a pox viral promoter.

54. The multi-HBV immunogen viral vector vaccine according to claim 46, wherein
(i) the HBV Core comprises or consists of a full length wild-type HBV Core sequence; or
(ii) the modified HBV polymerase (Pₘᵤₜ) is not a truncated form of HBV polymerase; or
(iii) the modified HBV polymerase (Pₘᵤₜ) comprises or consists of the sequence of SEQ ID NO: 8 or a variant thereof; or
(iv) the HbsAg comprises or consists of a full length wild-type HbsAg sequence, or a variant thereof.

55. The multi-HBV immunogen viral vector vaccine according to claim 46, wherein the immunogen expression cassette encodes a truncated form of HBV PreS1 and the truncated PreS1 is arranged to be expressed a fusion protein with the HBV surface antigen (S/HbsAg).

56. The multi-HBV immunogen viral vector vaccine according to claim 55, further comprising a linker sequence provided between the truncated PreS1 and surface antigen (S/HbsAg).

57. The multi-HBV immunogen viral vector vaccine according to claim 55, wherein the truncated PreS1 with a fused surface antigen is encoded downstream (3′) of the intergenic sequence.

58. The multi-HBV immunogen viral vector vaccine according to claim 46, wherein the expression cassette encodes a NΔPreS1 and PreS2 fusion sequence.

59. The multi-HBV immunogen viral vector vaccine according to claim 58, wherein the encoded NΔPreS1 and PreS2 fusion sequence is encoded upstream (5′) of the intergenic sequence.

60. The multi-HBV immunogen viral vector vaccine according to claim 59, wherein the encoded NΔPreS1 and PreS2 fusion sequence is further fused with the modified polymerase (Pₘᵤₜ).

61. The multi-HBV immunogen viral vector vaccine according to claim 60, wherein a linker sequence is provided between the PreS2 and modified polymerase (Pₘᵤₜ).

62. A nucleic acid comprising or consisting of an HBV immunogen expression cassette, wherein the immunogen expression cassette encodes:
a) HBV Core;
b) a modified HBV polymerase (Pₘᵤₜ), wherein the modification is a mutation to wild-type HBV polymerase to substantially remove polymerase function;
c) HBV surface antigen (HbsAg); and
d) an intergenic sequence that is arranged to cause expression of at least the HBV surface antigen (HbsAg) as a separate protein from the HBV core and the modified HBV polymerase (Pₘᵤₜ),
wherein the intergenic sequence is downstream (3′) of the sequences encoding the HBV core and the modified HBV polymerase (Pₘᵤₜ) and upstream (5′) of the sequence encoding the HBV surface antigen (HbsAg).

63. The nucleic acid according to claim 62, wherein
(i) the immunogen expression cassette further encodes a promoter; and/or
(ii) the immunogen expression cassette is isolated or provided in a non-viral vector.

64. A method of treatment or prophylaxis of HBV infection comprising the administration of the viral vector according to claim 46, to a subject.

65. The method according to claim 64, wherein the use or administration is in combination with the use or administration of another therapeutically or prophylactically active ingredient.

66. The multi-HBV immunogen viral vector vaccine of claim 46, wherein the intergenic sequence allows for transcriptional, translational or post-translational control of the expression of the protein.

67. The multi-HBV immunogen viral vector vaccine of claim 47, wherein the cleavage domain is selected from the group consisting of a post-translation cleavage domain and a ribosome skipping cleavage domain, or the cleavage domain comprises a Furin-2A peptide sequence or a functional variant thereof.
